(19) United States
(12) Patent Application Publication
(10) Pub. No.: US 2004/0082551 A1
(43) Pub. Date: Apr. 29, 2004

(54) Novel pyrazoles and their use as p38 kinase inhibitors

(76) Inventors: Alan G. Benson, Chesterfield, MO (US); Thomas P. Fraher, Chicago, IL (US); Michael E. Hepperle, Boston, MA (US); Kevin D. Jerome, Maryland Heights, MO (US); Win Naing, Chesterfield, MO (US); Shaun R. Selness, Chesterfield, MO (US); John K. Walker, Maryland Heights, MO (US)

Correspondence Address: HARNESS, DICKEY, & PIERCE, P.L.C, 7700 BONHOMME, STE 400, ST. LOUIS, MO, 63105, US

(21) Appl. No.: 10/456,933

(22) Filed: Jun. 5, 2003

Related U.S. Application Data
(60) Provisional application No. 60/386,415, filed on Jun. 5, 2002.

(51) Int. Cl. A61K 31/655; A61K 31/5377; A61K 31/454; A61K 31/4152; C07D 413/02; C 07D 4 3/02

(52) U.S. Cl. 514/150; 514/235.8; 514/254.05; 514/326; 514/406; 544/140; 546/211; 548/366.1; 548/367.4; 534/769

(57) ABSTRACT

This invention is directed generally to pyrazoles that, inter alia, inhibit p38 kinase, TNF, and/or cyclooxygenase-2 activity. Such pyrazoles include compounds generally corresponding in structure to the following formula:

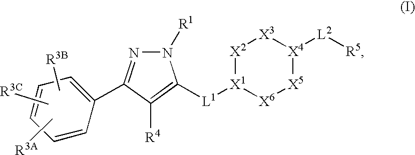

wherein L¹, L², X¹, X², X³, X⁴, X⁵, X⁶, R¹, R^{3A}, R^{3B}, R^{3C}, R⁴, and R⁵ are as defined in this specification. The pyrazoles further include tautomers of such compounds, as well as salts of such compounds and tautomers. This invention also is directed to compositions of such pyrazoles, intermediates for the syntheses of such pyrazoles, methods for making such pyrazoles, and methods for treating (including preventing) conditions (particularly pathological conditions) associated with p38 kinase, TNF, and/or cyclooxygenase-2 activity.

### CROSS-REFERENCE TO RELATED PATENT APPLICATION

This patent claims priority to U.S. Provisional Application Serial No. 60/386,415 (filed Jun. 5, 2002). The entire text of that application is incorporated by reference into this application.

### FIELD OF THE INVENTION

This invention is directed to pyrazole compounds (including tautomers of the compounds, and salts of the compounds and tautomers) that, inter alia, generally tend to inhibit p38 kinase (particularly p38α kinase), TNF (particularly TNF-α), and/or cyclooxygenase (particularly cyclooxygenase-2 or “COX-2”) activity. This invention also is directed to compositions of such pyrazoles (particularly pharmaceutical compositions), intermediates for the syntheses of such pyrazoles, methods for making such pyrazoles, and methods for treating (including preventing) conditions (typically pathological conditions) associated with p38 kinase activity, TNF activity, and/or cyclooxygenase-2 activity.

### BACKGROUND OF THE INVENTION

Mitogen-activated protein kinases (MAP) constitute a family of proline-directed serine/threonine kinases that activate their substrates by dual phosphorylation. The kinases are activated by a variety of signals, including nutritional and osmotic stress, UV light, growth factors, endotoxin, and inflammatory cytokines. The p38 MAP kinase group is a MAP family of various isoforms, including p38α, p38β, and p38γ. These kinases are responsible for phosphorylating and activating transcription factors (e.g., ATF2, CHOP, and MEF2C), as well as other kinases (e.g., MAPKAP-2 and MAPKAP-3). The p38 isoforms are activated by bacterial lipopolysaccharide, physical and chemical stress, and pro-inflammatory cytokines, including tumor necrosis factor (“TNF”) and interleukin-1 (“IL-1”). The products of the p38 phosphorylation mediate the production of inflammatory cytokines, including TNF, IL-1, and cyclooxygenase-2.

It is believed that p38α kinase can cause or contribute to the effects of, for example, inflammation generally; arthritis; neuroinflammation; pain; fever; pulmonary disorders; cardiovascular diseases; cardiomyopathy; stroke; ischemia; reperfusion injury; renal reperfusion injury; brain edema; neurotrauma and brain trauma; neurodegenerative disorders; central nervous system disorders; liver disease and nephritis; gastrointestinal conditions; ulcerative diseases; ophthalmic diseases; ophthalmological conditions; glaucoma; acute injury to the eye tissue and ocular traumas; diabetes; diabetic nephropathy; skin-related conditions; viral and bacterial infections; myalgias due to infection; influenza; endotoxic shock; toxic shock syndrome; autoimmune disease; bone resorption diseases; multiple sclerosis; disorders of the female reproductive system; pathological (but non-malignant) conditions, such as hemaginomas, angiofibroma of the nasopharynx, and avascular necrosis of bone; benign and malignant tumors/neoplasia including cancer; leukemia; lymphoma; systemic lupus erthrematosis (SLE); angiogenesis including neoplasia; and metastasis.

TNF is a cytokine produced primarily by activated monocytes and macrophages. Excessive or unregulated TNF production (particularly TNF-α) has been implicated in mediating a number of diseases. It is believed, for example, that TNF can cause or contribute to the effects of inflammation (e.g., rheumatoid arthritis and inflammatory bowel disease), asthma, autoimmune disease, graft rejection, multiple sclerosis, fibrotic diseases, cancer, fever, psoriasis, cardiovascular diseases (e.g., post-ischemic reperfusion injury and congestive heart failure), pulmonary diseases (e.g., hyperoxic alveolar injury), hemorrhage, coagulation, radiation damage, and acute phase responses like those seen with infections and sepsis and during shock (e.g., septic shock and hemodynamic shock). Chronic release of active TNF can cause cachexia and anorexia. And TNF can be lethal.

TNF also has been implicated in infectious diseases. These include, for example, malaria, mycobacterial infection, meningitis. These also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

IL-8 is another pro-inflammatory cytokine, which is produced by mononuclear cells, fibroblasts, endothelial cells, and keratinocytes. This cytokine is associated with conditions including inflammation.

IL-1 is produced by activated monocytes and macrophages, and is involved in inflammatory responses. IL-1 plays a role in many pathophysiological responses, including rheumatoid arthritis, fever, and reduction of bone resorption.

TNF, IL-1, and IL-8 affect a wide variety of cells and tissues, and are important inflammatory mediators of a wide variety of conditions. The inhibition of these cytokines by inhibition of the p38 kinase is beneficial in controlling, reducing, and alleviating many of these disease states.

Various pyrazoles have previously been described:

In U.S. Pat. No. 4,000,281, Beiler and Binon report 4,5-aryl/heteroaryl substituted pyrazoles with antiviral activity against both RNA and DNA viruses, such as myxoviruses, adenoviruses, rhinoviruses, and various viruses of the herpes group.

WIPO Int'l Publ. No. WO 92/19615 (published Nov. 12, 1992) describes pyrazoles as novel fungicides.

In U.S. Pat. No. 3,984,431, Cueremy and Renault report derivatives of pyrazole-5-acetic acid as having anti-inflammatory activity, with [1-isobutyl-3,4-diphenyl-1H-pyrazol-5-yl]acetic acid being specifically described.

In U.S. Pat. No. 3,245,093, Hinsgen et al report a process for preparing pyrazoles.

WIPO Int'l Publ. No. WO 83/00330 (published Feb. 3, 1983) describes a process for preparing diphenyl-3,4-methyl-5-pyrazole derivatives.

WIPO Int'l Publ. No. WO 95/06036 (published Mar. 2, 1995 reports a process for preparing pyrazole derivatives.

In U.S. Pat. No. 5,589,439, T. Goto, et al. report tetrazole derivatives and their use as herbicides.

EP 515,041 reports pyrimidinyl substituted pyrazole derivatives as novel agricultural fungicides.

Japanese Patent 4,145,081 reports pyrazolecarboxylic acid derivatives as herbicides.

Japanese Patent 5,345,772 reports novel pyrazole derivatives as inhibiting acetylcholinesterase.

Pyrazoles have been reported as useful in treating inflammation.

Japanese Patent 5,017,470 reports synthesis of pyrazole derivatives as anti-inflammatory, anti-rheumatic, anti-bacterial, and anti-viral drugs.

EP 115640 (published Dec. 30, 1983) reports 4-imidazolyl-pyrazole derivatives as inhibitors of thromboxane synthesis, with 3-(4-Isopropyl-1-methylcyclohex-1-yl)-4-(imidazol-1-yl)-1H-pyrazole being specifically described.

WIPO Int'l Publ. No. WO 97/01551 (published Jan. 16, 1997) reports pyrazole compounds as adenosine antagonists, with 4-(3-Oxo-2,3-dihydropyridazin-6-yl)-3-phenylpyrazole being specifically described.

In U.S. Pat. No. 5,134,142, Matsuo et al. report 1,5-diaryl pyrazoles as having anti-inflammatory activity.

In U.S. Pat. No. 5,559,137, Adams et al. report pyrazoles (1,3,4,-substituted) as inhibitors of cytokines used in the treatment of cytokine diseases, with 3-(4-fluorophenyl)-1-(4-methylsulfinylphenyl)-4-(4-pyridyl)-5H-pyrazole being specifically described.

WIPO Int'l Publ. No. WO 96/03385 (published Feb. 8, 1996) reports 3,4-substituted pyrazoles as having anti-inflammatory activity, with 3-methylsulfonylphenyl-4-aryl-pyrazoles and 3-aminosulfonylphenyl-4-aryl-pyrazoles being specifically described.

Laszlo et al., Bioorg. Med. Chem. Letters, 8 (1998) 2689-2694, describes certain furans, pyrroles, and pyrazolones (particularly 3-pyridyl-2,5-diaryl-pyrroles) as inhibitors of p38 kinase.

WIPO Int'l Publ. No. WO 98/52940 (PCT Patent Application No. US98/10436 published on Nov. 26, 1998) reports pyrazoles, compositions containing those pyrazoles, and methods for treating p38-mediated disorders using those pyrazoles.

WIPO Int'l Publ. No. WO 00/31063 (PCT Patent Application No. US99/26007 published on Jun. 2, 2000) also reports pyrazoles, compositions containing those pyrazoles, and methods for treating p38-mediated disorders using those pyrazoles.

In view of the importance of pyrazoles in the treatment of several pathological conditions (particularly those associated with p38 kinase activity, TNF activity, and/or cyclooxygenase-2 activity), there continues to be a need for pyrazole compounds exhibiting an improved safety profile, solubility, and/or potency. The following disclosure describes pyrazole compounds that tend to exhibit one or more such desirable qualities.

### SUMMARY OF THE INVENTION

This invention is directed to pyrazole compounds that tend to inhibit p38 kinase activity, TNF activity, and/or cyclooxygenase-2 activity. This invention also is directed to, for example, a method for inhibiting p38 kinase, TNF, and/or cyclooxygenase-2 activity, and particularly to a method for treating a condition (typically a pathological condition) mediated by p38 kinase activity, TNF activity, and/or cyclooxygenase-2 activity. Such a method is typically suitable for use with mammals, such as humans, other primates (e.g., monkeys, chimpanzees. etc.), companion animals (e.g., dogs, cats, horses. etc.), farm animals (e.g., goats, sheep, pigs, cattle, etc.), laboratory animals (e.g., mice, rats, etc.), and wild and zoo animals (e.g., wolves, bears, deer, etc.).

Briefly, therefore, this invention is directed, in part, to compounds that generally fall within structure of Formula I:

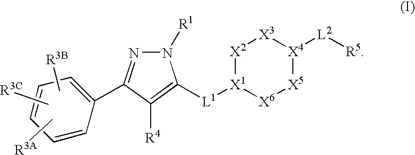

This invention also is directed to tautomers of such compounds, as well as salts (particularly pharmaceutically-acceptable salts) of such compounds and tautomers.

In Formula (I):

L¹ is a bond, —O—, —S—, —S(O)—, —S(O)₂—, —N(R^{a})—, —C(O)—, —C(O)—N(R^{a})—, —N(R^{a})—, —C(O)—O—, —O—C(O)—, —O—C(O)—O—, —C(H)═C(H)—, —C≡C—, —N═N—, —N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

X¹ is nitrogen or carbon bonded to hydrogen, except that X¹ is carbon bonded to hydrogen if any of X², X³, X⁵, or X⁶ is —NH— or —O—.

X² is —CH₂—, —NH—, or —O—, except that X² is —CH₂— if X³ is —O— or —NH—.

X³ is —CH₂—, —NH—, or —O—, except that X³ is —CH₂— if X² is —O— or —NH—.

X⁴ is nitrogen or carbon bonded to hydrogen.

X⁵ is —CH₂— or —NH—, except that X⁵ is —CH₂— if X³ is —O— or X⁶ is —NH—.

X⁶ is —CH₂— or —NH—, except that X⁶ is —CH₂— if X² is —O— or X⁵ is —NH—.

R¹ is hydrogen, hydroxyalkyl, carboxyalkyl, aminoalkyl, aminocarbonylalkyl, or aminocarbonylaminoalkyl. The amino nitrogen(s) of the aminoalkyl, aminocarbonylalkyl, or aminocarbonylaminoalkyl optionally is/are substituted with up to two independently selected alkyl.

R^{3A} is halogen, hydroxy, cyano, amino, alkyl, aminoalkyl, monoalkylamino, dialkylamino, cycloalkylamino, alkoxy, or alkoxyalkyl. Any carbon of the alkyl, aminoalkyl, monoalkylamino, dialkylamino, cycloalkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, cycloalkylamino, alkoxy, or alkoxyalkyl. Any carbon of the alkyl, aminoalkyl, monoalkylamino, dialkylamino, cycloalkylamino, alkoxy, or alkoxyalkyl optionally is substituted with one or more substituents independently selected from the group consisting of halogen, hydroxy, and cyano.

R^{3C} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

L² is a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

Each R^{a} is independently selected from the group consisting of hydrogen and alkyl.

R⁵ is hydrogen, hydroxy, alkyl, alkenyl, alkynyl, alkoxy, alkoxyalkyl, alkoxyalkoxyalkyl, alkylcarbonyloxyalkyl, alkylcarbonylalkyl, alkoxycarbonylalkyl, carbocyclyl, carbocyclylalkyl, heterocyclyl, or heterocyclylalkyl. Any such substituent is, in turn, optionally substituted with one or more substituents independently selected from the group consisting of halogen, hydroxy, alkyl, haloalkyl, hydroxyalkyl, alkoxy, and haloalkoxy.

This invention also is directed, in part, to a method for treating a condition mediated by pathological p38 kinase activity (particularly p38α activity) in a mammal. The method comprises administering an above-described compound, tautomer, or salt to the mammal in an amount that is therapeutically-effective to treat the condition.

This invention also is directed, in part, to a method for treating a condition mediated by pathological TNF activity (particularly TNF-α activity) in a mammal. The method comprises administering an above-described compound, tautomer, or salt to the mammal in an amount that is therapeutically-effective to treat the condition.

This invention also is directed, in part, to a method for treating a condition mediated by pathological cyclooxygenase-2 activity in a mammal. The method comprises administering an above-described compound, tautomer, or salt to the mammal in an amount that is therapeutically-effective to treat the condition.

This invention also is directed, in part, to pharmaceutical compositions comprising a therapeutically-effective amount of an above-described compound, tautomer, or salt.

This invention also is directed, in part, to a use of an above-described compound, tautomer, or salt to prepare a medicament for treating a condition mediated by p38 kinase activity.

This invention also is directed, in part, to a use of an above-described compound, tautomer, or salt to prepare a medicament for treating a condition mediated by TNF activity.

This invention also is directed, in part, to a use of an above-described compound, tautomer, or salt to prepare a medicament for treating a condition mediated by cyclooxygenase-2 activity.

Further benefits of Applicants' invention will be apparent to one skilled in the art from reading this specification.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

### A. Compounds of this Invention

In accordance with this invention, it has been found that certain pyrazole compounds tend to be effective for inhibiting the activity (particularly pathological activity) of p38 kinase, TNF, and/or cyclooxygenase-2. Such compounds tend to exhibit desirable safety profiles, solubilities, and/or potencies.

As noted above, the compounds of this invention generally have a structure corresponding to Formula I:

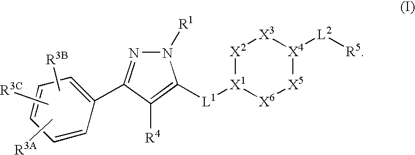

L¹, L², X¹, X², X³, X⁴, X⁵, X⁶, R¹, R^{3A}, R^{3B}, R^{3C}, R⁴, and R⁵ are defined as follows:

### General Description of Preferred L¹ Substituents

L¹ is a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—N(R^{a})—C(S)—, —CH₂—, —O—CH₂—, —CH₂—O—, —S—CH₂—, or —CH₂—S—.

In some preferred embodiments, L¹ is a bond.

Each R^{a} is independently selected from the group consisting of hydrogen and alkyl

In some preferred embodiments, each R^{a} is alkyl.

In some preferred embodiments, each R^{a} is hydrogen.

### General Description of Preferred X¹, X², X³, X⁴, X⁵, and X⁶ Substituents

X¹ is nitrogen or carbon bonded to hydrogen, except that X¹ is carbon bonded to hydrogen if any of X², X³, X⁵, or X⁶ is —NH— or —O—. X² is —CH₂—, —NH—, or —O—, except that X² is —CH₂— if X³ is —O— or —NH—. X³ is —CH₂—, —NH—, or —O—, except that X³ is —CH₂— if X² is —O— or —NH—. X⁴ is nitrogen or carbon bonded to hydrogen. X⁵ is —CH₂— or —NH—, except that X⁵ is —CH₂— if X³ is —O— or X⁶ is —NH—. And X⁶ is —CH₂— or —NH—, except that X⁶ is —CH₂— if X² is —O— or X⁵ is —NH—.

In some preferred embodiments, X², X³, X⁵, and X⁶ are each —CH₂—.

In some preferred embodiments, X¹ and X⁴ are each carbon bonded to hydrogen.

In some preferred embodiments, the ring formed by X¹, X², X¹, X⁴, X⁵, and X⁶ is cyclohexyl. In such embodiments, X², X³, X⁵, and X⁶ are each —CH₂—; and X¹ and X⁴ are each carbon bonded to hydrogen. In other words, the compound corresponds in structure to the following general formula:

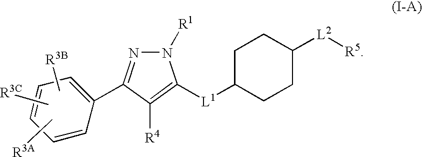

In some preferred embodiments, the compound has a cis configuration with respect to the cyclohexyl group:

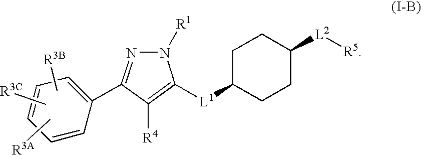

In typically more preferred embodiments, the compound has a trans configuration with respect to the cyclohexyl group:

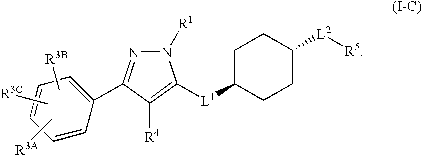

In some preferred embodiments, the ring formed by X¹, X², X³, X⁴, X⁵, and X⁶ is piperidinyl. In some such embodiments, it is particularly preferred for X², X³, X⁵, and X⁶ to each be —CH₂—; X¹ to be nitrogen; and X⁴ to be carbon bonded to hydrogen. In other words, the compound corresponds in structure to the following general formula:

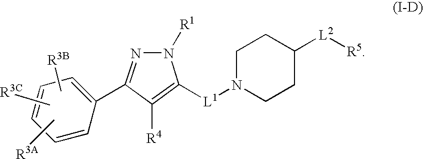

In other embodiments wherein the ring formed by X¹, X², X³, X⁴, X⁵, and X⁶ is piperidinyl, it is particularly preferred for X², X³, X⁵, and X⁶ to each be —CH₂—; X¹ to be carbon bonded to hydrogen; and X⁴ to be nitrogen. In other words, the compound corresponds in structure to the following general formula:

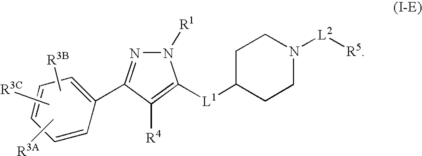

In some preferred embodiments, the ring formed by X¹, X², X³, X⁴, X⁵, and X⁶ is piperazinyl. In some such embodiments, it is particularly preferred for X², X³, X⁵, and X⁶ to each be —CH₂—; and X¹ and X⁴ to each be nitrogen. In other words, the compound corresponds in structure to the following general formula:

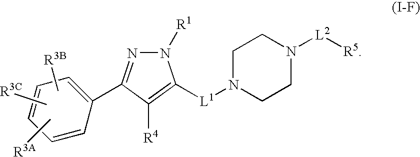

### General Description of Preferred R¹ Substituents

R¹ is hydrogen, hydroxyalkyl, carboxyalkyl, aminoalkyl, aminocarbonylalkyl, or aminocarbonylaminoalkyl. The amino nitrogen(s) of the aminoalkyl, aminocarbonylalkyl, or aminocarbonylaminoalkyl optionally is/are substituted with up to two independently selected alkyl.

In some preferred embodiments, R¹ is hydrogen.

In some preferred embodiments, R¹ is a non-hydrogen substituent that enhances solubility of the compound relative to the solubility of the compound if R¹ is hydrogen. One such particularly preferred R¹ substituent for enhancing solubility is hydroxyalkyl.

### General Description of Preferred R^{3A} and R^{3B} Substituents

R^{3A} is halogen, hydroxy, cyano, amino, alkyl, aminoalkyl, monoalkylamino, dialkylamino, cycloalkylamino, alkoxy, or alkoxyalkyl. Any carbon of the alkyl, aminoalkyl, monoalkylamino, dialkylamino, cycloalkylamino, alkoxy, or alkoxyalkyl optionally is substituted with one or more substituents independently selected from the group consisting of halogen, hydroxy, and cyano.

In some preferred embodiments, R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

In some preferred embodiments, R^{3A} is at the ortho position of the 3position phenyl ring in Formula I, i.e., the compound corresponds in structure to the following formula:

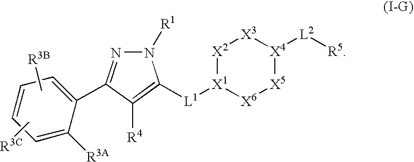

In some preferred embodiments, R^{3A} is at the meta position of the 3position phenyl ring in Formula I, i.e., the compound corresponds in structure to the following formula:

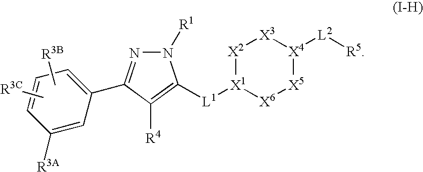

In some preferred embodiments, R^{3A} is at the para position of the 3-position phenyl ring in Formula I, i.e., the compound corresponds in structure to the following formula:

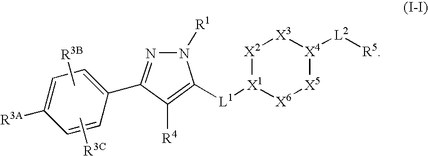

In some preferred embodiments, R^{3A} is hydroxy, cyano, amino, monomethylamino, monoethylamino, dimethylamino, diethylamino, N-methyl-N-ethyl-amino methyl, ethyl, haloethyl, propyl, halopropyl, aminomethyl, aminoethyl, hydroxymethyl, hydroxyethyl, methoxy, halomethoxy, ethoxy, haloethoxy, methoxymethyl, or halomethoxymethyl.

In some preferred embodiments, R^{3A} is hydroxy, cyano, amino, monomethylamino, monoethylamino, dimethylamino, diethylamino, N-methyl-N-ethyl-amino, methyl, ethyl, haloethyl, propyl, halopropyl, aminomethyl, aminoethyl, hydroxymethyl, hydroxyethyl, methoxy, ethoxy, haloethoxy, methoxymethyl, or halomethoxymethyl.

In some preferred embodiments, R^{3A} is halogen, methyl, methoxy, halomethyl, or halomethoxy.

In some preferred embodiments, R^{3A} is chloro, chloromethyl, or chloromethoxy.

In some preferred embodiments, R^{3A} is fluoro, fluoromethyl, or fluoromethoxy.

R^{3B} is hydrogen, halogen, hydroxy, cyano, amino, alkyl, aminoalkyl, monoalkylamino, dialkylamino, cycloalkylamino, alkoxy, or alkoxyalkyl. Any carbon of the alkyl, aminoalkyl, monoalkylamino, dialkylamino, cycloalkylamino, alkoxy, or alkoxyalkyl optionally is substituted with one or more substituents independently selected from the group consisting of halogen, hydroxy, and cyano.

In some preferred embodiments, R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

In some preferred embodiments, R^{3D} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

In some preferred embodiments, R^{3A} and R^{3B} are independently selected from the group consisting of fluoro, chloro, methyl, trifluoromethyl, ethyl, hydroxy, methoxy, trifluoromethoxy, amino, monomethylamino, and dimethylamino.

In some preferred embodiments, R^{3A} and R^{3B} are independently selected from the group consisting of halogen, methyl, methoxy, halomethyl, and halomethoxy.

In some preferred embodiments, R^{3A} and R^{3B} are independently selected from the group consisting of chloro, fluoro, methyl, methoxy, chloromethyl, fluoromethyl, chloromethoxy, and fluoromethoxy.

In some preferred embodiments, R^{3A} and R^{3B} are independently selected from the group consisting of chloro, methyl, methoxy, chloromethyl, and chloromethoxy.

In some preferred embodiments, R^{3A} and R^{3B} are independently selected from the group consisting of fluoro, methyl, methoxy, fluoromethyl, and fluoromethoxy.

In some preferred embodiments, R^{3A} and R^{3B} are independently selected from the group consisting of chloro, chloromethyl, and chloromethoxy.

In some preferred embodiments, R^{3A} and R^{3B} are independently selected from the group consisting of fluoro, fluoromethyl, and fluoromethoxy.

In some embodiments, R^{3A} is halogen or haloalkyl; and R^{3B} is hydrogen, halogen, or haloalkyl.

### General Description of Preferred R^{3C} Substituents

R^{3C} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

In some preferred embodiments, R^{3C} is hydrogen. In other words, the compound corresponds in structure to the following formula:

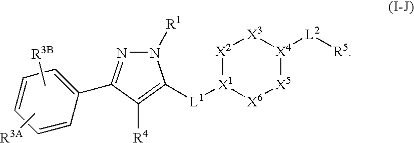

In some such embodiments, for example, the compound corresponds in structure to the following formula:

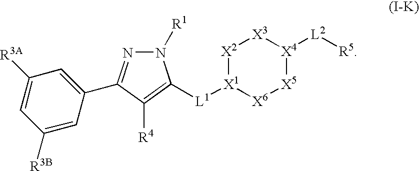

In other such embodiments, for example, the compound corresponds in structure to the following formula:

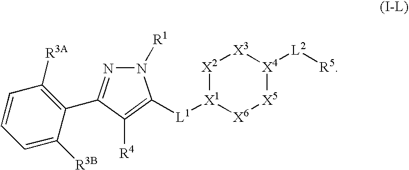

In other such embodiments, for example, the compound corresponds in structure to the following formula:

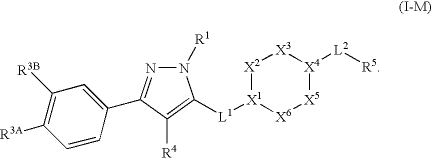

In other such embodiments, for example, the compound corresponds in structure to the following formula:

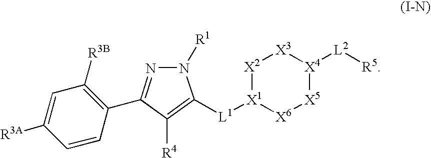

In other such embodiments, for example, the compound corresponds in structure to the following formula:

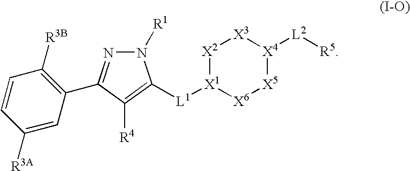

In other such embodiments, for example, the compound corresponds in structure to the following formula:

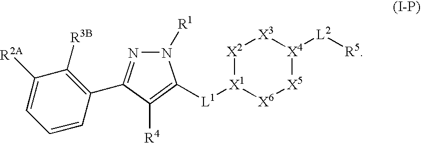

### General Description of Preferred R⁴ Substituents

R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some such preferred embodiments, R⁴ is pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted as discussed above.

In some such preferred embodiments, R⁴ is pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted as discussed above.

In some such preferred embodiments, R⁴ is pyridazinyl, pyrazinyl, pyrimidinyl, triazinyl, tetrazinyl, benzazinyl, benzodiazinyl, naphthyridinyl, pyridopyridinyl, pyrinyl, maleimidyl, pyridonyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted as discussed above.

In some such preferred embodiments, R⁴ is pyrimidinyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted as discussed above.

In some such preferred embodiments, R⁴ is a 6-member, nitrogen-containing ring that is optionally substituted as discussed above.

In some such preferred embodiments, R⁴ is pyrimidinyl or pyrimidinyl. The pyrimidinyl or pyridinyl optionally is substituted as discussed above.

In some such preferred embodiments, R⁴ is pyridinyl optionally substituted as discussed above.

In some such preferred embodiments, R⁴ is pyrimidinyl optionally substituted as discussed above.

In some such preferred embodiments, R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with alkylthio, mono-alkylamino, di-alkylamino, alkoxy, or haloalkoxy.

In some such preferred embodiments, R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl.

In some preferred embodiments, R⁴ is pyrimidinyl optionally substituted with halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, or carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. These ring structures are:

substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. In these embodiments, any such substituent is:

substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyrimidinyl. In these embodiments, the pyrimidinyl is:

substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent optionally is, in turn, substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyrimidinyl substituted with heterocyclyloxy, heterocyclylalkoxy, cycloalkylamino, cyanoaryloxy, alkylaminoalkylamino, or carbocyclylalkylheterocyclylamino. Any such substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyrimidinyl substituted with heterocyclyloxy, heterocyclylalkoxy, cycloalkylamino, cyanoaryloxy, dialkylaminoalkylamino, or carbocyclylalkylheterocyclylamino.

In some preferred embodiments, R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent is substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyridinyl substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ is pyrimidinyl substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R⁴ corresponds in structure to the following formula:

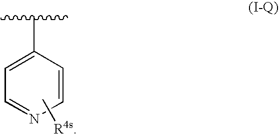

In some preferred embodiments, R⁴ corresponds in structure to the following formula:

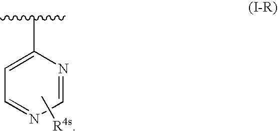

In some preferred embodiments, R⁴ corresponds in structure to the following formula:

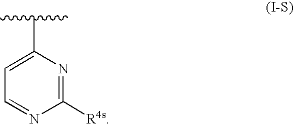

In so me preferred embodiments, R⁴ corresponds in structure to the following formula:

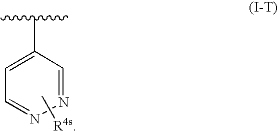

In some preferred embodiments, R⁴ corresponds in structure to the following formula:

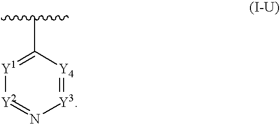

Here, two of Y¹, Y², Y³, and are each nitrogen, one of Y¹, Y², Y³, and Y⁴ is carbon bonded to R^{4s}, and one of Y¹, Y², Y³, and Y⁴ is carbon bonded to hydrogen.

In some preferred embodiments, R⁴ corresponds in structure to the following formula:

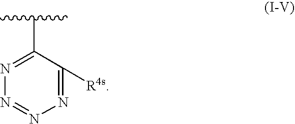

In the above embodiments, R^{4s} may be hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, or carbocyclylhydrazinyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R^{4s} is hydrogen, alkyl, aminoalkyl, alkoxyalkyl, cycloalkylalkyl, hetercycloalkylalkyl, heteroarylalkyl, amino, alkylamino, aminoalkylamino, alkoxyalkylamino, cycloalkylamino, heterocycloalkylamino, heteroarylamino, hydroxy, alkoxy, aminoalkoxy, alkoxyalkoxy, cycloalkyloxy, heterocycloalkyloxy, or heteroaryloxy. Any such substituent optionally is substituted with one or more substituents independently selected from the group consisting of hydroxy and alkyl.

In some preferred embodiments, R^{4s} is hydrogen, C₁-C₄-alkyl, aminopropyl, monomethylaminopropyl, dimethylaminopropyl, hydroxypropyl, methoxypropyl, cyclopentylmethyl, pyrrolidinylmethyl, tetrahydrofuranylmethyl, piperidinylmethyl, tetrahydropyranylmethyl, pyridinylmethyl, C₁-C₃-alkylamino, aminoethylamino, monomethylaminoethylamino, dimethylaminoethylamino, hydroxyethylamino, methoxyethylamino, cyclopentylamino, pyrrolidinylamino, tetrahydrofuranylamino, piperidinylamino, tetrahydropyranylamino, pyridinylamino, C₁-C₃-alkoxy, aminoethoxy, monomethylaminoethoxy, dimethylaminoethoxy, hydroxyethoxy, methoxyethoxy, cyclopentyloxy, pyrrolidinyloxy, tetrahydrofuranyloxy, piperidinyloxy, tetrahydropyranyloxy, or pyridinyloxy. Any pyrrolidinyl nitrogen or piperidinyl nitrogen optionally is substituted with methyl.

In some preferred embodiments, R^{4s} is C₁-C₄-alkyl, aminopropyl, monomethylaminopropyl, dimethylaminopropyl, hydroxypropyl, methoxypropyl, cyclopentylmethyl, pyrrolidinylmethyl, tetrahydrofuranylmethyl, piperidinylmethyl, tetrahydropyranylmethyl, pyridinylmethyl, C₁-C₃-alkylamino, aminoethylamino, monomethylaminoethylamino, dimethylaminoethylamino, hydroxyethylamino, methoxyethylamino, cyclopentylamino, pyrrolidinylamino, tetrahydrofuranylamino, piperidinylamino, tetrahydropyranylamino, pyridinylamino, C₁-C₃-alkoxy, aminoethoxy, monomethylaminoethoxy, dimethylaminoethoxy, hydroxyethoxy, methoxyethoxy, cyclopentyloxy, pyrrolidinyloxy, tetrahydrofuranyloxy, piperidinyloxy, tetrahydropyranyloxy, or pyridinyloxy. Any pyrrolidinyl nitrogen or piperidinyl nitrogen optionally is substituted with methyl.

In some preferred embodiments, R^{4s} is hydrogen, alkyl, aminoalkyl, alkoxyalkyl, cycloalkylalkyl, arylalkyl, hetercycloalkylalkyl, heteroarylalkyl, amino, alkylamino, aminoalkylamino, alkoxyalkylamino, cycloalkylamino, arylamino, heterocycloalkylamino, heteroarylamino, hydroxy, alkoxy, aminoalkoxy, alkoxyalkoxy, cycloalkyloxy, aryloxy, heterocycloalkyloxy, heteroaryloxy, thiol, alkylthio, cycloalkylthio, arylthio, heterocycloalkylthio, heteroarylthio, aminosulfonyl, alkylsulfonyl, cycloalkylsulfonyl, arylsulfonyl, heterocycloalkylsulfonyl, or heteroarylsulfonyl. Any such substituent optionally is substituted with one or more substituents independently selected from the group consisting of hydroxy, cyano, and alkyl.

In some preferred embodiments, R^{4s} is hydrogen, C₁-C₆-alkyl, aminoalkyl, alkoxyalkyl, cycloalkylalkyl, hetercycloalkylalkyl, heteroarylalkyl, aminoalkylamino, alkoxyalkylamino, cycloalkylamino, heterocycloalkylamino, heteroarylamino, hydroxy, C₂-C₆-alkoxy, aminoalkoxy, alkoxyalkoxy, cycloalkyloxy, heterocycloalkyloxy, heteroaryloxy, thiol, C₂-C₆-alkylthio, cycloalkylthio, heterocycloalkylthio, heteroarylthio, aminosulfonyl, C₂-C₆-alkylsulfonyl, cycloalkylsulfonyl, heterocycloalkylsulfonyl, or heteroarylsulfonyl. Any such substituent optionally is substituted with one or more substituents independently selected from the group consisting of hydroxy, cyano, and alkyl.

In some preferred embodiments, R^{4s} is C₁-C₆-alkyl, aminoalkyl, alkoxyalkyl, cycloalkylalkyl, hetercycloalkylalkyl, heteroarylalkyl, aminoalkylamino, alkoxyalkylamino, cycloalkylamino, heterocycloalkylamino, heteroarylamino, hydroxy, C₂-C₆-alkoxy, aminoalkoxy, alkoxyalkoxy, cycloalkyloxy, heterocycloalkyloxy, heteroaryloxy, thiol, C₂-C₆-alkylthio, cycloalkylthio, heterocycloalkylthio, heteroarylthio, aminosulfonyl, C₂-C₆-alkylsulfonyl, cycloalkylsulfonyl, heterocycloalkylsulfonyl, or heteroarylsulfonyl. Any such substituent optionally is substituted with one or more substituents independently selected from the group consisting of hydroxy, cyano, and alkyl.

In some preferred embodiments, R^{4s} is alkyl, aminoalkyl, alkoxycarbonyl, carbocyclyloxycarbonyl, heterocyclyloxycarbonyl, or alkylaminocarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R^{4s} is alkoxycarbonyl, carbocyclyloxycarbonyl, or heterocyclyloxycarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R^{4s} is alkylaminocarbonyl optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some preferred embodiments, R^{4s} is —CH₂OH, —C(CH₃)(H)—OH, or —C(CH₃)₂—OH.

In some preferred embodiments, R^{4s} is aminomethyl. In these embodiments, the amino nitrogen optionally is substituted with up to two substituents independently selected from the group consisting of alkyl, alkenyl, hydroxy, haloalkyl, alkylsulfonyl, alkoxyalkyl, and heterocyclyl.

In some preferred embodiments, R^{4s} is hydrogen, alkylthio, mono-alkylamino, di-alkylamino, alkoxy, or haloalkoxy.

In some preferred embodiments, R^{4s} is alkylthio, mono-alkylamino, di-alkylamino, alkoxy, or haloalkoxy.

In some preferred embodiments, R^{4s} is heterocyclyloxy, heterocyclylalkoxy, cycloalkylamino, cyanoaryloxy, dialkylaminoalkylamino, or carbocyclylalkylheterocyclylamino.

In some preferred embodiments, R^{4s} is tetrahydrofuranyloxy, cyanophenyloxy, morpholinylethyloxy, cyclopentylamino, dimethylaminoethylamino, or phenylmethylpiperidinylamino.

In some preferred embodiments, R^{4s} is alkylaminoalkoxy.

In some preferred embodiments, R^{4s} is dialkylaminoalkoxy.

In some preferred embodiments, R⁴, is dimethylaminoethoxy.

In some preferred embodiments, R^{4s} is hydrogen.

### General Description of Preferred L² and R⁵ Substituents

L² is a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

Each R^{a} is independently selected from the group consisting of hydrogen and alkyl.

In some preferred embodiments, each R^{a} is hydrogen.

In other preferred embodiments, each R^{a} is alkyl.

In some preferred embodiments, L² is —O—, —S—, —S(O)—, —S(O)₂—, —N(R^{a})—, —C(O)—, —C(O)—N(R^{a}), —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

In some preferred embodiments, L² is a bond, —O—, —S—, —S(O)—, —N(R^{a})—, —N(R^{a})—C(O)—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

In some preferred embodiments, -L² is —C(O)—.

In some preferred embodiments, -L² is —O—.

In some preferred embodiments, L² is —N(R^{a})—.

In some preferred embodiments, L² is a bond.

R⁵ is hydrogen, hydroxy, alkyl, alkenyl, alkynyl, alkoxy, alkoxyalkyl, alkoxyalkoxyalkyl, alkylcarbonylalkyl, alkylcarbonyloxyalkyl, alkoxycarbonylalkyl, carbocyclyl, carbocyclylalkyl, heterocyclyl, or heterocyclylalkyl. Any such substituent is optionally substituted with one or more substituents independently selected from the group consisting of halogen, hydroxy, alkyl, haloalkyl, hydroxyalkyl, alkoxy, and haloalkoxy.

In some preferred embodiments, R⁵ is hydrogen, hydroxy, alkyl alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent is optionally substituted with one or more substituents independently selected from the group consisting of halogen, hydroxy, alkyl, haloalkyl, hydroxyalkyl, alkoxy, and haloalkoxy.

In some preferred embodiments, R⁵ is hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

In some preferred embodiments, R⁵ is alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

In some preferred embodiments, R⁵ is hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl.

In some preferred embodiments, R⁵ is hydrogen, alkenyl, or alkylcarbonylalkyl. Any such substituent optionally is substituted with one or more substituents independently selected from the group consisting of halogen, hydroxy, alkoxy, and haloalkoxy.

In some preferred embodiments, R⁵ is alkyl, alkoxyalkyl, alkoxyalkoxyalkyl, alkylcarbonyloxyalkyl, or tetrahydrofuranylalkyl. Any such substituent optionally is substituted with one or more substituents independently selected from the group consisting of hydroxy and halogen.

In some preferred embodiments, R⁵ is alkyl, alkoxyalkyl, alkoxyalkoxyalkyl, or tetrahydrofuranylalkyl. Any such substituent optionally is substituted with one or more substituents independently selected from the group consisting of hydroxy and halogen.

In some preferred embodiments, R⁵ is haloalkyl, hydroxyalkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

In some preferred embodiments, R⁵ is phosphonooxyalkyl, monoalkylphosphonooxyalkyl, dialkylphosphonooxyalkyl, aminoalkylcarbonyloxyalkyl, monoalkylaminoalkylcarbonyloxyalkyl, dialkylaminoalkylcarbonyloxyalkyl, phenylalkyl substituted with alkylcarbonyloxy, or tetrahydrofuranyl.

In some preferred embodiments, R⁵ is hydrogen, alkenyl, or alkylcarbonylalkyl.

In some preferred embodiments, R⁵ is hydroxyalkyl, i.e., alkyl substituted with one or more hydroxy radicals (often only one hydroxy radical).

In some preferred embodiments, R⁵ is C₁-C₆-hydroxyalkyl.

In some preferred embodiments, R⁵ is hydroxymethyl.

In some preferred embodiments, R⁵ is alkylcarbonyloxyalkyl.

In some preferred embodiments, R⁵ is methylcarbonyloxymethyl.

In some preferred embodiments, R⁵ is hydrogen.

In some preferred embodiments, R⁵ is heterocyclyl optionally substituted with one or more substituents independently selected from the group consisting of halogen, hydroxy, alkyl, haloalkyl, hydroxyalkyl, alkoxy, and haloalkoxy.

In some preferred embodiments, R⁵ is carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

In some preferred embodiments, R⁵ is substituted methyl. The methyl is substituted with:

two substituents independently selected from the group consisting of hydroxy, alkoxy, hydroxymethyl, hydroxyethyl, alkoxymethyl, alkoxyethyl, tetrahydrofuranyl, and tetrahydrofuranylmethyl, wherein any such substituent is, in turn, optionally substituted with one or more substituents independently selected from the group consisting of halogen, hydroxy, alkyl, haloalkyl, hydroxyalkyl, alkoxy, and haloalkoxy; or a substituent selected from the group consisting of alkoxyethoxy, hydroxyethoxy, alkoxypropoxy, and hydroxypropoxy, wherein any such substituent is, in turn, optionally substituted with one or more substituents independently selected from the group consisting of halogen, hydroxy, alkyl, haloalkyl, hydroxyalkyl, alkoxy, and haloalkoxy.

The methyl optionally is further substituted with hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl. Any such optional substituent is, in turn, optionally substituted with one or more substituents independently selected from the group consisting of halogen, hydroxy, alkyl, haloalkyl, hydroxyalkyl, alkoxy, and haloalkoxy.

In some preferred embodiments, R⁵ is a radical corresponding in structure to one of the following formulas:

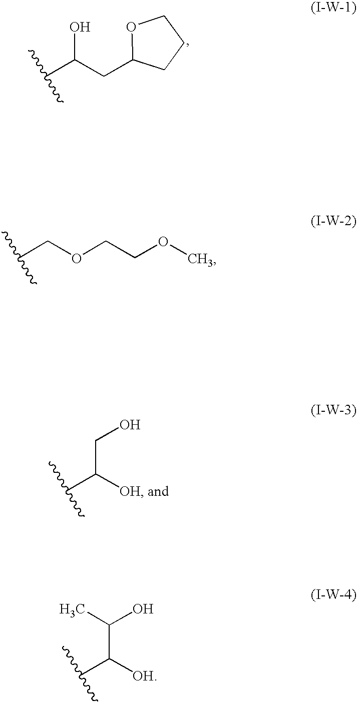

In some preferred embodiments, -L²-R⁵ is a radical corresponding in structure to one of the following formulas:

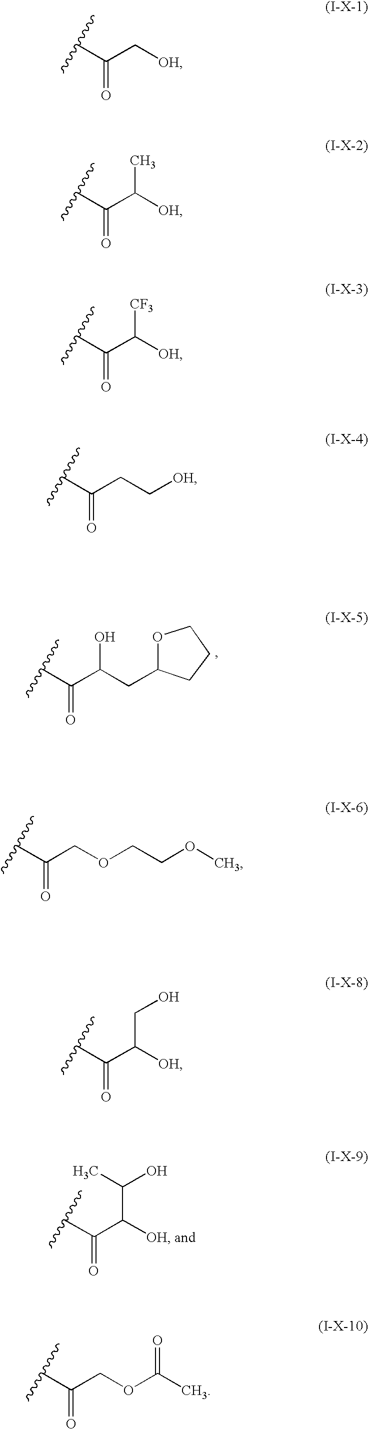

In some preferred embodiments, -L²-R⁵ is a radical corresponding in structure to one of the following formulas:

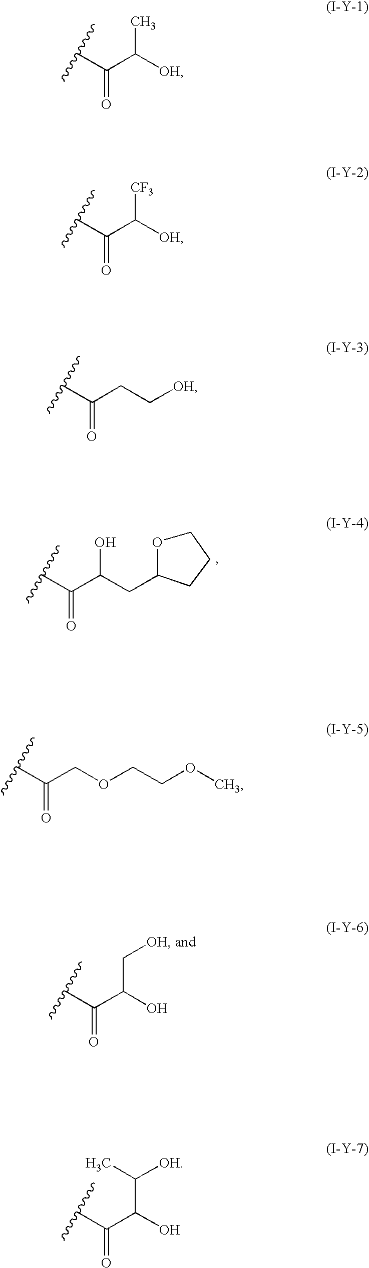

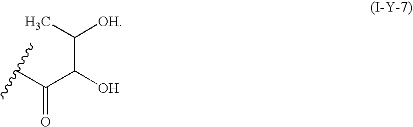

In some preferred embodiments, L² is a bond; and R⁵ is heterocyclyl optionally substituted with one or more substituents independently selected from the group consisting of halogen, hydroxy, alkyl, haloalkyl, hydroxyalkyl, alkoxy, and haloalkoxy.

In some preferred embodiments, the compound corresponds in structure to the following formula:

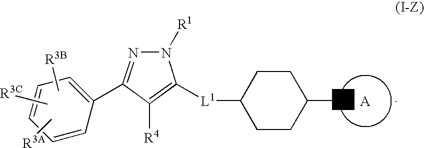

Here, the ring structure A is a heterocyclyl ring that contains a nitrogen bonded to the cyclohexyl. The is heterocyclyl ring also is optionally substituted with one or more substituents independently selected from the group consisting of halogen, hydroxy, alkyl, haloalkyl, hydroxyalkyl, alkoxy, and haloalkoxy.

In some preferred embodiments, L² is —N(R^{a})—; and R⁵ is alkyl, carbocyclyl, or carbocyclylalkyl. The alkyl, carbocyclyl, or carbocyclylalkyl optionally is substituted with one or more substituents independently selected from the group consisting of halogen, hydroxy, alkyl, haloalkyl, hydroxyalkyl, alkoxy, and haloalkoxy.

In some preferred embodiments, -L²-R⁵ is hydroxyalkylcarbonyl, i.e., alkylcarbonyl substituted with one or more hydroxy radicals (often only one hydroxy radical).

In some preferred embodiments, -L²-R⁵ is hydroxymethylcarbonyl.

In some preferred embodiments, -L²-R⁵ is alkylcarbonyloxyalkylcarbonyl.

In some preferred embodiments, -L²-R⁵ is methylcarbonyloxymethylcarbonyl.

In some preferred embodiments, -L²-R⁵ is hydroxy.

In some preferred embodiments, -L²-R⁵ is hydrogen, methyl, or butyloxycarbonyl.

In some preferred embodiments, -L²-R⁵ is hydrogen or alkyl.

### DETAILED DESCRIPTION OF SEVERAL PREFERRED EMBODIMENTS

The above discussion describes the compounds of this invention in general terms. The following discussion, in turn, describes in detail several specific preferred and particularly preferred embodiments.

### Preferred Embodiment No. 1

In some preferred embodiments:

L² is —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

R^{3A} and R^{3B} are independently selected from the group consisting of halogen, hydroxy, cyano, amino, alkyl, aminoalkyl, monoalkylamino, dialkylamino, alkoxy, and alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

### Particularly Preferred Compounds of Embodiment No. 1

In some particularly preferred embodiments, R¹ is hydroxyalkyl.

In some particularly preferred embodiments, R¹ is hydrogen.

In some particularly preferred embodiments, L¹ is a bond.

In some particularly preferred embodiments, R^{3C} is hydrogen.

In some particularly preferred embodiments, X², X³, X⁵, and X⁶ are each —CH₂—.

In some particularly preferred embodiments, R^{3A} and R^{3b} are independently selected from the group consisting of fluoro, chloro, methyl, trifluoromethyl, ethyl, hydroxy, trifluoromethoxy, amino, monomethylamino, and dimethylamino.

In some particularly preferred embodiments, R^{3A} and R^{3B} are independently selected from the group consisting of halogen, methyl, methoxy, halomethyl, and halomethoxy.

In some particularly preferred embodiments, R^{3A} and R^{3B} are independently selected from the group consisting of chloro, fluoro, methyl, methoxy, chloromethyl, fluoromethyl, chloromethoxy, and fluoromethoxy.

In some particularly preferred embodiments, R^{3A} and R^{3B} are independently selected from the group consisting of chloro, methyl, methoxy, chloromethyl, and chloromethoxy.

In some particularly preferred embodiments, R^{3A} and R^{3B} are independently selected from the group consisting of fluoro, methyl, methoxy, fluoromethyl, and fluoromethoxy.

In some particularly preferred embodiments, R^{3A} and R^{3B} are independently selected from the group consisting of chloro, chloromethyl, and chloromethoxy.

In some particularly preferred embodiments, R^{3A} and R^{3B} are independently selected from the group consisting of fluoro, fluoromethyl, and fluoromethoxy.

In some particularly preferred embodiments, the compound corresponds in structure to the following formula:

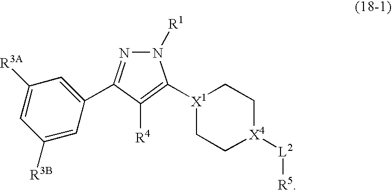

In some particularly preferred embodiments, the compound corresponds in structure to the following formula:

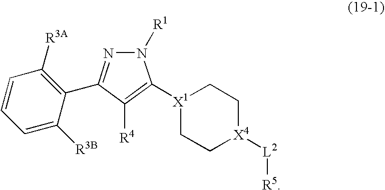

In some particularly preferred embodiments, the compound corresponds in structure to the following formula:

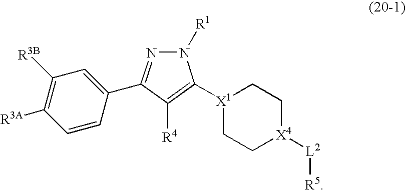

In some particularly preferred embodiments, the compound corresponds in structure to the following formula:

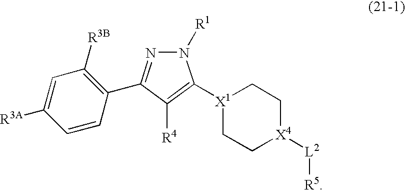

In some particularly preferred embodiments, the compound corresponds in structure to the following formula:

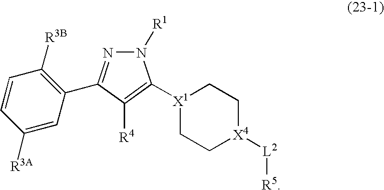

In some particularly preferred embodiments, the compound corresponds in structure to the following formula:

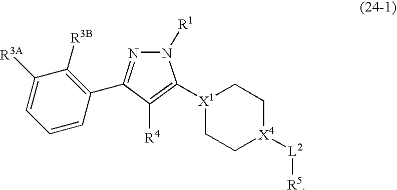

In some particularly preferred embodiments, X¹ and X⁴ are each carbon bonded to hydrogen. In some such embodiments, the compound corresponds in structure to the following formula:

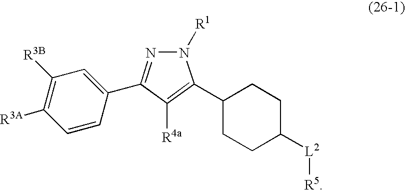

Examples of such compounds include those corresponding in structure to the following formulas:

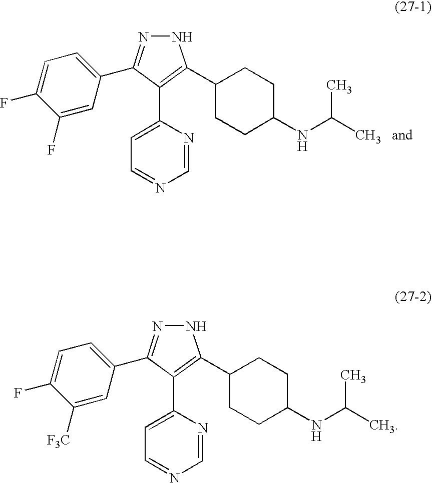

In some embodiments, the preferred geometrical isomers have the trans configuration with respect to the cyclohexyl group. Thus, for example, the preferred geometrical isomers of the compounds of Formulas (27-1) and (27-2) are the following, respectively:

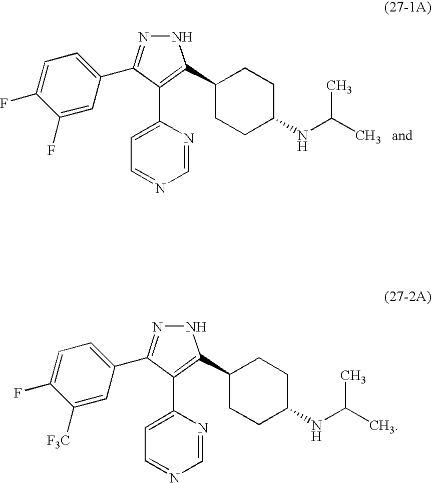

In some particularly preferred embodiments, -L² is —C(O)—.

In some particularly preferred embodiments, R⁴ is pyrimidinyl optionally substituted with halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, or carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, the compound corresponds in structure to the following formula:

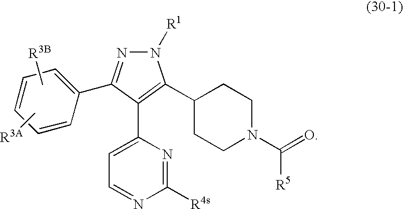

In some such embodiments, R^{4s} is hydrogen, alkyl, aminoalkyl, alkoxyalkyl, cycloalkylalkyl, hetercycloalkylalkyl, heteroarylalkyl, amino, alkylamino, aminoalkylamino, alkoxyalkylamino, cycloalkylamino, heterocycloalkylamino, heteroarylamino, hydroxy, alkoxy, aminoalkoxy, alkoxyalkoxy, cycloalkyloxy, heterocycloalkyloxy, or heteroaryloxy. Any such substituent optionally is substituted with one or more substituents independently selected from the group consisting of hydroxy and alkyl.

In other such embodiments, R^{4s} is hydrogen, C₁-C₄-alkyl, aminopropyl, monomethylaminopropyl, dimethylaminopropyl, hydroxypropyl, methoxypropyl, cyclopentylmethyl, pyrrolidinylmethyl, tetrahydrofuranylmethyl, piperidinylmethyl, tetrahydropyranylmethyl, pyridinylmethyl, C₁-C₃-alkylamino, aminoethylamino, monomethylaminoethylamino, dimethylaminoethylamino, hydroxyethylamino, methoxyethylamino, cyclopentylamino, pyrrolidinylamino, tetrahydrofuranylamino, piperidinylamino, tetrahydropyranylamino, pyridinylamino, C₁-C₃-alkoxy, aminoethoxy, monomethylaminoethoxy, dimethylaminoethoxy, hydroxyethoxy, methoxyethoxy, cyclopentyloxy, pyrrolidinyloxy, tetrahydrofuranyloxy, piperidinyloxy, tetrahydropyranyloxy, or pyridinyloxy. Any pyrrolidinyl nitrogen or piperidinyl nitrogen optionally is substituted with methyl.

In some particularly preferred embodiments, R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent is substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, R⁴ is pyridinyl substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, R⁴ is pyrimidinyl substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, the compound corresponds in structure to the following formula:

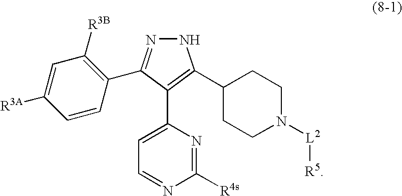

In these embodiments, R^{4s} is alkyl, aminoalkyl, alkoxycarbonyl, carbocyclyloxycarbonyl, heterocyclyloxycarbonyl, or alkylaminocarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some such embodiments, R^{4s} is alkoxycarbonyl, carbocyclyloxycarbonyl, or heterocyclyloxycarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some other such embodiments, R^{4s} is alkylaminocarbonyl optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some other such embodiments, R^{4s} is —CH₂OH, —C(CH₃)(H)—OH, or —C(CH₃)₂—OH.

In some other such embodiments, R^{4s} is aminomethyl. In these embodiments, the amino nitrogen optionally is substituted with up to two substituents independently selected from the group consisting of alkyl, alkenyl, hydroxy, haloalkyl, alkylsulfonyl, alkoxyalkyl, and heterocyclyl.

In some particularly preferred embodiments, R⁵ is alkyl, alkoxyalkyl, alkoxyalkoxyalkyl, alkylcarbonyloxyalkyl, or tetrahydrofuranylalkyl. Any such substituent optionally is substituted with one or more substituents independently selected from the group consisting of hydroxy and halogen.

In some particularly preferred embodiments, -L²-R⁵ is a radical corresponding in structure to one of the following formulas:

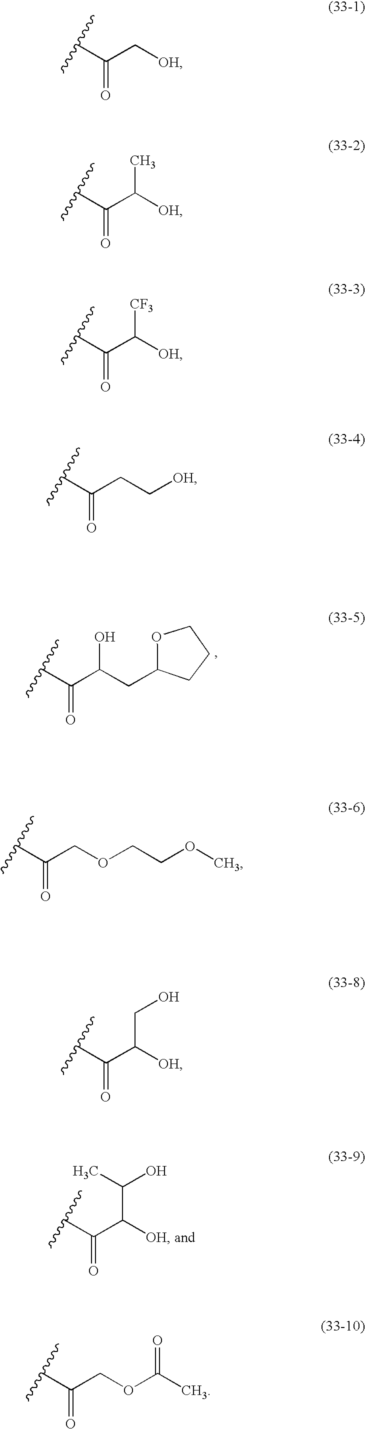

In some particularly preferred embodiments, -L²-R⁵ is hydroxyalkylcarbonyl.

In some particularly preferred embodiments, -L²-R⁵ is hydroxymethylcarbonyl.

In some particularly preferred embodiments, the compound corresponds in structure to the following formula:

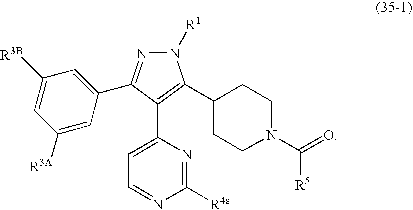

Examples of such compounds include those corresponding in structure to the following formulas:

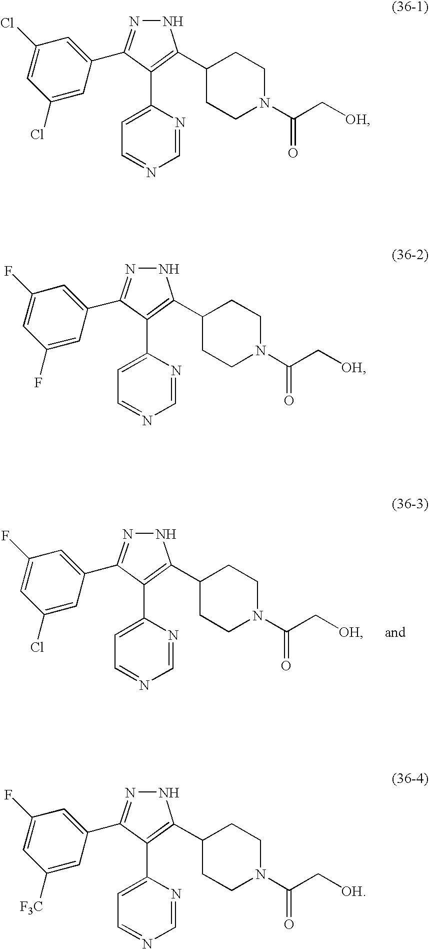

Additional examples of such compounds include those corresponding in structure to the following formulas:

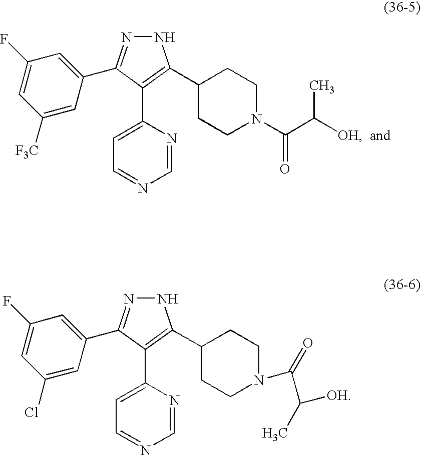

In some embodiments, the preferred optical isomer of the compound of Formula (36-5) corresponds in structure to the following formula:

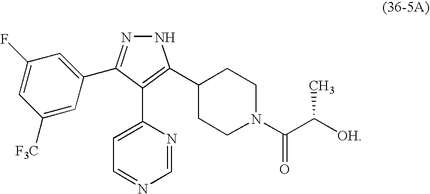

In some embodiments, the preferred optical isomer of the compound of Formula (36-6) corresponds in structure to the following formula:

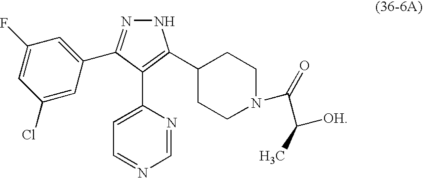

In some particularly preferred embodiments, the compound corresponds in structure to the following formula:

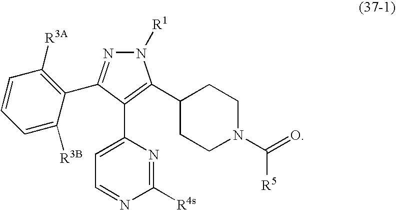

One such compound, for example, corresponds in structure to the following formula:

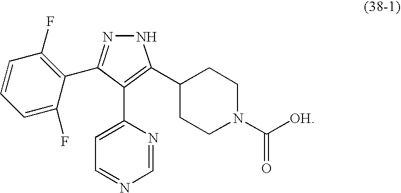

In some particularly preferred embodiments, the compound corresponds in structure to the following formula:

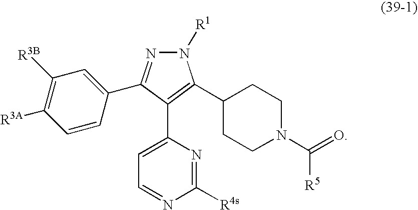

Examples of such compounds include those corresponding in structure to the following formulas:

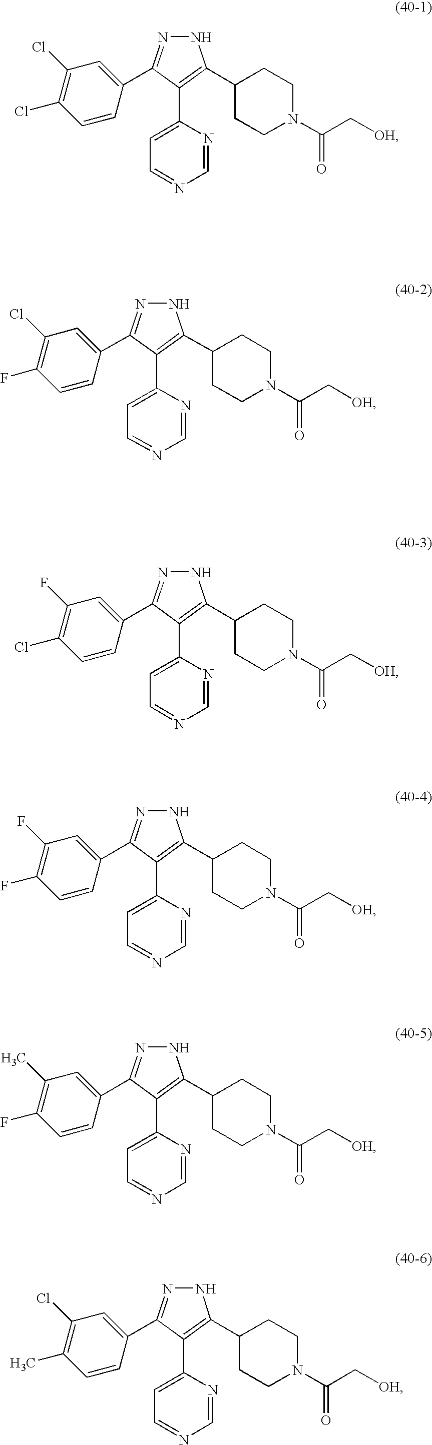

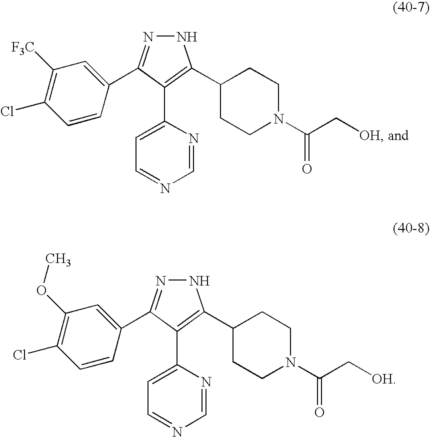

Additional examples of such compounds include those corresponding in structure to the following formulas:

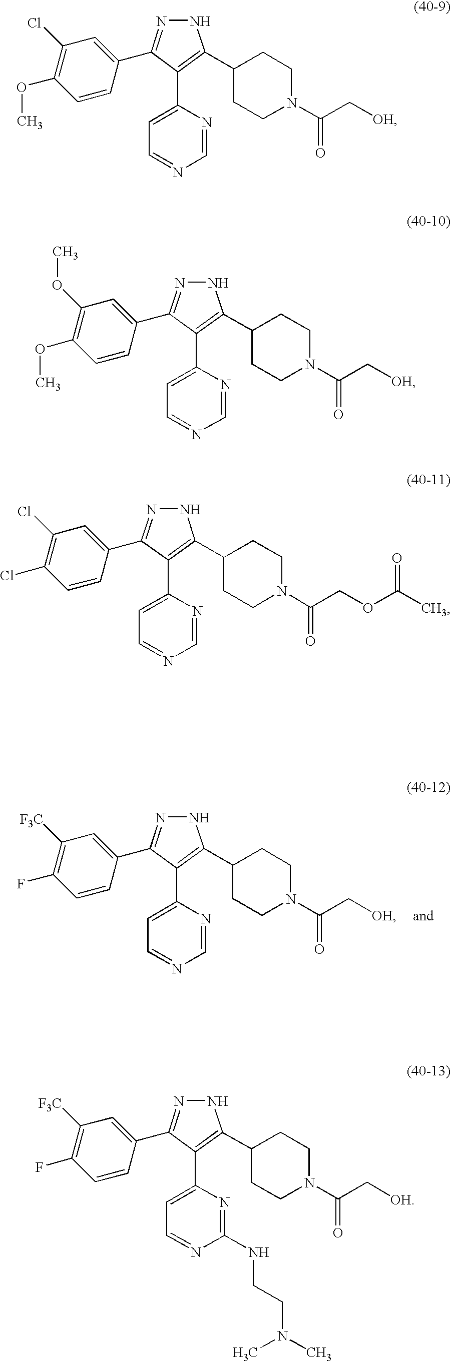

In some particularly preferred embodiments, the compound corresponds in structure to the following formula:

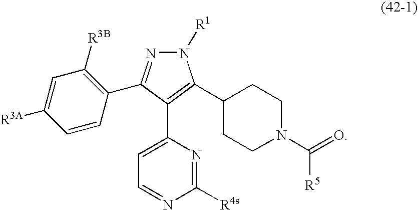

Examples of such compounds include those corresponding in structure to the following formulas:

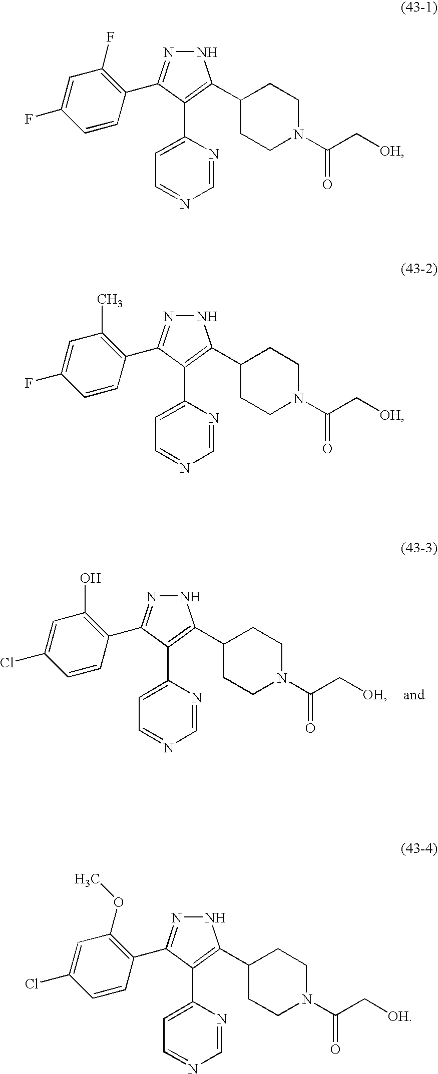

Examples of such compounds also include those corresponding in structure to the following formulas:

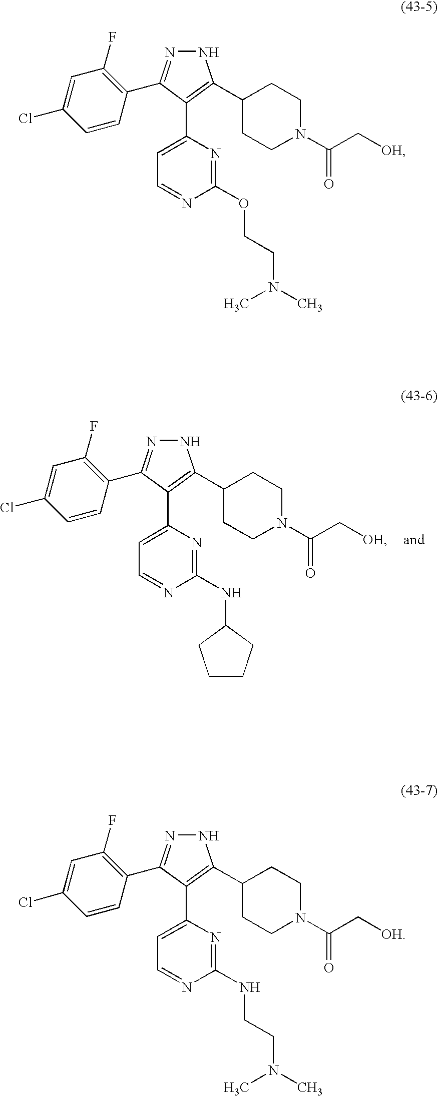

An additional example of such compounds includes the compound corresponding in structure to the following formula:

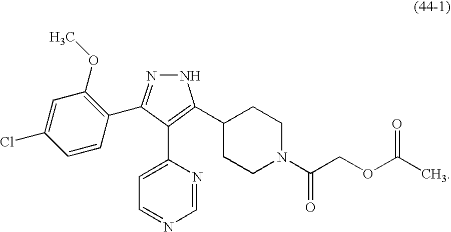

An additional example of such compounds includes the compound corresponding in structure to the following formula:

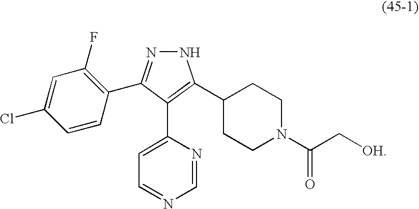

An additional example of such compounds includes the compound corresponding in structure to the following formula:

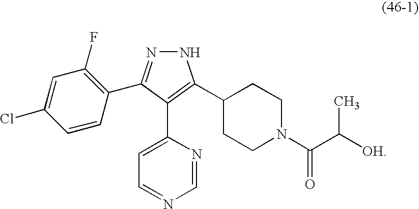

In some embodiments, this compound corresponds in structure to the following optical isomer:

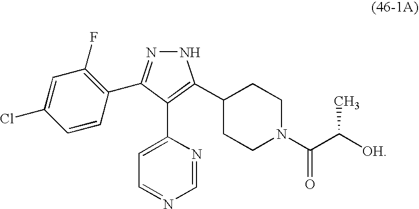

In other embodiments, the compound corresponds in structure to the following optical isomer:

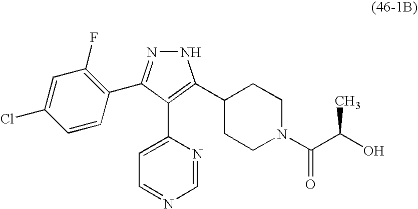

In some particularly preferred embodiments, the compound corresponds in structure to the following formula:

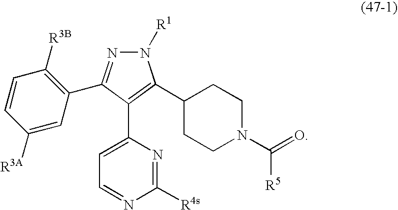

Examples of such compounds include those corresponding in structure to the following formulas:

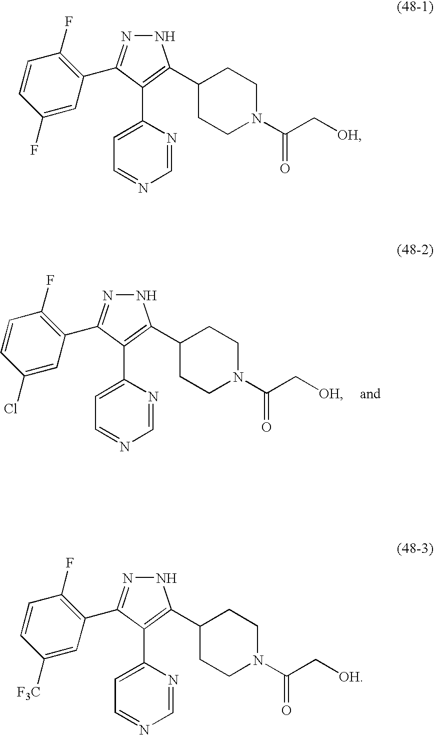

Additional examples of such compounds include those corresponding in structure to the following formulas:

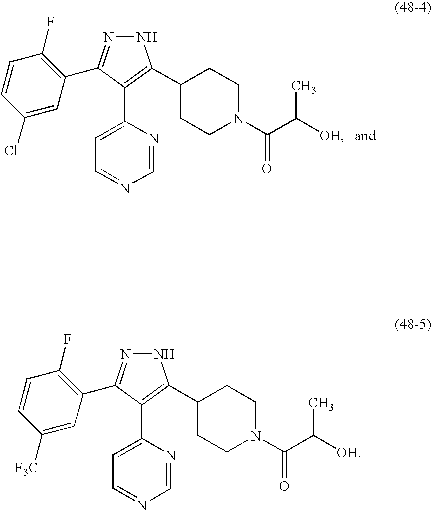

In some embodiments, the preferred optical isomer of the compound of Formulas (48-4) corresponds in structure to the following formula:

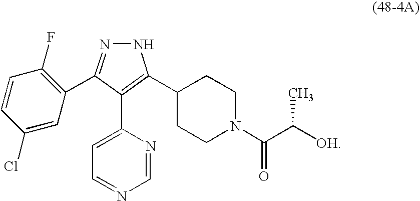

In some embodiments, the preferred optical isomer of the compound of Formulas (48-5) corresponds in structure to the following formula:

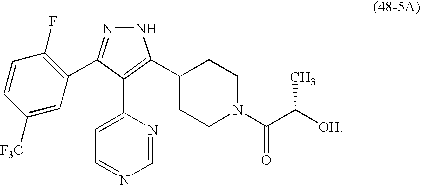

In some particularly preferred embodiments, the compound corresponds in structure to the following formula:

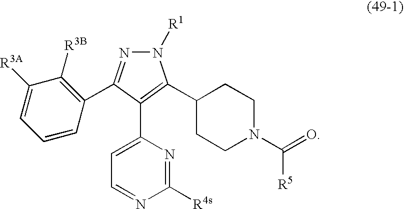

Examples of such compounds include those corresponding in structure to the following formulas:

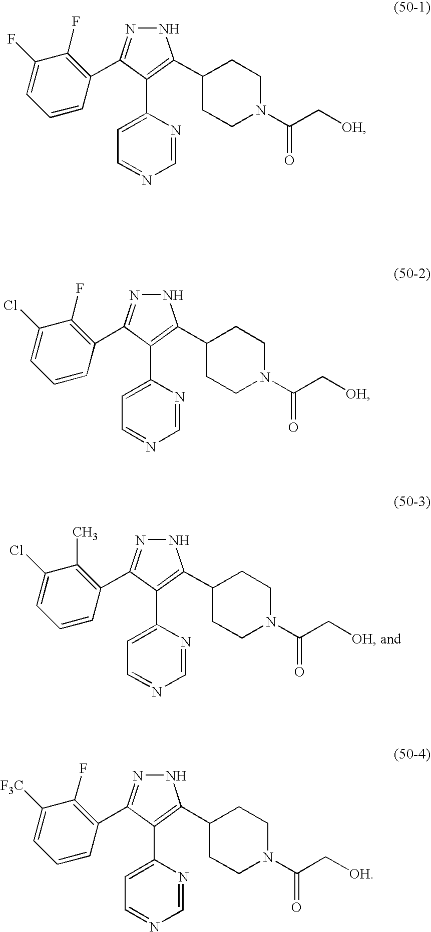

Other examples of such compounds include those corresponding in structure to the following formulas:

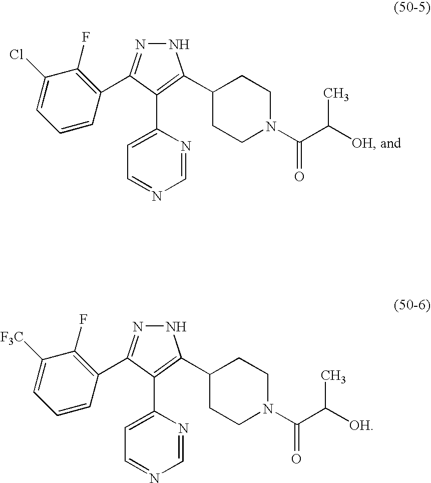

In some embodiments, the preferred optical isomer of the compound of Formula (50-5) corresponds in structure to the following formula:

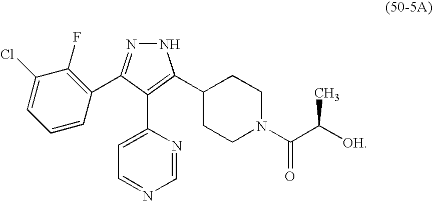

In some embodiments, the preferred optical isomer of the compound of Formula (50-5) corresponds in structure to the following formula:

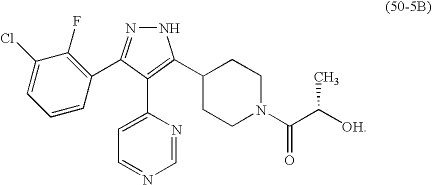

In some embodiments, the preferred isomer of the compound of Formula (50-6) corresponds in structure to the following formula:

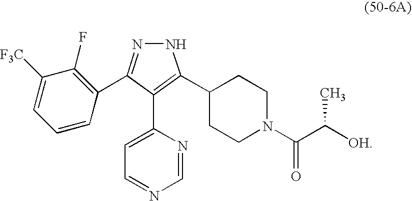

### Preferred Embodiment No. 2

In some preferred embodiments:

R^{3A} and R^{3B} are independently selected from the group consisting of halogen, hydroxy, cyano, amino, alkyl, aminoalkyl, monoalkylamino, dialkylamino, cycloalkylamino, alkoxy, and alkoxyalkyl. Any carbon of the alkyl, aminoalkyl, monoalkylamino, dialkylamino, cycloalkylamino, alkoxy, or alkoxyalkyl optionally is substituted with one or more substituents independently selected from the group consisting of halogen, hydroxy, and cyano.

R⁴ is pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 2

In some particularly preferred embodiments, R¹ is hydrogen.

In some particularly preferred embodiments, L¹ is a bond.

In some particularly preferred embodiments, R^{3C} is hydrogen.

In some particularly preferred embodiments, X², X³, X⁵, and X⁶ are each —CH₂—.

In some particularly preferred embodiments, R^{3A} and R^{3B} are independently selected from the group consisting of halogen, hydroxy, cyano, amino, alkyl, aminoalkyl, monoalkylamino, dialkylamino, alkoxy, and alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

In some particularly preferred embodiments, R^{3A} and R^{3B} are independently selected from the group consisting of halogen, methyl, methoxy, halomethyl, and halomethoxy.

In some particularly preferred embodiments, R^{3A} and R^{3B} are independently selected from the group consisting of chloro, fluoro, methyl, methoxy, chloromethyl, fluoromethyl, chloromethoxy, and fluoromethoxy.

In some particularly preferred embodiments, R^{3A} and R^{3B} are independently selected from the group consisting of chloro, methyl, methoxy, chloromethyl, and chloromethoxy.

In some particularly preferred embodiments, R^{3A} and R^{3B} are independently selected from the group consisting of fluoro, methyl, methoxy, fluoromethyl, and fluoromethoxy.

In some particularly preferred embodiments, R^{3A} and R^{3B} are independently selected from the group consisting of chloro, chloromethyl, and chloromethoxy.

In some particularly preferred embodiments, R^{3A} and R^{3B} are independently selected from the group consisting of fluoro, fluoromethyl, and fluoromethoxy.

In some particularly preferred embodiments, R⁴ is pyrimidinyl substituted with alkyl, aminoalkyl, alkoxycarbonyl, carbocyclyloxycarbonyl, heterocyclyloxycarbonyl, or alkylaminocarbonyl. Any such substituent, in turn, is optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, the compound corresponds in structure to the following formula:

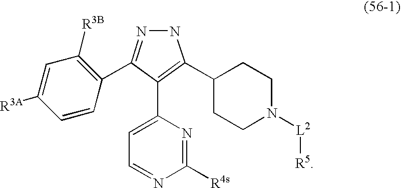

In these embodiments, R^{4s} is alkyl, aminoalkyl, alkoxycarbonyl, carbocyclyloxycarbonyl, heterocyclyloxycarbonyl, or alkylaminocarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some such embodiments, R^{4s} is alkoxycarbonyl, carbocyclyloxycarbonyl, or heterocyclyloxycarbonyl. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some other such embodiments, R^{4s} is alkylaminocarbonyl optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some other such embodiments, R^{4s} is —CH₂OH, —C(CH₃)(H)—OH, or —C(CH₃)₂—OH.

In some other such embodiments, R^{4s} is aminomethyl. In these embodiments, the amino nitrogen optionally is substituted with up to two substituents independently selected from the group consisting of alkyl, alkenyl, hydroxy, haloalkyl, alkylsulfonyl, alkoxyalkyl, and heterocyclyl.

In some particularly preferred embodiments, the compound corresponds in structure to the following formula:

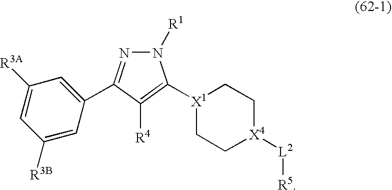

In some particularly preferred embodiments, the compound corresponds in structure to the following formula:

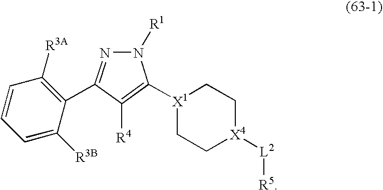

In some particularly preferred embodiments, the compound corresponds in structure to the following formula:

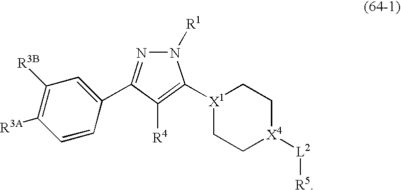

Examples of such compounds include, for example, those corresponding in structure to the following formulas:

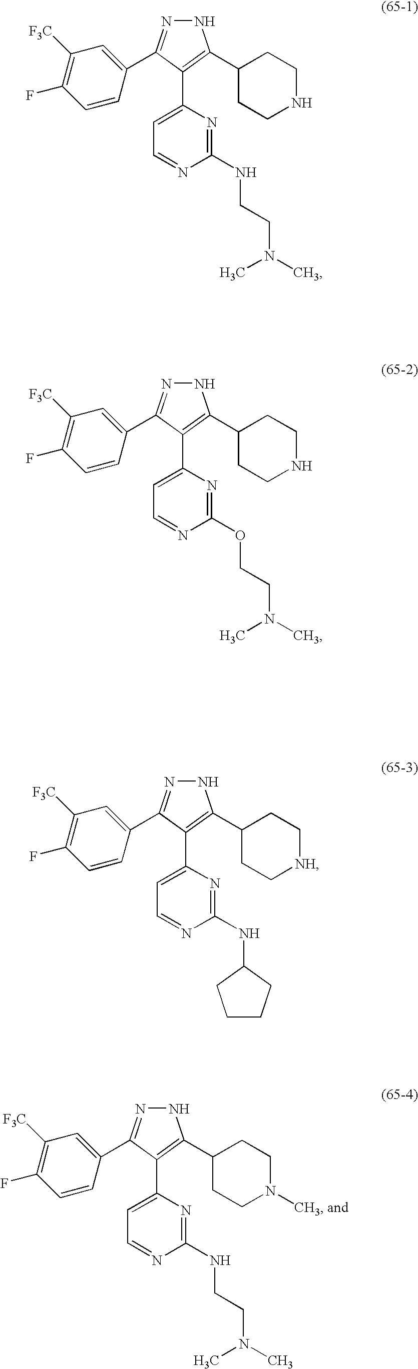

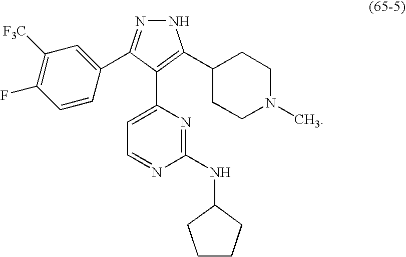

In some particularly preferred embodiments, the compound corresponds in structure to the following formula:

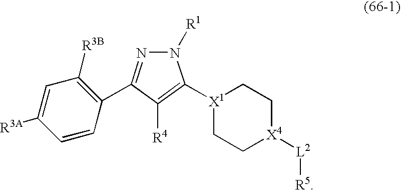

Examples of such compounds include, for example, those corresponding in structure to the following formulas:

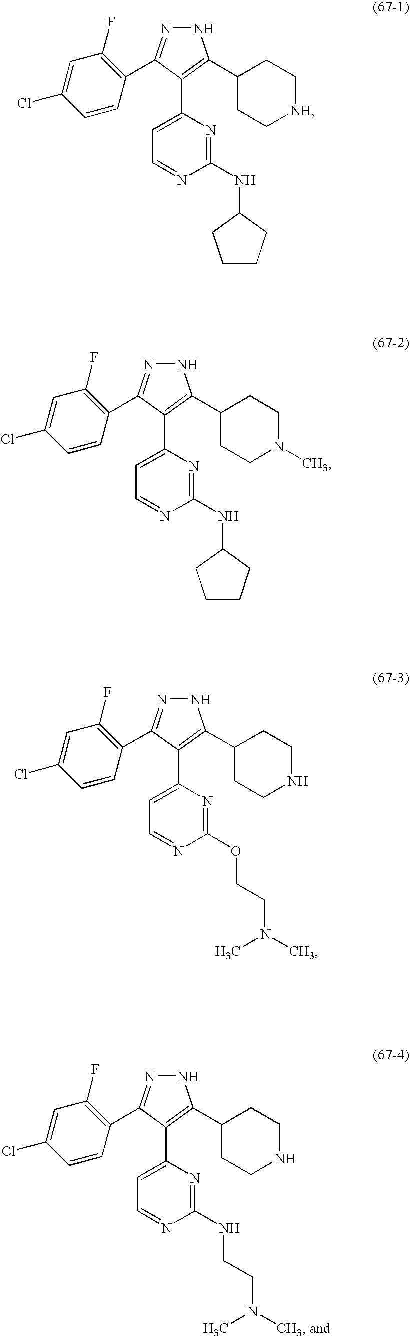

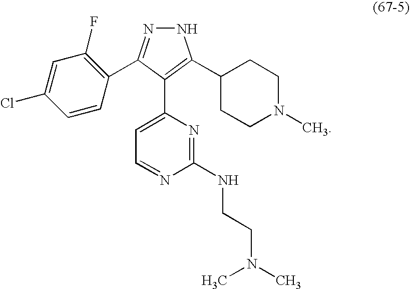

Another such compound, for example, corresponds in structure to the following formula:

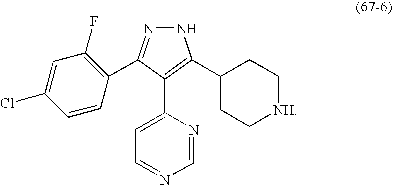

In some particularly preferred embodiments, the compound corresponds in structure to the following formula:

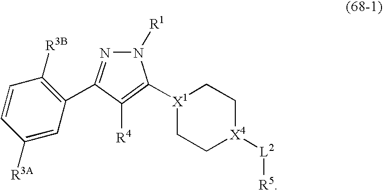

In some particularly preferred embodiments, the compound corresponds in structure to the following formula:

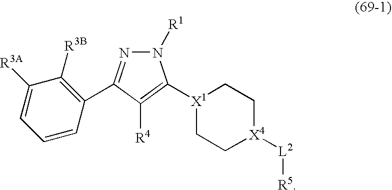

In some particularly preferred embodiments, -L²-R⁵ is hydrogen, methyl, or butyloxycarbonyl.

In some particularly preferred embodiments, -L²-R⁵ is hydrogen or alkyl.

### Preferred Embodiment No. 3

In some preferred embodiments, the compound corresponds in structure to the following formula:

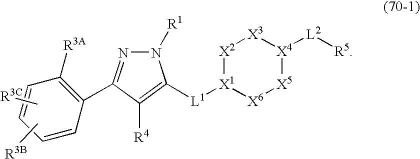

Here, R^{3A} and R^{3B} are independently selected from the group consisting of halogen, hydroxy, cyano, amino, alkyl, aminoalkyl, monoalkylamino, dialkylamino, alkoxy, and alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

### Particularly Preferred Compounds of Embodiment No. 3

In some particularly preferred embodiments, R¹ is hydroxyalkyl.

In some particularly preferred embodiments, R¹ is hydrogen.

In some particularly preferred embodiments, L¹ is a bond.

In some particularly preferred embodiments, R^{3C} is hydrogen.

In some particularly preferred embodiments, R^{3A} is halogen, methyl, methoxy, halomethyl, or halomethoxy.

In some particularly preferred embodiments, R^{3A} is chloro, chloromethyl, or chloromethoxy.

In some particularly preferred embodiments, R^{3A} is fluoro, fluoromethyl, or fluoromethoxy.

In some particularly preferred embodiments, R^{3A} and R^{3B} are independently selected from the group consisting of halogen, methyl, methoxy, halomethyl, and halomethoxy.

In some particularly preferred embodiments, R^{3A} and R^{3B} are independently selected from the group consisting of chloro, fluoro, methyl, methoxy, chloromethyl, fluoromethyl, chloromethoxy, and fluoromethoxy.

In some particularly preferred embodiments, R^{3A} and R^{3B} are independently selected from the group consisting of chloro, methyl, methoxy, chloromethyl, and chloromethoxy.

In some particularly preferred embodiments, R^{3A} and R^{3B} are independently selected from the group consisting of fluoro, methyl, methoxy, fluoromethyl, and fluoromethoxy.

In some particularly preferred embodiments, R^{3A} and R^{3B} are independently selected from the group consisting of chloro, chloromethyl, and chloromethoxy.

In some particularly preferred embodiments, R^{3A} and R^{3B} are independently selected from the group consisting of fluoro, fluoromethyl, and fluoromethoxy.

In some particularly preferred embodiments, X², X³, X⁵, and X⁶ are each —CH₂—.

In some particularly preferred embodiments, the compound corresponds in structure to the following formula:

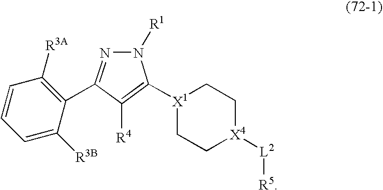

In some particularly preferred embodiments, the compound corresponds in structure to the following formula:

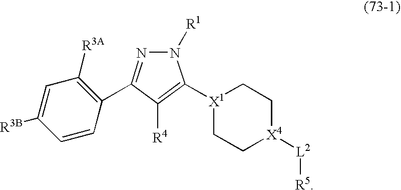

In some particularly preferred embodiments, the compound corresponds in structure to the following formula:

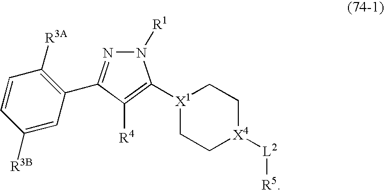

In some particularly preferred embodiments, the compound corresponds in structure to the following formula:

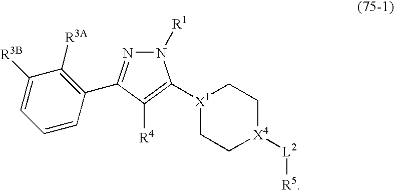

In some particularly preferred embodiments, -L²-R⁵ is hydrogen, methyl, or butyloxycarbonyl.

### Preferred Embodiment No. 4

In some preferred embodiments:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 4

In some particularly preferred embodiments, R¹ is hydroxyalkyl.

In some particularly preferred embodiments, R¹ is hydrogen.

In some particularly preferred embodiments, L¹ is a bond.

In some particularly preferred embodiments, R^{3C} is hydrogen.

In some particularly preferred embodiments, X², X³, X⁵, and X⁶ are each —CH₂—.

In some particularly preferred embodiments, -L² is —C(O)—.

In some particularly preferred embodiments, -L² is —O—.

In some particularly preferred embodiments, R⁵ is alkyl, alkoxyalkyl, alkoxyalkoxyalkyl, or tetrahydrofuranylalkyl. Any such substituent optionally is substituted with one or more substituents independently selected from the group consisting of hydroxy and halogen.

In some particularly preferred embodiments, R⁵ is hydrogen, alkenyl, or alkylcarbonylalkyl.

In some particularly preferred embodiments, -L²-R⁵ corresponds in structure to the of the following formulas:

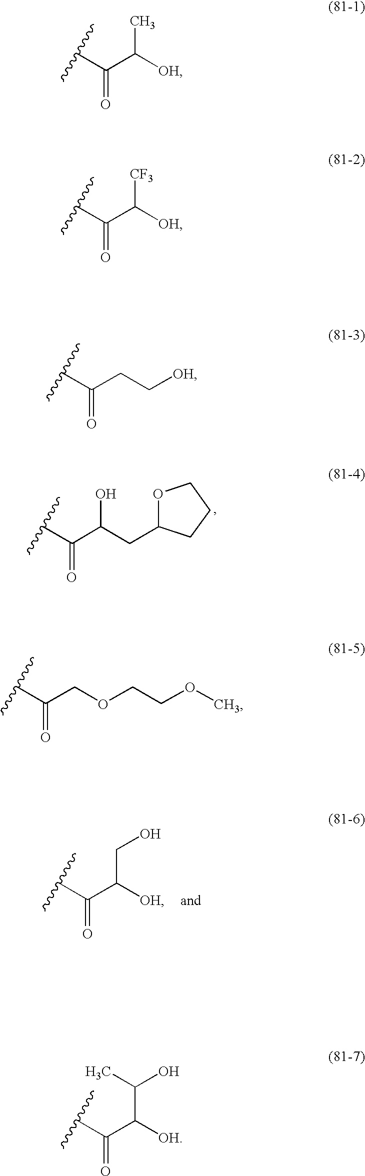

In some particularly preferred embodiments, -L²-R⁵ is alkylcarbonyl substituted with one or more hydroxy.

In some particularly preferred embodiments, the compound corresponds in structure to the following formula:

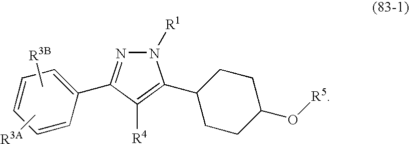

In some particularly preferred embodiments, the compound corresponds in structure to the following formula:

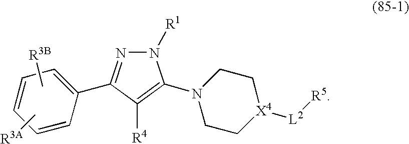

In some particularly preferred embodiments, the compound corresponds in structure to the following formula:

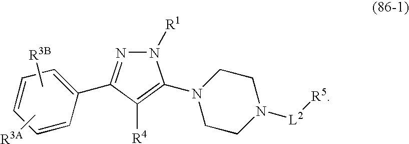

In some particularly preferred embodiments, the compound corresponds in structure to the following formula:

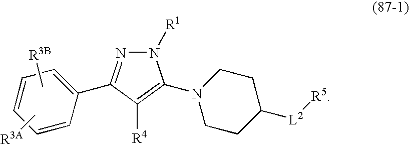

In some particularly preferred embodiments, the compound corresponds in structure to the following formula:

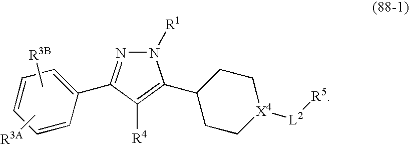

In some particularly preferred embodiments, the compound corresponds in structure to the following formula:

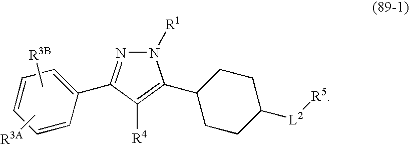

In some particularly preferred embodiments, L² is a bond; and R⁵ is heterocyclyl optionally substituted with one or more substituents independently selected from the group consisting of halogen, hydroxy, alkyl, haloalkyl, hydroxyalkyl, alkoxy, and haloalkoxy.

In some particularly preferred embodiments, the compound corresponds in structure to the following formula:

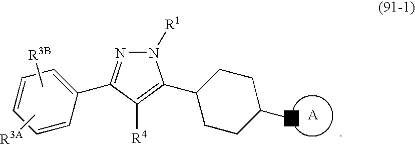

Here, the ring structure A is a heterocyclyl ring containing a nitrogen bonded to the cyclohexyl. The heterocyclyl ring also is optionally substituted with one or more substituents independently selected from the group consisting of halogen, hydroxy, alkyl, haloalkyl, hydroxyalkyl, alkoxy, and haloalkoxy.

In some particularly preferred embodiments, L² is —N(R^{a})—; and R⁵ is alkyl, carbocyclyl, or carbocyclylalkyl. The alkyl, carbocyclyl, or carbocyclylalkyl optionally is substituted with one or more substituents independently selected from the group consisting of halogen, hydroxy, alkyl, haloalkyl, hydroxyalkyl, alkoxy, and haloalkoxy.

In some particularly preferred embodiments, the compound corresponds in structure to the following formula:

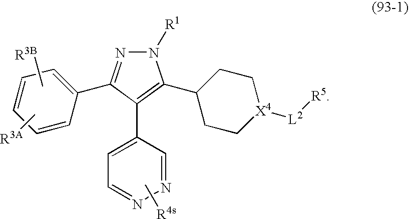

In some such particularly preferred embodiments, R^{4s} is hydrogen, alkylthio, mono-alkylamino, di-alkylamino, alkoxy, or haloalkoxy.

In some particularly preferred embodiments, the compound corresponds in structure to the following formula:

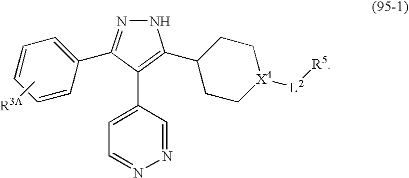

In some such embodiments, the compound corresponds in structure to the following formula:

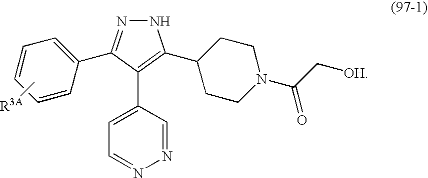

One such compound, for example, corresponds in structure to the following formula:

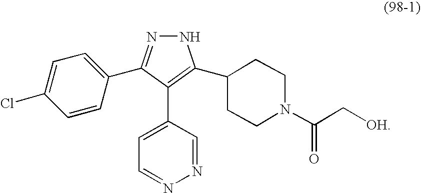

In some particularly preferred embodiments, the compound corresponds in structure to the following formula:

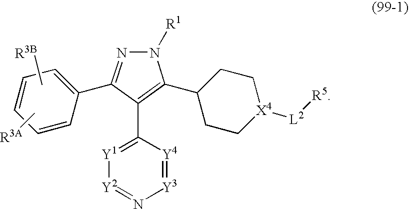

Here, two of Y¹, Y², Y³, and Y⁴ are each nitrogen, one of Y¹, Y², Y³, and Y⁴ is carbon bonded to R^{4s}, and one of Y¹, Y², Y³, and Y⁴ is carbon bonded to hydrogen. In some such embodiments, the compound corresponds in structure to one of the following formulas:

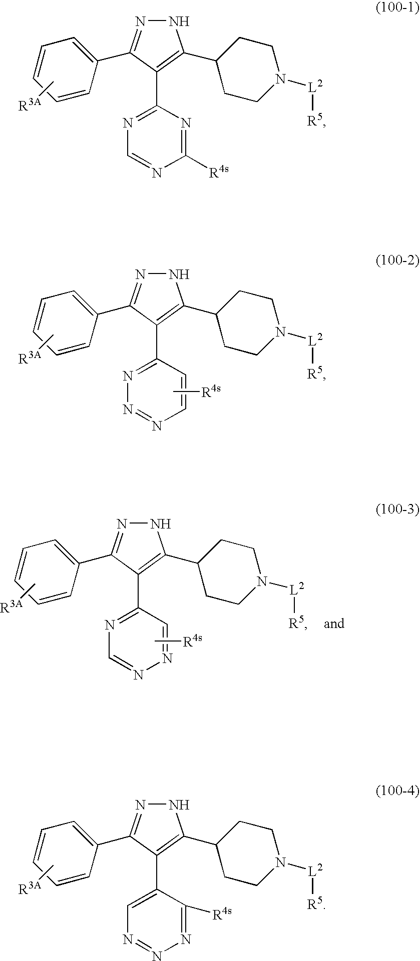

In such embodiments, for example, the compound corresponds in structure to one of the following formulas:

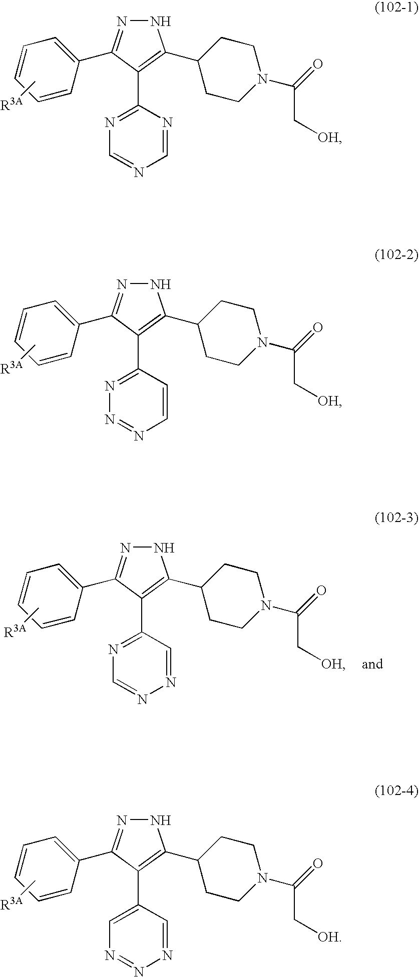

In some particularly preferred embodiments, the compound corresponds in structure to the following formula:

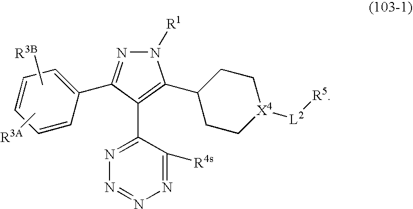

In some such embodiments, the compound corresponds in structure to the following formula:

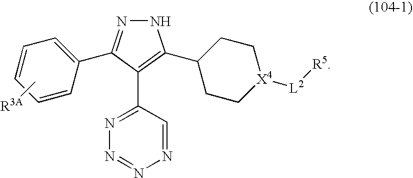

These embodiments include, for example, compounds corresponding in structure to the following formula:

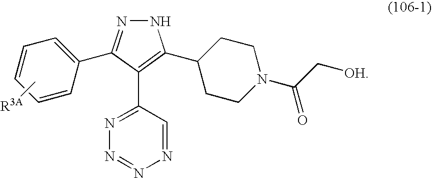

### Preferred Embodiment No. 5

In some preferred embodiments, the compound corresponds in structure to the following formula:

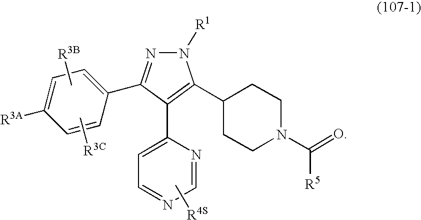

Here:

R^{3A} is hydroxy, cyano, amino, monomethylamino, monoethylamino, dimethylamino, diethylamino, N-methyl-N-ethyl-amino, methyl, ethyl, haloethyl, propyl, halopropyl, aminomethyl, aminoethyl, hydroxymethyl, hydroxyethyl, methoxy, ethoxy, haloethoxy, methoxymethyl, or halomethoxymethyl.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁵ is hydroxyalkyl.

### Particularly Preferred Compounds of Embodiment No. 5

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, R^{4s} is hydrogen.

In some particularly preferred embodiments, R⁵ is C₁-C₆-hydroxyalkyl.

In some particularly preferred embodiments, R⁵ is hydroxymethyl. Examples such compounds include, for example, those corresponding in structure to the following formulas:

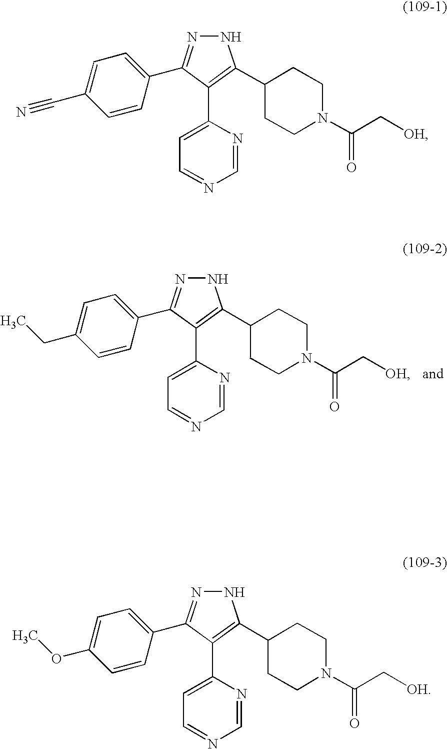

### Preferred Embodiment No. 6

In some preferred embodiments, the compound corresponds in structure to the following formula:

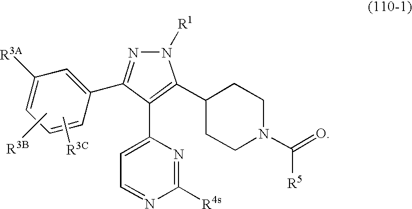

Here:

R^{3A} is hydroxy, cyano, amino, monomethylamino, monoethylamino, dimethylamino, diethylamino, N-methyl-N-ethyl-amino, methyl, ethyl, haloethyl, propyl, halopropyl, aminomethyl, aminoethyl, hydroxymethyl, hydroxyethyl, methoxy, halomethoxy, ethoxy, haloethoxy, methoxymethyl, or halomethoxymethyl.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁵ is hydroxyalkyl.

### Particularly Preferred Compounds of Embodiment No. 6

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, R^{4s} is hydrogen.

In some particularly preferred embodiments, R⁵ is C₁-C₆-hydroxyalkyl.

In some particularly preferred embodiments, R⁵ is hydroxymethyl.

Examples of particularly preferred compounds include those corresponding in structure to the following formulas:

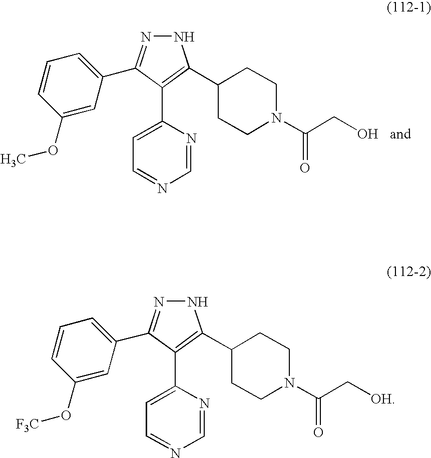

### Preferred Embodiment No. 7

In some preferred embodiments, the compound corresponds in structure to the following formula:

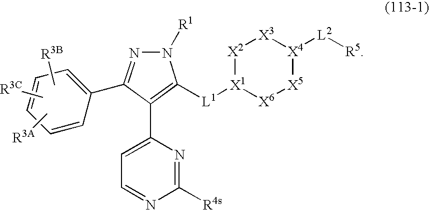

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁵ is phosphonooxyalkyl, monoalkylphosphonooxyalkyl, dialkylphosphonooxyalkyl, aminoalkylcarbonyloxyalkyl, monoalkylaminoalkylcarbonyloxyalkyl, dialkylaminoalkylcarbonyloxyalkyl, phenylalkyl substituted with alkylcarbonyloxy, or tetrahydrofuranyl.

### Particularly Preferred Compounds of Embodiment No. 7

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, R^{4s} is hydrogen.

Examples of particularly preferred compounds include those corresponding in structure to the following formulas:

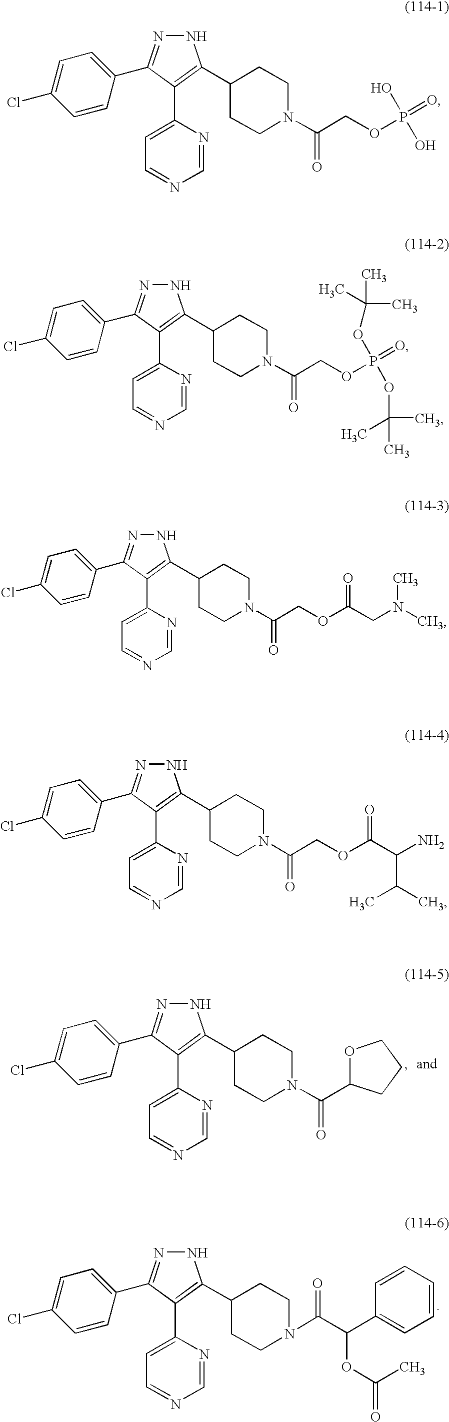

In some embodiments, the preferred optical isomer of the compound of Formula (114-6) corresponds in structure to the following formula:

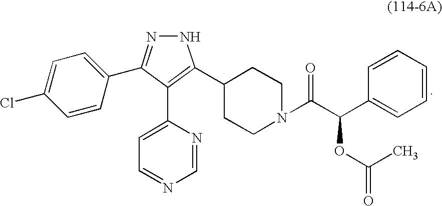

### Preferred Embodiment No. 8

In some preferred embodiments, the compound corresponds in structure to formula:

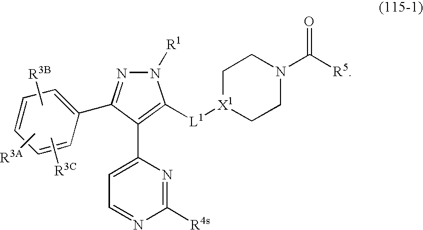

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{4s} is hydrogen, alkyl, aminoalkyl, alkoxyalkyl, cycloalkylalkyl, arylalkyl, hetercycloalkylalkyl, heteroarylalkyl, amino, alkylamino, aminoalkylamino, alkoxyalkylamino, cycloalkylamino, arylamino, heterocycloalkylamino, heteroarylamino, hydroxy, alkoxy, aminoalkoxy, alkoxyalkoxy, cycloalkyloxy, aryloxy, heterocycloalkyloxy, heteroaryloxy, thiol, alkylthio, cycloalkylthio, arylthio, heterocycloalkylthio, heteroarylthio, aminosulfonyl, alkylsulfonyl, cycloalkylsulfonyl, arylsufonyl, heterocycloalkylsulfonyl, or heteroarylsulfonyl. Any such substituent optionally substituted with one or more substituents independently selected from the group consisting of hydroxy, cyano, and alkyl.

R⁵ is alkylcarbonyloxyalkyl.

### Particularly Preferred Compounds of Embodiment No. 8

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, R^{4s} is hydrogen.

In some particularly preferred embodiments, X¹ is carbon bonded to hydrogen.

In some particularly preferred embodiments, L¹ is a bond.

In some particularly preferred embodiments, R⁵ is methylcarbonyloxymethyl. Examples of such compounds include those corresponding in structure to the following formulas:

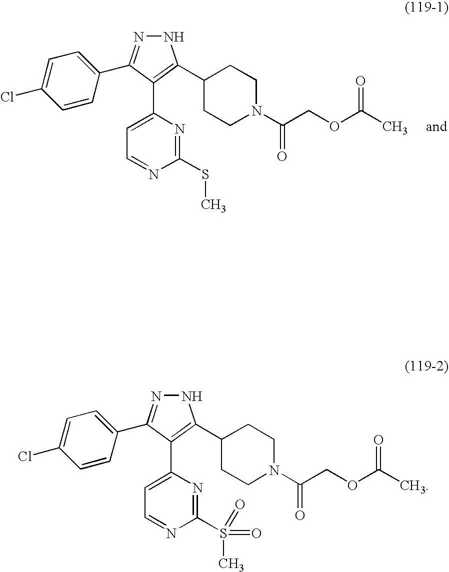

### Preferred Embodiment No. 9

In some preferred embodiments, the compound corresponds in structure to the following formula:

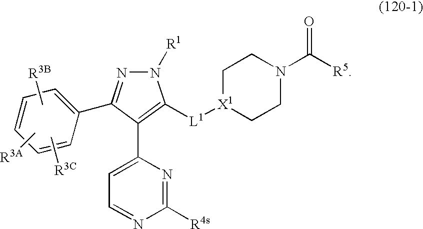

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{4s} is hydrogen, C₁-C₆-alkyl, aminoalkyl, alkoxyalkyl, cycloalkylalkyl, hetercycloalkylalkyl, heteroarylalkyl, aminoalkylamino, alkoxyalkylamino, cycloalkylamino, heterocycloalkylamino, heteroarylamino, hydroxy, C₂-C₆-alkoxy, aminoalkoxy, alkoxyalkoxy, cycloalkyloxy, heterocycloalkyloxy, heteroaryloxy, thiol, C₂-C₆-alkylthio, cycloalkylthio, heterocycloalkylthio, heteroarylthio, aminosulfonyl, C₂-C₆-alkylsulfonyl, cycloalkylsulfonyl, heterocycloalkylsulfonyl, or heteroarylsulfonyl. Any such substituent optionally is substituted with one or more substituents independently selected from the group consisting of hydroxy, cyano, and alkyl.

R⁵ is hydroxyalkyl.

### Particularly Preferred Compounds of Embodiment No. 9

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, R^{4s} is hydrogen.

In some particularly preferred embodiments, X¹ is carbon bonded to hydrogen.

In some particularly preferred embodiments, L¹ is a bond.

In some particularly preferred embodiments, R⁵ is hydroxymethyl. Examples such compounds include those corresponding in structure to the following formulas:

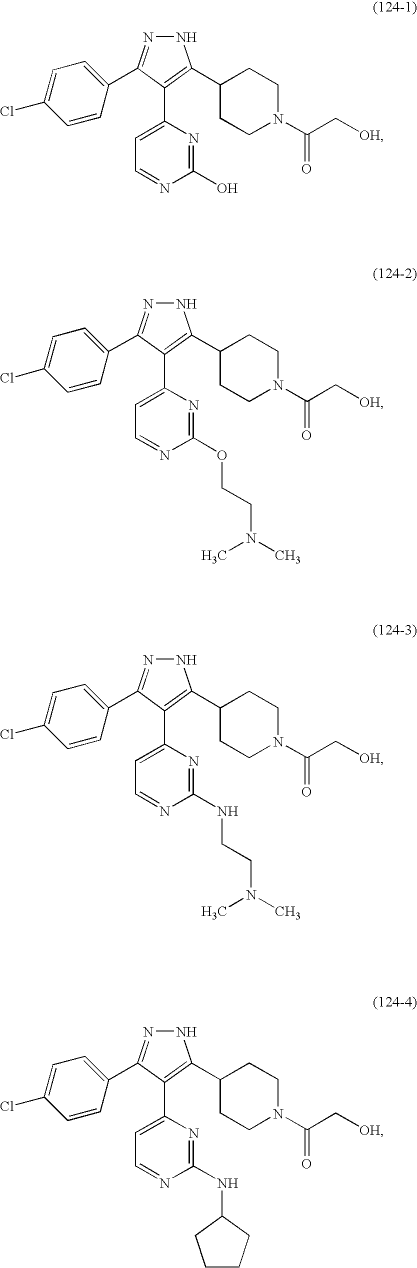

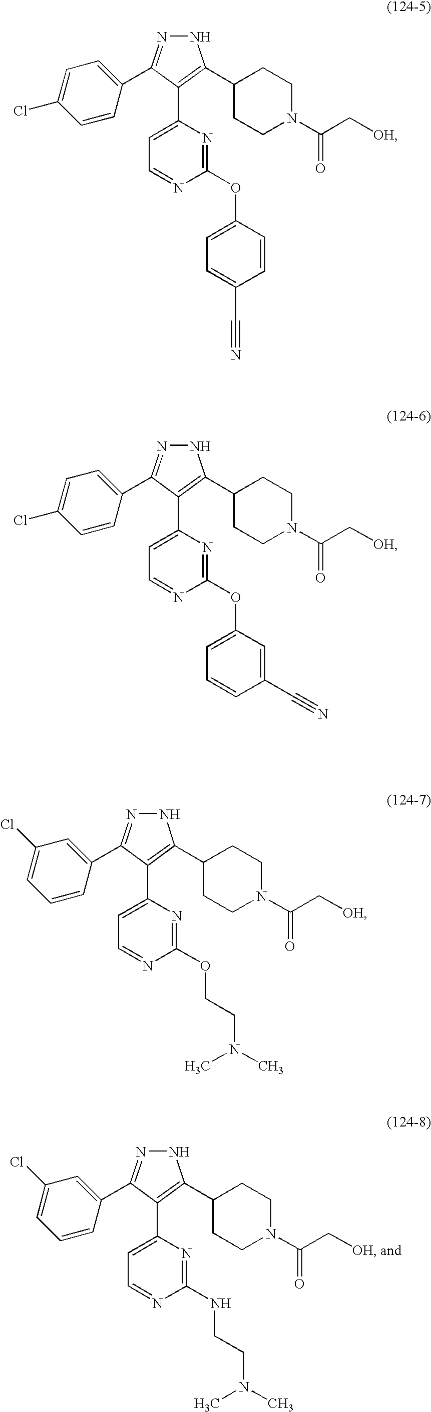

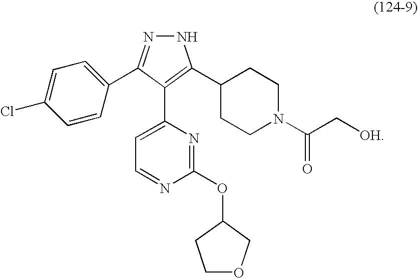

In some embodiments, the preferred optical isomer of the compound of Formula (124-9) corresponds in structure to the following formula:

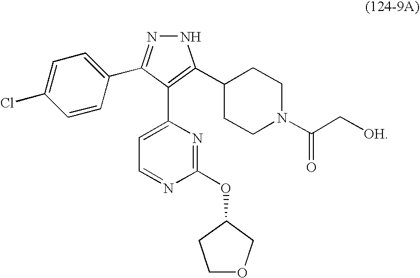

In some embodiments, the preferred optical isomer of the compound of Formula (124-9) corresponds in structure to the following formula:

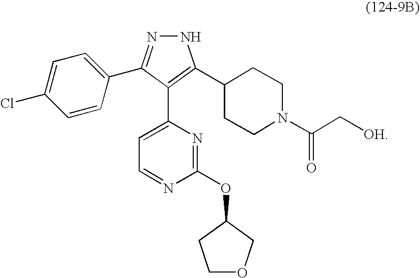

### Preferred Embodiment No. 10

In some preferred embodiments, the compound corresponds in structure to the following formula:

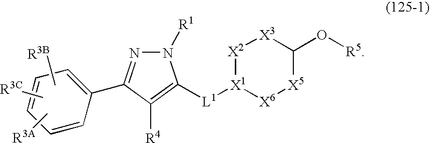

Here,

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridazinyl, pyrazinyl, pyrimidinyl, triazinyl, tetrazinyl, benzazinyl, benzodiazinyl, naphthyridinyl, pyridopyridinyl, pyrinyl, maleimidyl, pyridonyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

R⁵ is hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

### Particularly Preferred Compounds of Embodiment No. 10

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, X¹ is carbon bonded to hydrogen.

In some particularly preferred embodiments, L¹ is a bond.

In some particularly preferred embodiments, the compound corresponds in structure to the following formula:

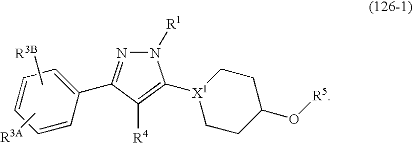

In some particularly preferred embodiments, the compound corresponds in structure to the following formula:

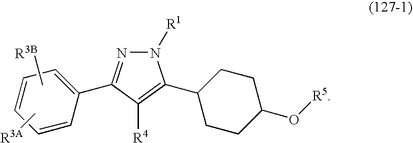

In some particularly preferred embodiments, the compound corresponds in structure to the following formula:

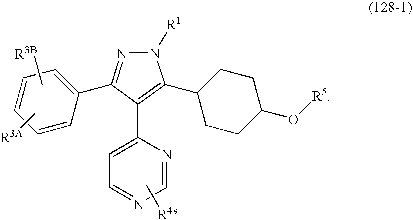

In some particularly preferred embodiments, the compound corresponds in structure to the following formula:

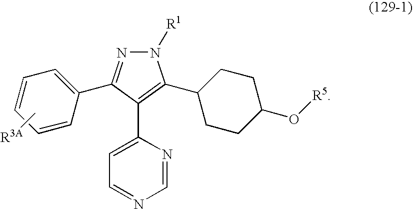

In some particularly preferred embodiments, R⁵ is hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl.

In some particularly preferred embodiments, R⁵ is hydrogen, alkenyl, or alkylcarbonylalkyl. Any such substituent optionally is substituted with one or more substituents independently selected from the group consisting of halogen, hydroxy, alkoxy, and haloalkoxy.

In some particularly preferred embodiments, the compound corresponds in structure to one of the following formulas:

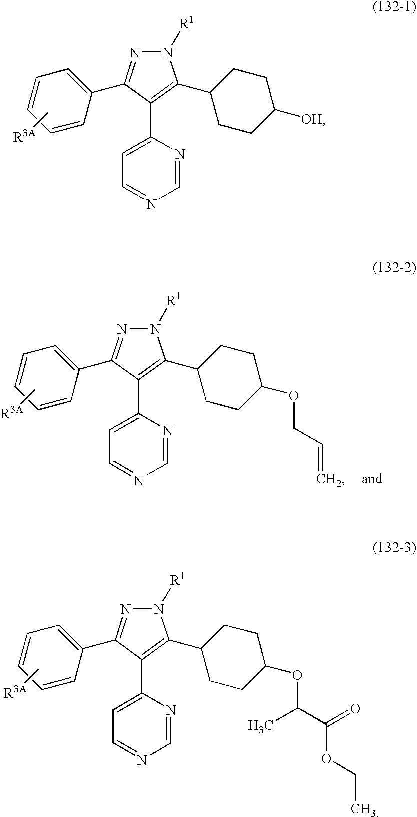

In some embodiments, the preferred isomer of the compound of Formula (132-1) corresponds in structure to the following formula:

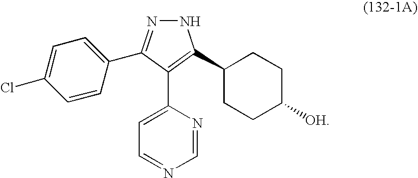

In some embodiments, the preferred isomer of the compound of Formula (132-2) corresponds in structure to the following formula:

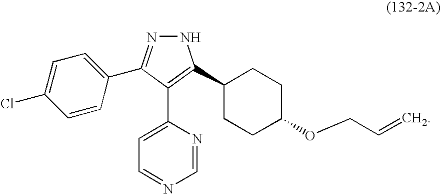

In some embodiments, the preferred isomer of the compound of Formula (132-3) corresponds in structure to the following formula:

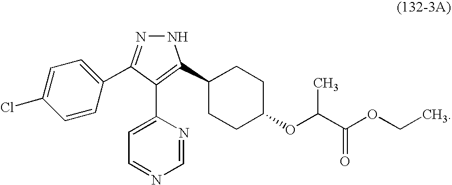

### Preferred Embodiment No. 11

In some preferred embodiments, the compound corresponds in structure to the following formula:

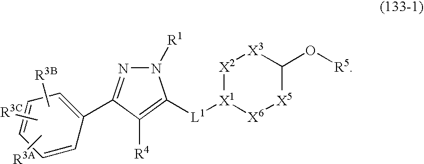

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

R⁵ is alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

### Particularly Preferred Compounds of Embodiment No. 11

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, X¹ is carbon bonded to hydrogen.

In some particularly preferred embodiments, L¹ is a bond.

In some particularly preferred embodiments, the compound corresponds in structure to the following formula:

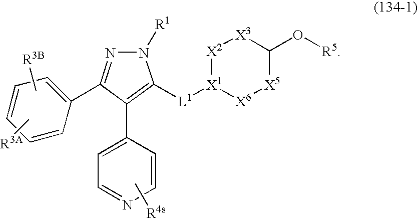

In some particularly preferred embodiments, the compound corresponds in structure to the following formula:

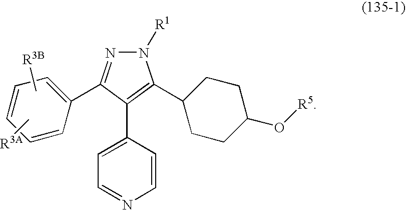

### Preferred Embodiment No. 12

In some preferred embodiments, the compound corresponds in structure to the following formula:

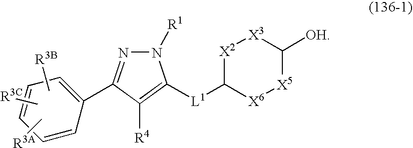

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 12

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, L¹ is a bond.

In some particularly preferred embodiments, the compound corresponds in structure to the following formula:

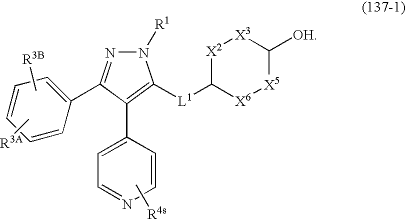

In some particularly preferred embodiments, the compound corresponds in structure to the following formula:

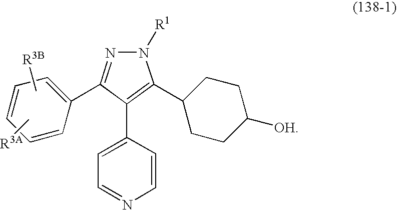

### Preferred Embodiment No. 13

In some preferred embodiments, the compound corresponds in structure to the following formula:

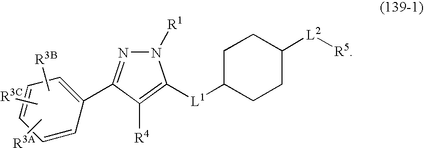

Here:

L² is —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyrimidinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

R⁵ is haloalkyl, hydroxyalkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

### Particularly Preferred Compounds of Embodiment No. 13

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, L¹ is a bond.

In some particularly preferred embodiments, the compound corresponds in structure to the following formula:

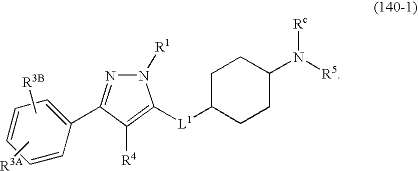

In some particularly preferred embodiments, the compound corresponds in structure to the following formula:

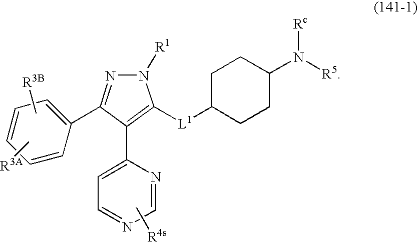

In some particularly preferred embodiments, the compound corresponds in structure to the following formula:

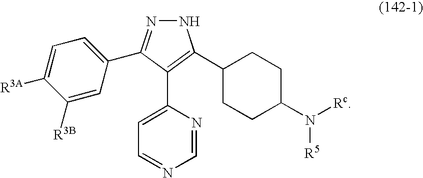

In some such particularly preferred embodiments, R^{a} is alkyl. The compound may, for example, correspond in structure to the following formula:

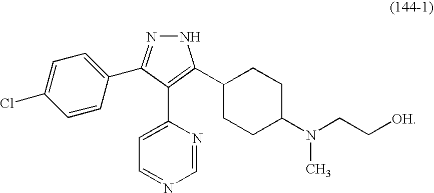

In some embodiments, the preferred isomer of the compound of Formula (144-1) corresponds in structure to the following formula:

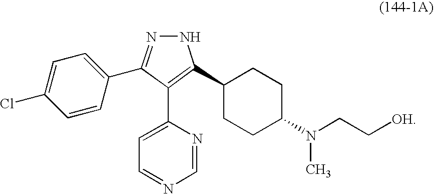

In other such particularly preferred embodiments, R^{a} is hydrogen. The compound may, for example, correspond in structure to one of the following formulas:

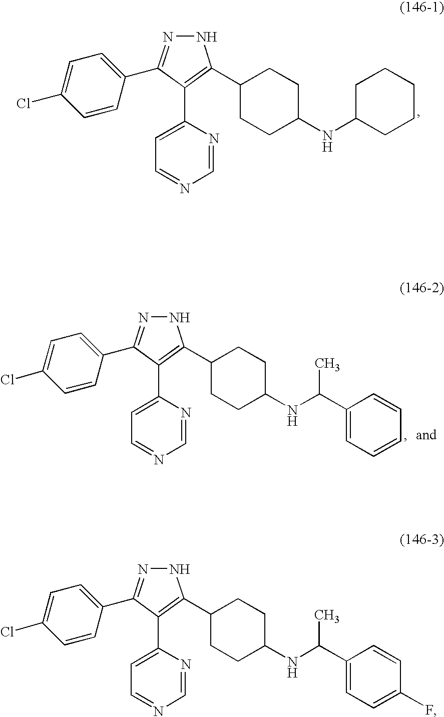

In some embodiments, the preferred isomer of the compound of Formula (146-1) corresponds in structure to the following formula:

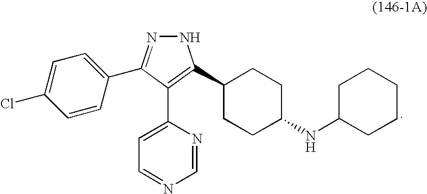

In some embodiments, the preferred isomer of the compound of Formula (146-1) corresponds in structure to the following formula:

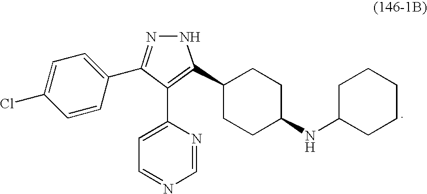

In some embodiments, the preferred isomer of the compound of Formula (146-2) corresponds in structure to the following formula:

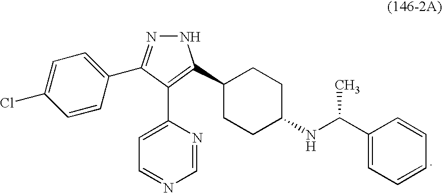

In some embodiments, the preferred isomer of the compound of Formula (146-2) corresponds in structure to the following formula:

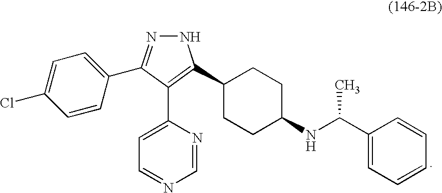

In some embodiments, the preferred isomer of the compound of Formula (146-2) corresponds in structure to the following formula:

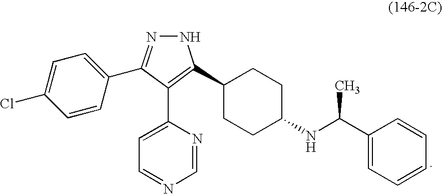

In some embodiments, the preferred isomer of the compound of Formula (146-2) corresponds in structure to the following formula:

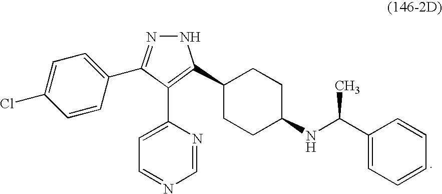

In some embodiments, the preferred isomer of the compound of Formula (146-3) corresponds in structure to the following formula:

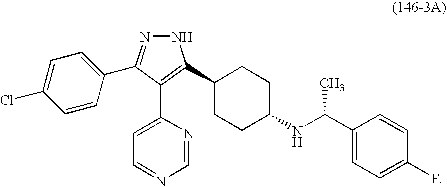

In some embodiments, the preferred isomer of the compound of Formula (146-3) corresponds in structure to the following formula:

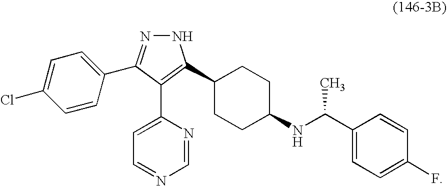

In some embodiments, the preferred isomer of the compound of Formula (146-3) corresponds in structure to the following formula:

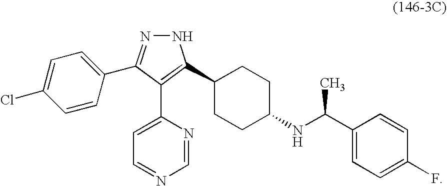

In some embodiments, the preferred isomer of the compound of Formula (146-3) corresponds in structure to the following formula:

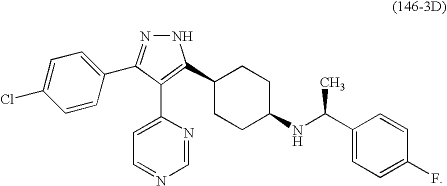

### Preferred Embodiment No. 14

In some preferred embodiments, the compound corresponds in structure to the following formula:

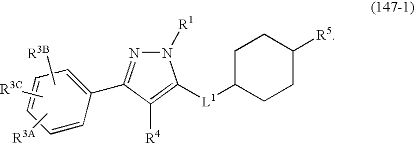

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyrimidinyl or pyridinyl. The pyrimidinyl or pyridinyl optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, or carbocyclylhydrazinyl. Any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 14

In some particularly preferred embodiments, R¹ is hydroxyalkyl.

In some particularly preferred embodiments, R¹ is hydrogen.

In some particularly preferred embodiments, R^{3A} is halogen or haloalkyl; and R^{3B} hydrogen, halogen, or haloalkyl.

In some particularly preferred embodiments, R^{3C} is hydrogen.

In some particularly preferred embodiments, L¹ is a bond.

In some particularly preferred embodiments, R⁵ is hydroxyalkyl.

In some particularly preferred embodiments, the compound corresponds in structure to the following formula:

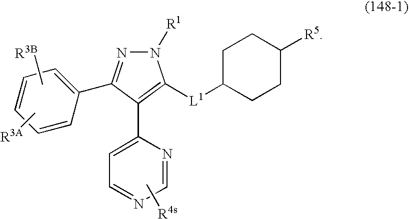

Such compounds include, for example, those corresponding in structure to the following formula:

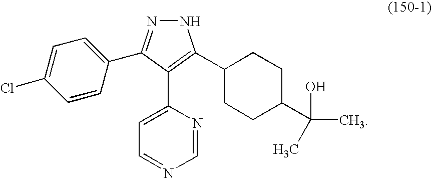

In some embodiments, the preferred isomer of the compound of Formula (150-1) corresponds in structure to the following formula:

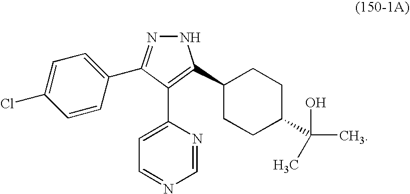

In some particularly preferred embodiments, R⁵ is heterocyclyl optionally substituted with one or more substituents independently selected from the group consisting of halogen, hydroxy, alkyl, haloalkyl, hydroxyalkyl, alkoxy, and haloalkoxy.

In some particularly preferred embodiments, the compound corresponds in structure to the following formula:

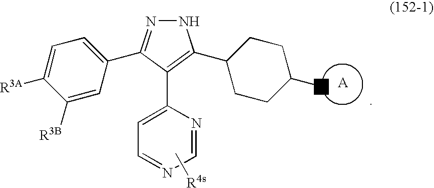

Here, the ring structure A is a heterocyclyl ring that contains a nitrogen bonded to the cyclohexyl, and optionally is substituted with one or more substituents independently selected from the group consisting of halogen, hydroxy, alkyl, haloalkyl, hydroxyalkyl, alkoxy, and haloalkoxy. The compound may, for example, correspond in structure to the following formula:

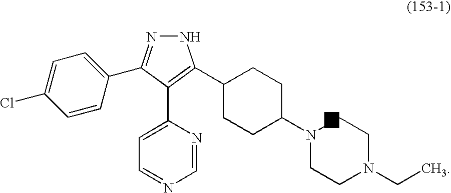

In some embodiments, the preferred isomer of the compound of Formula (153-1) corresponds in structure to the following formula:

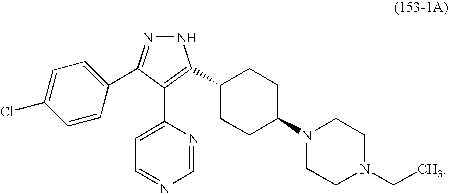

In some particularly preferred embodiments, the compound corresponds in structure to the following formula:

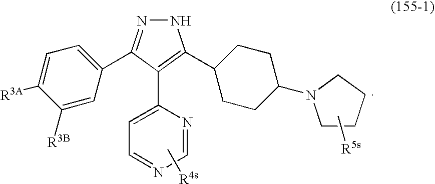

Here, R^{5s} is halogen, hydroxy, alkyl, haloalkyl, hydroxyalkyl, alkoxy, or haloalkoxy. The compound may, for example, correspond in structure to the following formula:

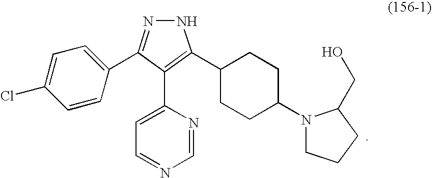

In some embodiments, the preferred isomer of the compound of Formula (156-1) corresponds in structure to the following formula:

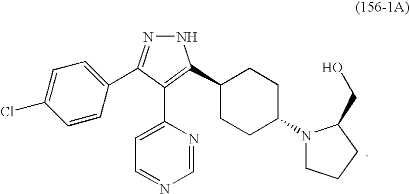

In some particularly preferred embodiments, the compound corresponds in structure to the following formula:

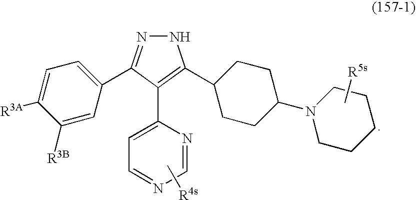

Here, R^{5s} is halogen, hydroxy, alkyl, haloalkyl, hydroxyalkyl, alkoxy, or haloalkoxy. The compound may, for example, correspond in structure to the following formula:

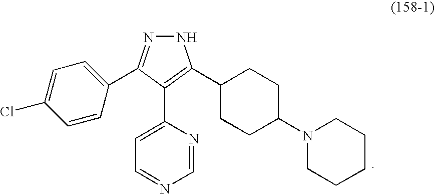

In some embodiments, the preferred isomer of the compound of Formula (158-1) corresponds in structure to the following formula:

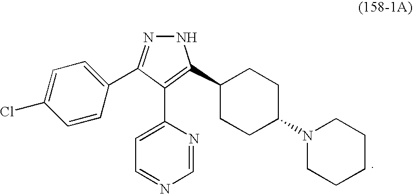

### Preferred Embodiment No. 15

In some preferred embodiments:

L² is a bond, —O—, —S—, —S(O)—, —N(R^{a})—, —N(R^{a})—C(O)—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, or —CH₂—S—.

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyrimidinyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. Any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl. Any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

R⁵ is carbocyclyl, carbocyclylalkyl, heterocyclyl, or heterocyclylalkyl. Any such substituent optionally is substituted with one or more substituents independently selected from the group consisting of halogen, hydroxy, alkyl, haloalkyl, hydroxyalkyl, alkoxy, and haloalkoxy.

### Particularly Preferred Compounds of Embodiment No. 15

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, L¹ is a bond.

In some particularly preferred embodiments, the compound corresponds in structure to the following formula:

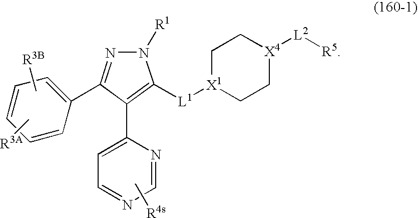

In some particularly preferred embodiments, the compound corresponds in structure to the following formula:

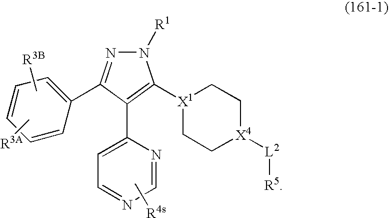

In some particularly preferred embodiments, the compound corresponds in structure to the following formula:

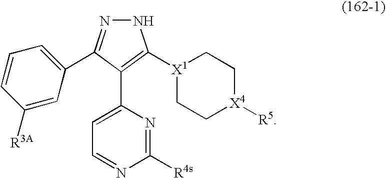

In some particularly preferred embodiments, the compound corresponds in structure to the following formula:

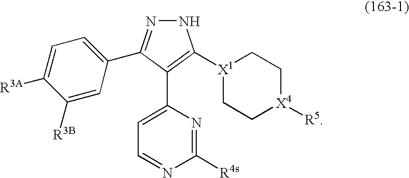

In some particularly preferred embodiments, the compound corresponds in structure to the following formula:

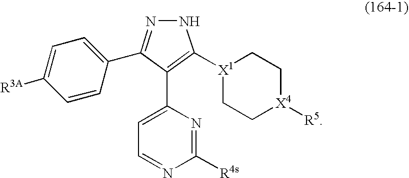

In some particularly preferred embodiments, the compound corresponds in structure to the following formula:

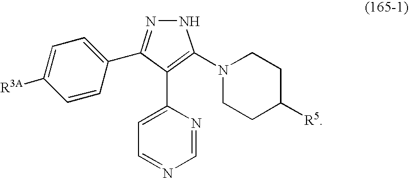

In some particularly preferred embodiments, the compound corresponds in structure to the following formula:

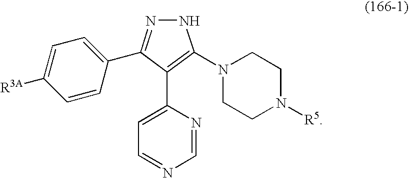

In some particularly preferred embodiments, the compound corresponds in structure to the following formula:

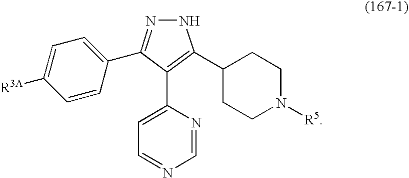

Such compounds include, for example, those corresponding in structure to the following formulas:

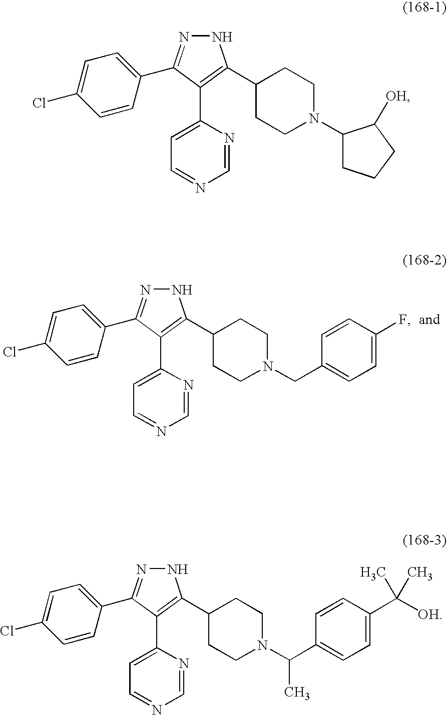

### Preferred Embodiment No. 16

In some preferred embodiments:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁵ is substituted methyl. The methyl is substituted with:

two substituents independently selected from the group consisting of hydroxy, alkoxy, hydroxymethyl, hydroxyethyl, alkoxymethyl, alkoxyethyl, tetrahydrofuranyl, and tetrahydrofuranylmethyl, wherein any such substituent optionally is substituted with one or more substituents independently selected from the group consisting of halogen, hydroxy, alkyl, haloalkyl, hydroxyalkyl, alkoxy, and haloalkoxy; or

a substituent selected from the group consisting of alkoxyethoxy, hydroxyethoxy, alkoxypropoxy, and hydroxypropoxy, wherein any such substituent optionally is substituted with one or more substituents independently selected from the group consisting of halogen, hydroxy, alkyl, haloalkyl, hydroxyalkyl, alkoxy, and haloalkoxy.

The methyl optionally is further substituted with hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl. Any such optional substituent optionally is substituted with one or more substituents independently selected from the group consisting of halogen, hydroxy, alkyl, haloalkyl, hydroxyalkyl, alkoxy, and haloalkoxy.

### Particularly Preferred Compounds of Embodiment No. 16

In some particularly preferred embodiments, R¹ is hydroxyalkyl.

In some particularly preferred embodiments, R¹ is hydrogen.

In some particularly preferred embodiments, R^{3C} is hydrogen.

In some particularly preferred embodiments, L¹ is a bond.

In some particularly preferred embodiments, the compound corresponds in structure to the following formula:

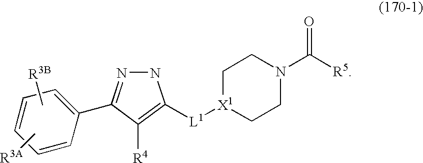

In some particularly preferred embodiments, the compound corresponds in structure to the following formula:

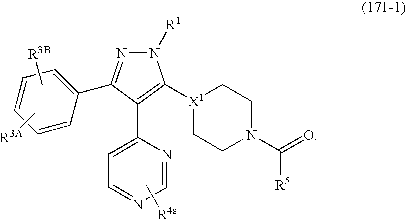

In some particularly preferred embodiments, R⁵ is a radical corresponding in structure to one of the following formulas

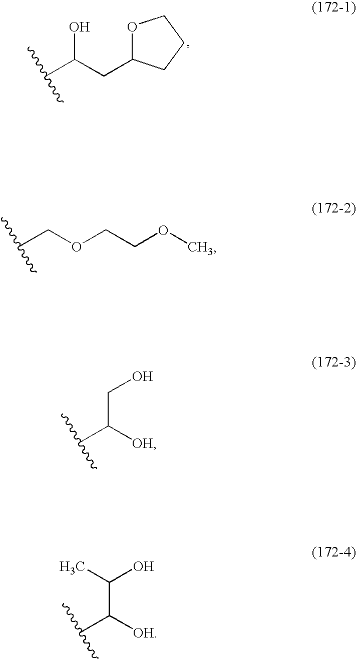

Examples of such compounds include those corresponding in structure to the following formulas:

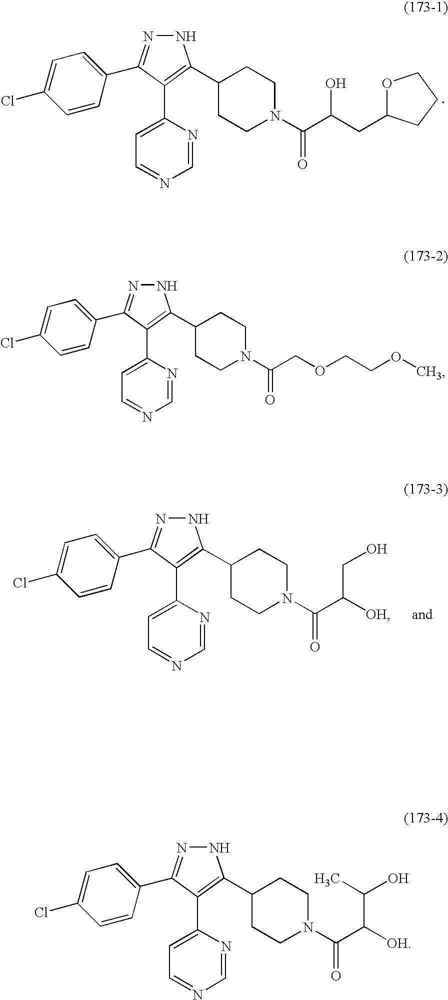

In some embodiments, the preferred isomer of the compound of Formula (173-3) corresponds in structure to the following formula:

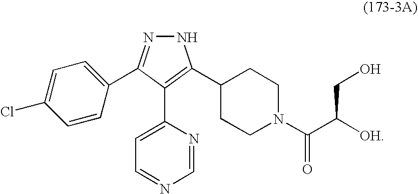

In some embodiments, the preferred isomer of the compound of Formula (173-3) corresponds in structure to the following formula:

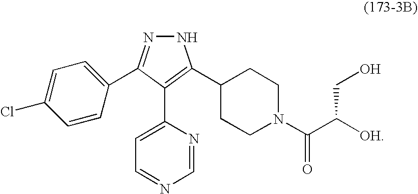

In some embodiments, the preferred isomer of the compound of Formula (173-4) corresponds in structure to the following formula:

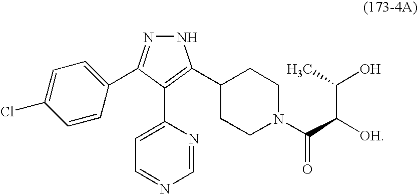

In some embodiments, the preferred isomer of the compound of Formula (173-4) corresponds in structure to the following formula:

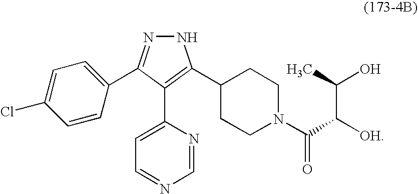

### Preferred Embodiment No. 17

In some preferred embodiments:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. These ring structures are:

substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 17

In some particularly preferred embodiments, R¹ is hydroxyalkyl.

In some particularly preferred embodiments, R¹ is hydrogen.

In some particularly preferred embodiments, R^{3A} is halogen.

In some particularly preferred embodiments, R^{3A} is chloro.

In some particularly preferred embodiments, R^{3B} is hydrogen.

In some particularly preferred embodiments, R^{3C} is hydrogen.

In some particularly preferred embodiments, L¹ is a bond.

In some particularly preferred embodiments, R⁴ is pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. In these embodiments:

any such substituent is substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

any such substituent optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, R⁴ is pyrimidinyl. In these embodiments:

the pyrimidinyl is substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:

any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

the pyrimidinyl optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, R⁴ is pyrimidinyl substituted with heterocyclyloxy, heterocyclylalkoxy, cycloalkylamino, cyanoaryloxy, alkylaminoalkylamino, or carbocyclylalkylheterocyclylamino. Any such substituent optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

In some particularly preferred embodiments, R⁴ is pyrimidinyl substituted with a substituent independently selected from the group consisting of heterocyclyloxy, heterocyclylalkoxy, cycloalkylamino, cyanoaryloxy, dialkylaminoalkylamino, and carbocyclylalkylheterocyclylamino.

In some particularly preferred embodiments, the compound corresponds in structure to the following formula:

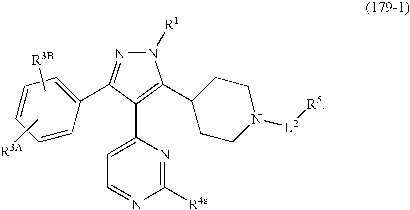

In some such embodiments, for example, the compound corresponds in structure to the following formula:

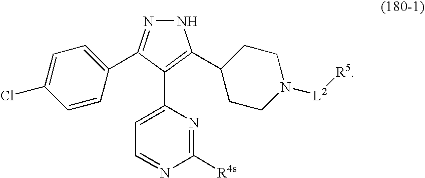

In these embodiments, R^{4s} is heterocyclyloxy, heterocyclylalkoxy, cycloalkylamino, cyanoaryloxy, dialkylaminoalkylamino, or carbocyclylalkylheterocyclylamino. In some such embodiments, for example, R^{4s} is tetrahydrofuranyloxy, cyanophenyloxy, morpholinylethyloxy, cyclopentylamino, dimethylaminoethylamino, or phenylmethylpiperidinylamino.

In some particularly preferred embodiments, -L²-R⁵ is hydrogen, methyl, or butyloxycarbonyl.

In some particularly preferred embodiments, -L²-R⁵ is hydroxymethylcarbonyl.

Examples of particularly preferred compounds include those corresponding in structure to the following formulas:

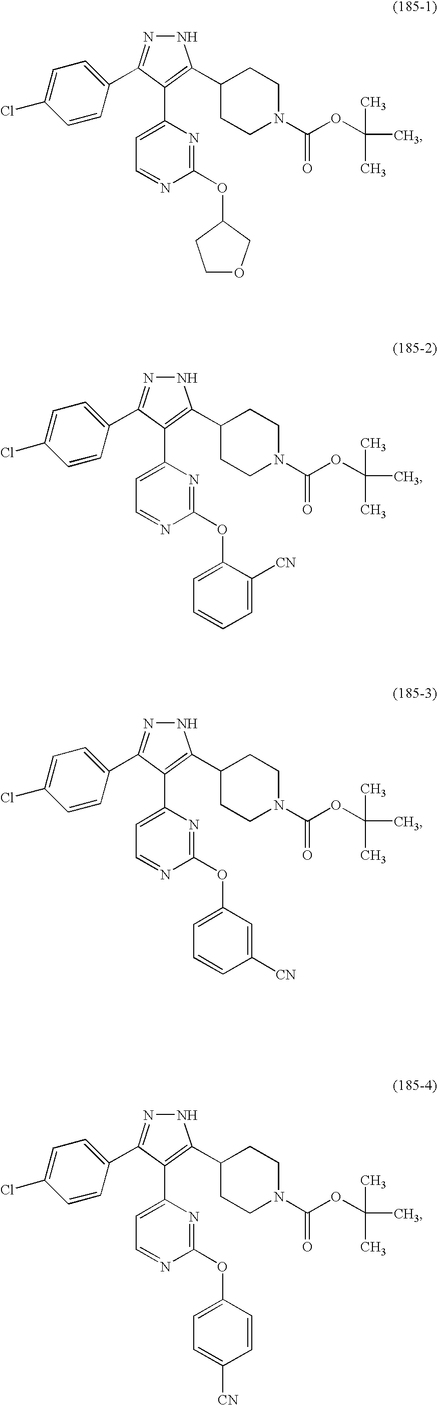

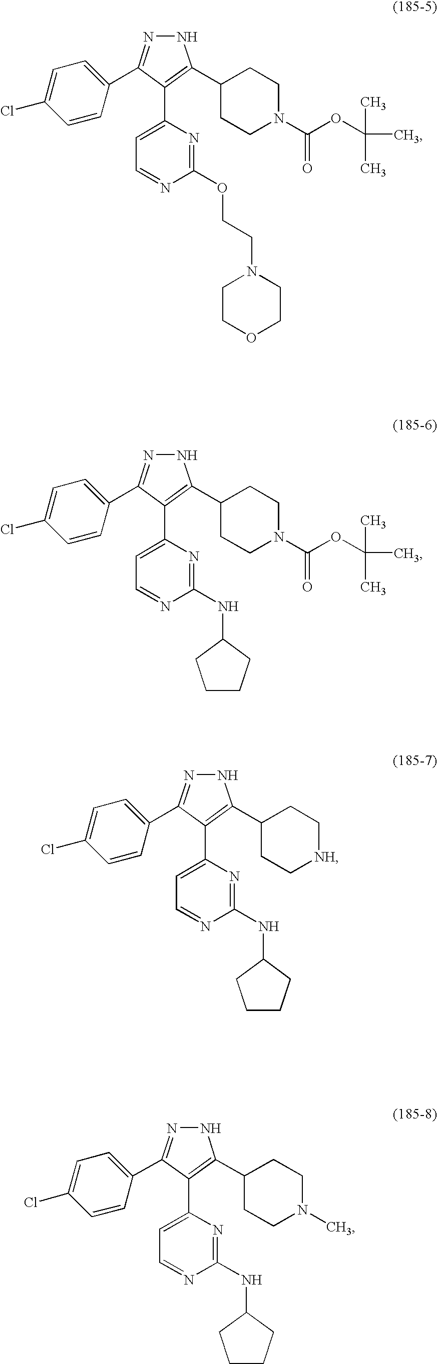

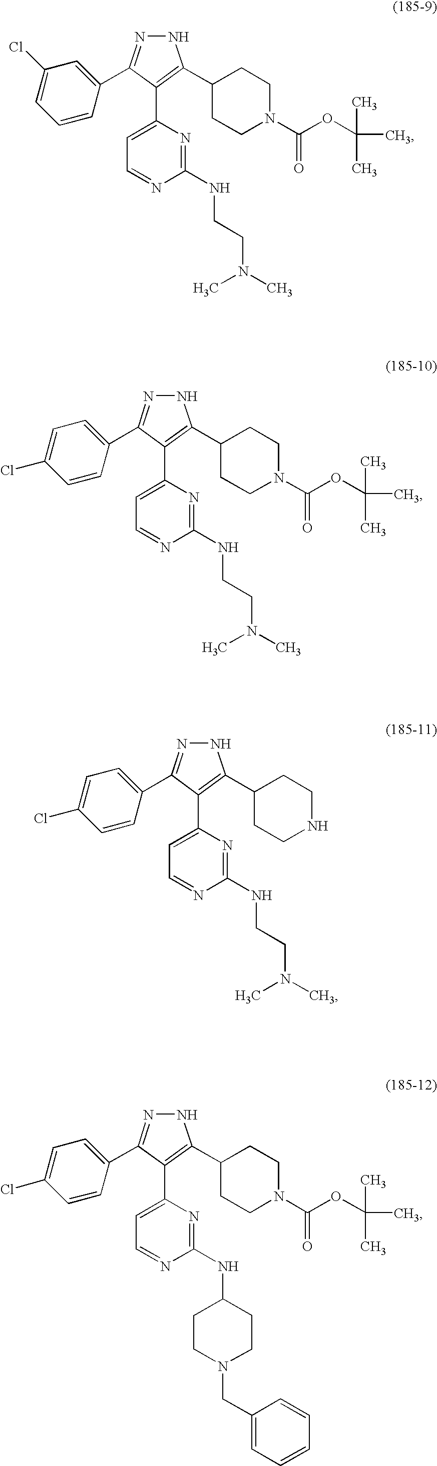

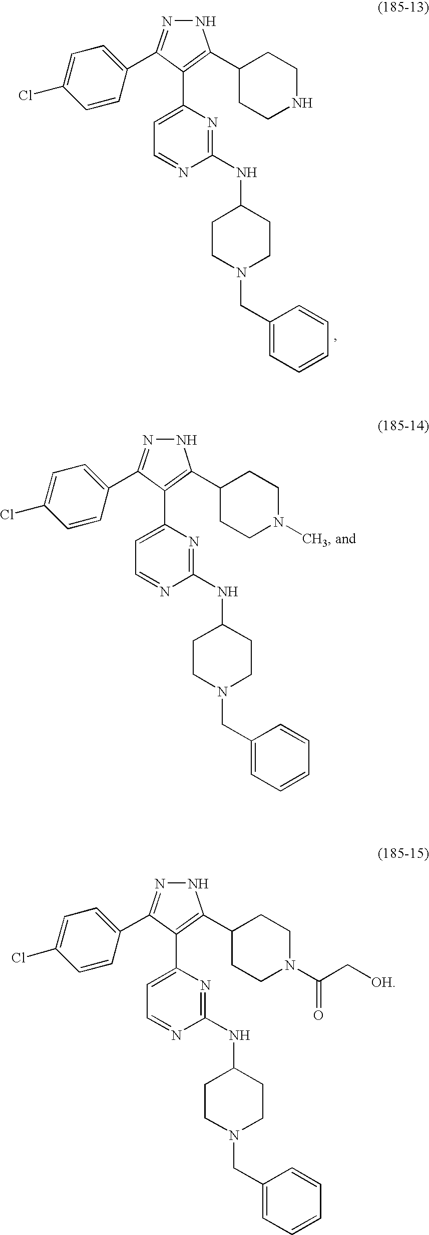

In some embodiments, the preferred optical isomer of the compound of Formula (185-1) corresponds in structure to the following formula:

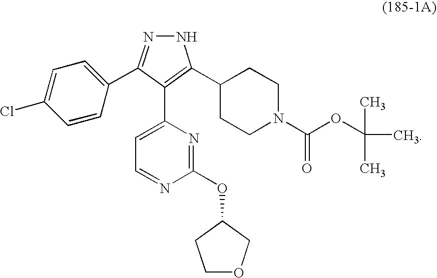

In some embodiments, the preferred optical isomer of the compound of Formula (185-1) corresponds in structure to the following formula:

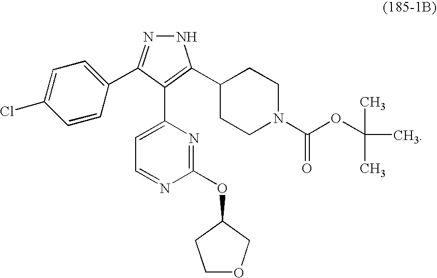

### Preferred Embodiment No. 18

In some preferred embodiments, the compound corresponds in structure to the following formula:

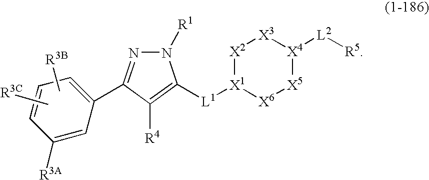

Here:

R^{3A} is halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R^{3B} is hydrogen, halogen, hydroxy, cyano, amino, alkyl, aminoalkyl, monoalkylamino, dialkylamino, alkoxy, or alkoxyalkyl. Any carbon of the alkyl, aminoalkyl, monoalkylamino, dialkylamino, alkoxy, or alkoxyalkyl optionally is substituted with one or more substituents independently selected from the group consisting of halogen, hydroxy, and cyano.

R⁴ is pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, or the sulfone of thiomorpholinyl. These ring structure are:

substituted with one or more independently selected alkylaminoalkoxy optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and

optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclyl alkyl amino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:

any such optional substituent is, in turn, optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

### Particularly Preferred Compounds of Embodiment No. 18

In some particularly preferred embodiments, R¹ is hydroxyalkyl.

In some particularly preferred embodiments, R¹ is hydrogen.

In some particularly preferred embodiments, R^{3A} is halogen.

In some particularly preferred embodiments, R^{3A} is chloro.

In some particularly preferred embodiments, R^{3B} is hydrogen.

In some particularly preferred embodiments, R^{3C} is hydrogen.

In some particularly preferred embodiments, L¹ is a bond.

In some particularly preferred embodiments, the compound corresponds in structure to the following formula:

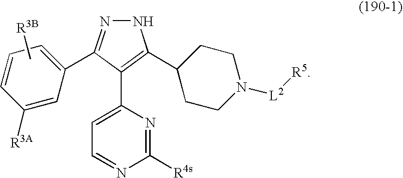

Here, R^{4s} is dialkylaminoalkoxy. In some such embodiments, for example, R^{4s} is dimethylaminoethyloxy.

In some particularly preferred embodiments, -L²-R⁵ is hydrogen, methyl, or butyloxycarbonyl.

In some particularly preferred embodiments, -L²-R⁵ is hydroxymethylcarbonyl.

Examples of particularly preferred compounds include those corresponding in structure to the following formulas:

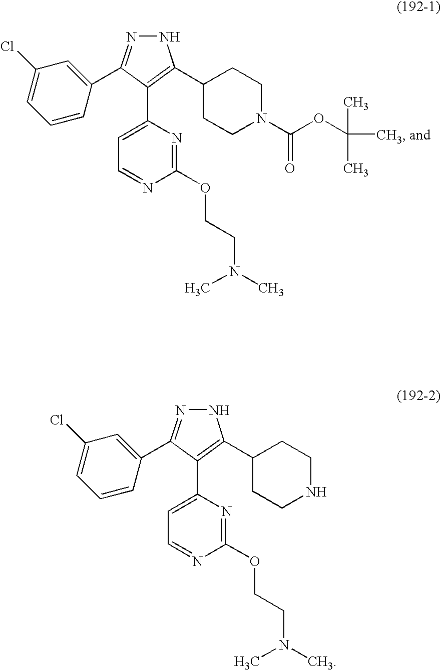

### B. Tautomeric Forms of the Compounds of this Invention

The present invention comprises the tautomeric forms of compounds of Formulas (1-1), (51-1), (70-1), (76-1), (107-1), (110-1), (113-1), (115-1), (120-1), (125-1), (133-1), (136-1), (139-1), (147-1), (159-1), and (169-1). As illustrated below, the pyrazoles of Formula I and I′ are magnetically and structurally equivalent because of the prototropic tautomeric nature of the hydrogen:

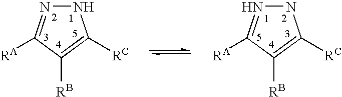

### C. Compounds of this Invention Having One or More Asymmetric Carbons

The present invention also comprises compounds of Formulas (1-1), (51-1), (70-1), (76-1), (107-1), (110-1), (113-1), (115-1), (120-1), (125-1), (133-1), (136-1), (139-1), (147-1), (159-1), and (169-1) having one or more asymmetric carbons. It is known to those skilled in the art that those pyrazoles of the present invention having asymmetric carbon atoms may exist in diastereomeric, racemic, or optically active forms. All of these forms are contemplated within the scope of this invention. More specifically, the present invention includes enantiomers, diastereomers, racemic mixtures, and other mixtures thereof.

### D. Salts of the Compounds of this Invention

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroionic, nitric, carbonic, sulfuric, and phosphoric acid. Suitable organic acids generally include, for example, aliphatic, cycloaliphatic, aromatic, araliphatic, heterocyclyl, carboxyic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically-acceptable base addition salts of the compounds of this invention include, for example, metallic salts and organic salts. Preferred metallic salts include alkali metal (group Ia) salts, alkaline earth metal (group IIa) salts, and other physiological acceptable metal salts. Such salts may be made from aluminum, calcium, lithium, magnesium, potassium, sodium, and zinc. Preferred organic salts may be made from tertiary amines and quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

Particularly preferred salts of the compounds of this invention include hydrochloric acid (HCl) salts, trifluoroacetate (CF₃COOH or “TFA”) salts, mesylate salts, and tosylate salts.

### E. Treating Conditions Using the Compounds of this Invention

This invention is directed, in part, to a method for treating a condition (typically a pathological condition) in a mammal (e.g., a human, companion animal, farm animal, laboratory animal, zoo animal, or wild animal) having or disposed to having such a condition.

In this specification, the phrase “treating a condition” means ameliorating, suppressing, eradicating, reducing the severity of, decreasing the frequency of incidence of, preventing, reducing the risk of, or delaying the onset of the condition.

Some embodiments of this invention are directed to a method for treating a p38-mediated condition. As used herein, the term “p38-mediated condition” refers to any condition (particularly pathological conditions, i.e., diseases and disorders) in which p38 kinase (particularly p38α kinase) plays a role, either by control of p38 kinase itself, or by p38 kinase causing another factor to be released, such as, for example, IL-1, IL-6, or IL-8. A disease state in which, for instance, IL-1 is a major component, and whose production or action is exacerbated or secreted in response to p38, would therefore be considered a disorder mediated by p38.

The compounds of this invention generally tend to be useful for treating pathological conditions that include, but are not limited to:

(a) inflammation;

(b) arthritis, such as rheumatoid arthritis, spondyloarthropathies, gouty arthritis, osteoarthritis, systemic lupus erythematosus arthritis, juvenile arthritis, osteoarthritis, and gouty arthritis;

(c) neuroinflammation;

(d) pain (i.e., use of the compounds as analgesics), such as neuropathic pain;

(e) fever (i.e., use of the compounds as antipyretics);

(f) pulmonary disorders or lung inflammation, such as adult respiratory distress syndrome, pulmonary sarcoisosis, asthma, silicosis, and chronic pulmonary inflammatory disease;

(g) cardiovascular diseases, such as atherosclerosis, myocardial infarction (such as post-myocardial infarction indications), thrombosis, congestive heart failure, cardiac reperfusion injury, and complications associated with hypertension and/or heart failure such as vascular organ damage;

(h) cardiomyopathy;

(i) stroke, such as ischemic and hemorrhagic stroke;

(j) ischemia, such as brain ischemia and ischemia resulting from cardiac/coronary bypass;

(k) reperfusion injury;

(l) renal reperfusion injury;

(m) brain edema;

(n) neurotrauma and brain trauma, such as closed head injury;

(o) neurodegenerative disorders;

(p) central nervous system disorders (these include, for example, disorders having an inflammatory or apoptotic component), such as Alzheimer's disease, Parkinson's disease, Huntington's Disease, amyotrophic lateral sclerosis, spinal cord injury, and peripheral neuropathy;

(q) liver disease and nephritis;

(r) gastrointestinal conditions, such as inflammatory bowel disease, Crohn's disease, gastritis, irritable bowel syndrome, and ulcerative colitis;

(s) ulcerative diseases, such as gastric ulcer;

(t) ophthalmic diseases, such as retinitis, retinopathies (such as diabetic retinopathy), uveitis, ocular photophobia, nonglaucomatous optic nerve atrophy, and age-related macular degeneration (ARMD) (such as ARMD-atrophic form);

(u) ophthalmological conditions, such as corneal graft rejection, ocular neovascularization, retinal neovascularization (such as neovascularization following injury or infection), and retrolental fibroplasia;

(v) glaucoma, such as primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation, and corticosteroid-induced glaucoma;

(w) acute injury to the eye tissue and ocular traumas, such as post-traumatic glaucoma, traumatic optic neuropathy, and central retinal artery occlusion (CRAO);

(x) diabetes;

(y) diabetic nephropathy;

(z) skin-related conditions, such as psoriasis, eczema, burns, dermatitis, keloid formation, scar tissue formation, and angiogenic disorders;

(aa) viral and bacterial infections, such as sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, and herpes virus;

(bb) myalgias due to infection;

(cc) influenza;

(dd) endotoxic shock;

(ee) toxic shock syndrome;

(ff) autoimmune disease, such as graft vs. host reaction and allograft rejections;

(gg) bone resorption diseases, such as osteoporosis;

(hh) multiple sclerosis;

(ii) disorders of the female reproductive system, such as endometriosis;

(jj) pathological, but non-malignant, conditions, such as hemaginomas (such as infantile hemaginomas), angiofibroma of the nasopharynx, and avascular necrosis of bone;

(kk) benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer, skin cancer such as squamus cell and basal cell cancers, prostate cancer, renal cell carcinoma, and other known cancers that affect epithelial cells throughout the body;

(ll) leukemia;

(mm) lymphoma, such as B cell lymphoma;

(nn) systemic lupus erthrematosis (SLE);

(oo) angiogenesis including neoplasia; and

(pp) metastasis.

Some embodiments of this invention are alternatively (or additionally) directed to a method for treating a TNF-mediated condition. As used herein, the term “TNF-mediated condition” refers to any condition (particularly any pathological conditions, i.e., diseases or disorders) in which TNF plays a role, either by control of TNF itself, or by TNF causing another monokine to be released, such as, for example, IL-1, IL-6, and/or IL-8. A disease state in which, for instance, IL-1 is a major component and whose production or action is exacerbated or secreted in response to TNF, would therefore be considered a disorder mediated by TNF.

Examples of TNF-mediated conditions include inflammation (e.g., rheumatoid arthritis), autoimmune disease, graft rejection, multiple sclerosis, a fibrotic disease, cancer, an infectious disease (e.g., malaria, mycobacterial infection, meningitis, etc.), fever, psoriasis, a cardiovascular disease (e.g., post-ischemic reperfusion injury and congestive heart failure), a pulmonary disease, hemorrhage, coagulation, hyperoxic alveolar injury, radiation damage, acute phase responses like those seen with infections and sepsis and during shock (e.g., septic shock, hemodynamic shock, etc.), cachexia, and anorexia. Such conditions also include infectious diseases. Such infectious diseases include, for example, malaria, mycobacterial infection, meningitis. Such infectious diseases also include viral infections, such as HIV, influenza virus, and herpes virus, including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

As TNF-β has close structural homology with TNF-α (also known as cachectin), and because each induces similar biologic responses and binds to the same cellular receptor, the synthesis of both TNF-α and TNF-β tend to be inhibited by the compounds of this invention and thus are herein referred to collectively as “TNF” unless specifically delineated otherwise.

Some embodiments of this invention are alternatively (or additionally) directed to a method for treating a cyclooxygenase-2-mediated condition. As used herein, the term “cyclooxygenase-2-mediated condition” refers to any condition (particularly pathological conditions, i.e., diseases and disorders) in which cyclooxygenase-2 plays a role, either by control of cyclooxygenase-2 itself, or by cyclooxygenase-2 causing another factor to be released. Many cyclooxygenase-2-mediated conditions are known in the art, and include, for example, inflammation and other cyclooxygenase-mediated disorders listed by Carter et al. in U.S. Pat. No. 6,271,253.

In some embodiments of particular interest, the condition treated by the methods of this invention comprises inflammation.

In some embodiments of particular interest, the condition treated by the methods of this invention comprises arthritis.

In some embodiments of particular interest, the condition treated by the methods of this invention comprises rheumatoid arthritis.

In some embodiments of particular interest, the condition treated by the methods of this invention comprises asthma.

In some embodiments of particular interest, the condition treated by the methods of this invention comprises a coronary condition.

In some embodiments of particular interest, the condition treated by the methods of this invention comprises bone loss.

In some embodiments of particular interest, the condition treated by the methods of this invention comprises B cell lymphoma.

A wide variety of methods may be used alone or in combination to administer the pyrazole compounds described above. For example, the compounds may be administered orally, intravascularly (IV), intraperitoneally, subcutaneously, intramuscularly (IM), by inhalation spray, rectally, or topically.

Typically, a compound described in this specification is administered in an amount effective to inhibit p38 kinase (particularly p38α kinase), TNF (particularly TNF-α), and/or cyclooxygenase (particularly cyclooxygenase-2). The preferred total daily dose of the pyrazole compound (administered in single or divided doses) is typically from about 0.01 to about 100 mg/kg, more preferably from about 0.1 to about 50 mg/kg, and even more preferably from about 0.5 to about 30 mg/kg (i.e., mg pyrazole compound per kg body weight). Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular pyrazole compound employed; whether a drug delivery system is utilized; and whether the pyrazole compound is administered as part of a drug combination. Thus, the dosage regimen actually employed can vary widely, and, therefore, can deviate from the preferred dosage regimen set forth above.

The present compounds may be used in co-therapies, partially or completely, in place of other conventional anti-inflammatory, such as together with steroids, cyclooxygenase-2 inhibitors, non-steroidal anti-inflammatory drugs (“NSAIDs”), disease-modifying anti-rheumatic drugs (“DMARDs”), immunosuppressive agents, 5-lipoxygenase inhibitors, leukotriene B4 (“LTB4”) antagonists, and leukotriene A4 (“LTA4”) hydrolase inhibitors.

### F. Pharmaceutical Compositions Containing the Compounds of this Invention

This invention also is directed to pharmaceutical compositions (or “medicaments”) comprising the pyrazole compounds described above (including tautomers of the compounds, and pharmaceutically-acceptable salts of the compounds and tautomers), and to methods for making pharmaceutical compositions comprising those compounds in combination with one or more conventional non-toxic, pharmaceutically-acceptable carriers, diluents, wetting or suspending agents, vehicles, and/or adjuvants (the carriers, diluents, wetting or suspending agents, vehicles, and adjuvants sometimes being collectively referred to in this specification as “carrier materials”); and/or other active ingredients. The preferred composition depends on the method of administration. Formulation of drugs is generally discussed in, for example, Hoover, John E., Remington's Pharmaceutical Sciences (Mack Publishing Co., Easton, Pa.: 1975) (incorporated by reference into this specification). See also, Liberman, H. A., Lachman, L., eds., Pharmaceutical Dosage Forms (Marcel Decker, New York, N.Y., 1980) (incorporated by reference into this specification). In many preferred embodiments, the pharmaceutical composition is made in the form of a dosage unit containing a particular amount of the active ingredient. Typically, the pharmaceutical composition contains from about 0.1 to 1000 mg (and more typically, 7.0 to 350 mg) of the pyrazole compound.

Solid dosage forms for oral administration include, for example, hard or soft capsules, tablets, pills, powders, and granules. In such solid dosage forms, the pyrazole compounds are ordinarily combined with one or more adjuvants. If administered per os, the pyrazole compounds may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation, as may be provided in a dispersion of the compound of this invention in hydroxypropylmethyl cellulose. In the case of capsules, tablets, and pills, the dosage forms also may comprise buffering agents, such as sodium citrate, or magnesium or calcium carbonate or bicarbonate. Tablets and pills additionally may be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the carriers materials mentioned for use in the formulations for oral administration. The pyrazole compounds may be dissolved in water, polyethylene glycol, propylene glycol, ethanol, com oil, cottonseed oil, peanut oil, sesame oil, benzyl alcohol, sodium chloride, and/or various buffers. The pH may be adjusted, if necessary, with a suitable acid, base, or buffer.

The compounds of this invention preferably make up from about 0.075 to about 30% (w/w) (more preferably 0.2 to 20% (w/w), and even more preferably 0.4 to 15% (w/w)) of a pharmaceutical composition used for topical or rectal administration.

Suppositories for rectal administration may be prepared by, for example, mixing a compound of this invention with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols.

“Topical administration” includes transdermal administration, such as via transdermal patches or iontophoresis devices. Compositions for topical administration also include, for example, topical gels, sprays, ointments, and creams.

When formulated in an ointment, the compounds of this invention may be employed with, for example, either a paraffinic or a water-miscible ointment base. When formulated in a cream, the active ingredient(s) may be formulated with, for example, an oil-in-water cream base. If desired, the aqueous phase of the cream base may include, for example at least about 30% (w/w) of a polyhydric alcohol, such as propylene glycol, butane-1,3-diol, mannitol, sorbitol, glycerol, polyethylene glycol, and mixtures thereof.

A topical formulation may include a compound which enhances absorption or penetration of the active ingredient through the skin or other affected areas. Examples of such dermal penetration enhancers include dimethylsulfoxide and related analogs.

When the compounds of this invention are administered by a transdermal device, administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise, for example, a mixture of at least one emulsifier with a fat or an oil or with both a fat and an oil. Preferably, a hydrophilic emulsifier is included together with a lipophilic emulsifier which acts as a stabilizer. It is also preferable to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, given that the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol di ester of coconut fatty acids, isopropyl myristate, decyl oleate, isopropyl palmitate, butyl stearate, 2-ethylhexyl palmitate or a blend of branched chain esters, for example, may be used. These may be used alone or in combination depending on the properties required. Alternatively, high melting point lipids such as white soft paraffin and/or liquid paraffin or other mineral oils may be used. Formulations suitable for topical administration to the eye also include eye drops wherein the compound of this invention is dissolved or suspended in suitable carrier, typically comprising an aqueous solvent. The compounds of this invention are preferably present in such formulations in a concentration of from about 0.5 to about 20% (w/w) (more preferably 0.5 to 10% (w/w), and often even more preferably about 1.5% (w/w)).

Other carrier materials and modes of administration known in the pharmaceutical art may also be used.

### G. Definitions

The term “alkyl” (alone or in combination with another term(s)) means a straight-or branched-chain saturated hydrocarbyl substituent (i.e., a substituent containing only carbon and hydrogen) typically containing from 1 to about 20 carbon atoms, more typically from 1 to about 12 carbon atoms, even more typically from 1 to about 8 carbon atoms, and still even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, and octyl.

The term “alkenyl” (alone or in combination with another term(s)) means a straight- or branched-chain hydrocarbyl substituent containing one or more double bonds and typically from 2 to about 20 carbon atoms, more typically from 2 to about 12 carbon atoms, even more typically from 2 to about 8 carbon atoms, and still even more typically from 2 to about 6 carbon atoms. Examples of such substituents include ethenyl (vinyl); 2-propenyl; 3-propenyl; 1,4-pentadienyl; 1,4-butadienyl; 1-butenyl; 2-butenyl; 3-butenyl; and decenyl.

The term “alkynyl” (alone or in combination with another term(s)) means a straight- or branched-chain hydrocarbyl substituent containing one or more triple bonds and typically from 2 to about 20 carbon atoms, more typically from 2 to about 12 carbon atoms, even more typically from 2 to about 8 carbon atoms, and still even more typically from 2 to about 6 carbon atoms. Examples of such substituents include ethynyl, 1-propynyl, 2-propynyl, decynyl, 1-butynyl, 2-butynyl, 3-butynyl, and 1-pentynyl.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated carbocyclyl substituent containing from 3 to about 14 carbon ring atoms, more typically from 3 to about 12 carbon ring atoms, and even more typically from 3 to about 8 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be 2 or 3 carbon rings fused together, such as, for example, decalinyl or norpinanyl.

The term “cycloalkylalkyl” (alone or in combination with another term(s)) means alkyl substituted with cycloalkyl. Examples of such substituents include cyclopropylmethyl, cyclobutylmethyl, cyclopentylmethyl, and cyclohexylmethyl.

The term “cycloalkenyl” (alone or in combination with another term(s)) means a partially unsaturated carbocyclyl substituent. Examples of such substituents include cyclobutenyl, cyclopentenyl, and cyclohexenyl.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl substituent (e.g., alkyl, alkenyl, alkynyl, cycloalkyl, cycloalkenyl, aryl, etc.) is indicated by the prefix “Cₓ-C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated carbocyclyl containing from 3 to 6 carbon ring atoms.

The term “arylalkyl” (alone or in combination with another term(s)) means alkyl substituted with aryl.

The term “benzyl” (alone or in combination with another term(s)) means a methyl radical substituted with phenyl, i.e., the following structure:

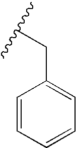

The term “benzene” means the following structure:

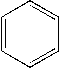

The term “hydrogen” (alone or in combination with another term(s)) means a hydrogen radical, and may be depicted as —H.

The term “hydroxy” or “hydroxyl” (alone or in combination with another term(s)) means —OH.

The term “hydroxyalkyl” (alone or in combination with another term(s)) means alkyl substituted with one more hydroxy.

The term “nitro” (alone or in combination with another term(s)) means —NO₂.

The term “cyano” (alone or in combination with another term(s)) means —CN, which also may be depicted:

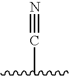

The term “keto” (alone or in combination with another term(s)) means an oxo radical, and may be depicted as ═O.

The term “carboxy” or “carboxyl” (alone or in combination with another term(s)) means —C(O)—OH, which also may be depicted as:

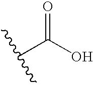

The term “amino” (alone or in combination with another term(s)) means —NH₂. The term “monosubstituted amino” (alone or in combination with another term(s)) means an amino substituent wherein one of the hydrogen radicals is replaced by a non-hydrogen substituent. The term “disubstituted amino” (alone or in combination with another term(s)) means an amino substituent wherein both of the hydrogen atoms are replaced by non-hydrogen substituents, which may be identical or different.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I). Typically, a fluorine radical or chlorine radical is preferred, with a fluorine radical often being particularly preferred.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Where there are more than one hydrogens replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 1,1,1-trifluoroethyl, difluoroethyl, pentafluoroethyl, difluoropropyl, dichloropropyl, and heptafluoropropyl. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), and 1,1,1,-trifluoroethoxy. It should be recognized that if a substituent is substituted by more than one halogen radical, those halogen radicals may be identical or different (unless otherwise stated).

The prefix “perhalo” indicates that each hydrogen radical on the substituent to which the prefix is attached is replaced with an independently selected halogen radical. If all the halogen radicals are identical, the prefix may identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” means an alkoxy substituent wherein each hydrogen radical is replaced with a fluorine radical. Examples of perfluoroalkoxy substituents include trifluoromethoxy (—O—CF₃), perfluorobutoxy, perfluoroisopropoxy, perfluorododecoxy, and perfluorodecoxy.

The term “carbonyl” (alone or in combination with another term(s)) means —C(O)—, which also may be depicted as:

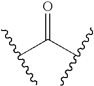

This term also is intended to encompass a hydrated carbonyl substituent, i.e., —C(OH)₂—.

The term “aminocarbonyl” (alone or in combination with another term(s)) means —C(O)—NH₂, which also may be depicted as:

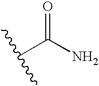

The term “oxy” (alone or in combination with another term(s)) means an ether substituent, and may be depicted as —O—.

The term “alkoxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylthio” (alone or in combination with another term(s)) means —S-alkyl. For example, “methylthio” is —S—CH₃. Other examples of alkylthio substituents include ethylthio, propylthio, butylthio, and hexylthio.

The term “alkylcarbonyl” or “alkanoyl” (alone or in combination with another term(s)) means —C(O)-alkyl. For example, “ethylcarbonyl” may be depicted as:

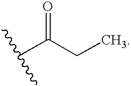

Examples of other often preferred alkylcarbonyl substituents include methylcarbonyl, propylcarbonyl, butylcarbonyl, pentylcarbonyl, and hexylcarbonyl.

The term “aminoalkylcarbonyl” (alone or in combination with another term(s)) means —C(O)-alkyl-NH₂. For example, “aminomethylcarbonyl” may be depicted as:

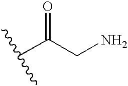

The term “alkoxycarbonyl” (alone or in combination with another term(s)) means —C(O)—O-alkyl. For example, “ethoxycarbonyl” may be depicted as:

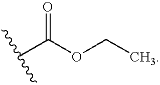

Examples of other often preferred alkoxycarbonyl substituents include methoxycarbonyl, ethoxycarbonyl, propoxycarbonyl, butoxycarbonyl, pentoxycarbonyl, and hexyloxycarbonyl.

The term “carbocyclylcarbonyl” (alone or in combination with another term(s)) means —C(O)-carbocyclyl. For example, “phenylcarbonyl” may be depicted as:

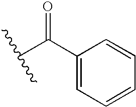

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl. For example, “phenylethylcarbonyl” may be depicted as:

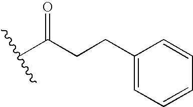

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl. For example, “phenyloxycarbonyl” may be depicted as:

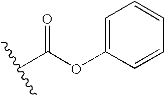

The term “carbocyclylalkoxycarbonyl” (alone or in combination with another term(s)) means —C(O)—O-alkyl-carbocyclyl. For example, “phenylethoxycarbonyl” may be depicted as:

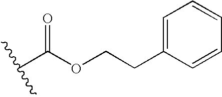

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl.

The term “thiol” (alone or in combination with another term(s)) means a sulfhydryl substituent, and may be depicted as —SH.

The term “sulfonyl” (alone or in combination with another term(s)) means —S(O)₂—, which also may be depicted as:

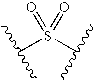

Thus, for example, “alkyl-sulfonyl-alkyl” means alkyl-S(O)₂-alkyl. Examples of typically preferred alkylsulfonyl substituents include methylsulfonyl, ethylsulfonyl, and propylsulfonyl.

The term “aminosulfonyl” (alone or in combination with another term(s)) means —S(O)₂—NH₂, which also may be depicted as:

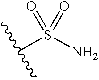

The term “sulfinyl” or “sulfoxido” (alone or in combination with another term(s)) means —S(O)—, which also may be depicted as:

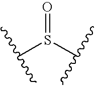

Thus, for example, “alkylsulfinylalkyl” or “alkylsulfoxidoalkyl” means alkyl-S(O)-alkyl. Typically preferred alkylsulfinyl groups include methylsulfinyl, ethylsulfinyl, butylsulfinyl, and hexylsulfinyl.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetradydrofurnayl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

A heterocyclyl alternatively may be 2 or 3 rings fused together, wherein at least one such ring contains a heteroatom as a ring atom (i.e., nitrogen, oxygen, or sulfur). Such substituents include, for example, indolizinyl, pyrindinyl, pyranopyrrolyl, 4H-quinolizinyl, purinyl, naphthyridinyl, pyridopyridinyl (including [3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, or “benzothiofuranyl”), isobenzothienyl (also known as “isobenzothiophenyl”, “isothionaphthenyl”, or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl , 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “2-fused′ring” heterocyclyl (alone or in combination with another term(s)) means a saturated, partially saturated, or aryl heterocyclyl containing 2 fused rings. Examples of 2-fused-ring heterocyclyls include indolizinyl, pyrindinyl, pyranopyrrolyl, 4H-quinolizinyl, purinyl, naphthyridinyl, pyridopyridinyl, pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as 1,3,5-, 1,2,4- or 1,2,3-tiiazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as 1,2-, 1,4-, 2,3- and 2,1-benzopyronyl, quinolinyl, isoquinolinyl, cinnolinyl, quinazolinyl, and 1,4-benzoxazinyl.

The term “heterocyclylalkyl” (alone or in combination with another term(s)) means alkyl substituted with a heterocyclyl.

The term “heterocycloalkyl” (alone or in combination with another term(s)) means a fully saturated heterocyclyl.

In some preferred embodiments, a carbocyclyl or heterocyclyl optionally is substituted with one or more substituents independently selected from the group consisting of halogen, hydroxy (—OH), cyano (—CN), nitro (—NO₂), thiol (—SH), carboxy (—C(O)—OH), amino (—NH₂), keto (═O), aminocarbonyl, alkyl, aminoalkyl, carboxyalkyl, alkylamino, alkylaminoalkyl, aminoalkylamino, alkylaminocarbonyl, aminocarbonylalkyl, alkoxycarbonylalkyl, alkenyl, alkynyl, alkylthioalkyl, alkylsulfinyl, alkylsulfinylalkyl, alkylsulfonyl, alkylsulfonylalkyl, alkylthio, carboxyalkylthio, alkylcarbonyl (also known as “alkanoyl”), alkylcarbonyloxy, alkoxy, alkoxyalkyl, alkoxycarbonyl, alkoxycarbonylalkoxy, alkoxyalkylthio, alkoxycarbonylalkylthio, carboxyalkoxy, alkoxycarbonylalkoxy, carbocyclyl, carbocyclylaminocarbonyl, carbocyclylaminoalkyl, carbocyclylalkoxy, carbocyclyloxyalkyl, carbocyclylalkoxyalkyl, carbocyclylthioalkyl, carbocyclylsulfinylalkyl, carbocyclylsulfonylalkyl, carbocyclylalkyl, carbocyclyloxy, carbocyclylthio, carbocyclylalkylthio, carbocyclylamino, carbocyclylalkylamino, carbocyclylcarbonylamino, carbocyclylcarbonyl, carbocyclylalkyl, carbocyclylcarbonyloxy, carbocyclyloxycarbonyl, carbocyclylalkoxycarbonyl, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylaminocarbonyl, heterocyclylaminoalkyl, heterocyclylalkoxy, heterocyclyloxyalkyl, heterocyclylalkoxyalkyl, heterocyclylthioalkyl, heterocyclylsulfinylalkyl, heterocyclylsulfonylalkyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyl, heterocyclylalkylcarbonyl, heterocyclyloxycarbonyl, heterocyclylcarbonyloxy, heterocyclylalkoxycarbonyl, heterocyclyloxyalkoxyheterocyclyl, heterocyclylthioalkylthioheterocyclyl, heterocyclylthioalkoxyheterocyclyl, and heterocyclyloxyalkylthioheterocyclyl.

In some preferred embodiments, a carbocyclyl or heterocyclyl optionally is substituted with one or more substituents independently selected from the group consisting of halogen, hydroxy, cyano, nitro, thiol, carboxy, amino, aminocarbonyl, C₁-C₆-alkyl, amino-C₁-C₆-alkyl, keto, carboxy-C₁-C₆-alkyl, C₁-C₆-alkylamino, C₁-C₆-alkylamino-C₁-C₆-alkyl, amino-C₁-C₆-alkylamino, C₁-C₆-alkylaminocarbonyl, aminocarbonyl-C₁-C₆-alkyl, C₁-C₆-alkoxycarbonyl-C₁-C₆-alkyl, C₂-C₆-alkenyl, C₂-C₆-alkynyl, C₁-C₆-alkylthio-C₁-C₆-alkyl, C₁-C₆-alkylsulfinyl, C₁-C₆-alkylsulfinyl-C₁-C₆-alkyl, C₁-C₆-alkylsulfonyl, C₁-C₆-alkylsulfonyl-C₁-C₆-alkyl, C₁-C₆-alkylthio, carboxy-C₁-C₆-alkylthio, C₁-C₆-alkylcarbonyl, C₁-C₆-alkylcarbonyloxy, C₁-C₆-alkoxy, C₁-C₆-alkoxy-C₁-C₆-alkyl, C₁-C₆-alkoxycarbonyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, arylaminocarbonyl, arylamino-C₁-C₆-alkyl, aryl-C₁-C₆-alkoxy, aryloxy-C₁-C₆-alkyl, aryl-C₁-C₆-alkoxy-C₁-C₆-alkyl, arylthio-C₁-C₆-alkyl, arylsulfinyl-C₁-C₆-alkyl, arylsulfonyl-C₁-C₆-alkyl, aryl-C₁-C₆-alkyl, aryloxy, arylthio, aryl-C₁-C₆-alkylthio, arylamino, aryl-C₁-C₆-alkylamino, arylcarbonylamino, arylcarbonyl, aryl-C₁-C₆-alkylcarbonyl, arylcarbonyloxy, aryloxycarbonyl, aryl-C₁-C₆-alkoxycarbonyl, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl aminocarbonyl, cycloalkyl amino-C₁-C₆-alkyl, cycloalkyl-C₁-C₆-alkoxy, cycloalkyl oxy-C₁-C₆-alkyl, cycloalkyl-C₁-C₆-alkoxy-C₁-C₆-alkyl, cycloalkyl thio-C₁-C₆-alkyl, cycloalkyl sulfinyl-C₁-C₆-alkyl, cycloalkyl sulfonyl-C₁-C₆-alkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyl, cycloalkyl-C₁-C₆-alkylcarbonyl, cycloalkylcarbonyloxy, cycloalkyloxycarbonyl, cycloalkyl-C₁-C₆-alkoxycarbonyl, heteroaryl, heteroarylaminocarbonyl, heteroarylamino-C₁-C₆-alkyl, heteroaryl-C₁-C₆-alkoxy, heteroaryloxy-C₁-C₆-alkyl, heteroaryl-C₁-C₆-alkoxy-C₁-C₆-alkyl, heteroarylthio-C₁-C₆-alkyl, heteroarylsulfinyl-C₁-C₆-alkyl, heteroarylsulfonyl-C₁-C₆-alkyl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, heteroarylcarbonyl, heteroaryl-C₁-C₆-alkylcarbonyl, heteroaryloxycarbonyl, heteroarylcarbonyloxy, and heteroaryl-C₁-C₆-alkoxycarbonyl. Here, any substitutable carbon optionally is substituted with one or more halogen. In addition, the cycloalkyl, aryl, and heteroaryl typically have 3 to 6 ring atoms, and more typically 5 or 6 ring atoms.

In some preferred embodiments, a carbocyclyl or heterocyclyl optionally is substituted with one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl.

In some preferred embodiments, a carbocyclyl or heterocyclyl optionally is substituted with one or more substituents independently selected from the group consisting of halogen, hydroxy, carboxy, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with one or more halogen. The aryls or cycloalkyls typically have from 3 to 6 ring atoms, and more typically from 5 to 6 ring atoms.

In some preferred embodiments, a carbocyclyl or heterocyclyl optionally is substituted with up to three substituents independently selected from the group consisting of halogen, hydroxy, alkyl, alkoxy, amino, alkylthio, keto, and alkylamino.

In some preferred embodiments, a carbocyclyl or heterocyclyl optionally is substituted with up to three substituents independently selected from the group consisting of halogen, hydroxy, C₁-C₆-alkyl, C₁-C₆-alkoxy, amino, C₁-C₆-alkylthio, keto, and C₁-C₆-alkylamino.

In some preferred embodiments, a carbocyclyl or heterocyclyl optionally is substituted with up to three substituents independently selected from the group consisting of halogen, nitro, alkyl, haloalkyl, alkoxy, haloalkoxy, and amino.

In some preferred embodiments, a carbocyclyl or heterocyclyl optionally is substituted with up to three substituents independently selected from the group consisting of halogen, nitro, C₁-C₆-alkyl, halo-C₁-C₆-alkyl, C₁-C₆-alkoxy, halo-C₁-C₆-alkoxy, and amino.

In some preferred embodiments, a carbocyclyl or heterocyclyl optionally is substituted with up to three substituents independently selected from the group consisting of halogen, alkyl, haloalkyl, alkoxy, and haloalkoxy.

In some preferred embodiments, a carbocyclyl or heterocyclyl optionally is substituted with up to three substituents independently selected from the group consisting of halogen, C₁-C₆-alkyl, halo-C₁-C₆-alkyl, C₁-C₆-alkoxy, and halo-C₁-C₆-alkoxy.

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted”, a non-hydrogen radical is in the place of a hydrogen radical on a carbon or nitrogen of the substituent. Thus, for example, a substituted alkyl substituent is an alkyl substituent wherein at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitutions on a substituent, each non-hydrogen radical may be identical or different (unless otherwise stated).

If a substituent is described as being “optionally substituted”, the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then the nitrogen will be optionally substituted with up to 2 non-hydrogen radicals if the amino nitrogen is a primary nitrogen, whereas the amino nitrogen will be optionally substituted with up to only 1 non-hydrogen radical if the amino nitrogen is a secondary nitrogen. Further illustrations of this definition may be found above at, for example, the sub-section entitled “General Description of Preferred A¹ and A² Substituents.”

This specification uses the terms “substituent” and “radical” interchangeably.

A prefix attached to a multi-component substituent only applies to the first component. To illustrate, the term “alkylcycloalkyl” contains two components: alkyl and cycloalkyl. Thus, the C₁-C₆- prefix on C₁-C₆-alkylcycloalkyl means that the alkyl component of the alkylcycloalkyl contains from 1 to 6 carbon atoms; the C₁-C₆- prefix does not describe the cycloalkyl component. To illustrate further, the prefix “halo” on haloalkoxyalkyl indicates that only the alkoxy component of the alkoxyalkyl substituent is substituted with one or more halogen radicals. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other substituent(s).

When words are used to describe a substituent, the rightmost-described component of the substituent is the component that has the free valence. To illustrate, benzene substituted with methoxyethyl has the following structure:

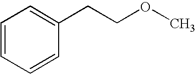

As can be seen, the ethyl is bound to the benzene, and the methoxy is the component of the substituent that is the component furthest from the benzene. As further illustration, benzene substituted with cyclohexanylthiobutoxy has the following structure:

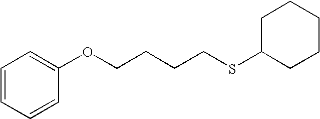

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-cyclohexanyl-methyl-Y.

When a chemical formula is used to describe a substituent, the dash on the left side of the formula indicates the portion of the substituent that has the free valence. To illustrate, benzene substituted with —C(O)—OH has the following structure:

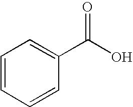

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

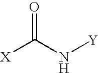

The term “pharmaceutically acceptable” is used adjectivally in this specification to mean that the modified noun is appropriate for use as a pharmaceutical product or as a part of a pharmaceutical product.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent, including the claims below.

The following are definitions for various abbreviations:

“HCl” is hydrochloric acid.

“MgSO₄” is magnesium sulfate.

“Na₂SO₄” is sodium sulfate.

“NaOH” is sodium hydroxide.

“Me” is methyl. “MeOH”, for example, is methanol.

“Et” is ethyl. “EtOH”, for example, is ethanol. And “Et₃N” is triethylamine.

“HOAc” or “AcOH” is acetic acid.

“EtOAc” is ethyl acetate.

“H₂O” is water.

“CH₂Cl₂” is methylene chloride.

“K₂CO₃” is potassium carbonate.

“LiHMDS” is lithium hexamethyldisilazide.

“THF” is tetrahydrofuran.

“DMF” is dimethylformamide. “DMF (OMe)₂” is N,N-dimethylformamide dimethyl acetal. And “DMF DMA” is dimethylformamide dimethyl acetal.

“EDC” is 1-(3-dimethylaminopropyl) 3-ethylcarbodiimide hydrochloride.

“HOBT” is 1-hydroxybenzotriazole.

“Ts” is tosyl.

The term “h” or “hr” is hour or hours.

The term “min” is minute or minutes.

“SEM” is 2-(trimethylsilyl)ethoxymethyl. “SEM-Cl” is 2-(trimethylsilyl)ethoxymethyl chloride.

“DSC” is differential scanning calorimetry.

“b.p.” is boiling point.

“m.p.” is melting point.

“eq.” is equivalent.

“boc” is tert-butoxycarbonyl.

“TFA” is trifluoroacetic acid.

“N₂” is nitrogen gas.

“LPS” is lipopolysaccharide.

### H. Compound Preparation

The detailed examples below illustrate preparation of compounds of this invention. Other compounds of this invention may be prepared using the methods illustrated in these examples, either alone or in combination with techniques generally known in the art. Such known techniques include, for example, those disclosed in WIPO Int'l Publ. No. WO 98/52940 (PCT Patent Application No. US98/10436 published on Nov. 26, 1998) (incorporated by reference into this specification). Such known techniques also include, for example, those disclosed in WIPO Int'l Publ. No. WO 00/31063 (PCT Patent Application No. US99/26007 published on Jun. 2, 2000) (incorporated by reference into this specification).

### EXAMPLES

The following examples are merely illustrative, and not limiting to the remainder of this disclosure in any way.

### Example 1

### Preparation of 4-[3-(4-chlorophenyl)-4-pyrimidin-4-yl-1H-pyrazol-5-yl]-N-[(1R)-1-phenylethyl]-trans-cyclohexanamine

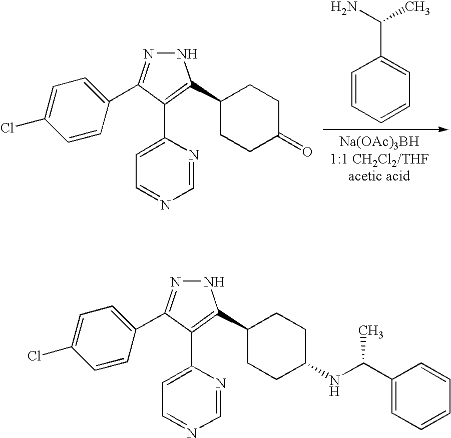

4-[3-(4-chlorophenyl)-4-pyrimidin-4-yl-1H-pyrazol-5-yl]cyclohexanone (2.0 g, 5.68 mmol) was dissolved in 100 ml of 1:1 CH₂Cl₂/THF in a 250 ml round bottom flask at room temperature. (R)-(+)-α-methylbenzylamine (1.44 ml, 11.36 mmol) was added, followed by sodium triacetoxyborohydride (3.61 g, 17.04 mmol) and 1 ml of acetic acid. The resulting mixture was then stirred at room temperature for 2.5 hr. The solvent was evaporated, and the residue was quenched with 100 ml of 2.5 N NaOH. The resulting suspension was extracted with ethyl acetate (3×100 ml). The combined organic layers were washed with brine (1×250 ml), dried over anhydrous MgSO₄, and filtered. The solvent was removed to produce a light yellow oil containing a mixture of the cis and trans isomers of the desired product. Silica gel flash chromatography was performed on a Biotage system (3% MeOH in CH₂Cl₂ with 0.3% NH₄OH, followed by 10% MeOH in CH₂Cl₂ with 1% NH₄OH) to afford the desired trans isomer as the second isomer off the column in the form of a colorless oil. The product was triturated with acetonitrile and dried under vacuum to produce 915 mg of product in the form of a white solid. ¹H NMR (300 MHz, d-DMSO) δ 13.06 (s, 1H), 9.15 (d, J=1.01 Hz, 1H), 8.62 (d, J=5.24 Hz, 1H), 7.39-7.20 (m, 9H), 7.09 (dd, J=5.34, 1.31 Hz, 1H), 3.91 (d, J=6.44 Hz, 1H), 3.03 (t, J=12.09, 1H), 2.22 (t, J=10.38, 1H), 2.07 (d, J=12.29, 1H), 1.83 (m, 4H), 1.45 (m, 2H), 1.21 (d, J=6.44 Hz, 3H), 1.20-0.97 (m, 2H); LC/MS, tᵣ=2.92 min (0 to 95% acetonitrile/water over 5 min at 1 mL/min, at 254 nm, at 50° C.), (M+H), Calculated=458, Found=458; HR/MS (M+H), Calculated=458.2111, Found=458.2121 (Δ mmu=1.0); DSC: melt onset=200.7° C., melt peak=203.3° C.; Elemental Analysis, Calculated: C, 70.81; H, 6.16; N, 15.29; Cl, 7.74. Found: C, 70.28; H, 6.20; N, 15.32; Cl, 8.18; [α]_{D}²³=+50.4°.

### Example 2

### Preparation of 4-[3-(4-chlorophenyl)-4-pyrimidin-4-yl-1H-pyrazol-5-yl]-N-[(1R)-1-phenylethyl]-trans-cyclohexanamine 4-methylbenzenesulfonate

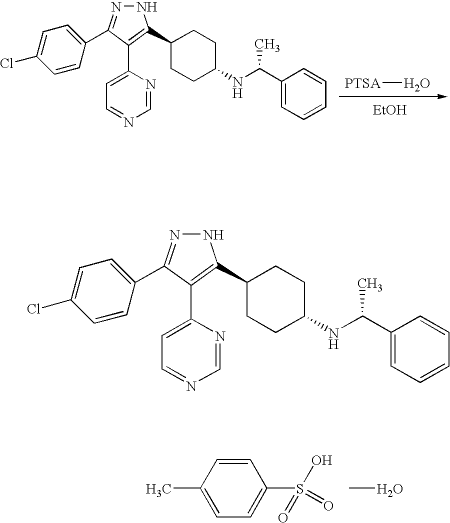

4-[3-(4-chlorophenyl)-4-pyrimidin-4-yl-1H-pyrazol-5-yl]-N-[(1R)-1-phenylethyl]-trans-cyclohexanamine (1.0 g, 2.18 mmol) was suspended in 12 ml of ethanol in a 50 ml round bottom flask at room temperature. Para-toluenesulphonic acid (415.3 mg, 2.18 mmol) was added, and a solution formed immediately. The resulting mixture was stirred at room temperature for 15 min. The mixture was then cooled, and a precipitate formed. The solid was filtered, washed with diethyl ether, and dried under vacuum to afford 1.22 g of a white solid. ¹H NMR (300 MHz, d-DMSO) δ 13.15 (s, 1H), 9.16 (d, J=1.20 Hz, 1H), 8.83 (br s, 2H), 8.64 (d, J=5.24 Hz, 1H), 7.58-7.35 (m, 11H), 7.12 (m, 3H), 4.58 (d, J=5.43 Hz, 1H), 3.08 (br s, 1H), 2.73 (br s, 1H), 2.29 (s, 3H), 2.22 (d, J=7.25, 1H), 2.08 (d, J=9.46, 1H), 1.94 (m, 2H), 1.56 (m, 3H), 1.49 (m, 3H); LC/MS, tᵣ=2.98 min (0 to 95% acetonitrile/water over 5 min at 1 ml/min, at 254 nm, at 50° C.), (M+H free base), Calculated=458, Found=458; HR/MS (M+H free base), Calculated=458.2106, Found=458.2136 (Δ mmu=3.0); DSC: melt onset=185.3° C. (Form II), 260.4° C. (FI), melt peak=194.0° C. (Form II), 262.4° C. (Form I); Elemental Analysis, Calculated: C, 64.80; H, 5.76; N, 11.11; Cl, 5.63; S, 5.09. Found: C, 64.64; H, 5.95; N, 10.97; Cl, 5.92; S, 5.08; [α]D²³=+35.1°.

### Example 3

### Preparation of 4-[3-(4-chlorophenyl)-4-pyrimidin-4-yl-1H-pyrazol-5-yl]-N-cyclohexyltrans-cyclohexanamine

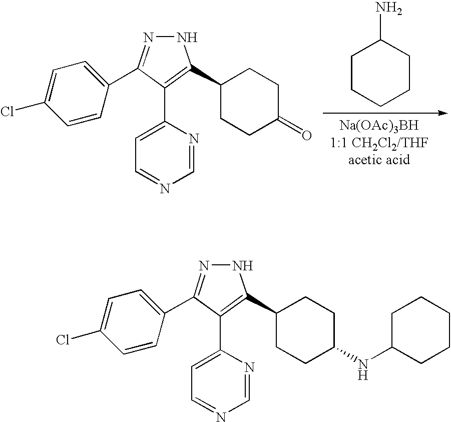

4-[3-(4-chlorophenyl)-4-pyrimidin-4-yl-1H-pyrazol-5-yl]cyclohexanone (4.0 g, 11.36 mmol) was dissolved in 200 ml of 1:1 CH₂Cl₂/THF in a 500 ml round-bottom flask at room temperature. Cyclohexylamine (2.61 ml, 22.72 mmol) was added, followed by sodium triacetoxyborohydride (7.22 g, 34.08 mmol) and 2 ml of acetic acid. The resulting mixture was stirred at room temperature for 2.5 hr. The solvent was evaporated, and the residue was quenched with 200 ml of 2.5 N NaOH. The resulting suspension was extracted with ethyl acetate (3×200 ml). The combined organic layers were washed with brine (1×500 ml), dried over anhydrous MgSO₄ and filtered. The solvent was removed to produce a light yellow oil containing a mixture of the cis and trans isomers of the desired product. Silica gel flash chromatography was performed on a Biotage system (1.5% MeOH in CH₂Cl₂ with 0.15% NH₄OH) to produce the desired trans isomer as the second isomer off the column in the form of a colorless oil. The product was triturated with acetonitrile and dried under vacuum to produce 1.46 g of the product in the form of a white solid. ¹H NMR (300 MHz, d-DMSO) δ 13.17 (br s, 1H), 9.15 (d, J=1.21 Hz, 1H), 8.62 (d, J=5.44 Hz, 1H), 7.43-7.33 (m, 4H), 7.13 (dd, J=5.35, 1.31 Hz, 1H), 3.33 (d, J=6.45, 1H), 3.05 (t, J=12.08, 1H), 1.95-1.52 (m, 11H), 1.29-0.90 (m, 8H); LC/MS, tᵣ=2.31 min (0 to 95% acetonitrile/water over 5 min at 1 ml/min, at 254 nm, at 50° C.), (M+H), Calculated=436, Found=436; HR/MS (M+H), Calculated=436.2268, Found=436.2250 (Δ mmu=-1.8); DSC: melt onset=194.0° C., melt peak=203.4° C.; Elemental Analysis, Calculated: C, 68.87; H, 6.94; N, 16.06; Cl, 8.13. Found: C, 67.71; H, 6.92; N, 16.00; Cl, 8.98.

### Example 4

### Preparation of 4-[3-(4-chlorophenyl)-4-pyrimidin-4-yl-1H-pyrazol-5-yl]-N-cyclohexyl]trans-cyclohexanamine 4-methylbenzenesulfonate

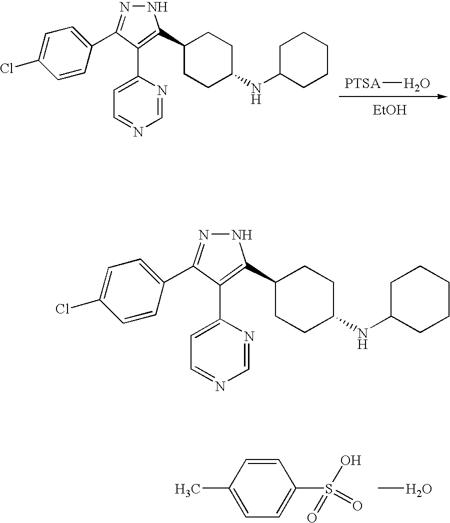

4-[3-(4-chlorophenyl)-4-pyrimidin-4-yl-1H-pyrazol-5-yl]-N-cyclohexyl]-trans-cyclohexanamine (1.0 g, 2.29 mmol) was suspended in 12 ml of ethanol in a 50 ml round-bottom flask at room temperature. Para-toluenesulphonic acid (436.3 mg, 2.29 mmol) was added, and a solution formed immediately. The reaction mixture was stirred at room temperature for 15 min. The reaction mixture was then cooled, and a precipitate formed. The solid was filtered, washed with diethyl ether, and dried under vacuum to afford 1.30 g of a white solid. ¹H NMR (300 MHz, d-DMSO) δ 9.17 (d, J=0.81 Hz, 1H), 8.64 (d, J=5.24 Hz, 1H), 8.22 (br s, 2H), 7.52-7.36 (m, 6H), 7.12 (m, 3H), 3.15 (m, 4H), 2.29 (s, 3H), 2.14-1.90 (m, 6H), 1.76-1.60 (m, 5H), 1.43 (q, J=11.07 Hz, 2H), 1.28 (m, 4H), 1.12 (m, 2H); LC/MS, tᵣ=2.26 min (0 to 95% acetonitrile/water over 5 min at 1 ml/min, at 254 nm, at 50° C.), (M+H free base), Calculated=436, Found=436; HR/MS (M+H free base), Calculated=436.2247, Found=436.2263 (Δ mmu=1.6); DSC: melt onset=330.9° C., melt peak=332.9° C.; Elemental Analysis, Calculated: C, 63.19; H, 6.30; N, 11.52; Cl, 5.83; S, 5.27. Found: C, 62.67; H, 6.43; N, 11.34; Cl, 6.28; S, 5.21.

### Example 5

### Preparation of 4-[3-(4-chlorophenyl)-4-pyrimidin-4-yl-1H-pyrazol-5-yl]-N-[(1R)-1-(4-fluorophenyl)ethyl]-trans-cyclohexanamine

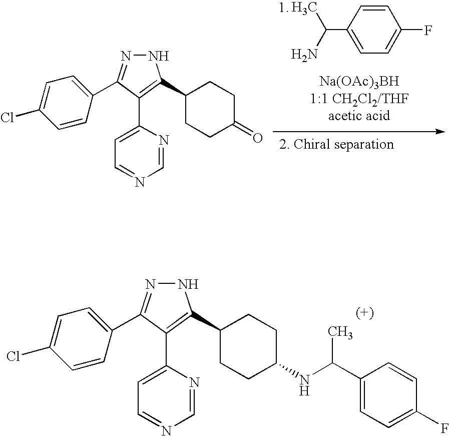

4-[3-(4-chlorophenyl)-4-pyrimidin-4-yl-1H-pyrazol-5-yl]cyclohexanone (10.0 g, 28.4 mmol) was dissolved in 500 ml of 1:1 CH₂Cl₂/THF in a 1 L round bottom flask at room temperature. 4-Fluoro-α-methylbenzylamine (7.46 ml, 56.8 mmol) was added, followed by sodium triacetoxyborohydride (18.07 g, 85.2 mmol) and 5 ml of acetic acid. The resulting mixture was stirred at room temperature for 12 hr. The solvent was then evaporated, and the residue was quenched with 500 ml of 2.5 N NaOH. The resulting suspension was extracted with ethyl acetate (3×500 ml). The combined organic layers were washed with brine (1×1 L), dried over anhydrous MgSO₄, and filtered. The solvent was removed to afford a light yellow oil containing a mixture of the cis and trans isomers of the racemic product. Silica gel flash chromatography was performed on a Biotage system (1.5% MeOH in CH₂Cl₂ with 0.15% NH₄OH) to afford the desired trans isomer as the second isomer off the column in the form of a colorless oil. The product was triturated with acetonitrile and dried under vacuum to afford 4.75 g of a white solid. The cis racemate was subjected to chiral separation (Chiracel OD column, 15% ethanol/85% heptane/0.2% diethyl amine) to produce 2.04 g of product in the form of a white solid. ¹H NMR (300 MHz, d-DMSO) δ 13.07 (s, 1H), 9.14 (d, J=1.00 Hz, 1H), 8.62 (d, J=5.23 Hz, 1H), 7.43-7.30 (m, 6H), 7.20-7.14 (m, 2H), 7.10 (dd, J=5.24, 1.21 Hz, 1H), 3.98 (br s, 1H), 3.03 (t, J=11.89, 1H), 2.22 (br s, 1H), 2.08 (d, J=11.48, 1H), 1.86 (br s, 4H), 1.47 (m, 2H), 1.20-1.05 (m, 5H); LC/MS, tᵣ=2.59 min (0 to 95% acetonitrile/water over 5 min at 1 ml/min, at 254 nm, at 50° C.), (M+H), Calculated=476, Found=476; HR/MS (M+H), Calculated=476.2017, Found=476.2057 (Δ mmu=4.0); DSC: melt onset=219.2° C., melt peak=224.1° C.; Elemental Analysis, Calculated: C, 68.13; H, 5.72; N, 14.71; Cl, 7.45; F, 3.99. Found: C, 67.86; H, 6.11; N, 12.74; Cl, 7.54; F, 3.19; [ ]D²³=+47.0°.

### Example 6

### Preparation of 4-[3-(4-chlorophenyl)-4-pyrimidin-4-yl-1H-pyrazol-5-yl]-trans-cyclohexanol

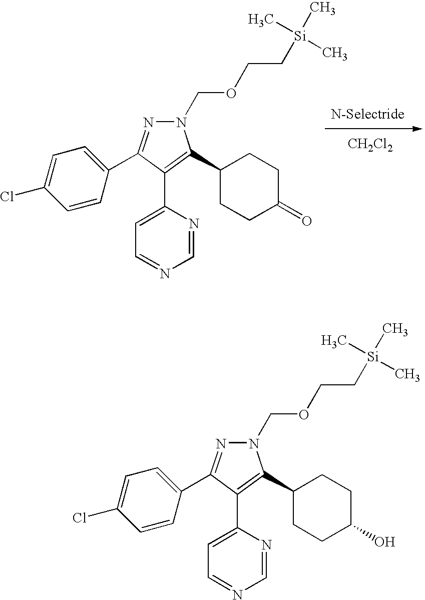

Part A. 4-(3-(4-chlorophenyl)-4-pyrimidin-4-yl-1-{[2-(trimethylsilyl)ethoxy]methyl}-1H-pyrazol-5-yl)cyclohexanone (20.0 g, 41.4 mmol) was dissolved in 400 ml of CH₂Cl₂ in a 1 L round-bottom flask. N-Selectride (49.7 ml, 49.7 mmol) was added, and the resulting mixture was stirred at room temperature for 18 hr. The solvent was evaporated, and the residue was quenched with 200 ml of 2.5 N NaOH. The resulting suspension was extracted with ethyl acetate (3×200 ml). The combined organic layers were washed with brine (1×500 ml), dried over anhydrous MgSO₄, and filtered. The solvent was removed to afford an orange oil containing a 9:1 mixture of trans to cis isomers of the desired product. Silica gel flash chromatography was performed on a Biotage system (1% MeOH in CH₂Cl₂ with 0.15% NH₄OH), followed by 3% MeOH in CH₂Cl₂ with 0.3% NH₄OH) to afford the desired trans isomer as the second isomer off the column in the form of a colorless oil. The product was triturated with acetonitrile and dried under vacuum to afford 5.21 g of the SEM protected alcohol in the form of a white solid. ¹H NMR (300 MHz, d-DMSO) δ 9.12 (s, 1H), 8.46 (d, J=5.63 Hz, 1H), 7.48-7.27 (m, 4H), 6.86 (d, J=5.64 Hz, 1H), 5.25 (s, 2H), 3.72 (m, 3H), 3.26 (m, 1H), 2.09 (t, J=13.29, 4H), 1.81-1.66 (m, 2H), 1.50-1.38 (m, 2H), 0.94 (m, 2H), 0.05 (m, 9H); LC/MS, tᵣ=4.37 min (0 to 95% acetonitrile/water over 5 min at 1 ml/min, at 254 nm, at 50° C.), (M+H), Calculated=485, Found=485; HR/MS (M+H), Calculated=485.2134, Found=485.2174 (Δ mmu=4.0).

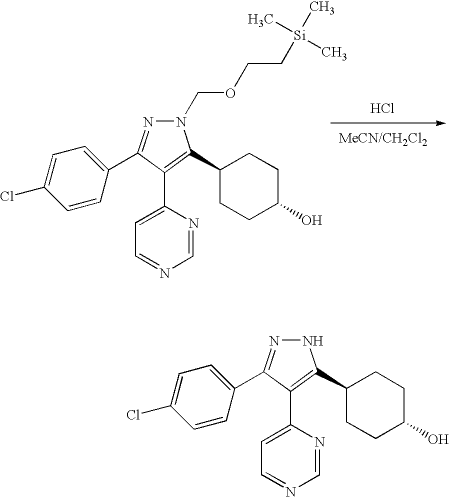

Part B. 4-(3-(4-chlorophenyl)-4-pyrimidin-4-yl-1-{[2-trimethylsilyl)ethoxy]methyl}-1H-pyrazol-5-yl)-trans-cyclohexanol (2.0 g, 4.12 mmol) was dissolved in 30 ml of 1:1 acetonitrile/CH₂Cl₂ in a 100 ml round-bottom flask. 2.0 ml of concentrated HCl was added, and the resulting mixture was stirred at room temperature for 18 hr. The solvent was then evaporated, and the resulting residue was dissolved in 100 ml of ethyl acetate and washed with brine (1×100 ml). A precipitate formed in the ethyl acetate layer, which was filtered and found to be pure desired product. The solid was dried under vacuum to afford 855 mg of product in the form of a white solid. ¹H NMR (300 MHz, d-DMSO) δ 13.18 (s, 1H), 9.17 (s, 1H), 8.63 (d, J=5.24 Hz, 1H), 7.52-7.34 (m, 4H), 7.14 (d, J=5.04 Hz, 1H), 4.63 (d, J=4.43, 1H), 3.43 (m, 1H), 3.03 (t, J=12.09, 1H), 1.96-1.84 (m, 4H), 1.69-1.52 (m, 2H) 1.28-1.15 (m, 2H); LC/MS, tᵣ=2.78 min (0 to 95% acetonitrile/water over 5 min at 1 ml/min, at 254 nm, at 50° C.), (M+H), Calculated=355, Found=355; HR/MS (M+H), Calculated=355.1320, Found=355.1320 (Δ mmu=0.0); Elemental Analysis, Calculated: C, 64.31; H, 5.40; N, 15.79; Cl, 9.99. Found: C, 64.05; H, 5.32; N, 15.84; Cl, 10.02.

### Example 7

### Preparation of 4-[3-(4-chlorophenyl)-5-(4-piperidin-1-yl-cyclohexyl)-1H-pyrazol-4-yl]pyrimidine Trifluoroacetate

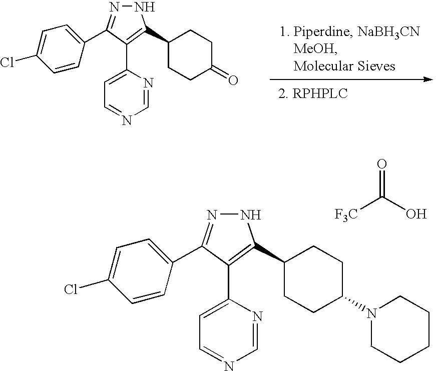

4-[3-(4-chlorophenyl)-4-pyrimidin-4-yl-1H-pyrazol-5-yl]cyclohexanone (704 mg, 2 mmol) was dissolved in methanol (40 mL) under N₂ at room temperature. 4 Molecular sieves were added (approximately 8 g), followed by piperidine (0.2 mL, 2 mmol) and sodium cyanoborohydride (251 mg, 4 mmol). After stirring the resulting heterogeneous mixture for 16 hr, it was filtered through a pad of Celite and the filtrate concentrated in vacuo. The crude stereoisomeric mixture was purified by flash chromatography (5:95 to 15:85=MeOH+0.4% NH₄OH:CH₂Cl₂+0.4% NH₄OH). The more polar trans isomer was further purified by reverse phase HPLC (80:20 to 40:60=H₂O-TFA:CH₃CN gradient). Peak isolated at 59:41, t_{R}=18.2. The sample was lyophilized and dried, and 35 mg of the product in the form of a light yellow solid was isolated. ¹H NMR (DMSO-d₆+TFA) 9.19 (s, 1H), 8.93 (br s, 1H), 8.65 (d, 1H), 7.46 (d, 2H), 7.37 (d, 2H), 7.13 (d, 1H), 3.41 (m, 2H), 3.22 (m, 1H), 3.09 (m, 1H), 2.98 (m, 2H), 2.07 (m, 4H), 1.83 (m, 2H), 1.64 (m, 7H), 1.41 (m, 1H). EI-MS m/z 422 (MH⁺). Anal. Calcd for C₂₄H₂₈N₅Cl+2.5 TFA: C, 49.26; H, 4.35; N, 9.91. Found: C, 49.07; H, 4.56; N, 10.05.

### Example 8

### Preparation of 4-[3-(4-chlorophenyl)-5-(4-piperidin-1-ylcyclohexyl)-1H-pyrazol-4-yl]pyrimidine Hydrochloride Hydrate

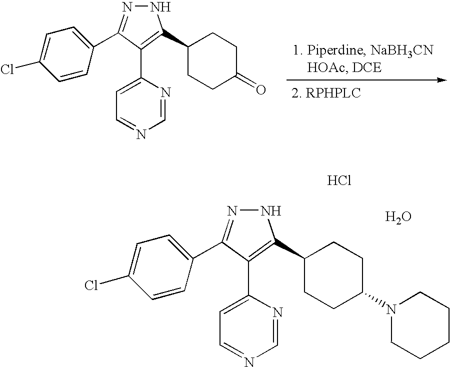

4-[3-(4-chlorophenyl)-4-pyrimidin-4-yl-1H-pyrazol-5-yl]cyclohexanone (10 g, 28.4 mmol) was dissolved in 1,2-dichloroethane (129 mL) under N₂ at room temperature. Glacial acetic acid (1.6 mL, 28.4 mmol) was added, followed by piperidine (2.8 mL, 28.4 mmol). After stirring 1 hr, sodium cyanoborohydride (1.8 g, 28.4 mmol) was added in one portion. After stirring the resulting heterogeneous mixture for 16 hr, it was filtered through a pad of Celite, and the filtrate concentrated in vacuo. The crude stereoisomeric mixture was purified by flash chromatography (5:95 to 15:85=MeOH+0.4% NH₄OH:CH₂Cl₂+0.4% NH₄OH). The more polar trans isomer was further purified by reverse phase HPLC (80:20 to 40:60=H₂O—HCl:CH₃CN gradient). The sample was lyophilized and dried, and 830 mg of the product in the form of a white powdery solid was isolated. ¹H NMR (DMSO-d₆) 9.94 (br s, 1H), 9.18 (s, 1H), 8.64 (d, 1H), 7.44 (d, 2H), 7.35 (d, 2H), 7.13 (d, 1H), 3.35 (m, 2H), 3.14 (m, 1H), 3.08 (m, 1H), 2.92 (m, 2H), 2.17 (m, 2H), 2.02 (m, 2H), 1.81 (m, 4H), 1.62 (m, 5H), 1.39 (m, 1H). EI-MS m/z 422 (MH⁺). Anal. Calcd for C₂₄H₂₈N₅Cl+1.5 HCl+0.5H₂O: C, 59.35; H, 6.33; N, 14.42. Found: C, 59.60; H, 6.67; N, 14.53.

### Example 9

### Preparation of ((2R)-1-{4-[3-(4-chlorophenyl)-4-pyrimidin-4-yl-1H-pyrazol-5-yl]cyclohexyl)pyrrolidin-2-yl)methanol Trifluoroacetate

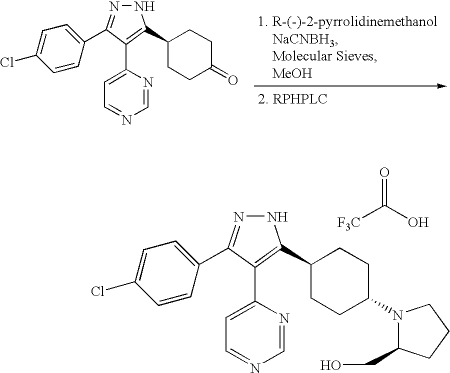

4-[3-(4-chlorophenyl)-4-pyrimidin-4-yl-1H-pyrazol-5-yl]cyclohexanone (704 mg, 2 mmol) was dissolved in methanol (40 mL) under N₂ at room temperature. 4 Molecular sieves were added (approximately 8 g), followed by R-(−)-2-pyrrolidinemethanol (0.2 mL, 2 mmol) and sodium cyanoborohydride (251 mg, 4 mmol). After stirring the resulting heterogeneous mixture for 16 hr, it was filtered through a pad of Celite, and the filtrate was concentrated in vacuo. The crude stereoisomeric mixture was purified by flash chromatography (5:95 to 20:80=MeOH+0.4%/NH₄OH:CH₂Cl₂+0.4% NH₄OH). The more polar trans isomer was further purified by reverse phase HPLC (80:20 to 40:60=H₂O--TFA:CH₃CN gradient). Peak isolated at 63:37, t_{R}=14.3. The sample was lyophilized and dried, and 157 mg of the product in the form of a white solid was isolated. ¹H NMR (DMSO-d₆+TFA) 9.19 (s, 1H), 9.10 (br s, 1H), 8.65 (d, 1H), 7.46 (d, 2H), 7.37 (d, 2H), 7.13 (d, 1H), 3.78 (m, 1H), 3.69 (dd, 1H), 3.58 (dd, 1H), 3.35 (m, 2H), 3.28 (m, 1H), 3.10 (m, 1H), 2.16 (m, 1H), 2.03 (m, 4H), 1.87 (m, 2H), 1.76 (m, 1H), 1.60 (m, 5H). EI-MS m/z 438 (MH⁺). Anal. Calcd for C₂₄H₂₈N₅OCl+2.1 TFA: C, 50.00; H, 4.48; N, 10.34. Found: C, 49.70; H, 4.63; N, 10.46.

### Example 10

### Preparation of ((2R)-1-{4-[3-(4-chlorophenyl)-4-pyrimidin-4-yl-1H-pyrazol-5yl]cyclohexyl}pyrrolidin-2-yl)methanol Hydrochloride Hydrate

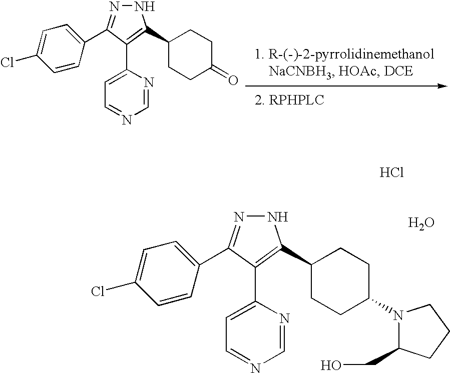

4-[3-(4-chlorophenyl)-4-pyrimidin-4-yl-1H-pyrazol-5-yl]cyclohexanone (8 g, 22.7 mmol) was dissolved in 1,2-dichloroethane (90 mL) under N₂ at room temperature. Glacial acetic acid (1.3 mL, 22.7 mmol) was added, followed by R-(−)-2-pyrrolidinemethanol (2.2 mL, 22.7 mmol). After stirring for 30 min, sodium cyanoborohydride (1.4 g, 22.7 mmol) was added in one portion. After stirring the resulting heterogeneous mixture for 16 hr, it was filtered through a pad of Celite, and the filtrate was concentrated in vacuo. The crude stereoisomeric mixture was purified by flash chromatography (5:95 to 15:85=MeOH+0.4% NH₄OH:CH₂Cl₂+0.4% NH₄OH). The more polar trans isomer was further purified by reverse phase HPLC (80:20 to 40:60=H₂O—HCl:CH₃CN gradient). The sample was lyophilized and dried, and 700 mg of product in the form of a white powdery solid was isolated. ¹H NMR (DMSO-d₆) 9.49 (br s, 1H), 9.17 (s, 1H), 8.64 (d, 1H), 7.46 (d, 2H), 7.37 (d, 2H), 7.13 (d, 1H), 3.78 (m, 1H), 3.69 (dd, 1H), 3.61 (dd, 1H), 3.24 (m, 3H), 3.09 (m, 1H), 2.21 (m, 1H), 2.10 (m, 1H), 2.03 (m, 3H), 1.87 (m, 2H), 1.77 (m, 1H), 1.61 (m, 5H). EI-MS m/z 438 (MH⁺). Anal. Calcd for C₂₄H₂₈N₅OCl+1.2 HCl+1.0H₂O: C, 57.68; H, 6.29; N, 14.01. Found: C, 57.42; H, 6.63; N, 13.94.

### Example 11

### Preparation of 2-[{4-[3-(4-chlorophenyl)-4-pyrimidin-4-yl-1 H-pyrazol-5-yl] cyclohexyl)(methyl)amino]ethanol Trifluoroacetate Hydrate

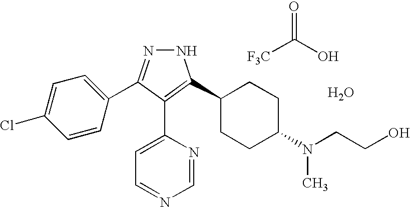

To a solution of 1.24 g (16.5 mmol, 6 eq.) 2-(methylamino)ethanol in 10 mL of methanol was added 1 mL of 6M HCl/CH₃OH, followed by 0.975 g (2.8 mmol) 4-[3-(4-chlorophenyl)-4-pyrimidin-4-yl-1H-pyrazol-5 yl]cyclohexanone, 0.149 g (2.4 mmol, 0.8 eq.) sodium cyanoborohydride, and 8 g powdered 3A sieves. The resulting mixture was stirred at room temperature overnight. After filtering through Celite, the solution was concentrated in vacuo to afford 2.31 g yellow oil. The crude oil was purified on silica gel, eluting with 10/89/1 CH₃OH/CH₂Cl₂/NH₄OH. The cis/trans mixture was separated by RP-HPLC using a gradient elution of 90/10H₂O:TFA/CH₃CN. The final product was isolated as a yellow solid (252 mg). Anal. Calcd for C₂₂H₂₆N₅O₁Cl₁.2.6 TFA.H₂O: C, 44.97; H, 4.25; N, 9.64. Found: C, 44.77; H, 4.40; N, 9.79. HRMS calcd for C₂₂H₂₆N₅O₁Cl₁ 412.1904, found 412.1920. ¹H NMR (CD₃OD d₄) br s), 8.50 (1H, d), 7.35 (2H, d), 7.29 (2H, d), 7.08 (1H, br d), 3.62 (2H, t), 3.16 (1H, m), 2.65 (3H, br m), 2.35 (3H, s), 1.98 (4H, m), 1.64 (2H, m), 1.41 (2H, m).

### Example 12

### Preparation of 2-[{4-[3-(4-chlorophenyl)-4-pyrimidin-4-yl-1H-pyrazol-5-yl]cyclohexyl)(methyl)amino]ethanol Hydrochloride Hydrate

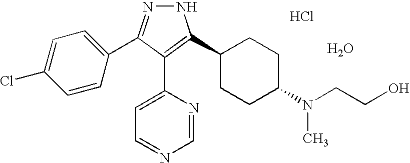

To a solution of 1.99 g (26.5 mmol) 2-(methylamino) ethanol in 25 mL dichloroethane was added 13.2 g (37.4 mmol, 1.4 eq.) 4-[3-(4-chlorophenyl)-4-pyrimidin-4-yl-1H-pyrazol-5-yl]cyclohexanone, 15.8 g (74.5 mmol, 2.8 eq.) sodium triacetoxyborohydride, and 5.5 mL acetic acid. The resulting mixture was stirred at room temperature overnight and then concentrated in vacuo to afford a yellow solid. The solid was purified on silica to remove impurities, eluting with 10/89/1 CH₃OH/CH₂Cl₂/NH₄OH. The resultant cis/trans mixture was chromatographed on a reverse phase column using a gradient elution of 90/10H₂O:HCl/CH₃CN. The trans isomer was isolated as a pale yellow solid (664 mg). Anal. Calcd for C₂₂H₂₆N₅O₁Cl₁-1.4 HCl: C, 55.88; H, 6.07; N, 14.81. Found: C, 55.56; H, 6.15; N, 15.19. HRMS calcd for C₂₂H₂₆N₅O₁Cl₁ 412.1904, found 412.1866. ¹H NMR (CD₃OD d₄) br s), 8.58 (1H, d), 7.46 (2H, d), 7.39 (2H, d), 7.19 (1H, br d), 3.90 (2H, br t), 3.50 (3H, m) 3.17 (1H, m), 2.91 (3H, s), 2.20 (4H, br m), 1.95 (2H, m), 1.80 (2H, m).

### Example 13

### Preparation of 4-[3-(3,4-difluorophenyl)-4-pyrimidin-4-yl-1H-pyrazol-5-yl]-N-isopropylcyclohexanamine

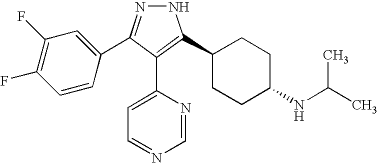

4-[3-(3,4-difluorophenyl)-4-pyrimidin-4-yl-1H-pyrazol-5-yl]cyclohexanone (1.96 g, 5.53 mmol) was dissolved in CH₂Cl₂ (25 mL). Iso-propylamine (0.94 mL, 11.06 mmol) was added, and the mixture was stirred for 16 hr. Afterward, Na(Ac)₃BH was added to the reaction mixture. The resulting mixture was stirred for 1 hr and then quenched with 0.1 N NaOH (75 mL). The CH₂Cl₂ layer was separated, and the aqueous layer was extracted with CH₂Cl₂ (3×75 ml). The organic layers were combined and then washed with deionized water (75 mL). The organic layer was separated, and the aqueous layer was extracted with CH₂Cl₂ (3×75 mL). The organic layers were combined and dried over Na₂SO₄. Solvent was removed in vacuo to yield 2 grams of a yellow oil. The crude material consisted of a 50:50 mixture of cis and trans isomers. The isomers were separated via flash chromatography, (gradient elution of 3%-5% MeOH/CH₂Cl₂, 0.1% NH₄OH) to produce a yellow oil containing the cis isomer (R_{f}=0.45; 3% MeOH/CH₂Cl₂, 0.1% NH₄OH), and a yellow oil containing the trans isomer (R_{f}=0.40; 3% MeOH/CH₂Cl₂, 0.1% NH₄OH). The oil containing the trans isomer was taken up in CH₃CN (50 mL) and sonicated until a solid white precipitate crashed out. The precipitate was collected on a pad and washed with CH₃CN. The material was dried in a vacuum oven to produce 0.65 g of product in the form of a white powder (30% yield). ¹H NMR (400 MHz, CD₃OD): 9.11 (d, J=1.1 Hz, 1H), 8.56 (dd, J=5.4, 0.04 Hz, 1H), 8.65 (m, 2H), 7.14 (dd, J=5.4, 1.5 Hz 2H), 7.1 (m, 1H), 3.18 (t, J=3.3 Hz, 1H), 3.0 (septet, J=6.3 Hz, 1H), 2.65 (t, J=3.6 Hz, 1H), 2.01 (m, 4H), 1.66 (dd, J=25.7, 10.1 Hz, 2H), 1.20 (dd, J=24.0, 12.0 Hz, 2H), 1.1 (d, J=6.3 Hz, 6H). HRMS (m/z): [M+H]⁺ calcd for C₂₂H₂₅F₂N₅, 398.2151; found, 398.2146.

### Example 14

### Preparation of 4-{3-[4-fluoro-3-(trifluoromethyl)phenyl]-4-pyrimidin-4-yl-1H-pyrazol-5-yl}-N-isopropylcyclohexanamine

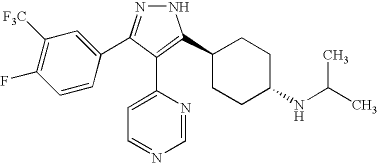

This compound was prepared according to a procedure analogous to the one in the above example for the synthesis of 4-[3-(3,4-difluorophenyl)-4-pyrimidin-4-yl-1H-pyrazol-5-yl]-N-isopropylcyclohexanamine. ¹H NMR (400 MHz, DMSO): 13.46 (s, 1H), 9.14 (s, 1H), 8.97 (br s, 1H), 8.64 (d, J=5.2 Hz, 1H), 7.71 (m, 1H), 7.67, (m, 1H), 7.47 (m, 1H), 7.20 (d, J=5.1, 1H), 3.36 (m, 1H), 3.00 (m, 2H), 2.12 (d, J=10.3 Hz, 2H), 1.93 (d, J=12.1, 2H), 1.70 (dd, J=24.3, 12.0 Hz, 2H), 1.51 (dd, J=22.7, 10.6 Hz, 2H), 1.24, (d, J=6.2 Hz, 6H). HRMS (m/z) [M+H]+calcd for C₂₃H₂₆F₄N₅, 448.2119; found, 448.2131.

### Example 15

### Preparation of 4-{3-[4-fluoro-3-(trifluoromethyl)phenyl]-4-pyrimidin-4-yl-1H-pyrazol-5-yl}-N-isopropylcyclohexanamine 4-methylbenzenesulfonate

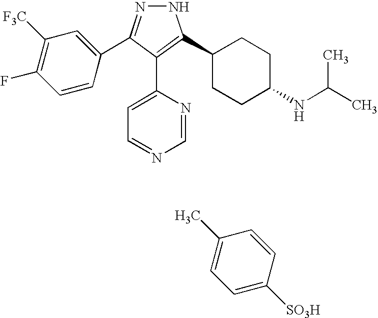

4-{3-[4 fluoro-3-(trifluoromethyl)phenyl]-4-pyrimidin-4-yl-1H-pyrazol-5-yl}-N-isopropylcyclohexanamine (0.79 g, 1.79 mmol) was dissolved in ethanol (15 mL). p-TsOH.H₂O (0.348, 1.79 mmol) was then added to the solution. The reaction mixture was stirred for 1 hr at 25° C. Half the ethanol was slowly removed with a stream of N₂. A white precipitate crashed out of solution upon plunging the reaction vessel into a 0° C. ice bath. The precipitate was collected on a pad and rinsed with diethyl ether (100 mL). A second crop of the white precipitate was obtained from the mother liquor. This white precipitate also was collected on a pad and rinsed with diethyl ether (100 mL). The compound was dried in a vacuum oven at 35° C. for 96 hr to produce the product in the form of a white powder (1.00 g, 90.0%; ¹H NMR (400 MHz, DMSO): 13.33 (br s, 1H), 9.14 (d, J=1.2 Hz, 1H), 8.65 (d, J=5.4 Hz, 1H), 8.15 (br s, 2H), 7.71 (d, J=6.3 Hz, 1H), 6.65 (m, 1H), 7.50 (d, J=10.2 Hz, 1H), 7.46 (d, J=8.1 Hz, 2H), 7.20 (dd, J=5.4, 1.3 Hz, 1H), 7.1 (d, J=7.9 Hz, 2H), 3.30 (m, 1H), 3.05 (m, 2H), 2.25 (s, 3H), 2.06 (d, J=10.6, 2H), 1.94 (d, J=11.9, 2H), 1.67 (dd, J=23.8, 12.8 Hz, 2H), 1.37 (dd, J=22.0, 11.8, Hz, 2H), 1.19 (d, J=6.4, 6H). Anal. Calcd for C₂₃H₂₅F₄N₅.C₇H₈O₃S: C, 58.15, H, 5.37, N, 11.30, S, 5.17. Found: C, 57.91, H, 5.22, N, 11.17, S, 5.17. HRMS (m/z) [M+H] calcd, 448.21; found, 448.20.

### Example 16

### Preparation of 2-{4-[3-(4-chlorophenyl)-4-pyrimidin-4-yl-1H-pyrazol-5-yl]cyclohexyl)propan-2-ol

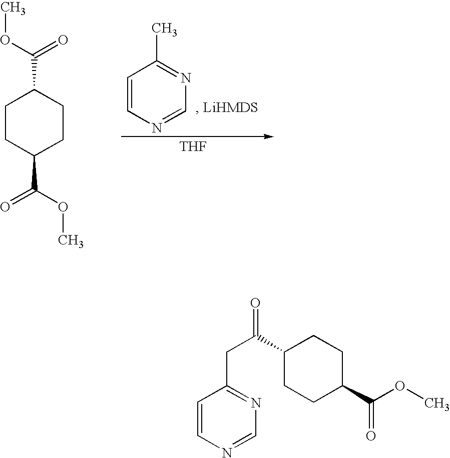

Part A. A solution of 4-methylpyrimidine (25 g, 0.27 mol) and trans-dimethyl-1,4-cyclohexane dicarboxylate (56 g, 0.28 mol) in 250 mL of THF was added to a cooled (−36° C.) solution of 800 mL of 1.0 M LiHMDS (lithium hexamethyldisilazide) in THF (tetrahydrofuran). The final temperature was −26° C. The mixture was re-cooled to −35° C. and stirred for 0.5 hr. The mixture was then quenched with 300 mL of saturated NH₄Cl. Water and MeOH were added to the mixture. The mixtures was then extracted with ethyl acetate (3×600 mL). The organic phase was washed with water (1×250 mL), dried over anhydrous Na₂SO₄, and filtered through a pad of silica gel. The solvent was removed to afford 60 g of a golden oil. The oil crystallized overnight. The oily solid was triturated with Et₂O. The solid was removed by filtration. The filtrate was concentrated to yield 58 g of the crude ketone.

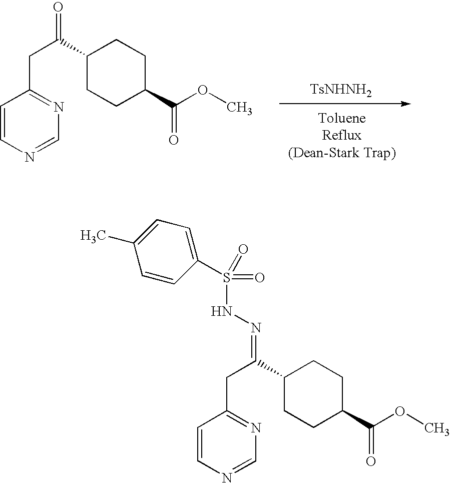

Part B. Solid tosyl hydrazide (42 g, 0.23 mol, “TsNHNH₂”) was added to a solution of the ketone from Part A (58 g, 0.22 mol) in 450 mL of toluene. The mixture was warmed to reflux for 2 hr. Most of the toluene was removed in vacuo to leave an oil. The oil began to solidify and was triturated with 100 mL of ethyl acetate. The suspension was allowed to stand overnight. The solid was removed by filtration, and the filtrate was concentrated to afford an oil. The oil was flashed (50% ethyl acetate/hexane, followed by 80% ethyl acetate/hexane) to afford 41 g of a yellow solid. The solid was triturated with 200 mL of diethyl ether. The suspension was filtered to afford 26 g of a pale yellow solid. The solid was dried in a vacuum oven overnight to produce 22 g of the desired hydrazone. ¹H NMR (CDCl₃) δ 10.97 (s, 1H), 9.01 (d, J=1.21 Hz, 1H), 8.71 (d, J=5.03 Hz, 1H), 7.89 (d, J=8.26 Hz, 2H), 7.31 (d, J=8.06 Hz, 2H), 7.25 (dd, J=5.03, 1.21 Hz, 1H), 3.74 (s, 2H), 3.68 (s, 3H), 2.43 (s, 2H), 2.19 (m, 2H), 2.02 (dd, J=13.49, 2.22 Hz, 2H), 1.80 (d, J=11.28 Hz, 2H), 1.33 (m, 4H); MS (M+H): 431 (base peak).

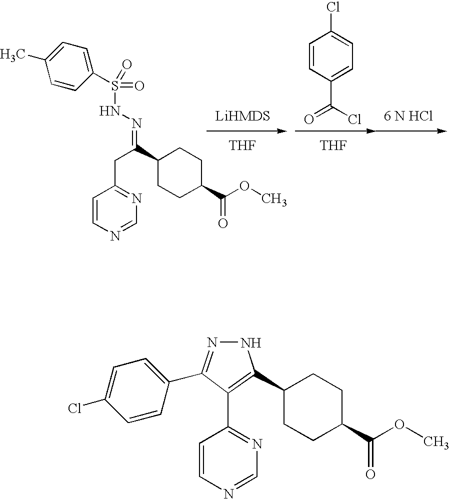

Part C. A cooled (−24° C.) solution of the hydrazone from Part B (10.2 g, 24 mmol) was treated with 25 mL (1 equiv.) of a 1 M solution of LiHMDS in THF. To the resulting solution was added a solution of 4-chlorobenzoylchloride (3 mL, 24 mmol) in 40 mL of THF. The mixture was stirred at −29° C. for 5 min. An additional 25 mL of a 1 M solution of LiHMDS in THF was added. The mixture was warmed to 20° C. Afterward, 100 mL of 6 N HCl was added. The temperature increased to 52° C. The mixture was stirred for 1.5 hr and then poured into 200 mL of water. The aqueous mixture was extracted with ethyl acetate (2×200 mL). The combined organic layers were washed with water (2×200 mL) and brine (1×150 mL), and then dried over anhydrous Na₂SO₄. The solution was filtered through a pad of silica gel. The solvent was then removed to leave 11 g of a crude oil. The oil was flashed (50% ethyl acetate/hexane, followed by 100% ethyl acetate) to afford 2 g of a mixture of the desired ester and the corresponding acid. The material was dissolved in 200 mL of MeOH and treated with 0.5 mL of concentrated H₂SO₄. The resulting solution was stirred at room temperature overnight. Afterward, the solution was concentrated and diluted with water. The mixture was extracted with ethyl acetate (1×250 mL). The organic phase was washed with brine and dried over anhydrous Na₂SO₄. The solution was filtered and concentrated to produce 1.5 g of a crude solid.

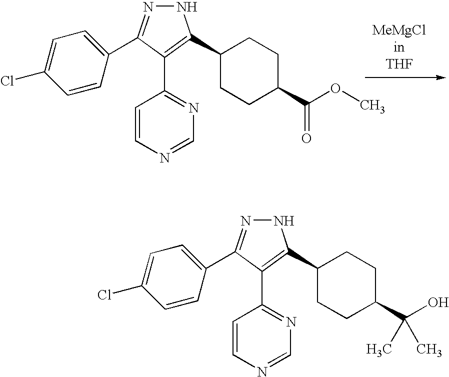

Part D. A solution of the crude ester from Part C (1.5 g, 3.8 mmol) in 40 mL of THF was added to 20 mL of a 3 M cooled (5° C.) solution of MeMgCl (60 mmol) in THF. The mixture was warmed to room temperature and stirred for 45 min. The mixture was then quenched carefully by slowly adding the mixture to 200 mL of saturated NH₄Cl with vigorous stirring. Afterward, the mixture was extracted with ethyl acetate and was washed with brine. The organic layer was dried over anhydrous Na₂SO₄, filtered, and concentrated to leave 1.6 g of an oil. The oil was flashed (80% ethyl acetate/hexane, followed by 100% ethyl acetate) to afford 0.8 g of a yellow solid. The solid was allowed to air dry to produce 610 mg of the desired tertiary alcohol. ¹H NMR (CDCl₃) δ 9.26 (d, J=1.21 Hz, 1H), 8.54 (d, J=5.24 Hz, 1H), 7.34 (s, 4H), 6.99 (dd, J=5.44, 1.21 Hz, 1H), 3.26 (m, 1H), 2.10 (d, J=10.68 Hz, 2H), 1.95 (d, J=11.08 Hz, 2H), 1.52 (m, 2H), 1.27 (m, 3H), 1.19 (s, 6H); MS (M+H): 397 (base peak).

### Example 17

### Preparation of 2-{4-[3-(4-chlorophenyl)-4-pyrimidin-4-yl-1H-pyrazol-5-yl]piperidin-1-yl}cyclopentanol Fumarate

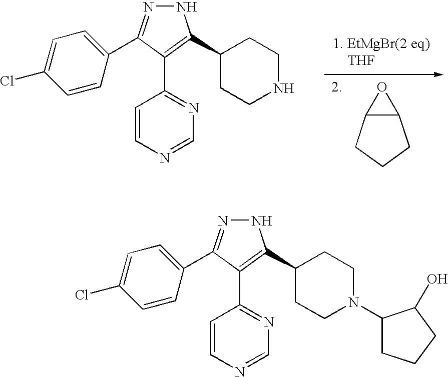

Part A. To a stirred solution of 830 mg of 4-[3-(4-chlorophenyl)-5 piperidin-4-yl-1H-pyrazol-4-yl]pyrimidine (2.45 mmol) in 10 mL of anhydrous THF held at room temperature was slowly added dropwise via syringe 2.7 mL of ethylmagnesium bromide (5.39 mmol, 1.0 M/THF). Immediate evolution of H₂ gas was observed. After stirring for 30 min, 320 mL of cyclopentene oxide (3.67 mmol) was added to the magnesium amide. The reaction mixture was allowed to stir at room temperature for 16 hr. The reaction mixture was then quenched by addition of 1N NH₄Cl. A saturated solution of Rochelles salt was also added to help break-up the resulting emulsion. The reaction mixture was diluted with CH₂Cl₂, and the layers were separated. The aqueous layer was extracted with CH₂Cl₂ (3×). The organics were combined, dried by filtering through Whatman 125 mm 1PS silicone treated filter paper, and finally concentrated in vacuo. The crude material was purified by flash column chromatography (45M biotage cartridge). Elution with (2:1) CH₂Cl₂-MeOH produced 390 mg (38%) of the product. ¹H-NMR (300 MHz, DMSO-d₆) 9.19 (s, 1H), 8.64 (d, J=5.4 Hz, 1H), 7.55-7.38 (m, 4H), 7.15 (d, J=5.1 Hz, 1H), 5.38 (s, 1H), 4.40 (s, 1H), 3.8-3.0 (m, 6H), 2.2-1.5 (m, 10H); Low-Resolution Ms (CI) calculated for C₂₃H₂₇ClN₅O (M+1)=424.18; found 424.

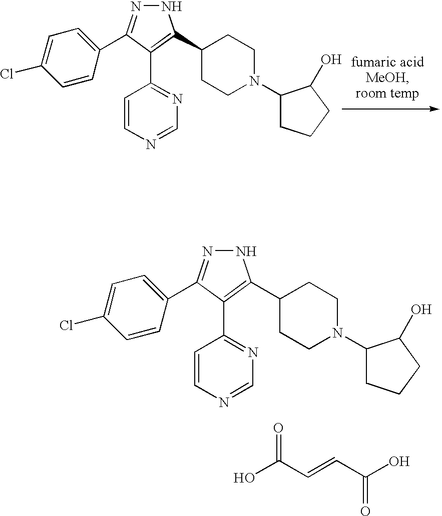

Part B. To a stirred mixture of 390 mg (0.82 mmol) of the 2-{4[3-(4-chlorophenyl)-4-pyrimidin-4-yl-1H-pyrazol-5-yl]piperidin-1-yl}cyclopentanol from Part A in 1 mL of MeOH was added 55 mg (0.41 mmol) of fumaric acid. Upon the addition of fumaric acid, the mixture became homogeneous. After stirring for 2 hr, a thick white precipitate formed. The mixture was filtered and washed with MeOH and then diethyl ether to produce the product in the form of a white solid. ¹H-NMR (400 MHz, DMSO-d₆) 9.12 (s, 1H), 8.58 (d, J=5.2 Hz, 1H), 7.44-7.42 (m, 4H), 7.08 (d, J=5.2 Hz, 1H), 6.57 (s, 1H), 4.38-4.25 (m, 1H), 3.8-2.8 (m, 6H); 2.09-1.94 (m, 4H), 1.86 (dt, J=14.8, 7.6 Hz, 1H), 1.74-1.64 (m, 1H), 1.58 (dq, J=13.2, 6.8 Hz, 2H), 1.52-1.46 (m, 1H). High-resolution MS (CI) calculated for C₂₃H₂₇ClN₅O (M+1)=424.18; found 424.19.

### Example 18

### Preparation of 2-[4-(1-{4-[3-(4-chlorophenyl)-4-pyrimidin-4-yl-1H-pyrazol-5-yl]piperidin-1-yl}ethyl)phenyl]propan-2-ol

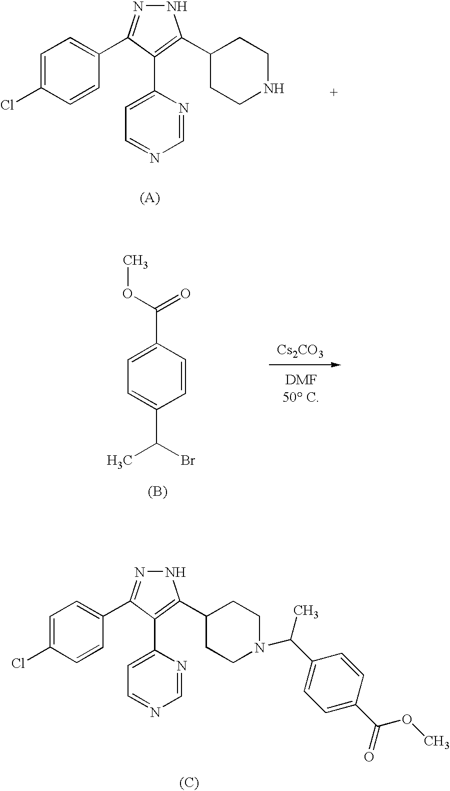

Part A. 4-[3-(4-chlorophenyl)-5-piperidin-4-yl-1H-pyrazol-4-yl]pyrimidine (3 g, 8.8 mmol, Compound A) was added to methyl 4-(1-bromoethyl)benzoate (2.1 g, 8.7 mmol, Compound B), 20 ml of dimethylformamide (DMF), and 5.7 g Cs₂CO₃ (17.6 mmol). The mixture was heated to 50° C. and stirred for 16 hr. The mixture was then cooled to room temperature and quenched it with 200 ml of water. The resulting suspension was extracted ethyl acetate (2×250 ml). The combined organic layers were washed with brine (1×300 ml), dried over MgSO₄, and evaporated to dryness. The resulting residue was crystallized from MeOH/ethyl acetate and hexane to afford 3.1 g of white solid. ¹H NMR (400 MHz, CDCl₃): 9.17 (d, J=1.34 Hz, 1H), 8.47 (d, J=5.24 Hz, 1H), 7.98 (d, J=8.32 Hz, 2H), 7.38 (d, J=8.32 Hz, 2H), 7.31 (s, 4H), 6.93 (dd, J=5.26, 1.4 Hz, 1H), 3.9 (s, 3H), 3.51 (m, 1H), 3.21 (m, 1H), 3.13 (d, J=10.7 Hz, 1H), 2.86 (d, J=11.1 Hz, 1H), 1.92 (m, 6H), 1.38 (d, J=6.7 Hz, 3H); LC/MS, tᵣ=2.36 min (5 to 95% AcCN/water over 6 min at 1 ml/min, at 254 nm, at 50 C), (M+H), Calculated=502, Found=502.

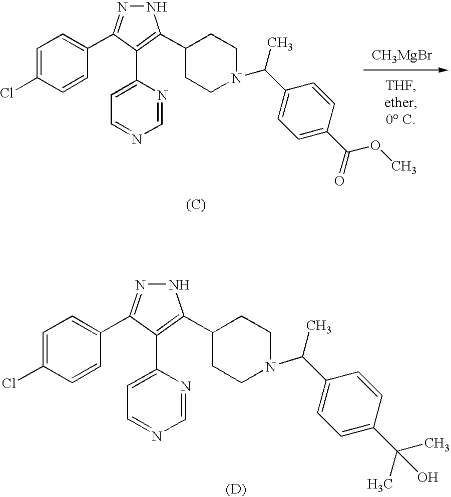

Part B. In a 100 ml round bottom flask under N₂, 5 ml of 3 M methyl magnesium bromide in ether (15 mmol) was added to 5 ml of anhydrous THF. This mixture was cooled to 0° C. In a separate addition funnel, methyl 4-(1-{4-[3-(4-chlorophenyl)-4-pyrimidin-4-yl-1H-pyrazol5-yl] piperidin-1-yl} ethyl) benzoate (300 mg, 0.6 mmol, Compound C) was dissolved in 20 ml of anhydrous THF and added to the 0° C. mixture drop wise at a rate of 0.2 ml/min. After the addition, the mixture was stirred at 0° C. for an additional 30 min. The mixture was then poured into a stirring 0° C. solution of saturated ammonium chloride (100 ml). The mixture was then extracted with ethyl acetate (2×150 ml). The combined organic layers were washed with water (2×100 ml), dried over MgSO₄, and evaporated to dryness. The residue was crystallized from MeOH/ethyl acetate and hexane to afford 260 mg of white solid. ¹H NMR (400 MHz, CDCl₃): 9.15 (d, J=1.07 Hz, 1H), 8.45 (d, J=5.37 Hz, 1H), 7.41 (d, J=8.33 Hz, 2H), 7.30 (br s, 3H), 7.23 (m, 3H), 6.917 (dd, J=6.24, 1.47 Hz, 1H), 3.47 (m, 3H), 3.25 (m, 1H), 3.13 (m, 1H), 2.90 (m, 1H), 1.91 (m, 6H), 1.56 (s, 6H), 1.37 (d, J=6.98 Hz, 3H). HRMS (m/z): [M+H]+calcd for C₂₉H₃₃ClN₅O, 502.2368; found, 502.2379.

### Example 19

### Preparation of 4-{3-(4-chlorophenyl)-5-[1-(4-fluorobenzyl)piperidin-4-yl]-1H-pyrazol-4-yl}pyrimidine

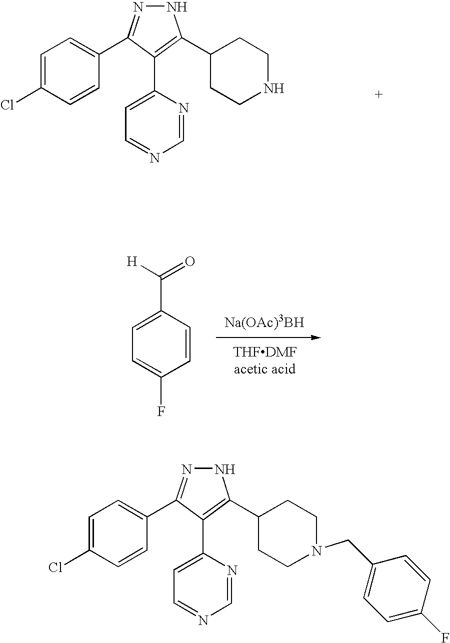

4-[3-(4-chlorophenyl)-5-piperidin-4-yl-1H-pyrazol-4-yl]pyrimidine (80 g, 235.2 mmol) was added to 300 ml 50/50 mixture of THF/DMF. The mixture was cooled to 0° C. To this cooled mixture was added 20 ml of acetic acid, 4-fluorobenzaldehyde (50 g, 403 mmol), and sodium triacetoxyborohydride (90 g, 424 mmol) in the order listed. After the addition, the mixture was warmed to room temperature and stirred for 16 hr. It was then quenched with 1.5 liter of saturated NaHCO₃, which was added slowly while the mixture was constantly stirred. The resulting white suspension was filtered, and the white precipitate was rinsed with water (10×500 ml). The precipitate was then crystallized from MeOH/ethylacetate and hexane to afford white solid. The resulting solid was re-crystallized from MeOH/water to afford 102 g of white solid. ¹H NMR (400 MHz, CDCl₃): 9.18 (d, J=1.2 Hz, 1H), 8.46 (d, J=5.37 Hz, 1H), 7.25 (m, 6H), 6.93 (m, 3H), 3.48 (s, 2H), 3.3 (m, 1H), 3.13 (m, 1H), 2.94 (br d, J=11.4 Hz 2H), 2.06 (m, 2H), 1.90 (m, 4H). HRMS (m/z): [M+H]+calcd for C₂₅H₂₃ClFN₅, 448.1699; found, 448.1687.

### Example 20

### Preparation of 4-{3-(4-chlorophenyl)-5-[1-(4-fluorobenzyl)piperidin-4-yl]-1H-pyrazol-4-yl}pyrimidine Hydrochloride

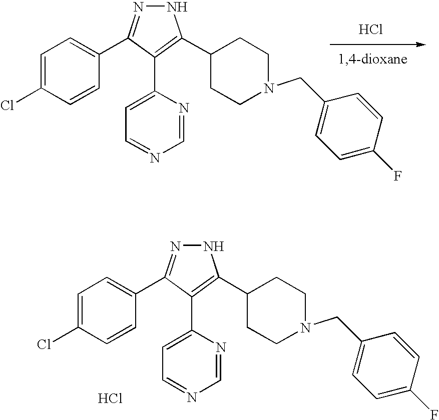

To 4-{3-(4-chlorophenyl)-5-[1-(4-fluorobenzyl)piperidin-4-yl]-1H-pyrazol-4-yllpyrimidine (2 g, 4.4 mmol) was added 4N HCl in 1,4-dioxane (10 ml). The mixture was stirred for 1 hr and evaporated to form a dried residue. The residue was crystallized from MeOH/ethylacetate and ether to afford 2 g of white solid. ¹H NMR (400 MHz, d-DMSO): 9.13 (s, 1H), 8.59 (d, J=5.24 Hz, 1H), 7.65 (m, 2H), 7.45 (m, 2H), 7.34 (m, 2H), 7.27 (m, 2H), 7.08 (m, 1H), 4.25 (m, 2H), 3.33 (m, 3H), 2.98 (m, 2H), 2.08 (m, 4H). HRMS (m/z): [M+H]⁺ calcd for C₂₅H₂₃ClFN₅, 448.1704; found, 448.1713.

### Example 21

### Preparation of di(tert-butyl) 2-{4-[3-(4-chlorophenyl)-4-pyrimidin-4-yl-1H-pyrazol-5-yl]piperidin-1-yl}-2-oxoethyl Phosphate

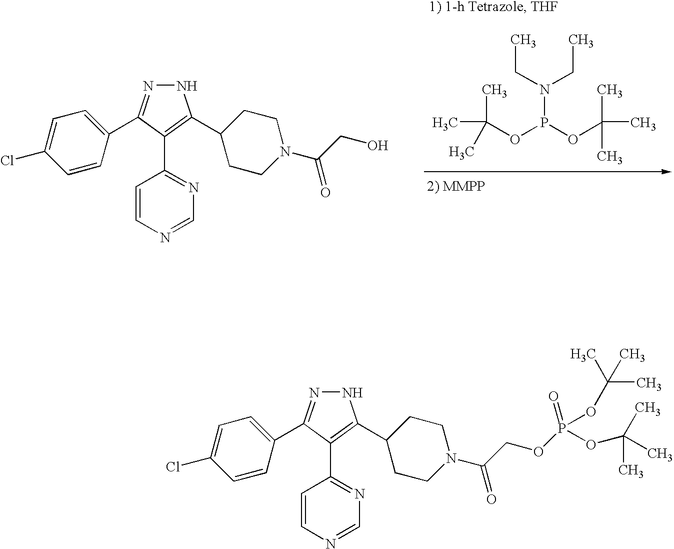

To a suspension of 20 g (50 mmol) of 2-{4-[3-(4-chlorophenyl)-4-pyrimidin-4-yl-1H-pyrazol-5-yl]piperidin-1-yl}-2-oxoethanol and 7.1 g (101 mmol) of 1-H tetrazole in 275 mL of THF was added 18.8 mL (63 mmol) of di-t-butyl-N,N-diethyl phosphoramidite. The reaction mixture was stirred at room temperature overnight. An additional 2 mL (7.2 mmol) of di-t-butyl-N,N-diethyl phosphoramidite was added, and the reaction mixture was stirred for 1 h. The mixture was cooled to 4° C., and 34.3 g of monomethyl magnesium perphthalate was added. The ice bath was removed and the reaction mixture was allowed to warm to room temperature overnight. Subsequently, the reaction mixture was poured onto 300 mL of ethyl acetate and a precipitate formed. The slurry was filtered, and the filter cake was washed with additional ethyl acetate. The filtrate was treated with 500 mL of water and the resulting biphasic mixture was stirred for 3 h. The phases were separated and the organic layer was washed with 400 mL of saturated NaHCO₃. The organic layer was dried over anhydrous Na₂SO₄, filtered, and concentrated in vacuo to leave 30 g of a yellow liquid. ¹H NMR (CDCl₃) δ 9.25 (s, 1H), 8.52 (d, J=5.4 Hz, 1H), 7.34 (s, 4H), 7.01 (dd, J=5.4 Hz and 1.21 Hz, 1H), 7.31 (d, J=8.06 Hz, 2H), 7.25 (dd, J=5.03, 1.21 Hz, 1H), 4.65 (m, 3H), 3.94 (bd, J=12.9 Hz, 1H), 3.64 (m, 1H), 3.17 (m, 1H), 2.73 (m, 1H), 2.02 (m, 3H), 1.87 (m, 1H), 1.50 (s, 9H); ³¹P NMR (CDCl₃) δ-9.52; MS (M−112+H): 478 (loss of two t-butyl groups).

### Example 22

### Preparation of 2-{4-[3-(4-chlorophenyl)-4-pyrimidin-4-yl-1H-pyrazol-5-yl]piperidin-1-yl}-2-oxoethyl Dihydrogen Phosphate

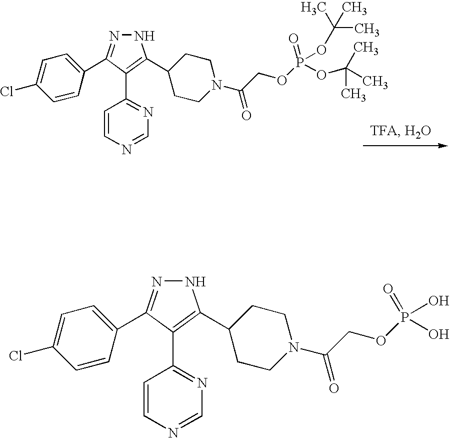

A stirred suspension of 15 g (26 mmol) of di(tert-butyl) 2-{4-[3-(4-chlorophenyl)-4-pyrimidin-4-yl-1H-pyrazol-5-yl]piperidin-1-yl}-2-oxoethyl phosphate in 25 mL of dioxane and 75 mL of water was treated with 0.5 mL of trifluoroacetic acid (TFA). The reaction mixture was allowed to stir at room temperature overnight. An additional 1 mL of TFA and 50 mL of dioxane were added, and the mixture was stirred overnight. The suspension was filtered and washed with dioxane and diethyl ether to leave a white solid. The solid was dried in a vacuum oven overnight to afford 8.1 g of the desired phosphate. ¹H NMR (DMF-d₇) δ 9.39 (d, J=1.23 Hz, 1H), 8.86 (d, J=5.24 Hz, 1H), 7.67 (s, 4H), 7.44 (dd, J=5.43 and 1.21 Hz, 1H), 4.88 (m, 2H), 4.74 (d, J=12.49 Hz, 1H), 4.17 (d, J=13.9 Hz, 1H), 3.72 (m, 1H), 3.33 (m, 1H), 2.16 (m, 3H), 2.03 (m, 1H); ³¹P NMR (DMF-d₇) δ 1.1; MS (M+H): 478 (base peak).

### Example 23

### Preparation of mono-sodium 2-{4-[3-(4-chlorophenyl)-4-pyrimidin-4-yl-1H-pyrazol-5-yl]piperidin-1-yl}-2-oxoethyl Dihydrogen Phosphate Dihydrate

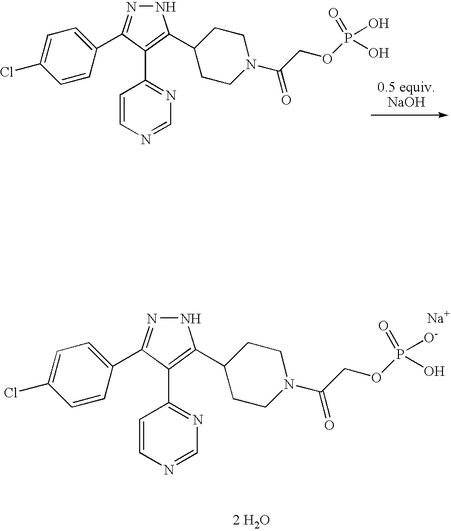

To a stirred suspension of 0.2 g (0.42 mmol) of 2-{4-[3-(4-chlorophenyl)-4-pyrimidin-4-yl-1H-pyrazol-5-yl]piperidin-1-yl}-2-oxoethyl dihydrogen phosphate in 1.7 mL of water was added 0.833 mL of 0.5 N NaOH. A precipitate began to form after 15 min. The mixture was diluted with 10 mL of water to re-dissolve the solid. The solution was transferred to two conical vials and placed in the Genevac overnight. The vials were removed from the Genevac to afford 0.19 g of the mono-sodium salt. ¹H NMR (D₂O) δ 8.94 (d, J=1.0 Hz, 1H), 8.49 (d, J=5.24 Hz, 1H), 7.25 (d, J=8.46 Hz, 2H), 7.16 (dd, J=5.44 and 1.21, 1H), 7.11 (d, J=8.46 Hz, 2H), 4.49 (m, 2H), 4.33 (d, J=13.3 Hz, 1H), 3.76 (d, J=13.7 Hz, 1H), 3.24 (m, 1H), 3.04 (m, 1H), 2.65 (m, 1H), 1.80 (m, 2H), 1.59 (m, 2H); ³¹P NMR (D₂O) δ 1.334; MS (M+2H): 478 (base peak); Anal. Calcd. for C₂₀H₂₀ClN₅NaO₅P: C, 48.06; H, 4.03; N, 14.01. Found: C, 45.07; H, 4.45; N, 13.13 (consistent with a dihydrate).

### Example 24

### Preparation of di-sodium 2-{4-[3-(4-chlorophenyl)-4-pyrimidin-4-yl-1H-pyrazol-5-yl]piperidin-1-yl}-2-oxoethyl Dihydrogen Phosphate Dihydrate

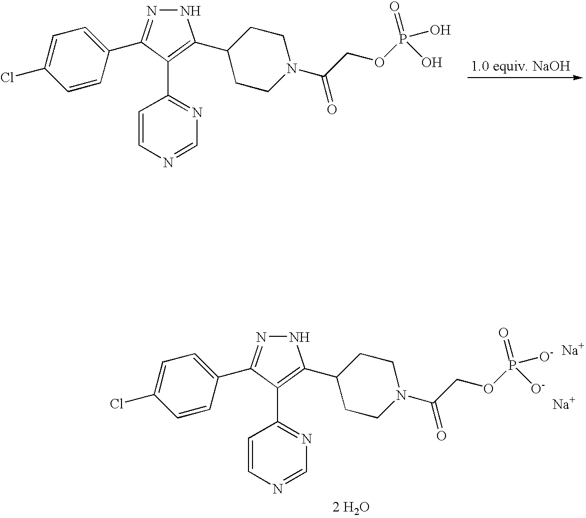

To a stirred suspension of 0.2 g (0.42 mmol) of 2-{4-[3-(4-chlorophenyl)-4-pyrimidin-4-yl-1H-pyrazol-5-yl]piperidin-1-yl}-2-oxoethyl dihydrogen phosphate in 1.7 mL of water was added 1.666 mL of 0.5 N NaOH. Complete dissolution occurred after 5 min. The solution was diluted with 10 mL of water. The solution was transferred to two conical vials and placed in the Genevac overnight. Afterward, the vials were removed from the Genevac to afford 0.22 g of the di-sodium salt. ¹H NMR (D₂O) δ 8.94 (d, J=0.81 Hz, 1H), 8.49 (d, J=5.44 Hz, 1H), 7.23 (d, J=8.46 Hz, 2H), 7.15 (dd, J=5.44 and 1.41, 1H), 7.10 (d, J=8.66 Hz, 2H), 4.39 (m, 3H), 3.83 (d, J=13.3 Hz, 1H), 3.23 (m, 1H), 3.03 (m, 1H), 2.64 (m, 1H), 1.81 (m, 2H), 1.49-1.74 (m, 2H); 31p NMR (D₂O) δ 4.72; MS (M+3H): 478 (base peak); Anal. Calcd. for C₂₀H₁₉ClN₅Na₂O₅P: C, 46.04; H, 3.67; N, 13.42. Found: C, 42.73; H, 4.12; N, 12.38 (consistent with a dihydrate).

### Example 25

### Preparation of 2-{4-[3-(4-chlorophenyl)-4-pyrimidin-4-yl-1H-pyrazol-5-yl]piperidin-1-yl}-2-oxoethyl N,N-dimethylglycinate Hydrochloride

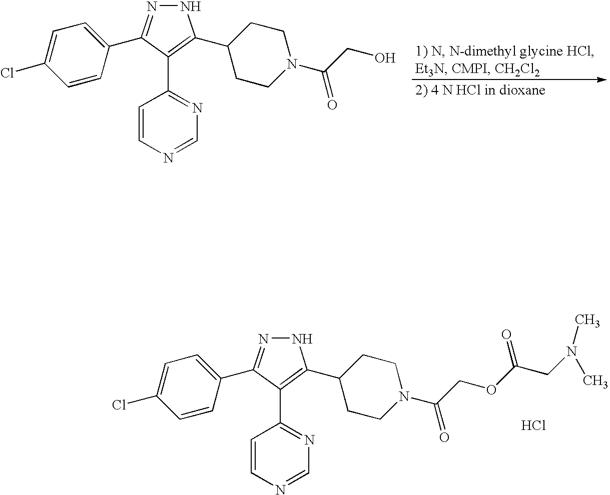

To a suspension of 10 g (25 mmol) of 2-{4-[3-(4-chlorophenyl)-4-pyrimidin-4-yl-1H-pyrazol-5-yl]piperidin-1-yl}-2-oxoethanol in 125 mL of CH₂Cl₂ was added (in the following order) 10.5 mL (76 mmol) of triethylamine (Et₃N), 3.5 g (25 mmol) of N,N-dimethyl glycine hydrochloride, and 7 g (28 mmol) of CMPI. The mixture was allowed to stir at room temperature overnight. The reaction mixture was washed with 250 mL of sat. NaHCO₃ (with 5 g of Na₂SO₃) and 250 mL of water. The CH₂Cl₂ layer was dried over anhydrous Na₂SO₄, filtered, and concentrated to leave 13 g of a thick oil. To this thick oil was added 100 mL of acetonitrile. A solid precipitated. The mixture was allowed to stand at room temperature overnight. Subsequently, the mixture was filtered to leave 5.6 g of a white solid. The solid was placed into a vacuum oven overnight to afford 5.4 g of a white solid. To a suspension of 1 g of this solid in 20 mL of THF was added 0.52 mL of 4 N HCl in dioxane. This mixture was stirred at room temperature for 2 h. The mixture was then concentrated in vacuo and residual solvent was chased in vacuo with toluene. The resulting solid was triturated with ethyl ether (Et₂O). The suspension was sonicated for 30 min and filtered to leave 1.04 g of a white solid. The solid was placed in a vacuum oven overnight to afford 0.98 g of the desired HCl salt. ¹H NMR (D₂O) δ 8.70 (s, 1H), 8.52 (d, J=5.0 Hz, 1H), 7.28 (d, J=8.9 Hz, 2H), 7.21 (d, J=5.2, 1H), 7.14 (d, J=8.9 Hz, 2H), 4.31 (m, 1H), 4.16 (s, 2H), 3.69 (m, 1H), 3.26 (m, 1H), 3.10 (m, 1H), 2.89 (s, 6H), 2.74 (m, 1H), 1.84 (m, 2H), 1.58 (m, 2H); MS (M+H): 483 (base peak).

### Example 26

### Preparation of 4-[3-(4-chloro-2-fluorophenyl)-5-piperidin-4-yl-1H-pyrazol-4-yl]pyrimidine

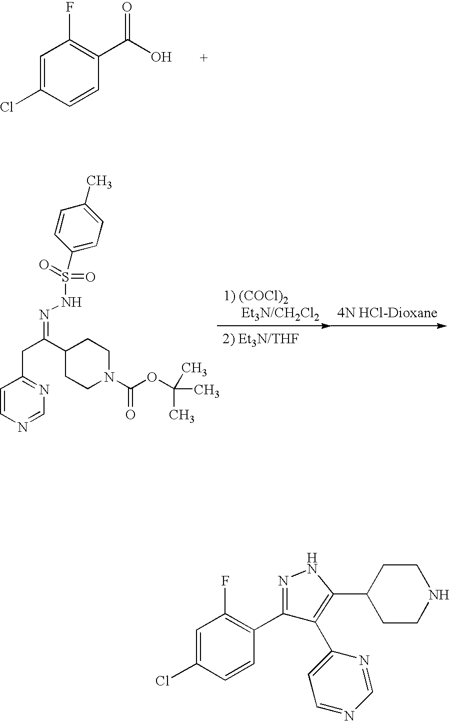

Part A. To a solution of 6.0 g (34 mmol) of 2-fluoro,4-chlorobenzoic acid in 120 mL of CH₂Cl₂ was added 7 mL (51 mmol) of Et₃N, followed by slow dropwise addition (ca 10 min) of 3.2 ml (37.4 mmol) of oxalyl chloride. The resulting black solution was allowed to stir at room temperature for 2 h. The CH₂Cl₂ was then removed under vacuum. The resulting residue was then re-dissolved in 150 ml of anhydrous tetrahydrofuran. To this mixture was added 7 mL (51 mmol) of Et₃N, followed by addition of 16 g (34 mmol) of tert-butyl 4-{(1E)-N-[(4-methylphenyl) sulfonyl]-2-pyrimidin-4-ylethanehydrazonoyl} piperidine-1-carboxylate. An additional 1 mL (11 mmol) of oxalyl chloride was added to the mixture, which was then stirred at room temperature overnight. The reaction mixture was then quenched with the addition of 50 mL of water. Afterward, the layers were separated, and the aqueous layer was extracted with CH₂Cl₂ (5×50 mL). The organics were combined, dried, and concentrated in vacuo.

Part B. The crude pyrazole from Part A was dissolved in 150 mL of dioxane. To this mixture was added 20 mL (80 mmol) of a 4N HCl-dioxane solution. The reaction mixture was then stirred at room temperature for 2 days. The mixture was then diluted with 250 ml of water. Afterward, the mixture was washed with diethyl ether. The aqueous layer was then neutralized to pH of approximately 9-11 by the addition of aqueous NaOH. The basic aqueous layer was then extracted with CH₂Cl₂ (5×50 ml). The organic layers were combined, dried, and concentrated in vacuo to produce a reddish-brown residue. This residue was then triturated with acetonitrile, followed by filtration to afford 3.5 g of the desired product as a slightly yellow solid. ¹H-NMR (DMSO_{d6}) δ 9.09 (d, J=1.6 Hz, 1H), 8.62 (d, J=7.2 Hz, 1H), 7.58-7.36 (m, 3H), 7.08 (dd, J=7.2, 1.6 Hz, 1H), 3.31-3.20 (m, 2H), 3.01-2.98 (bd, J=16.0 Hz, 2H), 2.53-2.46 (m, 3H), 1.77-1.60 (m, 4H); MS (M+H): 358.

### Example 27

### Preparation of 2-{4-[3-(4-chloro-2-fluorophenyl)-4-pyrimidin-4-yl-1H-pyrazol-5-yl]piperidin-1-yl}-2-oxoethanol

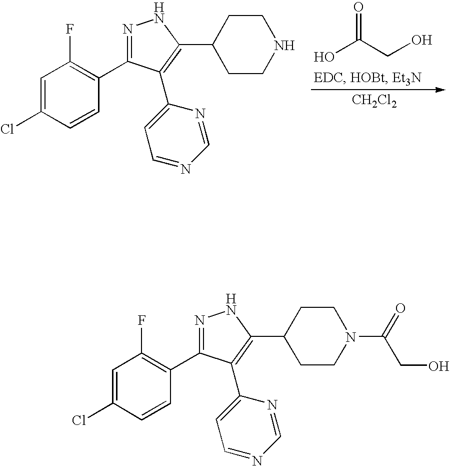

To a solution of 2.3 g (6.5 mmol) of 4-[3-(4-chloro-2-fluorophenyl)-5-piperidin-4-yl-1H-pyrazol-4-yl]pyrimidine in 32.5 ml of CH₂Cl₂ was added (in the following order) 1.3 ml (9.75 mmol) of Et₃N, 0.60 g (7.8 mmol) of glycolic acid, and 1 g (7.8 mmol) of hydroxybenzotriazole (HOBt). The mixture was allowed to stir together for 5 min, after which 1.3 g (7.15 mmol) of N-ethyl-N′-(3-dimethylaminopropyl)carbodiimide (EDC) was added. The reaction mixture was stirred at room temperature for 12 hr, and then quenched by addition of 1N aqueous solution of NH₄Cl. The layers were separated, and the aqueous layer was extracted with CH₂Cl₂ (4×10 ml). The organics were combined, dried, and concentrated in vacuo. The resulting solid was triturated with acetonitrile and filtered to give 1.6 g of the desired product as an off-white solid. ¹H-NMR (DMSO_{d6}) δ 9.10 (d, J=1.6 Hz, 1H), 8.63 (d, J=7.2 Hz, 1H), 7.58-7.37 (m, 3H), 7.11 (dd, J=7.2, 1.6 Hz, 1H), 4.54-4.45 (m, 2H), 4.11, (S, 2H), 3.80-3.76 (bd, J=18.0 Hz, 1H), 3.49 (t, J=15.5 HZ, 1H), 3.05 (t, J=16.4 Hz, 1H), 2.69 (t, J=16.4 Hz, 1H), 1.93-1.65 (m, 5H); ¹⁹F-NMR-112.01(s); MS (M+H): 416.

### Example 28

### Preparation of 2-(4-{3-[4-fluoro-3-(trifluoromethyl)phenyl]-4-pyrimidin-4-yl-1H-pyrazol-5-yl}piperidin-1-yl)-2-oxoethanol

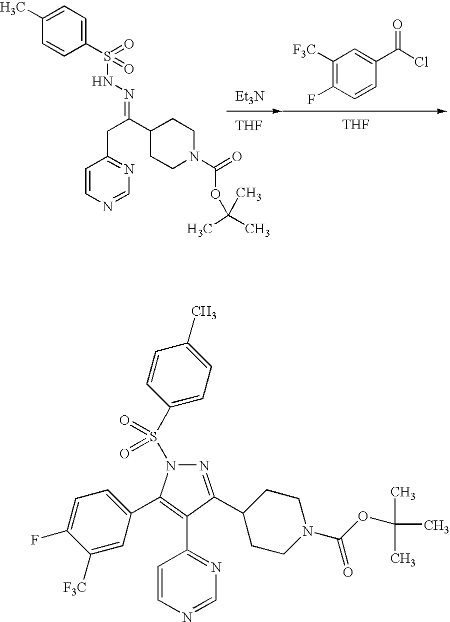

Part A. To a suspension of 28.5 g (60 mmol) of tert-butyl 4-{N-[(4-methylphenyl)sulfonyl]-2-pyrimidin-4-ylethanehydrazonoyl}piperidine-1-carboxylate, 17 mL of Et₃N, and 0.75 g (6 mmol) of dimethylaminopyridine (DMAP) in 120 mL of tetrahydrofuran (THF) was added 10 mL of 4-fluoro-3-trifluoromethylbenzoyl chloride. The temperature increased from 24° C. to 46° C. The reaction mixture was allowed to stir at room temperature overnight. The resulting slurry was filtered, and the filter cake was washed with THF. The filtrate was concentrated to leave a yellow solid. The solid was triturated with 100 mL of MeOH, and the suspension was sonicated for 45 min. The suspension was cooled to room temperature, and then filtered to afford 22.5 g of the desired pyrazole. ¹H NMR (CDCl₃) δ 9.19 (s, 1H), 8.51 (bs, 1H), 7.60 (d, J=8.26 Hz, 2H), 7.53 (m, 1H), 7.41 (dd, J=6.64 and 2.01 Hz, 1H), 7.31 (m, 3H), 6.67 (d, J=4.43 Hz, 1H), 4.17 (bd, J=13.09 Hz, 2H), 3.34 (m, 1H), 2.80 (m, 2H), 2.45 (s, 1H), 1.82 (bm, 4H), 1.50 (s, 9H); ¹⁹F NMR (CDCl₃) δ-61.8 (d); -111.51 (m); MS (M+Na): 668.

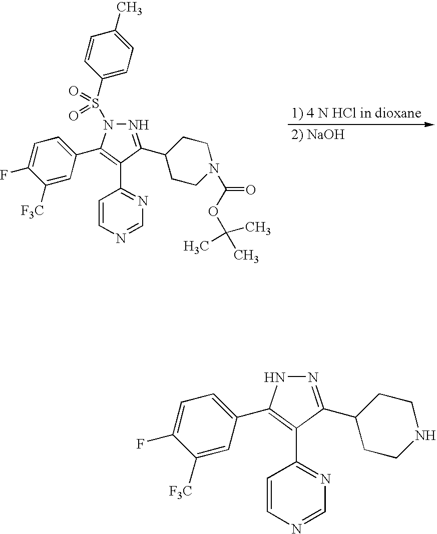

Part B. A stirred solution of 5 g (8 mmol) of tert-butyl 4-{5-[4-fluoro-3-(trifluoromethyl)phenyl]-1-[(4-methylphenyl)sulfonyl]-4-pyrimidin-4-yl-1H-pyrazol-3-yl}piperidine-1-carboxylate from Part A in 30 mL of THF was charged with 20 mL of 4N HCl in dioxane. The solution was allowed to stir at room temperature overnight, during which time a precipitate formed. The slurry was dissolved in 100 mL of water and then washed with 300 mL of diethyl ether. The aqueous phase was neutralized with aqueous NaOH to a pH of 11. The aqueous mixture was extracted with CH₂Cl₂ (2×250 mL), and the combined organic extracts were dried over anhydrous Na₂SO₄. The solution was filtered, and the solvent was removed in vacuo to afford 2.9 g of the desired amine. ¹H NMR (CD₃OD) δ 9.17 (d, J=1.2 Hz, 1H), 8.62 (d, J=5.43 Hz, 1H), 7.76 (dd, J=7.05 and 2.02 Hz, 2H), 7.65 (m, 1H), 7.37 (m, 1H), 7.22 (dd, J=5.44 and 1.41 Hz, 1H), 3.42 (m, 1H), 3.23 (bd, 2H), 2.80 (m, 2H), 2.01 (m, 2H), 1.88 (m, 2H); ¹⁹F NMR (CD₃OD) δ-63.42 (d), -117.56 (bs); MS (M+H): 392.

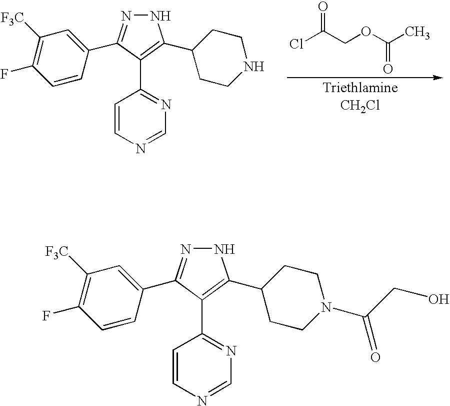

Part C. To a cooled (5° C.), stirred mixture of 1.5 g (4 mmol) of 4-{3-[4-fluoro-3-(trifluoromethyl)phenyl]-5-piperidin-4-yl-1H-pyrazol-4-yl}pyrimidine of Part B and 1.5 mL of Hunig's base in 20 mL of CH₂Cl₂ was added 0.5 mL of acetoxyacetyl chloride. The reaction mixture was allowed to stir overnight. To this mixture was added 15 mL of MeOH and 3 mL of 2.5 N NaOH. The mixture was stirred for 1.5 h, and then poured onto 200 mL of water. The mixture was extracted with 200 mL of CH₂Cl₂, and the solvent was removed in vacuo to give a yellow solid. Residual solvent was chased with diethyl ether to leave 1.4 g of a the desired amide as a pale yellow solid. ¹H NMR (CDCl₃) δ 9.25 (d, J=1.21 Hz, 1H), 8.56 (d, J=5.24 Hz, 1H), 7.75 (dd, J=6.65 and 1.81 Hz, 1H), 7.53 (m, 1H), 7.20 (m, 1H), 6.97 (dd, J=5.44 and 1.41 Hz, 1H), 4.73 (bd, J=13.06 Hz, 1H), 4.25 (s, 2H), 3.65 (m, 2H), 3.16 (m, 1H), 2.83 (m, 1H), 2.12 (bm, 2H), 1.81 (m, 2H); ¹⁹F NMR (CDCl₃) δ-61.88 (d); -114.17 (bs); MS (M+H): 450.

### Examples 29-55

### In Vitro p38 Kinase Inhibition Analysis

Several pyrazole compounds in Examples 1-28 were analyzed in vitro assays to determine their ability to inhibit p38α kinase.

### Cloning of Human p38α

The coding region of the human p38α cDNA was obtained by PCR-amplification from RNA isolated from the human monocyte cell line THP.1. First strand cDNA was synthesized from total RNA as follows: 2 μg of RNA was annealed to 100 ng of random hexamer primers in a 10 μl reaction by heating to 70° C. for 10 min, followed by 2 min on ice. cDNA was then synthesized by adding 1 μl of RNAsin (Promega, Madison Wis.), 2 μl of 50 mM dNTP's, 4 μl of 5× buffer, 2 μl of 100 mM DTT and 1 μl (200 U) of Superscript II™ AMV reverse transcriptase. Random primer, dNTP's and Superscript™ reagents were all purchased from Life-Technologies, Gaithersburg, Mass. The reaction was incubated at 42° C. for 1 hr. Amplification of p38 cDNA was performed by aliquoting 5 μl of the reverse transcriptase reaction into a 100 μl PCR reaction containing the following: 80 μl dH₂O, 2 μl 50 mM dNTP's, 1 μl each of forward and reverse primers (50 pmol/μl), 10 μl of 10× buffer, and 1 μl Expand™ polymerase (Boehringer Mannheim). The PCR primers incorporated Bam HI sites onto the 5′ and 3′ end of the amplified fragment, and were purchased from Genosys. The sequences of the forward and reverse primers were

5′-GATCGAGGATTCATGTCTCAGGAGAGGCCCA-3′ and

5′GATCGAGGATTCTCAGGACTCCATCTCTTC-3′, respectively. The PCR amplification was carried out in a DNA Thermal Cycler (Perkin Elmer) by repeating 30 cycles of 94° C. for 1 min, 60° C. for 1 min, and 68° C. for 2 min. After amplification, excess primers and unincorporated dNTP's were removed from the amplified fragment with a Wizard™ PCR prep (Promega), and digested with Bam HI (New England Biolabs). The Bam HI digested fragment was ligated into BamHI digested pGEX 2T plasmid DNA (PharmaciaBiotech) using T-4 DNA ligase (New England Biolabs) as described by T. Maniatis, Molecular Cloning: A Laboratory Manual, 2nd ed. (1989). The ligation reaction was transformed into chemically competent E. coli DH10B cells purchased from Life-Technologies following the manufacturer's instructions. Plasmid DNA was isolated from the resulting bacterial colonies using a Promega Wizard™ miniprep kit. Plasmids containing the appropriate Bam HI fragment were sequenced in a DNA Thermal Cycler (Perkin Elmer) with Prism™ (Applied Biosystems Inc.). cDNA clones were identified that coded for both human p38a isoforms (Lee et al. Nature 372, 739). One of the clones which contained the cDNA for p38a-2 (CSBP-2) inserted in the cloning site of pGEX 2T, 3′ of the GST coding region was designated pMON 35802. The sequence obtained for this clone is an exact match of the cDNA clone reported by Lee et al. This expression plasmid allows for the production of a GST-p38a fusion protein.

### Expression of Human p38α

GST/p38α fusion protein was expressed from the plasmid pMON 35802 in E. coli, stain DH10B (Life Technologies, Gibco-BRL). Overnight cultures were grown in Luria Broth (LB) containing 100 mg/ml ampicillin. The next day, 500 ml of fresh LB was inoculated with 10 ml of overnight culture, and grown in a 2 liter flask at 37° C. with constant shaking until the culture reached an absorbance of 0.8 at 600 nm. Expression of the fusion protein was induced by addition of isopropyl b-D-thiogalactosidse (IPTG) to a final concentration of 0.05 mM. The cultures were shaken for three hr at room temperature, and the cells were harvested by centrifugation. The cell pellets were stored frozen until protein purification.

### Purification of p38α Kinase

All chemicals were from Sigma Chemical Co. unless noted. Twenty grams of E. coli cell pellet collected from five 1 L shake flask fermentations were re-suspended in a volume of PBS (140 mM NaCl, 2.7 mM KCl, 10 mM Na₂HPO₄, 1.8 mM KH₂PO₄, pH 7.3) up to 200 ml. The cell suspension was adjusted to 5 mM DTT with 2 M DTT and then split equally into five 50 ml Falcon conical tubes. The cells were sonicated (Ultrasonics model W375) with a 1 cm probe for 3×1 min (pulsed) on ice. Lysed cell material was removed by centrifugation (12,000×g, 15 min), and the clarified supernatant applied to glutathione-sepharose resin (Pharmacia).

### Glutathione-Sepharose Affinity Chromatography

Twelve ml of a 50% glutathione sepharose-PBS suspension was added to 200 ml clarified supernatant, and then incubated batchwise for 30 min at room temperature. The resin was collected by centrifugation (600×g, 5 min) and washed with 2×150 ml PBS/1% Triton X-100, followed by 4×40 ml PBS. To cleave the p38 kinase from the GST-p38 fusion protein, the glutathione-sepharose resin was re-suspended in 6 ml PBS containing 250 units thrombin protease (Pharmacia, specific activity >7500 units/mg), and then mixed gently for 4 hr at room temperature. The glutathione-sepharose resin was removed by centrifugation (600×g, 5 min) and washed 2×6 ml with PBS. The PBS wash fractions and digest supernatant containing p38 kinase protein were pooled and adjusted to 0.3 mM PMSF.

### Mono Q Anion Exchange Chromatography

The thrombin-cleaved p38 kinase was further purified by FPLC-anion exchange chromatography. Thrombin-cleaved sample was diluted 2-fold with Buffer A (25 mM HEPES, pH 7.5, 25 mM beta-glycerophosphate, 2 mM DTT, 5% glycerol) and injected onto a Mono Q HR 10/10 (Pharmacia) anion exchange column equilibrated with Buffer A. The column was eluted with a 160 ml 0.1 M-0.6 M NaCl/Buffer A gradient (2 ml/min flowrate). The p38 kinase peak eluting at 200 mM NaCl was collected and concentrated to 3-4 ml with a Filtron 10 concentrator (Filtron Corp.).

### Sephacryl S100 Gel Filtration Chromatography

The concentrated Mono Q-p38 kinase purified sample was purified by gel filtration chromatography (Pharmacia HiPrep 26/60 Sephacryl S1100 column equilibrated with Buffer B (50 mM HEPES, pH 7.5, 50 mM NaCl, 2 mM DTT, 5% glycerol)). Protein was eluted from the column with Buffer B at a 0.5 ml/min flowrate and protein was detected by absorbance at 280 nm. Fractions containing p38 kinase (detected by SDS-polyacrylamide gel electrophoresis) were pooled and frozen at −80° C. Typical purified protein yields from 5 L E. coli shake flasks fermentations were 35 mg p38 kinase.

### In Vitro Assay

The ability of compounds to inhibit human p38 kinase alpha was evaluated using one of two in vitro assay methods. In the first method, activated human p38 kinase alpha phosphorylates a biotinylated substrate, PHAS-I (phosphorylated heat and acid stable protein-insulin inducible), in the presence of gamma ³²P-ATP (³²P-ATP). PHAS-I was biotinylated before the assay, and provided a means of capturing the substrate which was phosphorylated during the assay. p38 Kinase was activated by MKK6. Compounds were tested in 10 fold serial dilutions over the range of 100 μM to 0.001 μM using 1% DMSO. Each concentration of inhibitor was tested in triplicate.

All reactions were carried out in 96 well polypropylene plates. Each reaction well contained 25 mM HEPES pH 7.5, 10 mM magnesium acetate, and 50 μM unlabeled ATP. Activation of p38 was required to achieve sufficient signal in the assay. Biotinylated PHAS-I was used at 1-2 μg per 50 μl reaction volume, with a final concentration of 1.5 μM. Activated human p38 kinase alpha was used at 1 μg per 50 μl reaction volume, representing a final concentration of 0.3 μM. Gamma ³²P-ATP was used to follow the phosphorylation of PHAS-I. ³²P-ATP has a specific activity of 3000 Ci/mmol, and was used at 1.2 μCi per 50 μl reaction volume. The reaction proceeded either for one hr or overnight at 30° C.

Following incubation, 20 μl of reaction mixture was transferred to a high capacity streptavidin coated filter plate (SAM-streptavidin-matrix, Promega) prewetted with phosphate buffered saline. The transferred reaction mix was allowed to contact the streptavidin membrane of the Promega plate for 1-2 min. Following capture of biotinylated PHAS-I with ³²P incorporated, each well was washed to remove unincorporated ³²P-ATP three times with 2M NaCl, three washes of 2M NaCl with 1% phosphoric, three washes of distilled water, and finally a single wash of 95% ethanol. Filter plates were air dried and 20 μl of scintillant was added. The plates were sealed and counted.

A second assay format was alternatively employed. This assay is based on p38 kinase alpha being induced phosphorylation of EGFRP (epidermal growth factor receptor peptide, a 21 mer) in the presence of ³³P-ATP. Compounds were tested in 10 fold serial dilutions over the range of 100 μM to 0.001 μM in 10% DMSO. Each concentration of inhibitor was tested in triplicate. Compounds were evaluated in 50 μl reaction volumes in the presence of 25 mM HEPES pH 7.5, 10 mM magnesium acetate, 4% glycerol, 0.4% bovine serum albumin, 0.4 mM DTT, 50 μM unlabeled ATP, 25 μg EGFRP (200 μM), and 0.05 uCi gamma ³³P-ATP. Reactions were initiated by addition of 0.09 μg of activated, purified human GST-p38 kinase alpha. Activation was carried out using GST-MKK6 (5:1,p38:MKK6) for one hr at 30° C. in the presence of 50 μM ATP. Following incubation for 60 min at room temperature, the reaction was stopped by addition of 150 μl of AG 1×8 resin in 900 mM sodium formate buffer, pH 3.0 (1 volume resin to 2 volumes buffer). The mixture was mixed three times with pipetting. Afterward, the resin was allowed to settle. A total of 50 μl of clarified solution head volume was transferred from the reaction wells to Microlite-2 plates. 150 μl of Microscint 40 was then added to each well of the Microlite plate, and the plate was sealed, mixed, and counted.

The above protocol assays were used to determine the IC₅₀ values for compounds in Examples 1-28 above. The results are shown in Table 1.

TABLE 1

| | | | p38α Kinase |
|---|---|---|---|
| | | | Inhibition |
| Example | Compound | Structure | (IC₅₀ in μM) |
| 29 | Example 1 | 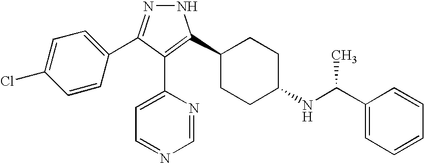 | 0.0314 |
| 30 | Example 2 | 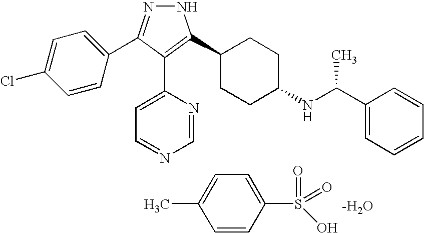 | 0.0279 |
| 31 | Example 3 | 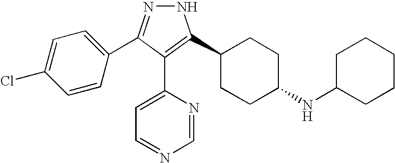 | 0.0452 |
| 32 | Example 4 | 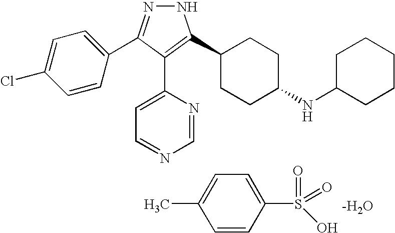 | 0.0379 |
| 33 | Example 5 | 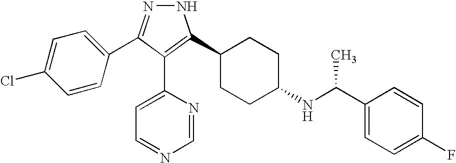 | 0.0232 |
| 34 | Example 6 | 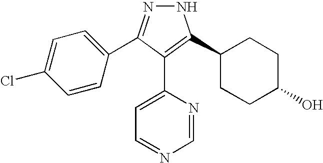 | 0.289 |
| 35 | Example 7 | 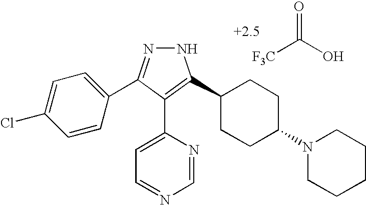 | 0.0238 |
| 36 | Example 8 | 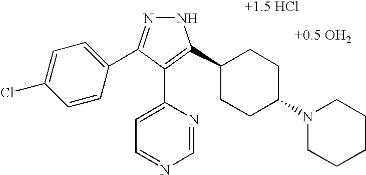 | 0.0582 |
| 37 | Example 9 | 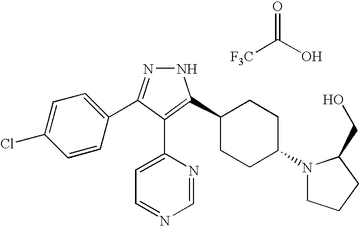 | 0.0285 |
| 38 | Example 10 | 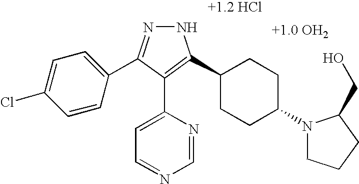 | 0.0354 |
| 39 | Example 11 | 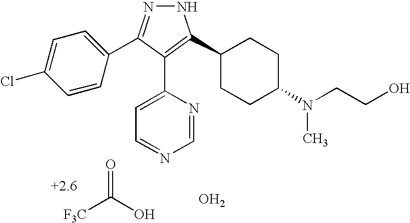 | 0.0271 |
| 40 | Example 12 | 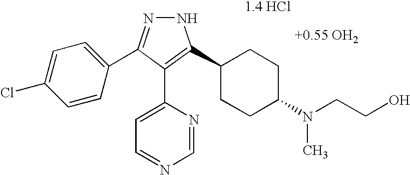 | 0.0379 |
| 41 | Example 13 | 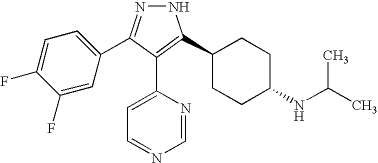 | 0.115 |
| 42 | Example 14 | 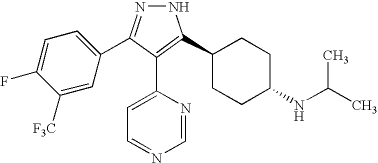 | 0.0723 |
| 43 | Example 16 | 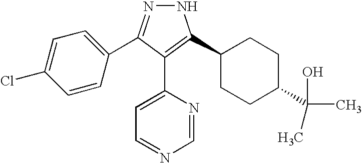 | 0.0926 |
| 44 | Example 17 | 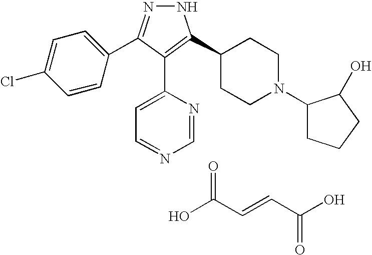 | 0.0189 |
| 45 | Example 18 | 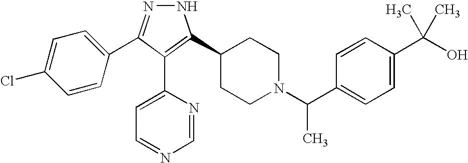 | 0.0106 |
| 46 | Example 19 | 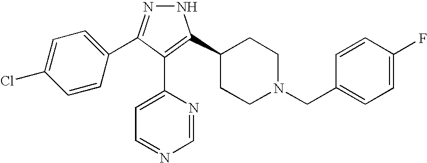 | 0.102 |
| 47 | Example 10 | 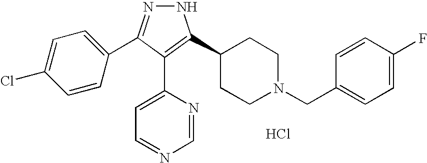 | 0.00927 |
| 48 | Example 21 | 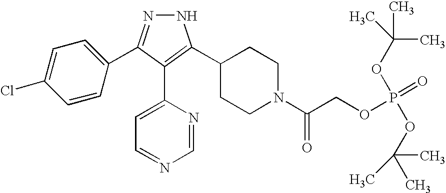 | 0.379 |
| 49 | Example 22 | 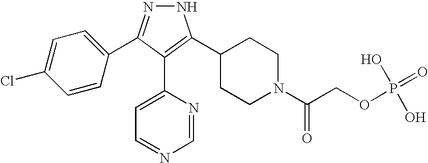 | 0.00607 |
| 50 | Example 23 | 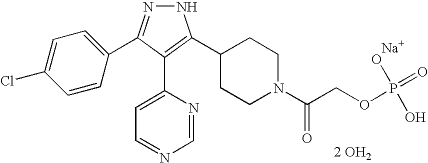 | 0.0681 |
| 51 | Example 24 | 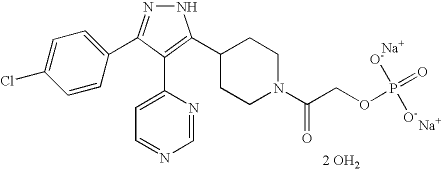 | 0.0608 |
| 52 | Example 25 | 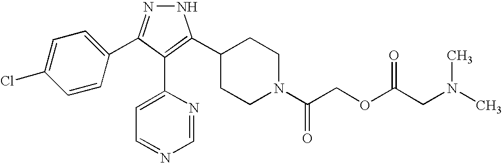 | 0.0118 |
| 53 | Example 26 | 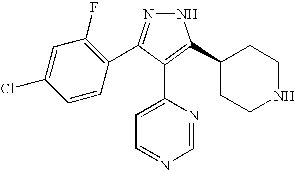 | 0.0641 |
| 54 | Example 27 | 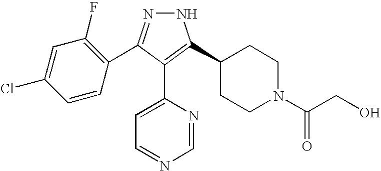 | 0.209 |
| 55 | Example 28 | 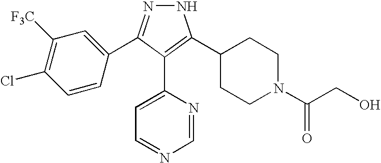 | 0.339 |

### Examples 56-169

Additional pyrazole compounds can be prepared by one skilled in the art using methods similar to those described in Examples 1-28 alone or in combination with techniques well known in the art. Such compounds include, for example, the compounds summarized in the following Table 2. Table 2 also summarizes in vitro p38 kinase inhibition results obtained by Applicants with the listed pyrazoles.

TABLE 2
Additional Example of Pyrazole Compounds

| | | | | p38α Kinase |
|---|---|---|---|---|
| | | Calculated | Observed | Inhibition |
| Ex. | Structure | Mass | Mass | (IC₅₀ in μM) |
| 56 | 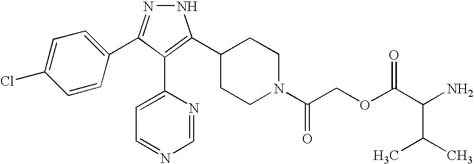 | 496.199 | 497.1 (M + H) | 0.0402 |
| 57 | 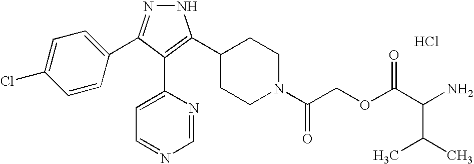 | 496.199 | 497 (M + H) | 0.0285 |
| 58 | 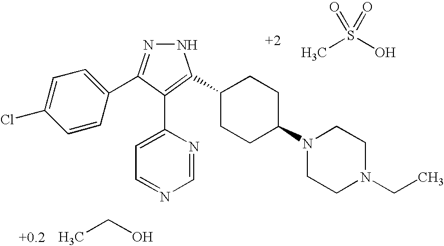 | 450.2299 | 451.2 (M + H) | 0.0649 |
| 59 | 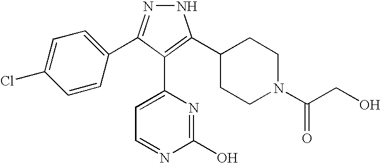 | 413.1255 | 414.1 (M + H) | 15.8 |
| 60 | 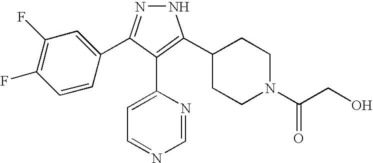 | 399.1507 | 400.1 (M + H) | 0.753 |
| 61 | 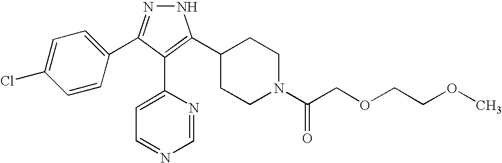 | 455.1724 | 456.1 (M + H) | 0.764 |
| 62 | 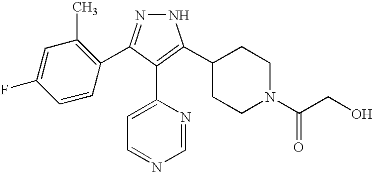 | 411.1462 | 412.1 (M + H) | 2.68 |
| 63 | 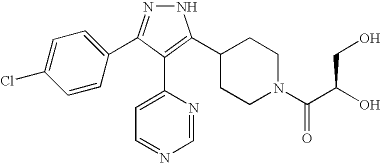 | 427.1411 | 428.1 (M + H) | 0.416 |
| 64 | 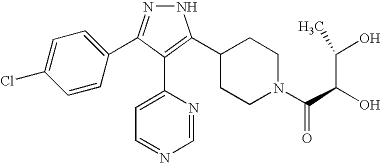 | 441.1568 | 442.1 (M + H) | 0.596 |
| 65 | 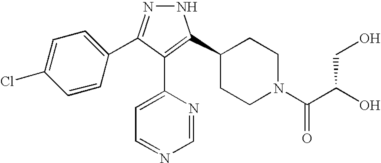 | 427.1411 | 428.1 (M + H) | 0.256 |
| 66 | 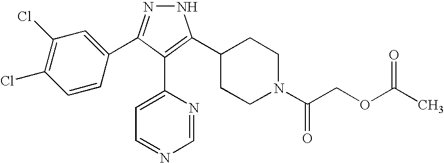 | 473.1021 | 474.1 (M + H) | 0.216 |
| 67 | 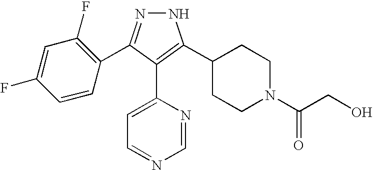 | 399.1507 | 400.1 (M + H) | 0.248 |
| 68 | 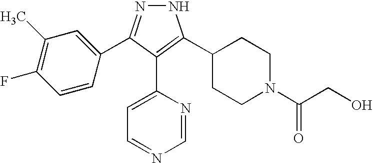 | 411.1462 | 412.1 (M + H) | 0.155 |
| 69 | 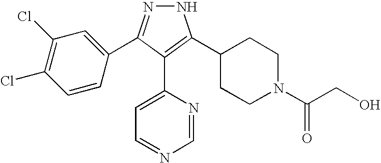 | 431.0916 | 432.0 (M + H) | 0.133 |
| 70 | 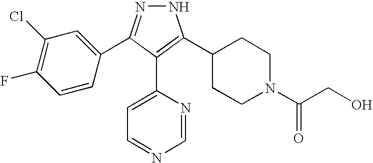 | 415.1211 | 416.1 (M + H) | 0.147 |
| 71 | 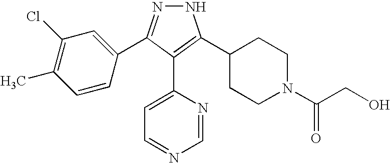 | 411.1462 | 412.1 (M + H) | 0.444 |
| 72 | 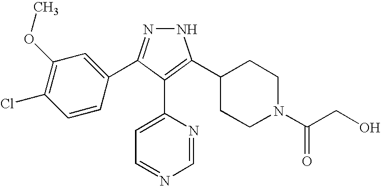 | 427.1411 | 428.1 (M + H) | 4.60 |
| 73 | 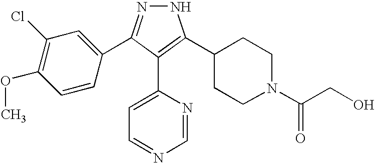 | 427.1411 | 428.1 (M + H) | 1.20 |
| 74 | 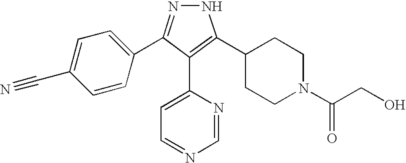 | 388.1648 | 389.1 (M + H) | 7.06 |
| 75 | 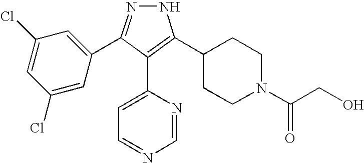 | 431.0916 | 432.0 (M + H) | 3.39 |
| 76 | 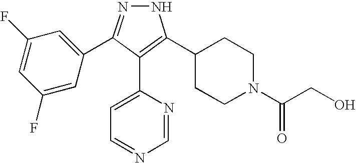 | 399.1507 | 400.1 (M + H) | 6.20 |
| 77 | 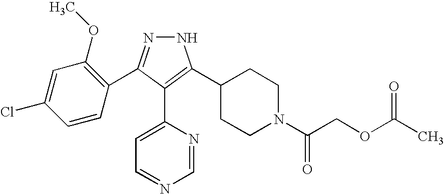 | 469.1517 | 470.1 (M + H) | 22.8 |
| 78 | 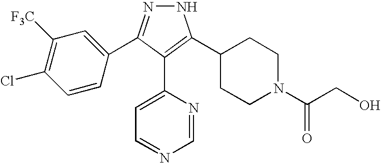 | 465.1179 | 466.1 (M + H) | 3.16 |
| 79 | 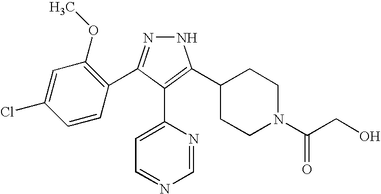 | 427.1411 | 428.1 (M + H) | 19.3 |
| 80 | 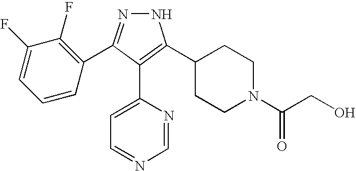 | 399.1507 | 400.1 (M + H) | 2.45 |
| 81 | 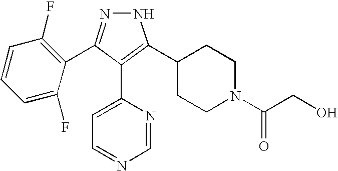 | 399.1507 | 400.1 (M + H) | 4.82 |
| 82 | 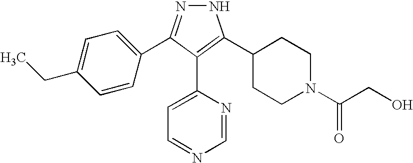 | 391.2008 | 392.2 (M + H) | 5.85 |
| 83 | 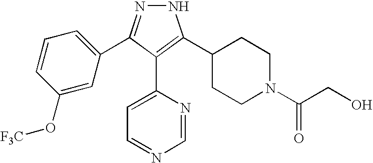 | 447.1518 | 448.1 (M + H) | 25.7 |
| 84 | 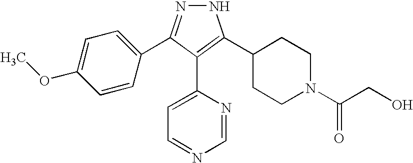 | 393.1801 | 394.1 (M + H) | 1.50 |
| 85 | 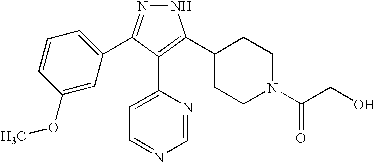 | 393.1801 | 394.1 (M + H) | 2.10 |
| 86 | 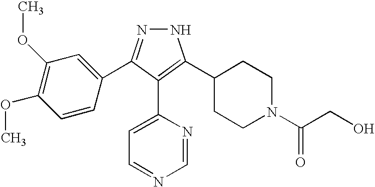 | 423.1907 | 424.1 (M + H) | >100 |
| 87 | 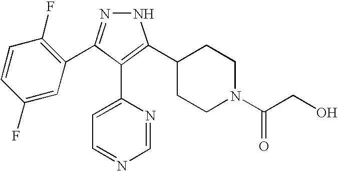 | 399.1507 | 400.1 (M + H) | 1.75 |
| 88 | 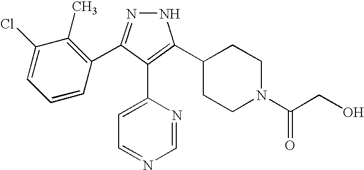 | 411.1462 | 412.1 (M + H) | 0.974 |
| 89 | 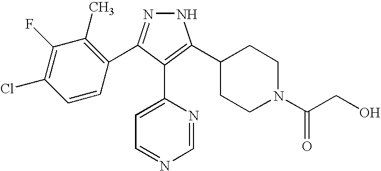 | 415.1211 | 416.1 (M + H) | 0.455 |
| 90 | 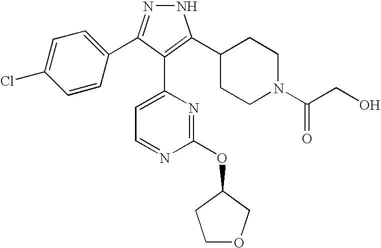 | 483.1673 | 484.1 (M + H) | 0.340 |
| 91 | 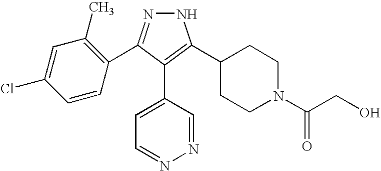 | 397.1306 | 398.1 (M + H) | 4.32 |
| 92 | 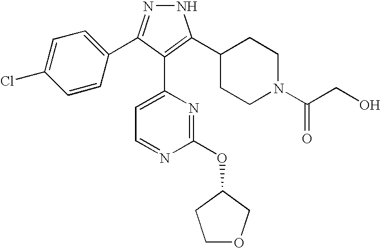 | 483.1673 | 484.1 (M + H) | 0.140 |
| 93 | 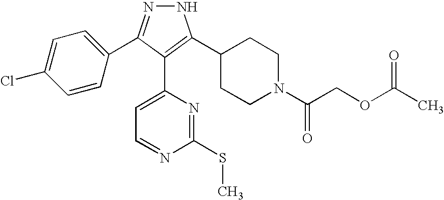 | 485.1288 | 486.1 (M + H) | 0.297 |
| 94 | 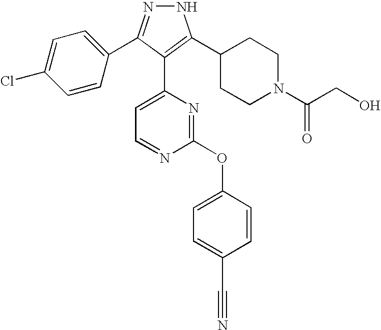 | 514.152 | 515.2 (M + H) | 0.108 |
| 95 | 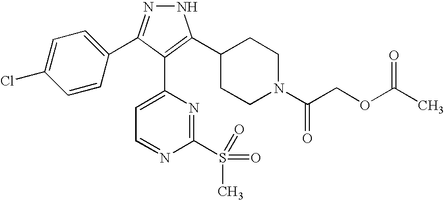 | 517.1187 | 518.1 (M + H) | 3.31 |
| 96 | 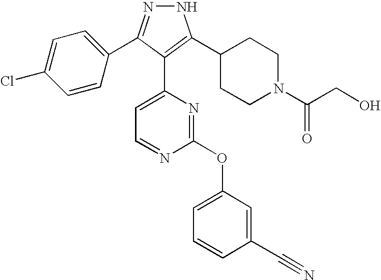 | 514.152 | 515.1 (M + H) | 0.0697 |
| 97 | 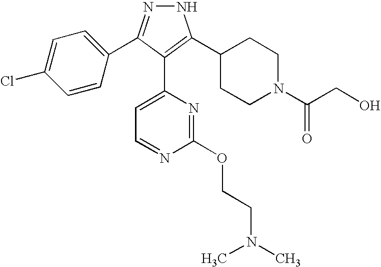 | 484.199 | 485.2 (M + H) | 0.377 |
| 98 | 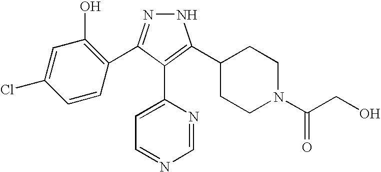 | 413.1255 | 414.1 (M + H) | 1.31 |
| 99 | 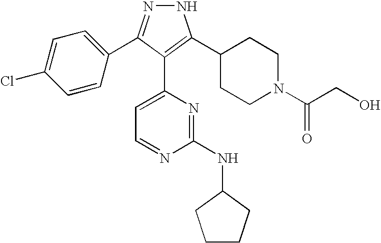 | 480.2041 | 484.1 (M + H) | 0.010 |
| 100 | 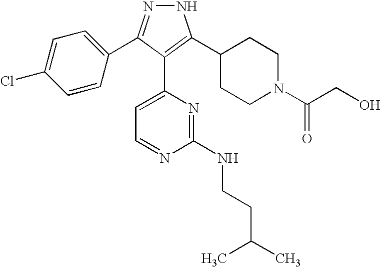 | 483 | 484.2 (M + H) | 0.859 |
| 101 | 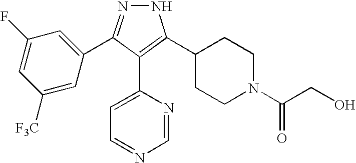 | 449.1475 | 450.1 (M + H) | 2.71 |
| 102 | 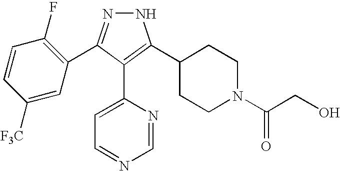 | 449.1475 | 450.1 (M + H) | 0.454 |
| 103 | 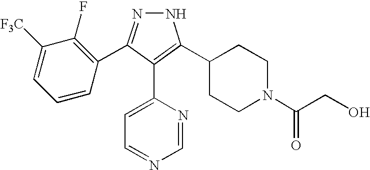 | 449.1475 | 450.1 (M + H) | 5.46 |
| 104 | 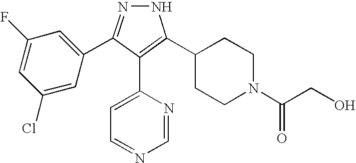 | 415.1211 | 416.1 (M + H) | 1.10 |
| 105 | 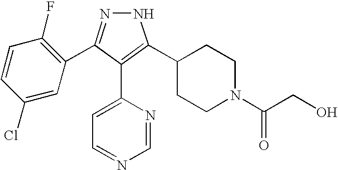 | 415.1211 | 416.1 (M + H) | 0.247 |
| 106 | 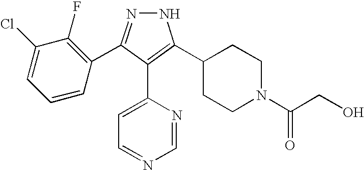 | 415.1211 | 416.1 (M + H) | 0.297 |
| 107 | 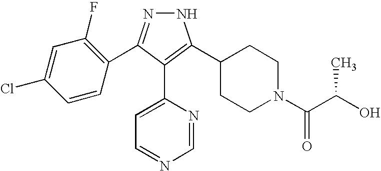 | 429.1368 | 430.1 (M + H) | 0.0777 |
| 108 | 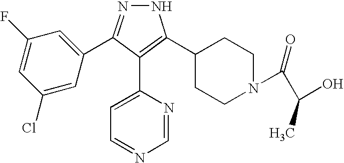 | 429.1368 | 430.1 (M + H) | 1.62 |
| 109 | 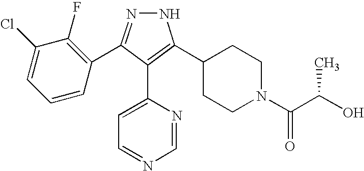 | 429.1368 | 430.1 (M + H) | 0.379 |
| 110 | 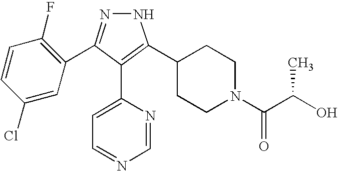 | 429.1368 | 430.1 (M + H) | 0.255 |
| 111 | 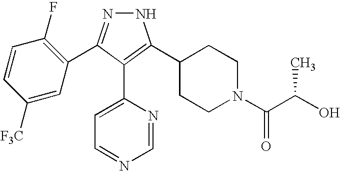 | 463.1631 | 464.1 (M + H) | 0.438 |
| 112 | 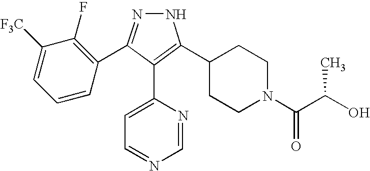 | 463.1631 | 464.1 (M + H) | 5.18 |
| 113 | 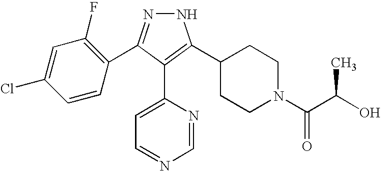 | 429.1368 | 430.1 (M + H) | 0.207 |
| 114 | 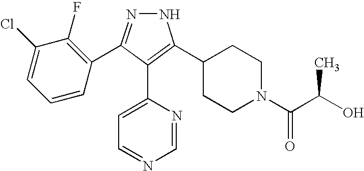 | 429.1368 | 430.1 (M + H) | 0.411 |
| 115 | 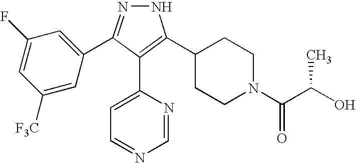 | 463.1631 | 464.1 (M + H) | 8.48 |
| 116 | 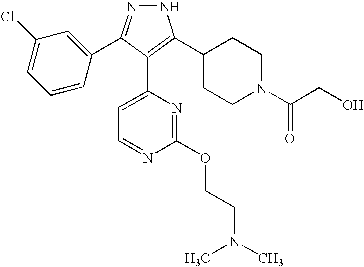 | 484.199 | 485.1 (M + H) | 0.727 |
| 117 | 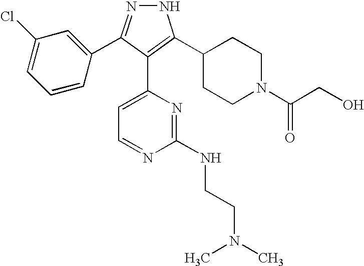 | 483.2149 | 482.4 (M + H) | 0.699 |
| 118 | 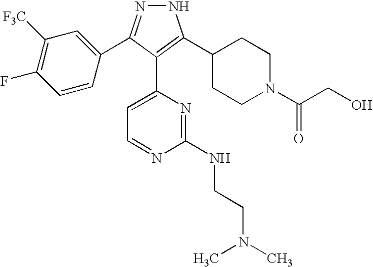 | 535.2319 | 536.2 (M + H) | 0.814 |
| 119 | 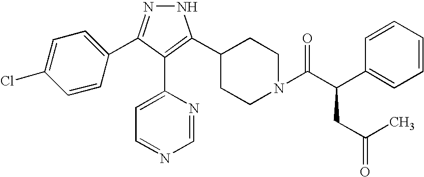 | 515.1724 | 516.1 (M + H) | 0.572 |
| 120 | 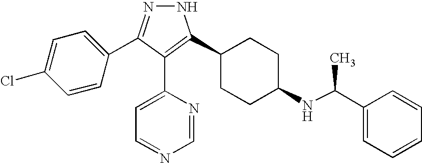 | 457.2033 | 457.21 | 0.112 |
| 121 | 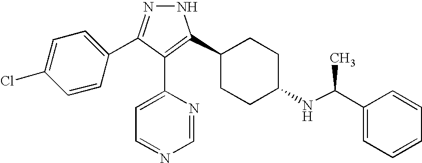 | 457.2033 | 457.208 | 0.00856 |
| 122 | 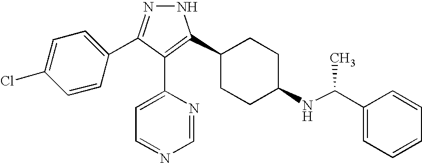 | 457.2033 | 457.211 | 0.0449 |
| 123 | 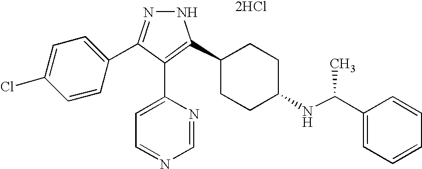 | 458 | 458 | 0.0427 |
| 124 | 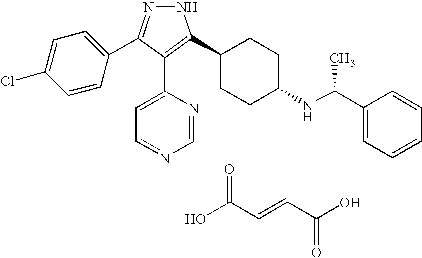 | 458 | 458 | 0.0452 |
| 125 | 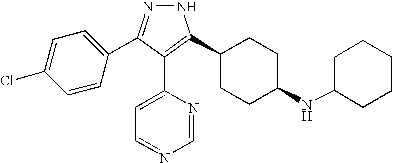 | 435.219 | 435.227 | 0.235 |
| 126 | 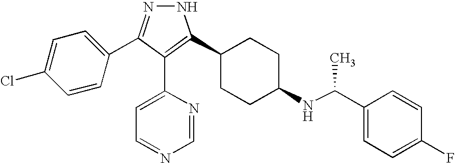 | 475.1939 | 475.203 | 0.130 |
| 127 | 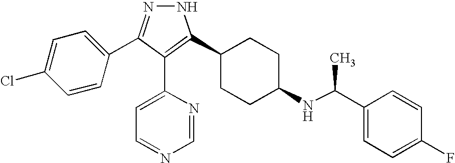 | 475.1939 | 475.203 | 0.0610 |
| 128 | 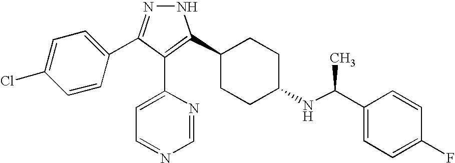 | 475.1939 | 475.204 | 0.00576 |
| 129 | 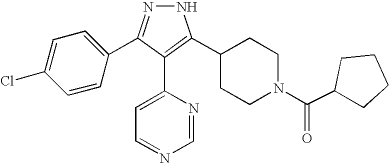 | 437.1619 | 438.1 (M + H) | 0.12 |
| 130 | 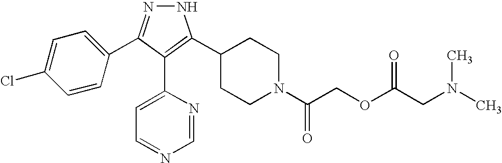 | 482.1833 | 483 (M + H) | 0.0438 |
| 131 | 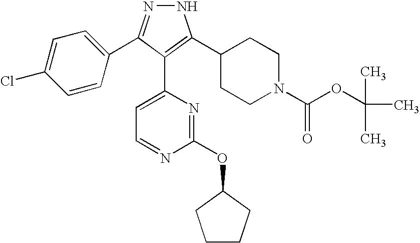 | 525 | 526 (M + H) | 1.68 |
| 132 | 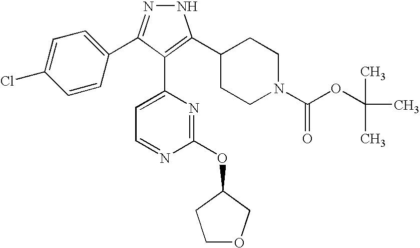 | 525 | 526 (M + H) | 3.34 |
| 133 | 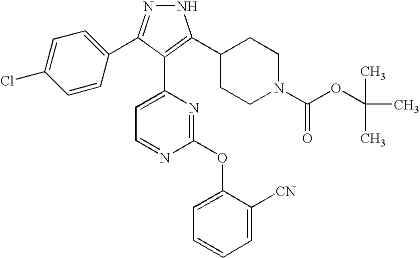 | 556 | 557 (M + H) | 0.848 |
| 134 | 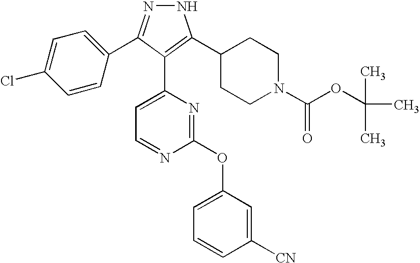 | 556 | 557 (M + H) | 1.59 |
| 135 | 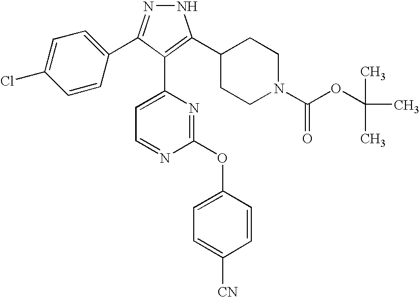 | 556 | 557 (M + H) | 1.5 |
| 136 | 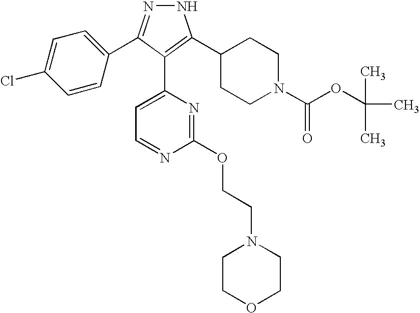 | 568 | 569 (M + H) | 16.9 |
| 137 | 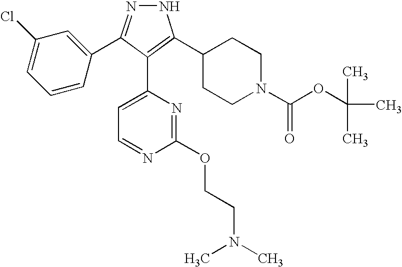 | 526 | 527 (M + H) | 9.34 |
| 138 | 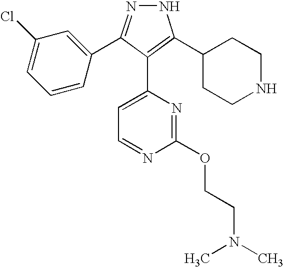 | 426 | 427 (M + H) | 0.162 |
| 139 | 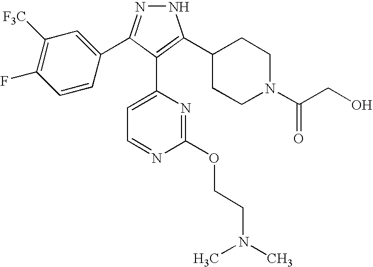 | 536 | 537 (M + H) | 0.854 |
| 140 | 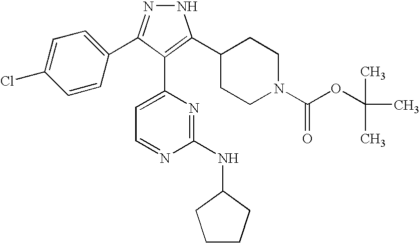 | 522.251 | 523 (M + H) | 0.01 |
| 141 | 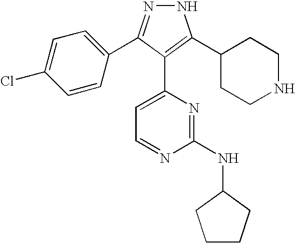 | 422.1986 | 423 (M + H) | 0.01 |
| 142 | 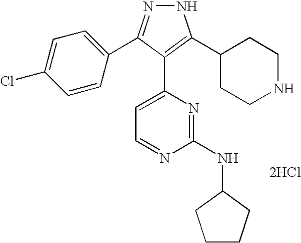 | 422.1986 | 423 (M + H) | |
| 143 | 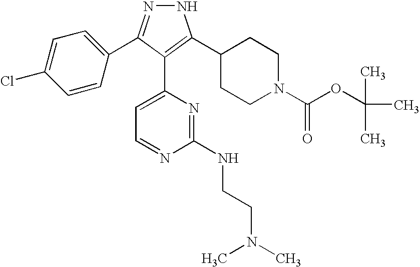 | 525.2619 | 526 (M + H) | 3.64 |
| 144 | 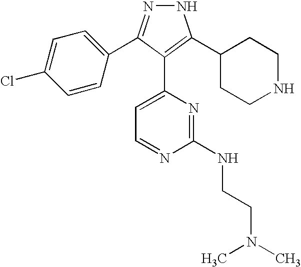 | 425.2095 | 426 (M + H) | 0.159 |
| 145 | 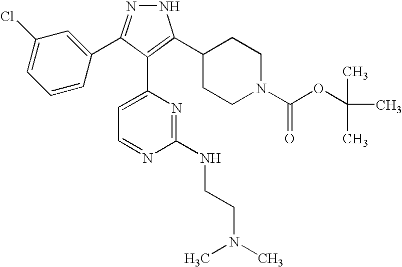 | 525.2619 | 526 (M + H) | 4.1 |
| 146 | 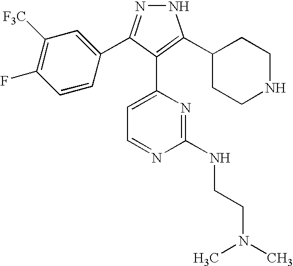 | 477.2264 | 478 (M + H) | 0.261 |
| 147 | 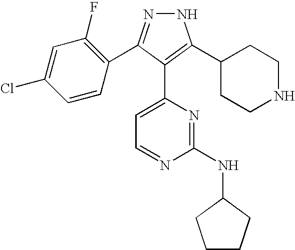 | 440.1891 | 441 (M + H) | 0.0159 |
| 148 | 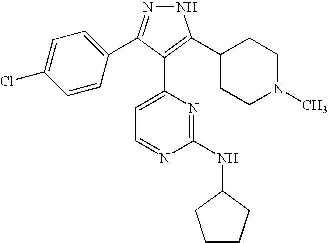 | 436.2142 | 437 (M + H) | 0.0148 |
| 149 | 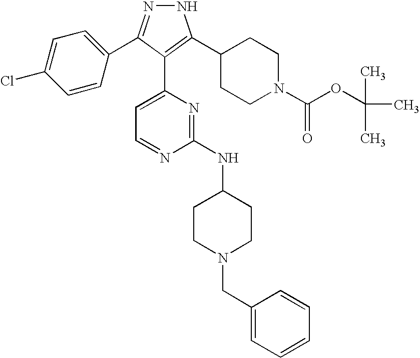 | 627.3089 | 628 (M + H) | |
| 150 | 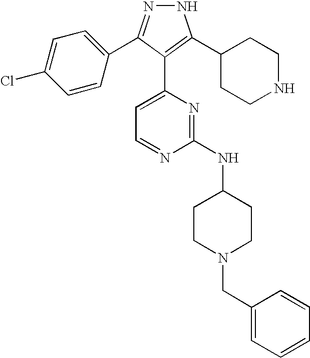 | 527.2564 | 528 (M + H) | 0.034 |
| 151 | 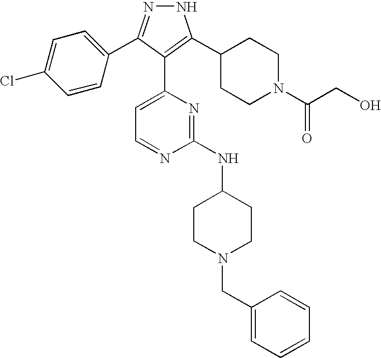 | 585.2619 | 586 (M + H) | 0.053 |
| 152 | 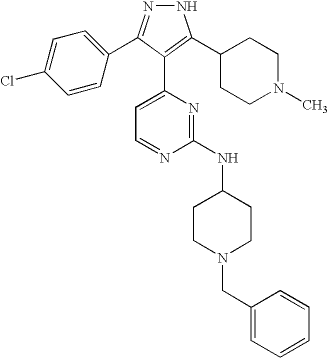 | 541.2721 | 542 (M + H) | 0.075 |
| 153 | 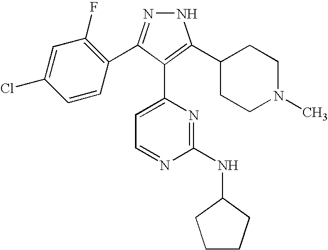 | 454.2048 | 455 (M + H) | 0.0182 |
| 154 | 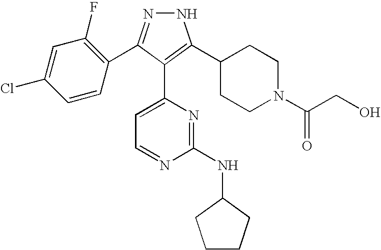 | 498.1946 | 499 (M + H) | 0.0176 |
| 155 | 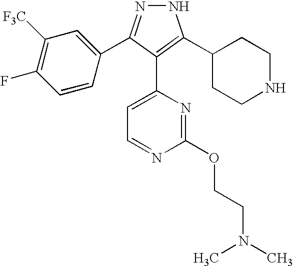 | 478 | 479 (M + H) | 0.527 |
| 156 | 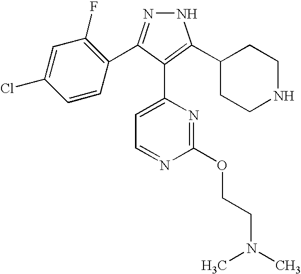 | 444 | 445 (M + H) | 0.637 0.627 |
| 157 | 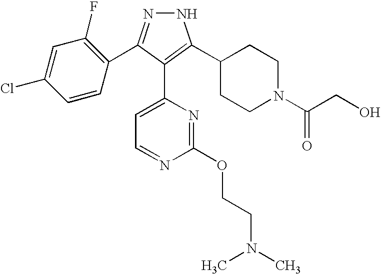 | 502 | 503 (M + H) | 1.07 0.847 |
| 158 | 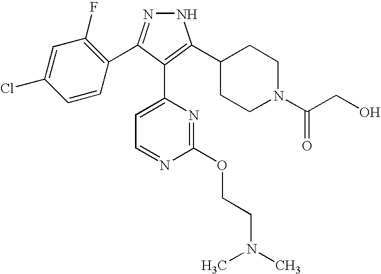 | 502 | 503 (M + H) | 1.3 0.647 |
| 159 | 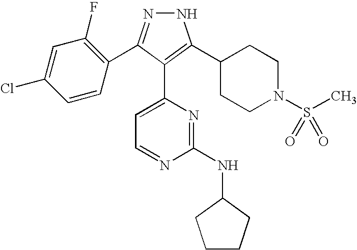 | 518.1667 | 519 (M + H) | 0.0047 |
| 160 | 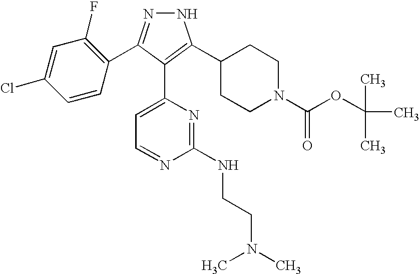 | 543.2525 | 544 (M + H) | 4.4 |
| 161 | 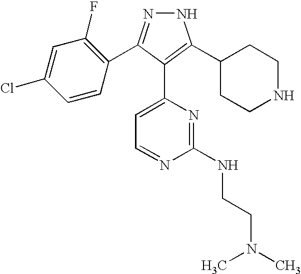 | 443.2 | 444 (M + H) | 0.202 |
| 162 | 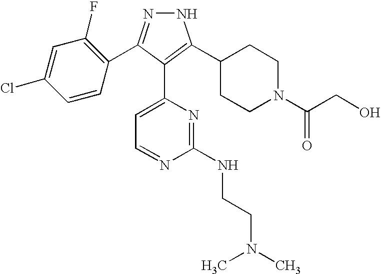 | 501.2055 | 502 (M + H) | 0.474 |
| 163 | 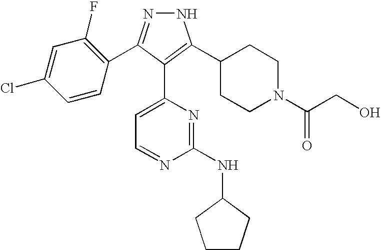 | 540.2416 | 541 (M + H) | 0.01 |
| 164 | 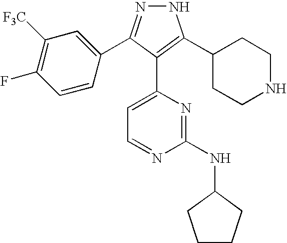 | 474.2155 | 475 (M + H) | 0.0225 |
| 165 | 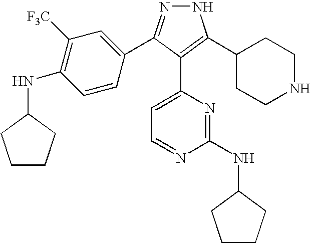 | 539.2984 | 540 (M + H) | 0.424 |
| 166 | 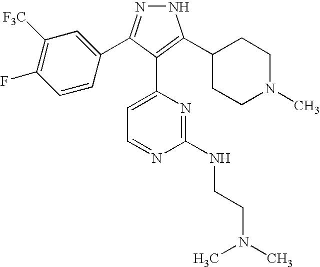 | 491.2421 | 492 (M + H) | 1.1 |
| 167 | 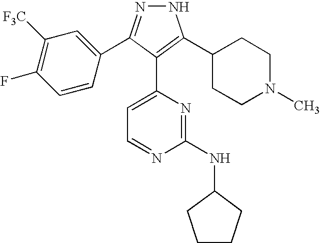 | 488.2312 | 489 (M + H) | 0.0063 |
| 168 | 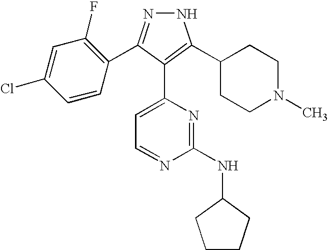 | 457.2157 | 458 (M + H) | 1 |
| 169 | 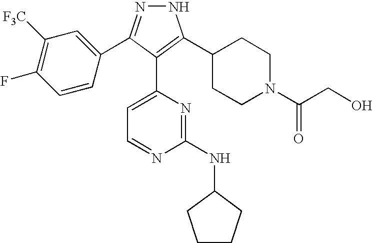 | 532.221 | 533 (M + H) | 0.0032 |

### Example 170

### Alternative Preparation of 2-{4-[3-(4-chloro-2-fluorophenyl)-4-pyrimidin-4-yl-1H-pyrazol-5-yl]piperidin-1-yl}-2-oxoethanol

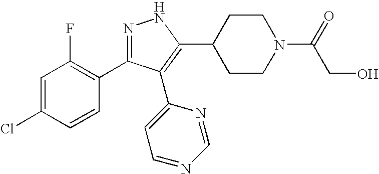

Part A. Preparation of 4-[5-(4-chloro-2-fluoro-phenyl)-4-pyrimidin-4-yl-1-(toluene-4-sulfonyl)-1H-pyrazol-3-yl]-piperidine-1-carboxylic Acid Tert-Butyl Ester (3).

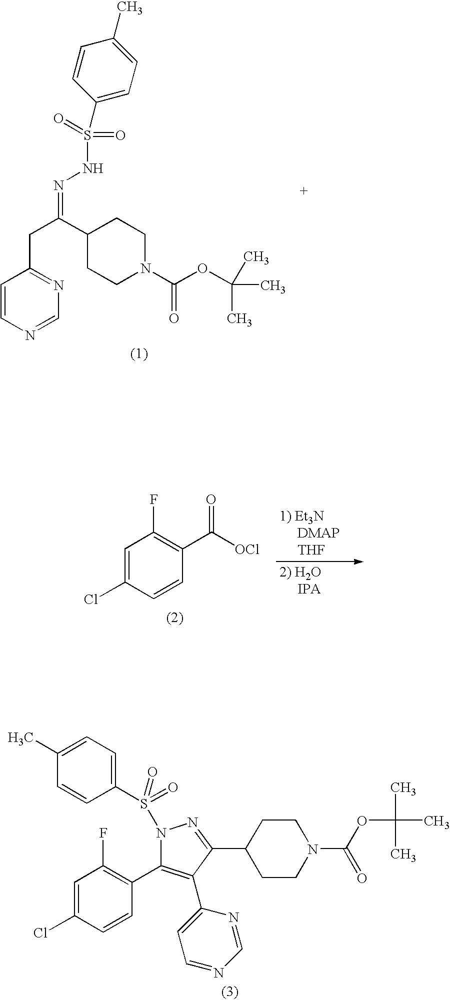

To a dry 4-neck, 250 mL flask was added tert-butyl 4-{(1E)-N-[(4-methylphenyl) sulfonyl]-2-pyrimidin-4-ylethanehydrazonoyl} piperidine-1-carboxylate (1) (20 g, 42.2 mmol), triethylamine (“Et₃N”, 7.22 g, 71.4 mmol, 1.69 equivalents relative to the hydrazonoyl reagent (1)), tetrahydrofuran (“THF”, 60 mL), and 4-dimethylaminopyridine (“DMAP”, 520 mg, 4.22 mmol, 0.1 equivalents relative to the hydrazonoyl reagent (1)). The resulting light yellow slurry was cooled with an ice/brine cooling bath to −5.5° C. Subsequently, 4-chloro-2-fluoro-benzoyl chloride (2) (11.55 g, 59.8 mmol, 1.42 equivalents relative to the hydrazonoyl reagent (1)) was added over a 1 hr period while maintaining the temperature at no greater than 2° C. to form an orange-yellow slurry. After stirring for 0.5 hr, the mixture was removed from the cooling bath, and stirred for an additional hour in the absence of the cooling bath. The temperature of the mixture was then increased from 24° C. to 55° C. over a 15 min period. After stirring the mixture at 55° C. for 0.5 hr, heating was ceased, and the mixture was allowed to naturally cool to room temperature while being stirred. One hour after heating ceased, water (20 g) was added to the mixture. Stirring was continued for another 50 min. The resulting mixture was transferred to a separatory funnel. Water (20 g) and THF (20 g) were then added, and the phases were allowed to separate into a yellow aqueous layer and an orange organic layer. After removing the aqueous layer (39.3 g), the remaining organic layer was washed with saturated NH₄Cl (38 g). After phase separation, the organic layer (93.3 g) was charged to a 500 mL round-bottom flask (equipped with a thermocouple) and heated to 55° C. A mixture of water (64 g) and isopropyl alcohol (“IPA”, 81 g) was then added at a rate that allowed the temperature to be maintained at greater than 50° C. After the addition, the mixture was maintained at 50° C. for 3 hr, and then stirred at room temperature overnight. The resulting clear yellow/orange solution was heated to 53° C. Deionized water (60 mL) was then added dropwise, resulting in the formation of a precipitate. The mixture was maintained at 53° C. for 1 hr, and then additional water (15 mL) was added dropwise. The resulting mixture was maintained at 55° C. for an additional 2 hr. Afterward, the mixture was allowed to cool to room temperature. This resulted in the formation of an oil. This oil was transferred to a 1 L flask and concentrated in vacuo on a rotary evaporator in a water bath to form a yellow foam. Methanol (100 mL) was then added, and the water bath was heated to 61° C. This temperature was maintained for 1 hr. The mixture was then allowed to cool naturally with stirring overnight. This resulted in the formation of a precipitate. The precipitate was filtered, washed with cold methanol (3×50 mL), and air-dried for 2 hr to form 20 g of a white solid containing yellow flakes. This solid was transferred to a 250 mL Erlenmeyer flask. After adding methanol (80 mL) and a stir bar, the flask was heated on a stirring hot plate to reflux under a stream of N₂. Additional methanol (10 mL) was added to form a clear, light-yellow solution. Subsequently, water (20.5 mL) was added while maintaining the solution at reflux. The heat was then turned off, and the mixture was allowed to stir overnight, resulting in the formation of a white solid. The solid was filtered, washed with a mixture of methanol and water (50 mL, 4:1 MeOH:water), air-dried for 1.5 hr, and dried under vacuum with N₂ purge to complete drying to afford 19.1 g of the desired ester intermediate (3).

Part B. Preparation of 4-[3-(4-chloro-2-fluorophenyl)-5-piperidin-4-yl-1H-pyrazol-4-yl]pyrimidine (4).

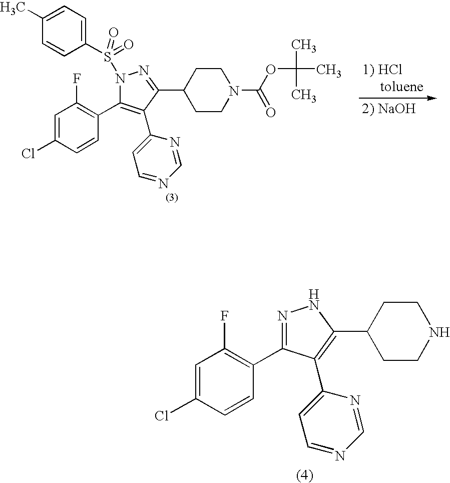

To a 4-neck, 100 mL round-bottom flask (equipped with a mechanical stirrer, thermocouple, addition funnel, reflux condenser, and N₂ purge) was added the ester intermediate (3) from Part A (5.0 g, 8.17 mmole) and toluene (10 g, 108.5 mmol, 12.9 equivalents relative to the ester intermediate (3)). Afterward, 37% HCl (6.44 g, 65.3 mmol, 8 equivalents relative to the ester intermediate (3)) was added dropwise over a 10 min period. After half the HCl was added, additional toluene (10 g, 108.5 mmol) was added to facilitate agitation. Gas evolution was observed during the HCl addition, and the temperature of the mixture increased from 20.4° C. to 22.4° C. After the HCl addition, the mixture was stirred at ambient temperature for 1 hr resulting in the formation of a solid. The mixture was then heated to 70° C. while continuing to be stirred. This resulted in the formation of a two-phase system. Stirring was continued for an additional 2 hr at 70° C. Water (10 mL) was then added, and the mixture was allowed to cool naturally to room temperature while being stirred. The resulting mixture was transferred to a separatory funnel where the layers were separated. The aqueous layer was placed back into the 100 mL reactor flask and heated to 65-70° C. While maintaining the mixture at this temperature, 6 N NaOH (14 g) was added dropwise over a 30 min period. Initially, solids would form and dissolve as each drop of NaOH was added. After about half of the NaOH was added, solids began to remain. Following addition of the base, the mixture was stirred at 65-70° C. for 1.5 hours, and then allowed to cool naturally to room temperature while being stirred. Stirring was continued overnight. This resulted in the formation of a while slurry having a pH of 12.9. The solids were filtered, washed with deionized water (70 g) to increase the pH to 8.5, air-dried for 1.5 hr, and dried in a vacuum oven under N₂ at 50° C. to afford 2.8 g (96% yield) of a white solid. Liquid chromatography analysis comparing this solid with a pre-formed standard indicated the formation of the desired intermediate (4).

Part C. Preparation of 2-{4-[3-(4-chloro-2-fluorophenyl)-4-pyrimidin-4-yl-1H-pyrazol-5-yl]piperidin-1-yl}-2-oxoethanol (6).

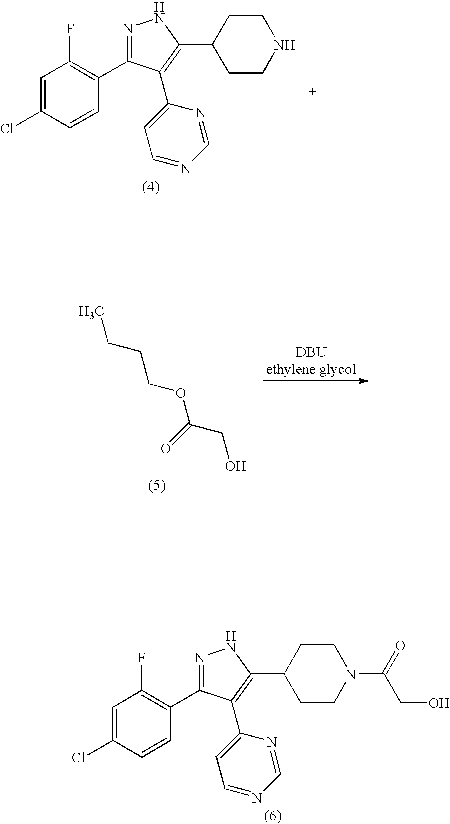

To a 3-neck, 50 mL flask (equipped with a thermocouple, magnetic stirrer, heating mantle, reflux condenser, and N₂ purge) was added the intermediate (4) from Part B (1.0 g, 2.79 mmol), ethylene glycol (7.0 g), 1,8-diazabicyclo[5.4.0]undec-7-ene (“DBU”, 42 mg, 0.28 mmol, 0.1 equivalents relative to the intermediate (4)), and butyl glycolate (5) (1.1 g, 8.38 mmol, 3.0 equivalents relative to the intermediate (4)). The resulting white slurry was heated to 80° C., and then stirred at this temperature for 4.9 hr. Although the slurry initially formed a light yellow solution during this heating, it formed a clear solution after 40 min of heating. Following the 4.9 hr heating period, deionized water was added over a 15 min period in an amount such that the mixture became slightly turbid. During this water addition, the temperature was maintained at 80° C. The resulting mixture was stirred for another hour at this temperature, and then allowed to cool naturally to room temperature. The resulting precipitate was filtered, washed with water (2×10 mL), and air-dried for 1.3 hr to afford 1.10 g of light yellow crystals. Liquid chromatography analysis comparing these crystals with a pre-formed standard indicated the formation of the desired product (6).

### Example 171

### TNF Cell Assays

The following cell assays illustrate methods for analyzing efficacy of the compounds of this invention in blocking the production of TNF. These assays are further illustrated in WIPO Int'l PCT Publ. No. WO 00/31063.

### Assay 1: LPS Stimulation of Human Peripheral Blood Mononuclear (PBM) Cells

### 1. Isolation of Cells

Human whole blood is collected in Vacutainer tubes containing EDTA as an anticoagulant. A blood sample (7 ml) is carefully layered over 5 ml PMN Cell Isolation Medium (Robbins Scientific) in a 15 ml round bottom centrifuge tube. The sample is centrifuged at 450-500×g for 30-35 min in a swing out rotor at room temperature. After centrifugation, the top band of cells are removed and washed 3 times with PBS without calcium or magnesium. The cells are then centrifuged at 400×g for 10 min at room temperature. Afterward, the cells are re-suspended in Macrophage Serum Free Medium (Gibco BRL) at a concentration of 2 million cells/ml.

### 2. Assay to Determine TNF Production in Presence of Pyrazole Compound

The isolated PBM cells (0.1 ml, 2 million/ml) are co-incubated with 0.1 ml the compound to be tested (10-0.41 μM, final concentration) for 1 hr in flat bottom 96 well microtiter plates (the compound is dissolved in DMSO initially, and diluted in TCM for a final concentration of 0.1% DMSO). LPS (Calbiochem, 20 ng/ml, final concentration) is then added at a volume of 0.010 ml. Cultures are incubated overnight at 37° C. Supernatants are then removed and tested by ELISA for TNF and IL1-b. Viability is analyzed using MTS. After 0.1 ml supernatant is collected, 0.020 ml MTS is added to the remaining 0.1 ml cells. The cells are incubated at 37° C. for 2-4 hr. The O.D. is then measured at 490-650 nM.

### Assay 2: LPS Stimulation of TNF Production by U937 Human Histiocytic Lymphoma Cells

### 1. Maintenance and Differentiation of Cell Line

U937 cells (ATCC) are propagated in RPMI 1640 containing 10% fetal bovine serum, 100 IU/ml penicillin, 100 μg/ml streptomycin, and 2 mM glutamine (Gibco). Fifty million cells in 100 ml media are induced to terminal monocytic differentiation by 24 hr incubation with 20 ng/ml phorbol 12-myristate 13-acetate (Sigma). The cells are then washed by centrifugation (200×g for 5 min) and re-suspended in 100 ml fresh medium. After 24-48 hr, the cells are harvested, centrifuged, and re-suspended in culture medium at 2 million cells/ml.

### 2. Assay to Determine TNF Production in Presence of Pyrazole Compound

U937 cells (0.1 ml, 2 million/ml) are incubated with 0.1 ml the compound to be tested (0.004-50 μM, final concentration) for 1 hr in 96 well microtiter plates (the compound is prepared as 10 mM stock solutions in DMSO, and diluted in culture medium to yield a final DMSO concentration of 0.1% in the cell assay). LPS (E coli, 100 ng/ml final concentration) is then added at a volume of 0.02 ml. After 4 hr incubation at 37° C., the amount of TNF released in the culture medium is quantitated by ELISA. Inhibitory potency is expressed as IC₅₀ (μM).

### Assay 3: Human Whole Blood TNF Inhibition Assay

Human peripheral blood is obtained in heparinized tubes. A 190 μL aliquot of blood is placed in each well of a 96 well u-bottom plate. A compound or control vehicle (phosphate buffered saline with dimethylsulfoxide and ethanol) is added to the blood in 10 μL aliquots for serial dilutions providing final concentrations of 25, 5, 1 and 0.25 μM. The final dimethylsulfoxide and ethanol concentrations are 0.1% and 1.5%, respectively. After 1 hr of incubation at 37° C., 10 mL of lipopolysaccharide (Salmonella typhosa, Sigma) in phosphate buffered saline is added resulting in a final concentration of 10 mg/mL. After 4-5 hr of incubation at 37° C., the supernatants are harvested and assayed at 1:10 or 1:20 dilutions for human TNF using ELISA.

### Example 172

### Rat Assay for TNF Inhibition

The efficacy of the compounds of this invention in blocking the production of TNF also may be evaluated using a model based on rats challenged with LPS. This assay is further illustrated in WIPO Int'l PCT Publ. No. WO 00/31063.

Male Harlen Lewis rats (Sprague Dawley Co.) are used in this model. Each rat should weigh approximately 300 g and fast overnight before testing. Compound administration is typically by oral gavage (although intraperitoneal, subcutaneous, and intravenous administration may also be used) 1-24 hr before the LPS challenge. Rats are administered 30 μg/kg LPS (salmonella typhosa, Sigma Co.) intravenously via the tail vein. Blood is collected via heart puncture 1 hr after the LPS challenge. Serum samples are stored at −20° C. until quantitative analysis of TNF by Enzyme Linked-Immuno-Sorbent Assay (“ELISA”) [Biosource]. Additional details of the assay are set forth in Perretti, M., et al., Br. J. Pharmacol., (1993), 110, 868-874 (incorporated by reference into this specification).

### Example 173

### Mouse Assay for TNF Inhibition

The efficacy of the compounds of this invention in blocking the production of TNF also may be evaluated using a model based on mice challenged with LPS. This assay is further illustrated in WIPO Int'l PCT Publ. No. WO 00/31063.

TNF production is induced in 10-12 week old BALB/c female mice by tail vein injection with 100 ng LPS (lipopolysaccharide, from S. Typhosa) in 0.2 ml saline. After 1 hr later, the mice are bled from the retroorbital sinus and TNF concentrations in serum from clotted blood are quantified by ELISA. Typically, peak levels of serum TNF range from 2-6 ng/ml 1 hr after LPS injection.

The pyrazole compounds tested are administered to fasted mice by oral gavage as a suspension in 0.2 ml of 0.5% methylcellulose and 0.025% Tween 20 in water at 1 hr or 6 hr before LPS injection. The 1 hr protocol allows evaluation of compound potency at Cₘₐₓ plasma levels, whereas the 6 hr protocol allows estimation of compound duration of action. Efficacy is determined at each time point as percent inhibition of serum TNF levels relative to LPS injected mice that receive vehicle only.

### Example 174

### Mouse Assay for Assessing the Effectiveness of the Pyrazole Compounds in Treating Arthritis

The efficacy of the compounds of this invention in treating arthritis may be evaluated using the following method. This assay is further illustrated in WIPO Int'l PCT Publ. No. WO 00/31063.

### Induction and Assessment of Collagen-Induced Arthritis in Mice

Arthritis is induced in mice according to the procedure set forth in J. M. Stuart, Collagen Autoimmune Arthritis, Annual Rev. Immunol. 2:199 (1984) (incorporated by reference into this specification). Specifically, arthritis is induced in 8-12 week-old DBA/1 male mice by injection of 50 μg of chick type II collagen (CII) (provided by Dr. Marie Griffiths, Univ. of Utah, Salt Lake City, Utah) in complete Freund's adjuvant (Sigma) on day 0 at the base of the tail. Injection volume is 100 μl. Animals are boosted on day 21 with 50 μg of CII in incomplete Freund's adjuvant (100 μl volume). Animals are evaluated several times each week for signs of arthritis. Any animal with paw redness or swelling is counted as arthritic. Scoring of arthritic paws is conducted in accordance with the procedure set forth in Wooley et al., “Genetic Control of Type II Collagen Induced Arthritis in Mice: Factors Influencing Disease Susceptibility and Evidence for Multiple MHC Associated Gene Control,” Trans. Proc., 15:180 (1983) (incorporated by referenced into this specification). Scoring of severity is carried out using a score of 1-3 for each paw (maximal score of 12/mouse). Animals displaying any redness or swelling of digits or the paw are scored as 1. Gross swelling of the whole paw or deformity is scored as 2. Ankylosis of joints is scored as 3. Animals are evaluated for 8 weeks. 8-10 animals per group are used.

### Preparation and Administration of Compounds

The pyrazole compounds are prepared as a suspensions in 0.5% methylcellulose (Sigma, St. Louis, Mo.), 0.025% Tween 20 (Sigma). The suspensions are administered by oral gavage in a volume of 0.1 ml b.i.d. Administration begins on day 20 post-collagen-injection, and continues daily until final evaluation on day 56. Scoring of arthritic paws is conducted as set forth above.

### Example 175

### Assay for Assessing the Effectiveness of the Pyrazole Compounds in Treating Arthritis Induced by the Streptococcal Cell Wall

The streptococcal cell wall (“SCW”) is a complement activator and a potent antigen for T cells. Upon intraperitoneal administration of peptidoglycan-polysaccaride complexes isolated from group A streptococcal cell walls in rats, serum hemolytic complement levels drop. Arthritis develops in two phases. First, an acute inflammatory arthritis phase develops within 1-3 days (non-T cell dependent). This is the result of SCW complexes sticking in specific tissues, such as the joint and liver. Specifically, at these sites of accumulation, the SCW continues to activate complement, resulting in edema and a large neutrophil influx. This acute phase is followed by the second phase, a chronic erosive arthritis (T cell dependent) that develops within 10-28 days. The chronic phase results from T cell activation against the SCW fragments that have accumulated in the joint. This assay permits analysis of the in vivo effect of the pyrazole compounds of this invention during both phases of the disease.

### Animals

The animals used in this assay are female Lewis rats having a weight of approximately 100-140 g at the initiation of the study. They are given food and water as needed.

### Arthritis

Arthritis is induced by a single intraperitoneal administration of peptidoglycan-polysaccaride complexes isolated from group A streptococcal cell walls. The suspension is in sterile saline, and administered at a dose of 15-60 μg rhamnose equivalents/g body weight. A 23 gauge needle or smaller is used for injections in a volume of 1 ml or less.

### Evaluation of Pyrazole Compounds on the Arthritis

Assessment of arthritis is conducted by measuring paw volume using plethysmometer and/or visually scoring the animals on a scale of 0-4 for each paw. The anti-inflammatory effect of the compounds is evaluated by comparison to vehicle (arthritic) and uninjected (non-arthritic) controls. Animals are observed for up to 28 days.

### Example 176

### Assays for Assessing the Effectiveness of the Pyrazole Compounds in Inhibiting the Production of Cyclooxygenase-2

Assays for evaluating the effectiveness of compounds in inhibiting the production of cyclooxygenase-2 are well known in the art. Such assays include, for example, those described by Carter et al. in U.S. Pat. No. 6,271,253 (incorporated by reference into this specification). Such assays also include, for example, those described by Talley et al. in U.S. Pat. No. 5,859,257 (incorporated by reference into this specification).

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

We claim:

1. A compound, a tautomer of the compound, or a salt of the compound or tautomer, wherein:
the compound corresponds in structure to the following formula: 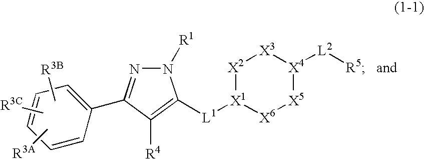
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X¹ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} and R^{3B} are independently selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3C} is selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R⁴ is selected from the group consisting of pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

2. A compound, tautomer of the compound, or salt of the compound or tautomer according to claim 1, wherein R^{3C} is hydrogen.

3. A compound, tautomer of the compound, or salt of the compound or tautomer according to claim 2, wherein R^{3A} and R^{3B} are independently selected from the group consisting of halogen, methyl, methoxy, halomethyl, and halomethoxy.

4. A compound, tautomer of the compound, or salt of the compound or tautomer according to claim 2, wherein R^{3A} and R^{3B} are independently selected from the group consisting of chloro, fluoro, methyl, methoxy, chloromethyl, fluoromethyl, chloromethoxy, and fluoromethoxy.

5. A compound, tautomer of the compound, or salt of the compound or tautomer according to claim 2, wherein R⁴ is selected from the group consisting of pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group is substituted with a substituent selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

6. A compound, tautomer of the compound, or salt of the compound or tautomer according to claim 5, wherein R⁴ is pyridinyl substituted with a substituent selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

7. A compound, tautomer of the compound, or salt of the compound or tautomer according to claim 5, wherein R⁴ is pyrimidinyl substituted with a substituent selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

8. A compound, tautomer of the compound, or salt of the compound or tautomer according to claim 7, wherein:
the compound corresponds in structure to the following formula: 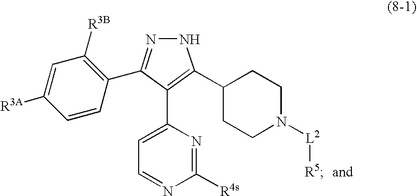
R^{4s} is selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

9. A compound, tautomer of the compound, or salt of the compound or tautomer according to claim 8, wherein R^{4s} is selected from the group consisting of alkoxycarbonyl, carbocyclyloxycarbonyl, and heterocyclyloxycarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

10. A compound, tautomer of the compound, or salt of the compound or tautomer according to claim 8, wherein R^{4s} is alkylaminocarbonyl optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

11. A compound, tautomer of the compound, or salt of the compound or tautomer according to claim 8, wherein R^{4s} is selected from the group consisting of —CH₂OH, —C(CH₃)(H)—OH, and —C(CH₃)₂—OH.

12. A compound, tautomer of the compound, or salt of the compound or tautomer according to claim 8, wherein R^{4s} is aminomethyl, wherein:
the amino nitrogen optionally is substituted with up to two substituents independently selected from the group consisting of alkyl, alkenyl, hydroxy, haloalkyl, alkylsulfonyl, alkoxyalkyl, and heterocyclyl.

13. A compound, tautomer of the compound, or salt of the compound or tautomer according to claim 8, wherein -L²-R⁵ is hydroxyalkylcarbonyl.

14. A compound, tautomer of the compound, or salt of the compound or tautomer according to claim 13, wherein -L²-R⁵ is hydroxymethylcarbonyl.

15. A compound, tautomer of the compound, or salt of the compound or tautomer according to claim 2, wherein X², X³, X⁵, and X⁶ are each —CH₂—.

16. A compound, tautomer of the compound, or salt of the compound or tautomer according to claim 15, wherein R¹ is selected from the group consisting of hydrogen and hydroxyalkyl.

17. A compound, tautomer of the compound, or salt of the compound or tautomer according to claim 16, wherein L¹ is a bond.

18. A compound, tautomer of the compound, or salt of the compound or tautomer according to claim 17, wherein the compound corresponds in structure to the following formula:

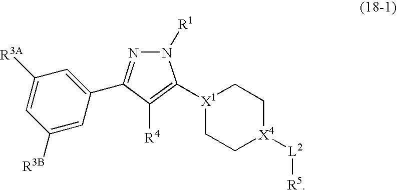

19. A compound, tautomer of the compound, or salt of the compound or tautomer according to claim 17, wherein the compound corresponds in structure to the following formula:

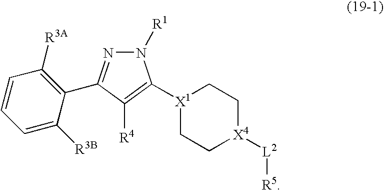

20. A compound, tautomer of the compound, or salt of the compound or tautomer according to claim 17, wherein the compound corresponds in structure to the following formula:

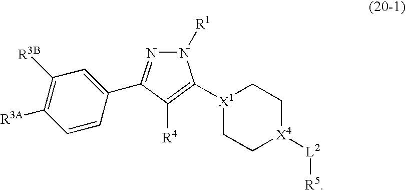

21. A compound, tautomer of the compound, or salt of the compound or tautomer according to claim 17, wherein the compound corresponds in structure to the following formula:

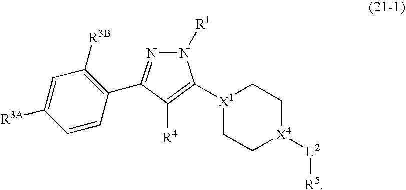

22. A compound, tautomer of the compound, or salt of the compound or tautomer according to claim 21, wherein the compound corresponds in structure a formula selected from the group consisting of:

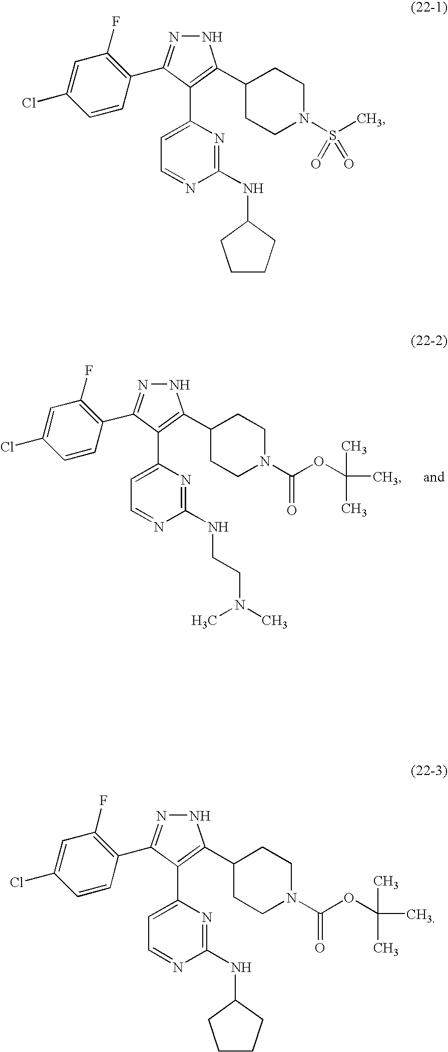

23. A compound, tautomer of the compound, or salt of the compound or tautomer according to claim 17, wherein the compound corresponds in structure to the following formula:

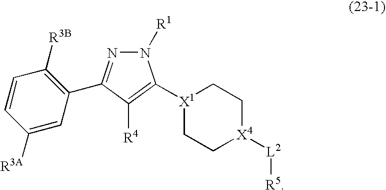

24. A compound, tautomer of the compound, or salt of the compound or tautomer according to claim 17, wherein the compound corresponds in structure to the following formula:

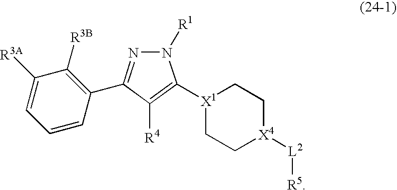

25. A compound, tautomer of the compound, or salt of the compound or tautomer according to claim 17, wherein X¹ and X⁴ are each carbon bonded to hydrogen.

26. A compound, tautomer of the compound, or salt of the compound or tautomer according to claim 25, wherein the compound corresponds in structure to the following formula:

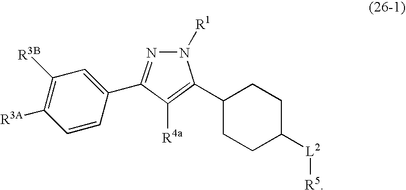

27. A compound, tautomer of the compound, or salt of the compound or tautomer according to claim 26, wherein the compound corresponds in structure a formula selected from the group consisting of:

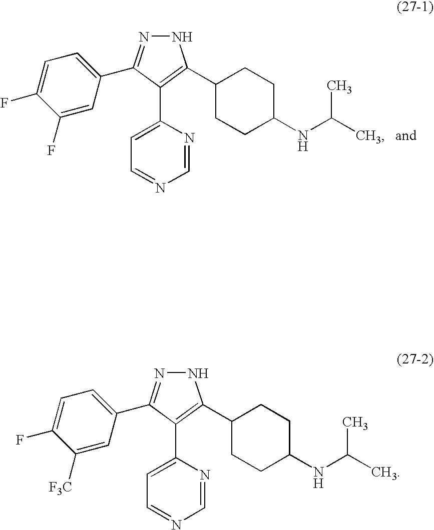

28. A compound, tautomer of the compound, or salt of the compound or tautomer according to claim 17, wherein -L² is —C(O)—.

29. A compound, tautomer of the compound, or salt of the compound or tautomer according to claim 28, wherein R⁴ is pyrimidinyl optionally substituted with a substituent selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

30. A compound, tautomer of the compound, or salt of the compound or tautomer according to claim 29, wherein:
the compound corresponds in structure to the following formula: 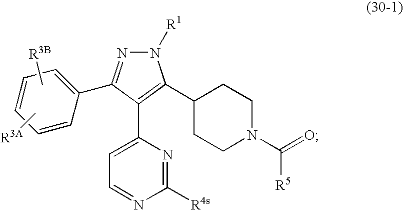
R^{4s} is selected from the group consisting of hydrogen, alkyl, aminoalkyl, alkoxyalkyl, cycloalkylalkyl, hetercycloalkylalkyl, heteroarylalkyl, amino, alkylamino, aminoalkylamino, alkoxyalkylamino, cycloalkylamino, heterocycloalkylamino, heteroarylamino, hydroxy, alkoxy, aminoalkoxy, alkoxyalkoxy, cycloalkyloxy, heterocycloalkyloxy, and heteroaryloxy, wherein:
any member of such group optionally is substituted with one or more substituents independently selected from the group consisting of hydroxy and alkyl.

31. A compound, tautomer of the compound, or salt of the compound or tautomer according to claim 30, wherein the R^{4s} is selected from the group consisting of hydrogen, C₁-C₄-alkyl, aminopropyl, monomethylaminopropyl, dimethylaminopropyl, hydroxypropyl, methoxypropyl, cyclopentylmethyl, pyrrolidinylmethyl, tetrahydrofuranylmethyl, piperidinylmethyl, tetrahydropyranylmethyl, pyridinylmethyl, C₁-C₃-alkylamino, aminoethylamino, monomethylaminoethylamino, dimethylaminoethylamino, hydroxyethylamino, methoxyethylamino, cyclopentylamino, pyrrolidinylamino, tetrahydrofuranylamino, piperidinylamino, tetrahydropyranylamino, pyridinylamino, C₁-C₃-alkoxy, aminoethoxy, monomethylaminoethoxy, dimethylaminoethoxy, hydroxyethoxy, methoxyethoxy, cyclopentyloxy, pyrrolidinyloxy, tetrahydrofuranyloxy, piperidinyloxy, tetrahydropyranyloxy, and pyridinyloxy, wherein:
any pyrrolidinyl nitrogen or piperidinyl nitrogen optionally is substituted with methyl.

32. A compound, tautomer of the compound, or salt of the compound or tautomer according to claim 31, wherein R⁵ is selected from the group consisting of alkyl, alkoxyalkyl, alkoxyalkoxyalkyl, alkylcarbonyloxyalkyl, and tetrahydrofuranylalkyl, wherein:
any member of such group optionally is substituted with one or more substituents independently selected from the group consisting of hydroxy and halogen.

33. A compound, tautomer of the compound, or salt of the compound or tautomer according to claim 32, wherein -L²-R⁵ is a radical selected from the group consisting of:

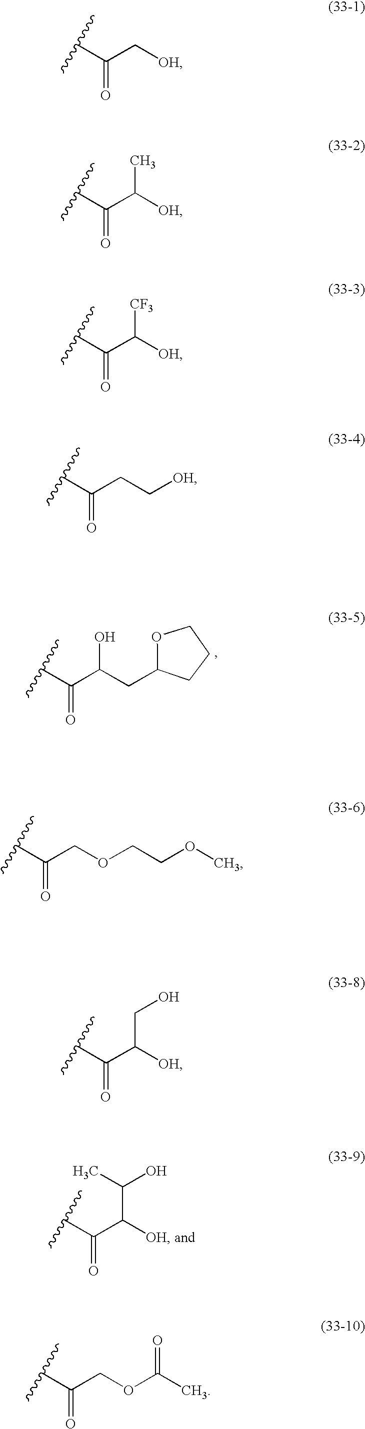

34. A compound, tautomer of the compound, or salt of the compound or tautomer according to claim 33, wherein -L²-R⁵ is hydroxymethylcarbonyl.

35. A compound, tautomer of the compound, or salt of the compound or tautomer according to claim 33, wherein the compound corresponds in structure to the following formula:

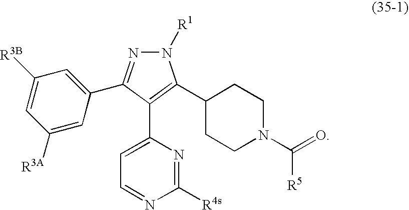

36. A compound, tautomer of the compound, or salt of the compound or tautomer according to claim 35, wherein the compound corresponds in structure to a formula selected from the group consisting of:

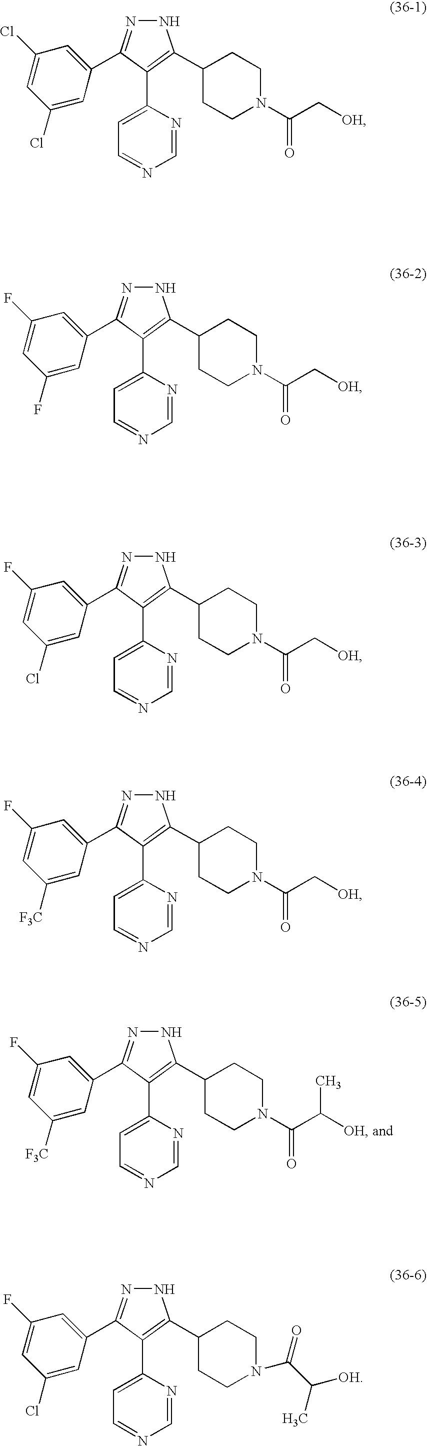

37. A compound, tautomer of the compound, or salt of the compound or tautomer according to claim 33, wherein the compound corresponds in structure to the following formula:

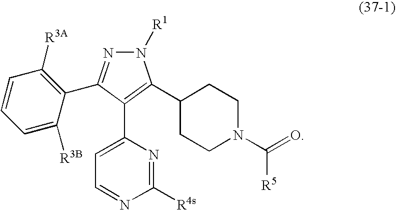

38. A compound, tautomer of the compound, or salt of the compound or tautomer according to claim 37, wherein the compound corresponds in structure to the following formula:

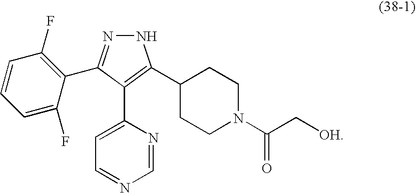

39. A compound, tautomer of the compound, or salt of the compound or tautomer according to claim 33, wherein the compound corresponds in structure to the following formula:

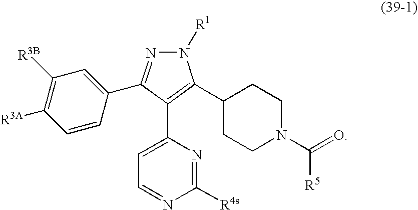

40. A compound, tautomer of the compound, or salt of the compound or tautomer according to claim 39, wherein the compound corresponds in structure to a formula selected from the group consisting of:

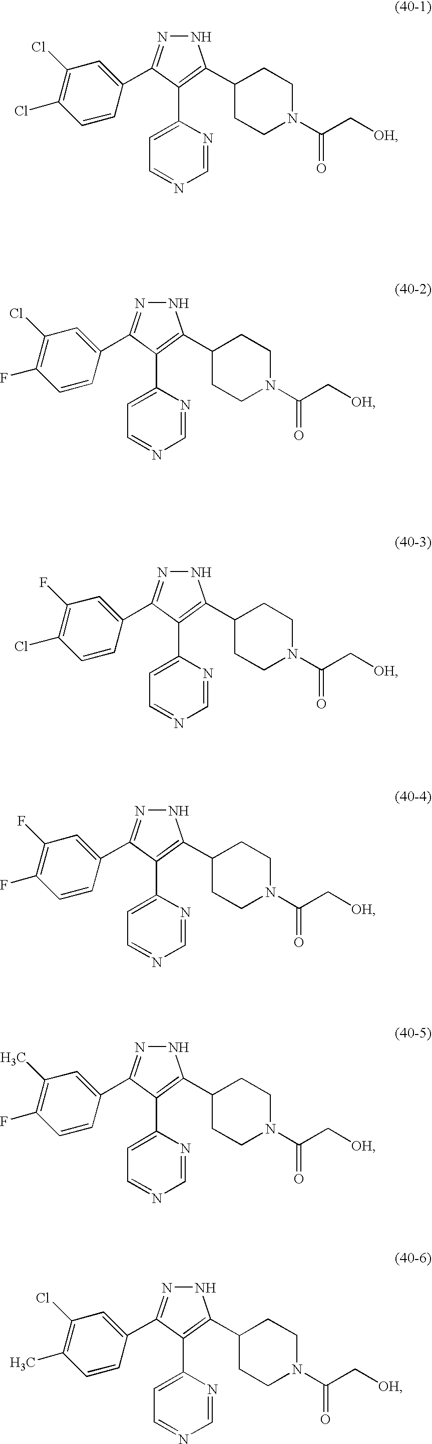

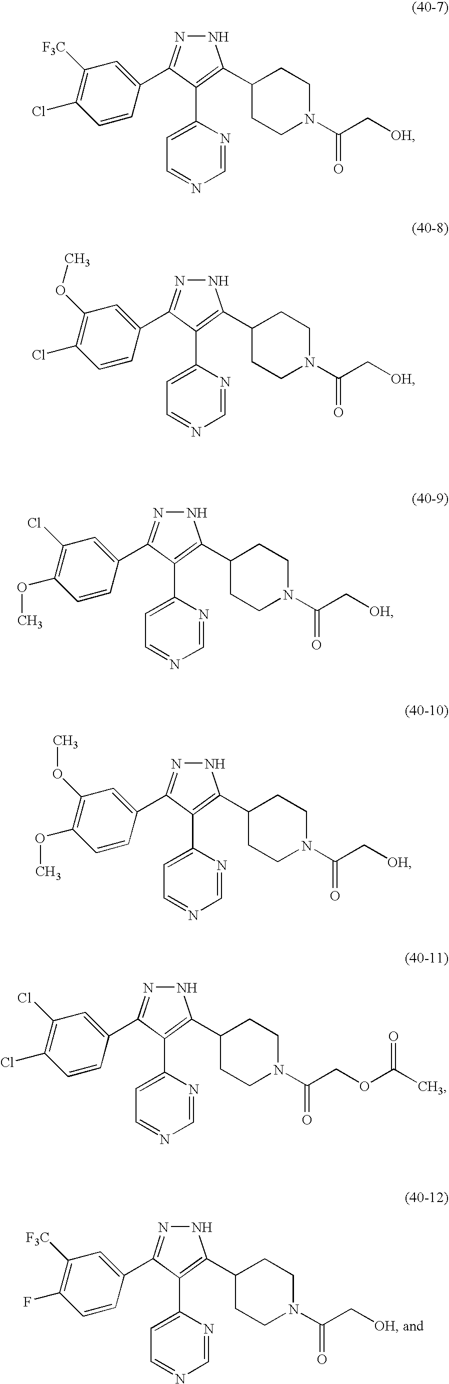

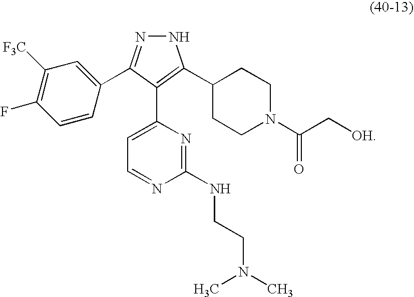

41. A compound, tautomer of the compound, or salt of the compound or tautomer according to claim 39, wherein the compound corresponds in structure to a formula selected from the group consisting of:

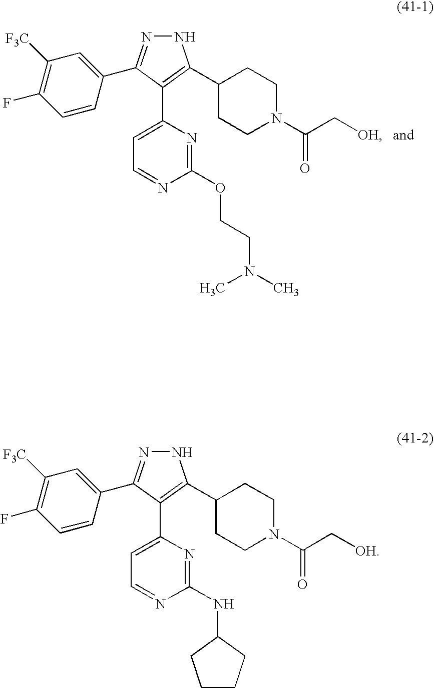

42. A compound, tautomer of the compound, or salt of the compound or tautomer according to claim 33, wherein the compound corresponds in structure to the following formula:

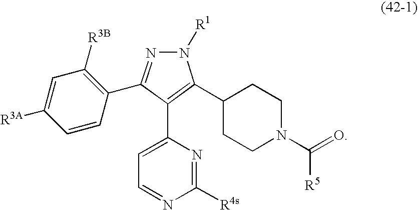

43. A compound, tautomer of the compound, or salt of the compound or tautomer according to claim 42, wherein the compound corresponds in structure to a formula selected from the group consisting of:

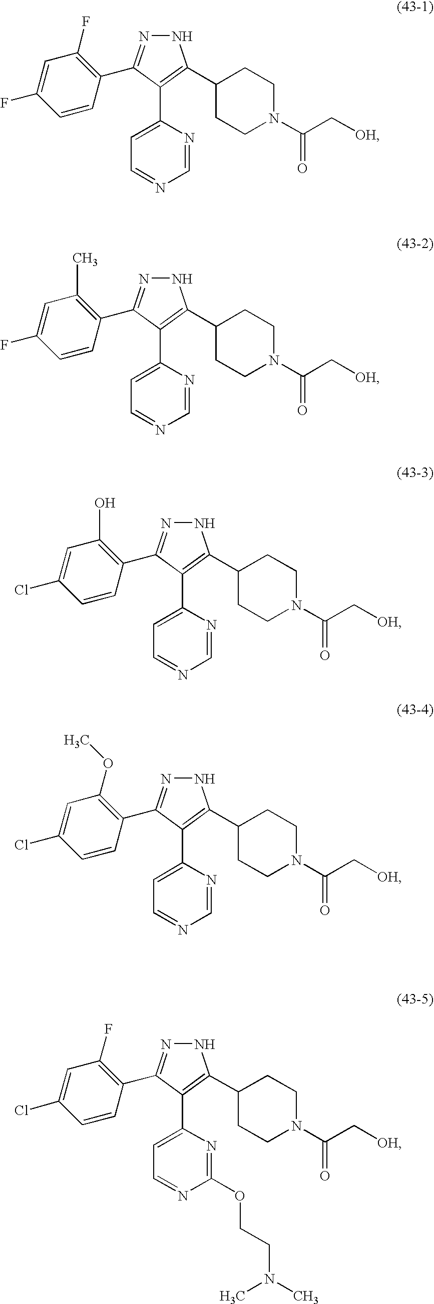

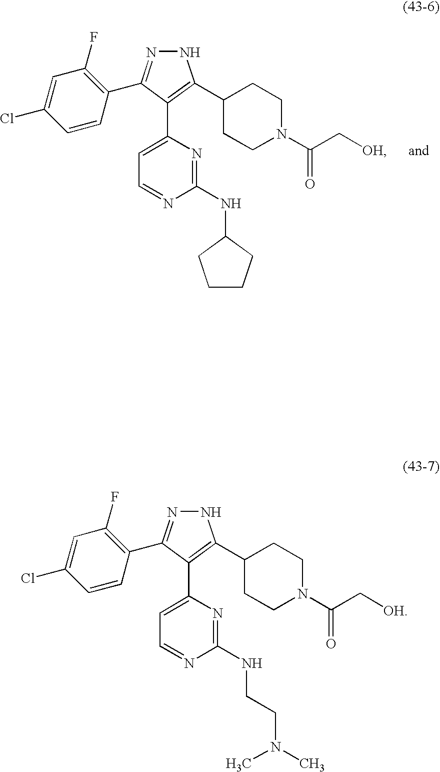

44. A compound, tautomer of the compound, or salt of the compound or tautomer according to claim 42, wherein the compound corresponds in structure to the following formula:

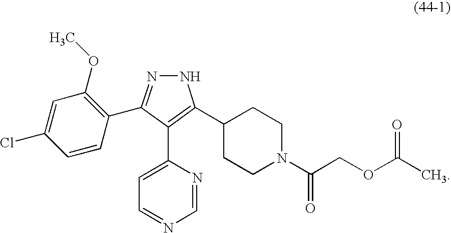

45. A compound, tautomer of the compound, or salt of the compound or tautomer according to claim 42, wherein the compound corresponds in structure to a formula selected from the group consisting of:

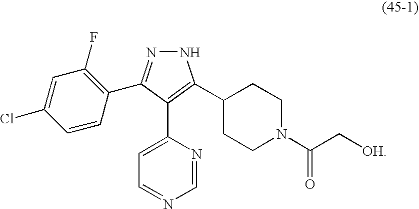

46. A compound, tautomer of the compound, or salt of the compound or tautomer according to claim 42, wherein the compound corresponds in structure to the following formula:

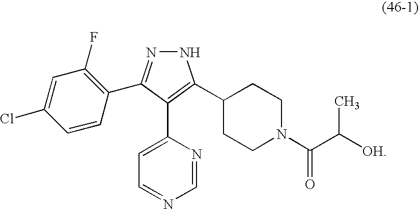

47. A compound, tautomer of the compound, or salt of the compound or tautomer according to claim 33, wherein the compound corresponds in structure to the following formula:

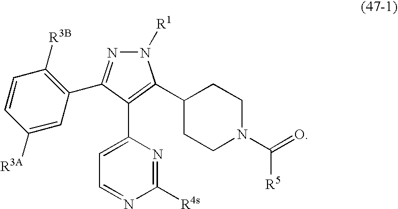

48. A compound, tautomer of the compound, or salt of the compound or tautomer according to claim 47, wherein the compound corresponds in structure to a formula selected from the group consisting of:

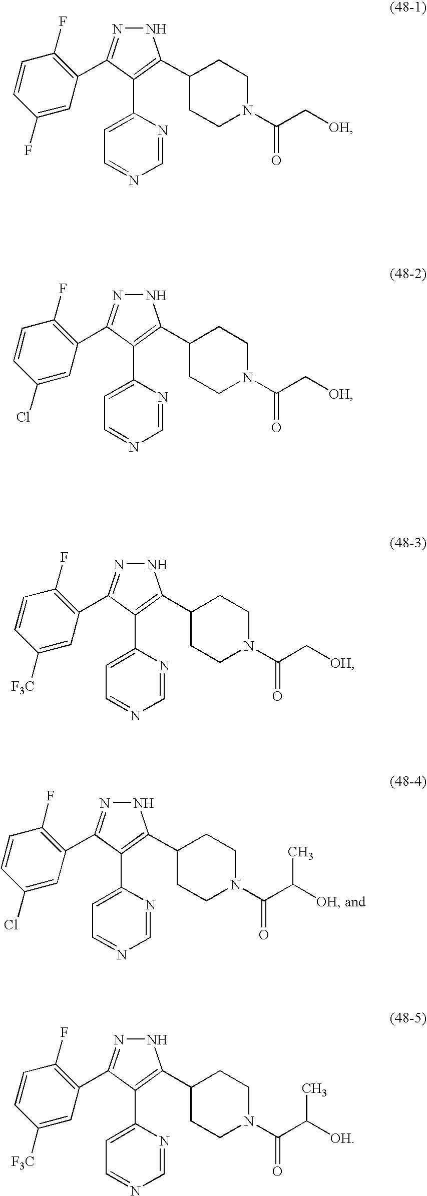

49. A compound, tautomer of the compound, or salt of the compound or tautomer according to claim 33, wherein the compound corresponds in structure to the following formula:

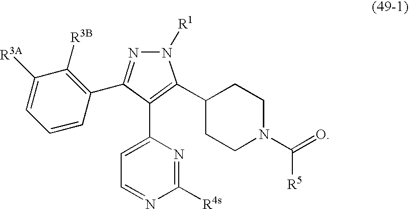

50. A compound, tautomer of the compound, or salt of the compound or tautomer according to claim 49, wherein the compound corresponds in structure to a formula selected from the group consisting of:

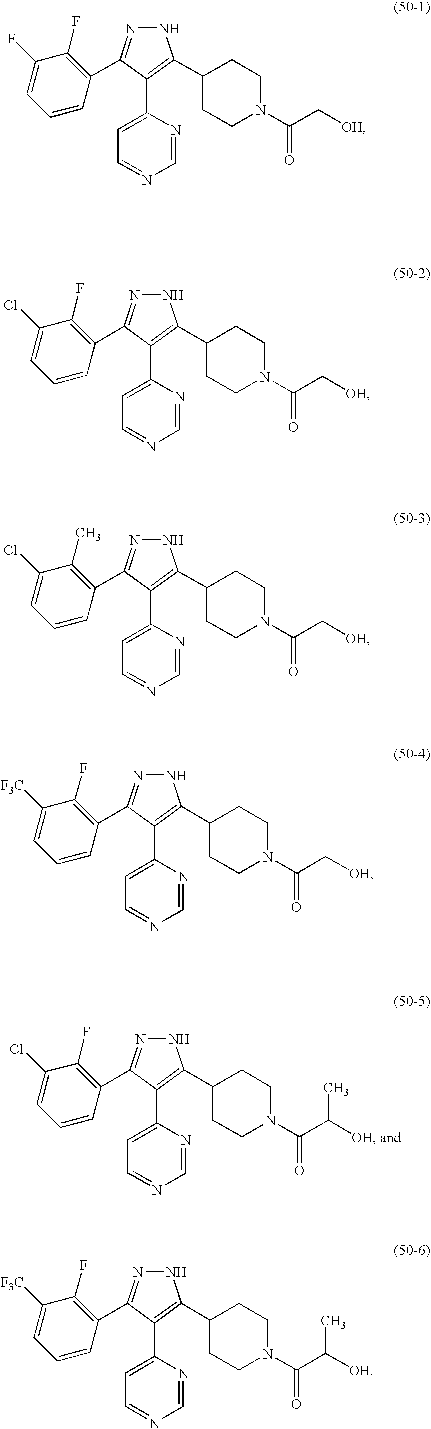

51. A compound, a tautomer of the compound, or a salt of the compound or tautomer, wherein:
the compound corresponds in structure to the following formula: 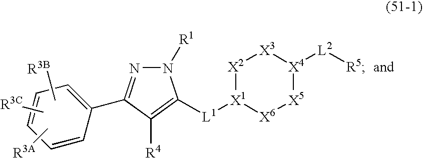
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} and R^{3B} are independently selected from the group consisting of halogen, hydroxy, cyano, amino, alkyl, aminoalkyl, monoalkylamino, dialkylamino, cycloalkylamino, alkoxy, and alkoxyalkyl, wherein:
any carbon of any member of such group optionally is substituted with one or more substituents independently selected from the group consisting of halogen, hydroxy, and cyano; and
R^{3C} is selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R⁴ is selected from the group consisting of pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

52. A compound, tautomer of the compound, or salt of the compound or tautomer according to claim 51, wherein the compound corresponds in structure to the following formula:

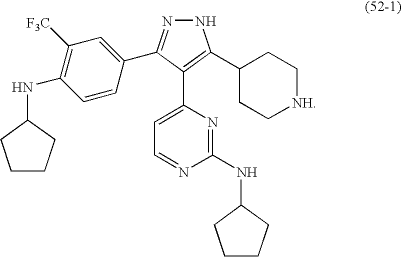

53. A compound, tautomer of the compound, or salt of the compound or tautomer according to claim 51, wherein R^{3A} and R^{3B} are independently selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano.

54. A compound, tautomer of the compound, or salt of the compound or tautomer according to claim 53, wherein R^{3A} and R^{3B} are independently selected from the group consisting of halogen, methyl, methoxy, halomethyl, and halomethoxy.

55. A compound, tautomer of the compound, or salt of the compound or tautomer according to claim 53, wherein R⁴ is pyrimidinyl substituted with a substituent selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

56. A compound, tautomer of the compound, or salt of the compound or tautomer according to claim 55, wherein:
the compound corresponds in structure to the following formula: 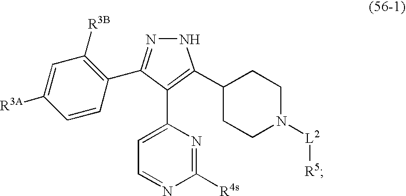
R^{4s} is selected from the group consisting of alkyl, aminoalkyl, alkoxycarbonyl, carbocyclyloxycarbonyl, heterocyclyloxycarbonyl, and alkylaminocarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

57. A compound, tautomer of the compound, or salt of the compound or tautomer according to claim 56, wherein R^{4s} is selected from the group consisting of alkoxycarbonyl, carbocyclyloxycarbonyl, and heterocyclyloxycarbonyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

58. A compound, tautomer of the compound, or salt of the compound or tautomer according to claim 56, wherein R^{4s} is alkylaminocarbonyl optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

59. A compound, tautomer of the compound, or salt of the compound or tautomer according to claim 56, wherein R^{4s} is selected from the group consisting of —CH₂OH, —C(CH₃)(H)—OH, and —C(CH₃)₂—OH.

60. A compound, tautomer of the compound, or salt of the compound or tautomer according to claim 56, wherein R⁴, is aminomethyl, wherein:
the amino nitrogen optionally is substituted with up to two substituents independently selected from the group consisting of alkyl, alkenyl, hydroxy, haloalkyl, alkylsulfonyl, alkoxyalkyl, and heterocyclyl.

61. A compound, tautomer of the compound, or salt of the compound or tautomer according to claim 56, wherein -L 2-R⁵ is selected from the group consisting of hydrogen and alkyl.

62. A compound, tautomer of the compound, or salt of the compound or tautomer according to claim 53, wherein the compound corresponds in structure to the following formula:

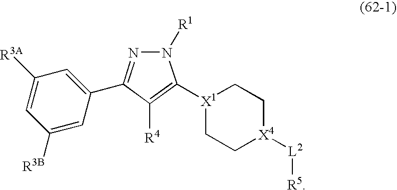

63. A compound, tautomer of the compound, or salt of the compound or tautomer according to claim 53, wherein the compound corresponds in structure to the following formula:

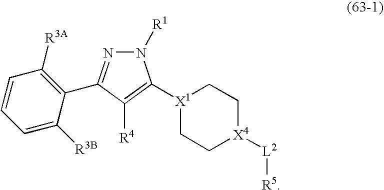

64. A compound, tautomer of the compound, or salt of the compound or tautomer according to claim 53, wherein the compound corresponds in structure to the following formula:

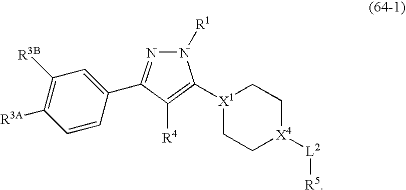

65. A compound, tautomer of the compound, or salt of the compound or tautomer according to claim 64, wherein the compound corresponds in structure to a formula selected from the group consisting of:

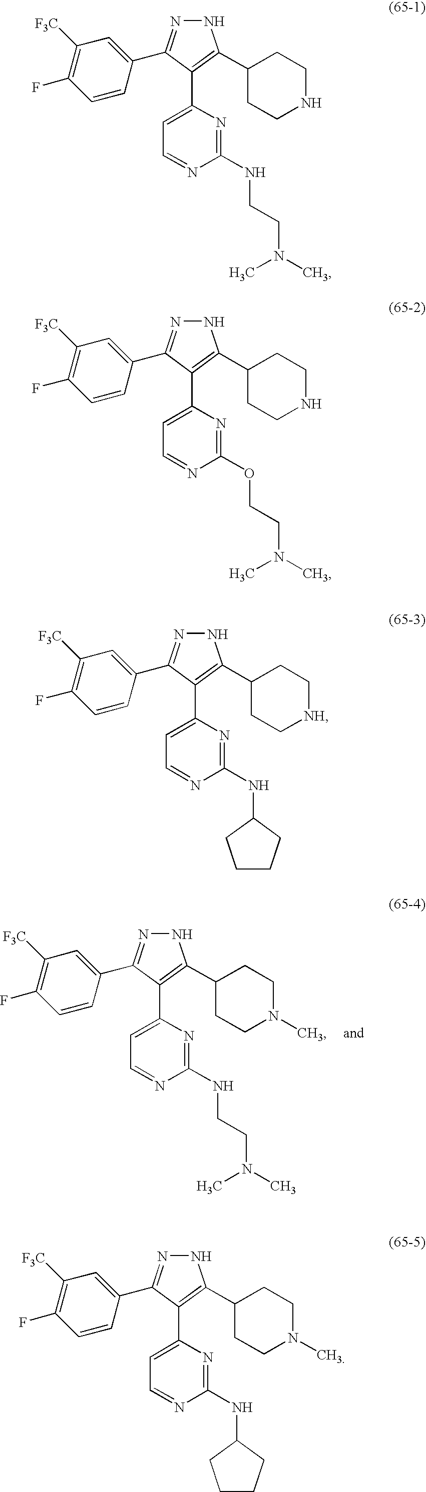

66. A compound, tautomer of the compound, or salt of the compound or tautomer according to claim 53, wherein the compound corresponds in structure to the following formula:

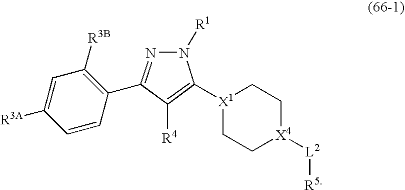

67. A compound, tautomer of the compound, or salt of the compound or tautomer according to claim 66, wherein the compound corresponds in structure to a formula selected from the group consisting of:

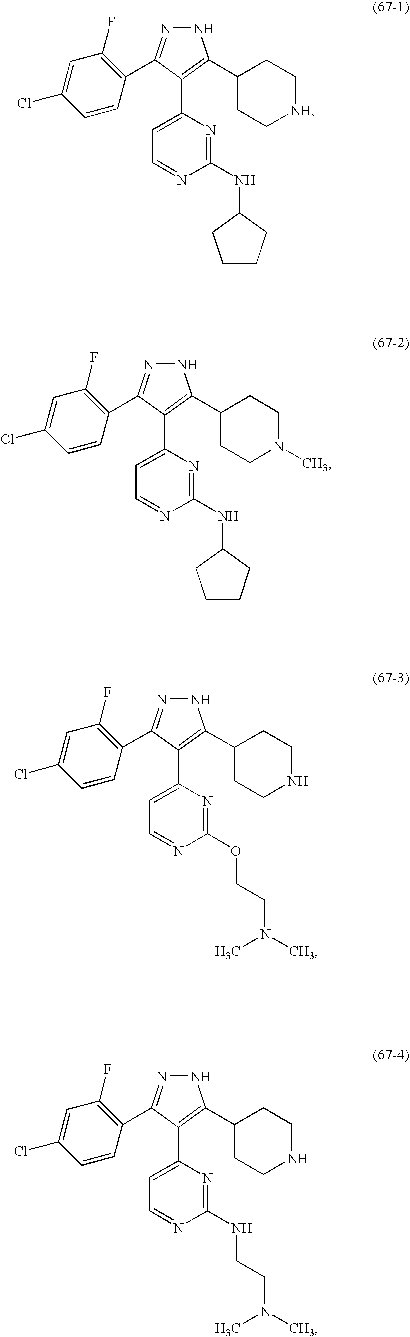

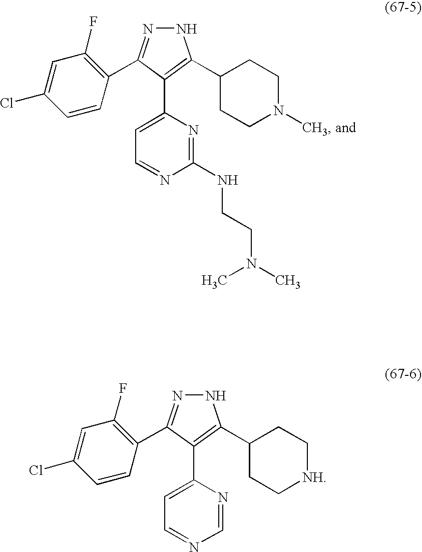

68. A compound, tautomer of the compound, or salt of the compound or tautomer according to claim 53, wherein the compound corresponds in structure to the following formula:

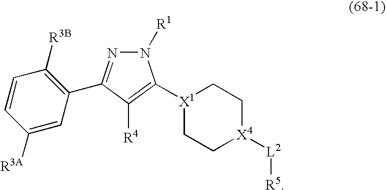

69. A compound, tautomer of the compound, or salt of the compound or tautomer according to claim 53, wherein the compound corresponds in structure to the following formula:

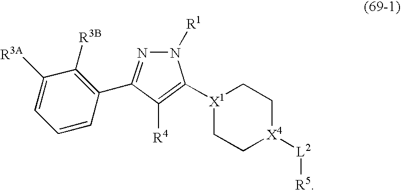

70. A compound, a tautomer of the compound, or a salt of the compound or tautomer, wherein:
the compound corresponds in structure to the following formula: 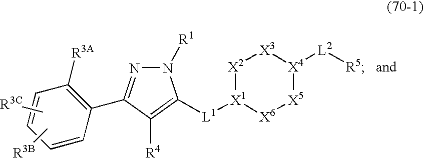
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a}), —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of-CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} and R^{3B} are independently selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3C} is selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R⁴ is selected from the group consisting of pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

71. A compound, tautomer of the compound, or salt of the compound or tautomer according to claim 70, wherein R^{3A} is selected from the group consisting of halogen, methyl, methoxy, halomethyl, and halomethoxy.

72. A compound, tautomer of the compound, or salt of the compound or tautomer according to claim 70, wherein the compound corresponds in structure to a formula selected from the group consisting of:

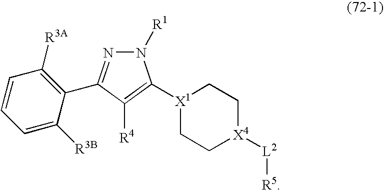

73. A compound, tautomer of the compound, or salt of the compound or tautomer according to claim 70, wherein the compound corresponds in structure to the following formula:

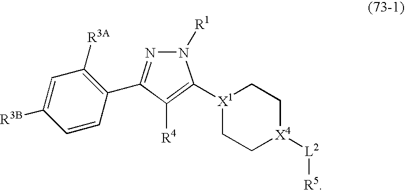

74. A compound, tautomer of the compound, or salt of the compound or tautomer according to claim 70, wherein the compound corresponds in structure to the following formula:

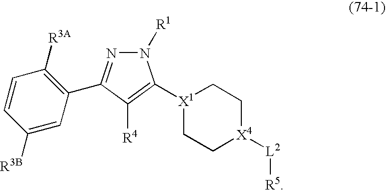

75. A compound, tautomer of the compound, or salt of the compound or tautomer according to claim 70, wherein the compound corresponds in structure to the following formula:

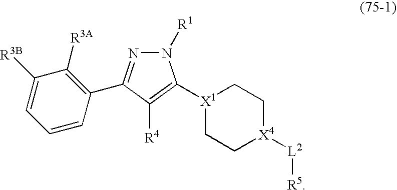

76. A compound, a tautomer of the compound, or a salt of the compound or tautomer, wherein:
the compound corresponds in structure to the following formula: 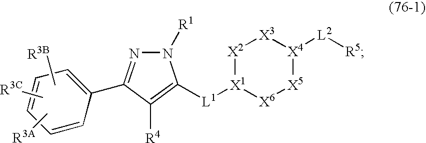
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—, —C(O)—, —C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R⁴ is selected from the group consisting of pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

77. A compound, tautomer of the compound, or salt of the compound or tautomer according to claim 76, wherein X², X³, X⁵, and X⁶ are each —CH₂—.

78. A compound, tautomer of the compound, or salt of the compound or tautomer according to claim 77, wherein L¹ is a bond.

79. A compound, tautomer of the compound, or salt of the compound or tautomer according to claim 78, wherein -L² is —C(O)—.

80. A compound, tautomer of the compound, or salt of the compound or tautomer according to claim 79, wherein R⁵ is selected from the group consisting of alkyl, alkoxyalkyl, alkoxyalkoxyalkyl, and tetrahydrofuranylalkyl, wherein:
any member of such group optionally is substituted with one or more substituents independently selected from the group consisting of hydroxy and halogen.

81. A compound, tautomer of the compound, or salt of the compound or tautomer according to claim 80, wherein -L²-R⁵ is a radical selected from the group consisting of:

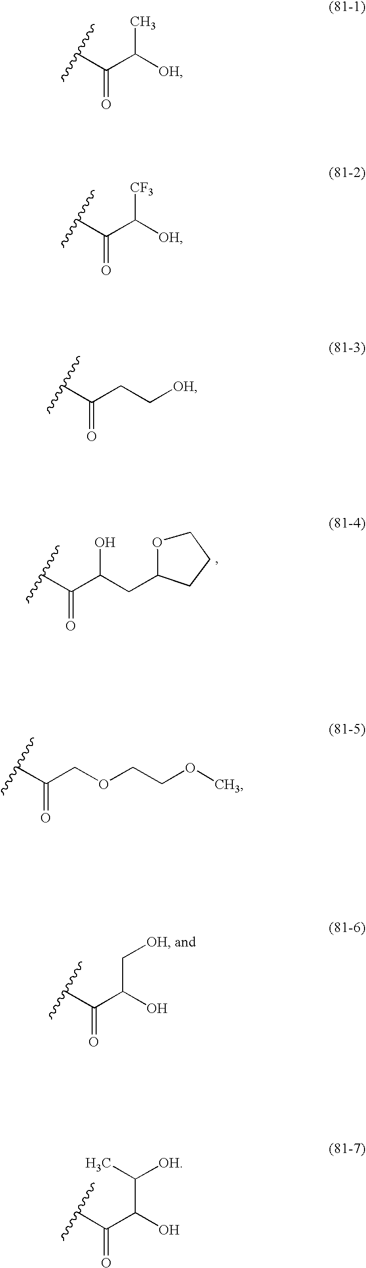

82. A compound, tautomer of the compound, or salt of the compound or tautomer according to claim 78, wherein L² is —O—.

83. A compound, tautomer of the compound, or salt of the compound or tautomer according to claim 82, wherein the compound corresponds in structure to the following formula:

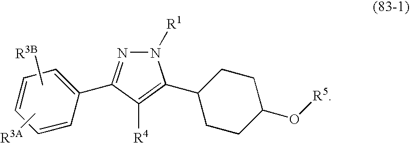

84. A compound, tautomer of the compound, or salt of the compound or tautomer according to claim 83, wherein R⁵ is selected from the group consisting of hydrogen, alkenyl, and alkylcarbonylalkyl.

85. A compound, tautomer of the compound, or salt of the compound or tautomer according to claim 78, wherein the compound corresponds in structure to the following formula:

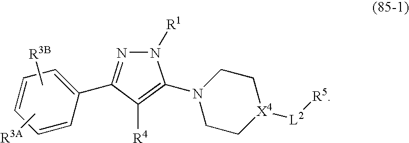

86. A compound, tautomer of the compound, or salt of the compound or tautomer according to claim 85, wherein the compound corresponds in structure to the following formula:

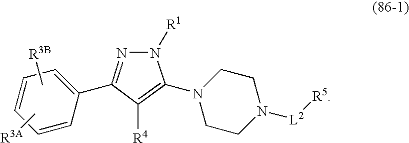

87. A compound, tautomer of the compound, or salt of the compound or tautomer according to claim 85, wherein the compound corresponds in structure to the following formula:

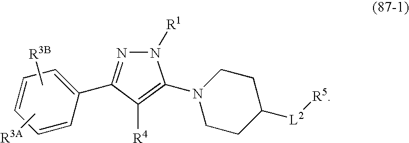

88. A compound, tautomer of the compound, or salt of the compound or tautomer according to claim 78, wherein the compound corresponds in structure to the following formula:

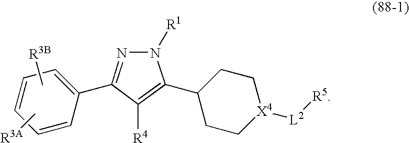

89. A compound, tautomer of the compound, or salt of the compound or tautomer according to claim 88, wherein the compound corresponds in structure to the following formula:

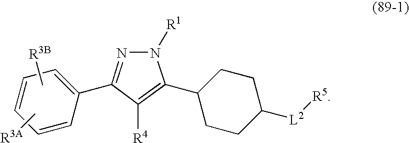

90. A compound, tautomer of the compound, or salt of the compound or tautomer according to claim 89, wherein:
L² is a bond; and
R⁵ is heterocyclyl optionally substituted with one or more substituents independently selected from the group consisting of halogen, hydroxy, alkyl, haloalkyl, hydroxyalkyl, alkoxy, and haloalkoxy.

91. A compound, tautomer of the compound, or salt of the compound or tautomer according to claim 90, wherein:
the compound corresponds in structure to the following formula: 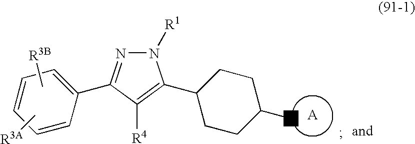
the ring structure A is a heterocyclyl ring that contains a nitrogen bonded to the cyclohexyl, and optionally is substituted with one or more substituents independently selected from the group consisting of halogen, hydroxy, alkyl, haloalkyl, hydroxyalkyl, alkoxy, and haloalkoxy.

92. A compound, tautomer of the compound, or salt of the compound or tautomer according to claim 89, wherein:
L² is —N(R^{a})—; and
R⁵ is selected from the group consisting of alkyl, carbocyclyl, and carbocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy.

93. A compound, tautomer of the compound, or salt of the compound or tautomer according to claim 88, wherein:
the compound corresponds in structure to the following formula: 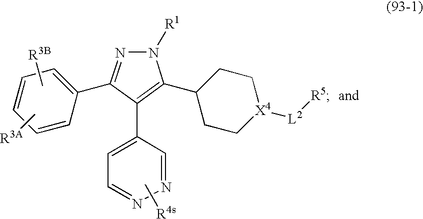
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

94. A compound, tautomer of the compound, or salt of the compound or tautomer according to claim 93, wherein R^{4s} is selected from the group consisting of hydrogen, alkylthio, mono-alkylamino, di-alkylamino, alkoxy, and haloalkoxy.

95. A compound, tautomer of the compound, or salt of the compound or tautomer according to claim 94, wherein the compound corresponds in structure to the following formula:

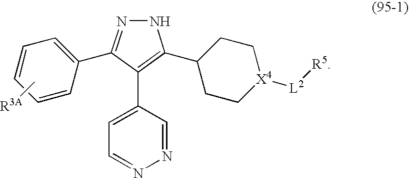

96. A compound, tautomer of the compound, or salt of the compound or tautomer according to claim 95, wherein -L²-R⁵ is alkylcarbonyl substituted with one or more hydroxy.

97. A compound, tautomer of the compound, or salt of the compound or tautomer according to claim 96, wherein the compound corresponds in structure to the following formula:

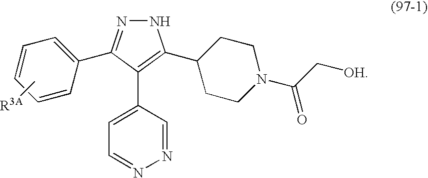

98. A compound, tautomer of the compound, or salt of the compound or tautomer according to claim 97, wherein the compound corresponds in structure to the following formula:

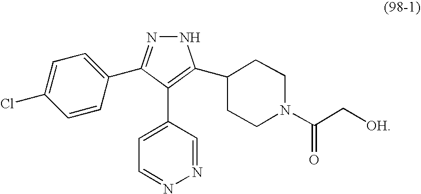

99. A compound, tautomer of the compound, or salt of the compound or tautomer according to claim 88, wherein:
the compound corresponds in structure to the following formula: 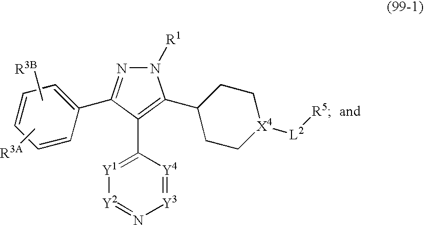
and
two of Y¹, Y², Y³, and Y⁴ are nitrogen, one of Y¹, Y², Y³, and Y⁴ is carbon bonded to R^{4s}, and one of Y¹, Y², Y³, and Y⁴ is carbon bonded to hydrogen; and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

100. A compound, tautomer of the compound, or salt of the compound or tautomer according to claim 99, wherein the compound corresponds in structure to a formula selected from the group consisting of:

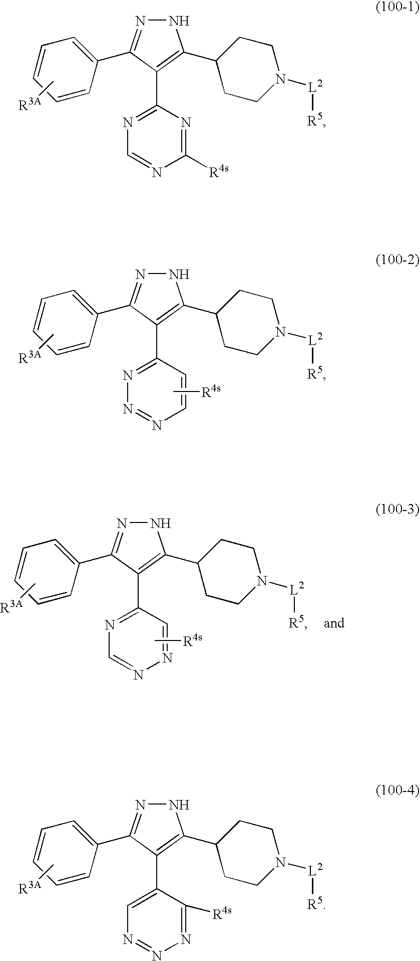

101. A compound, tautomer of the compound, or salt of the compound or tautomer according to claim 100, wherein -L²-R⁵ is alkylcarbonyl substituted with one or more hydroxy.

102. A compound, tautomer of the compound, or salt of the compound or tautomer according to claim 101, wherein the compound corresponds in structure to a formula selected from the group consisting of:

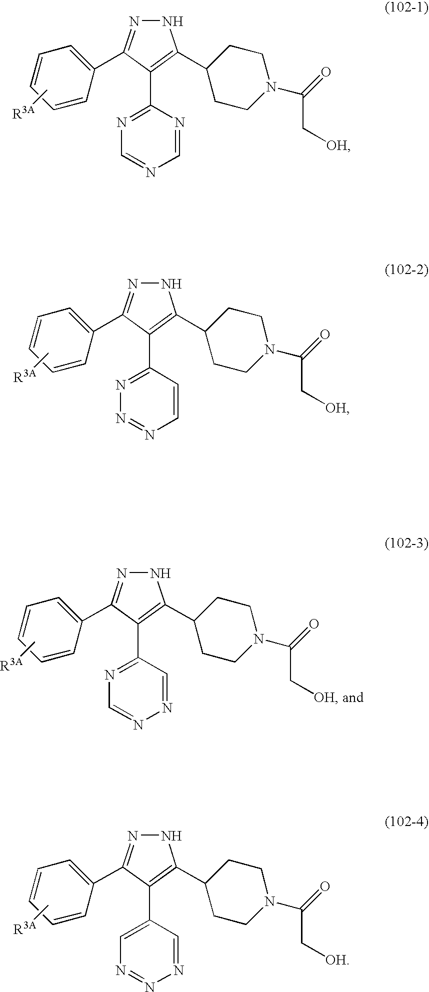

103. A compound, tautomer of the compound, or salt of the compound or tautomer according to claim 88, wherein:
the compound corresponds in structure to the following formula: 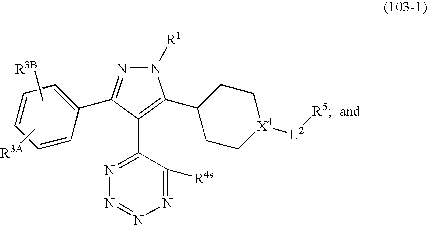
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

104. A compound, tautomer of the compound, or salt of the compound or tautomer according to claim 103, wherein the compound corresponds in structure to the following formula:

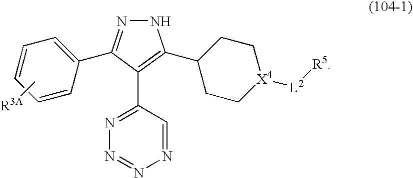

105. A compound, tautomer of the compound, or salt of the compound or tautomer according to claim 104, wherein -L²-R⁵ is hydroxyalkylcarbonyl.

106. A compound, tautomer of the compound, or salt of the compound or tautomer according to claim 105, wherein the compound corresponds in structure to the following formula:

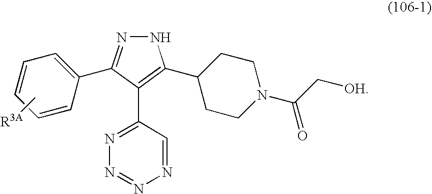

107. A compound, a tautomer of the compound, or a salt of the compound or tautomer, wherein:
the compound corresponds in structure to the following formula: 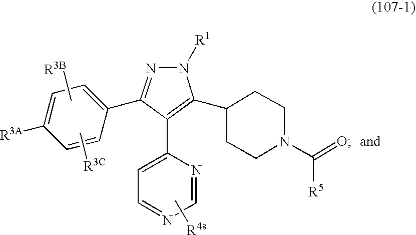
and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of hydroxy, cyano, amino, monomethylamino, monoethylamino, dimethylamino, diethylamino, N-methyl-N-ethyl-amino, methyl, ethyl, haloethyl, propyl, halopropyl, aminomethyl, aminoethyl, hydroxymethyl, hydroxyethyl, methoxy, ethoxy, haloethoxy, methoxymethyl, and halomethoxymethyl; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is hydroxyalkyl.

108. A compound, tautomer of the compound, or salt of the compound or tautomer according to claim 107, wherein R⁵ is C₁-C₆-hydroxyalkyl.

109. A compound, tautomer of the compound, or salt of the compound or tautomer according to claim 108, wherein the compound corresponds in structure to a formula selected from the group consisting of:

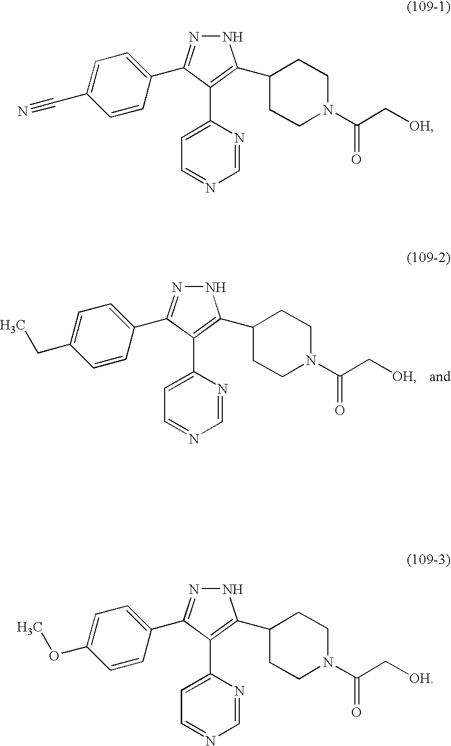

110. A compound, a tautomer of the compound, or a salt of the compound or tautomer, wherein:
the compound corresponds in structure to the following formula: 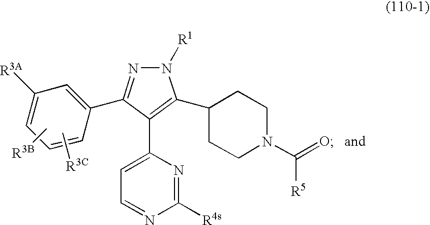
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of hydroxy, cyano, amino, monomethylamino, monoethylamino, dimethylamino, diethylamino, N-methyl-N-ethyl-amino, methyl, ethyl, haloethyl, propyl, halopropyl, aminomethyl, aminoethyl, hydroxymethyl, hydroxyethyl, methoxy, halomethoxy, ethoxy, haloethoxy, methoxymethyl, and halomethoxymethyl; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is hydroxyalkyl.

111. A compound, tautomer of the compound, or salt of the compound or tautomer according to claim 110, wherein R⁵ is C₁-C₆-hydroxyalkyl.

112. A compound, tautomer of the compound, or salt of the compound or tautomer according to claim 111, wherein the compound corresponds in structure to the following formula:

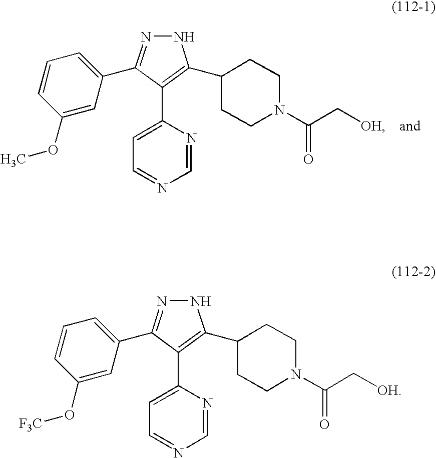

113. A compound, a tautomer of the compound, or a salt of the compound or tautomer, wherein:
the compound corresponds in structure to the following formula: 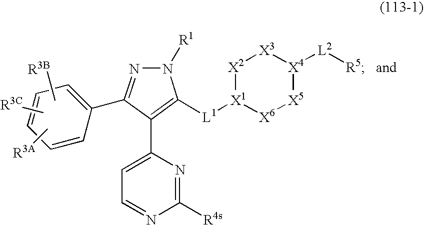
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a}), —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that x⁶ is —CH₂— if X is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
any of any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of phosphonooxyalkyl, monoalkylphosphonooxyalkyl, dialkylphosphonooxyalkyl, aminoalkylcarbonyloxyalkyl, monoalkylaminoalkylcarbonyloxyalkyl, dialkylaminoalkylcarbonyloxyalkyl, phenylalkyl substituted with alkylcarbonyloxy, and tetrahydrofuranyl; and
each R^{a} is independently selected from the group consisting of hydrogen and alkyl.

114. A compound, tautomer of the compound, or salt of the compound or tautomer according to claim 113, wherein the compound corresponds in structure to a formula selected from the group consisting of:

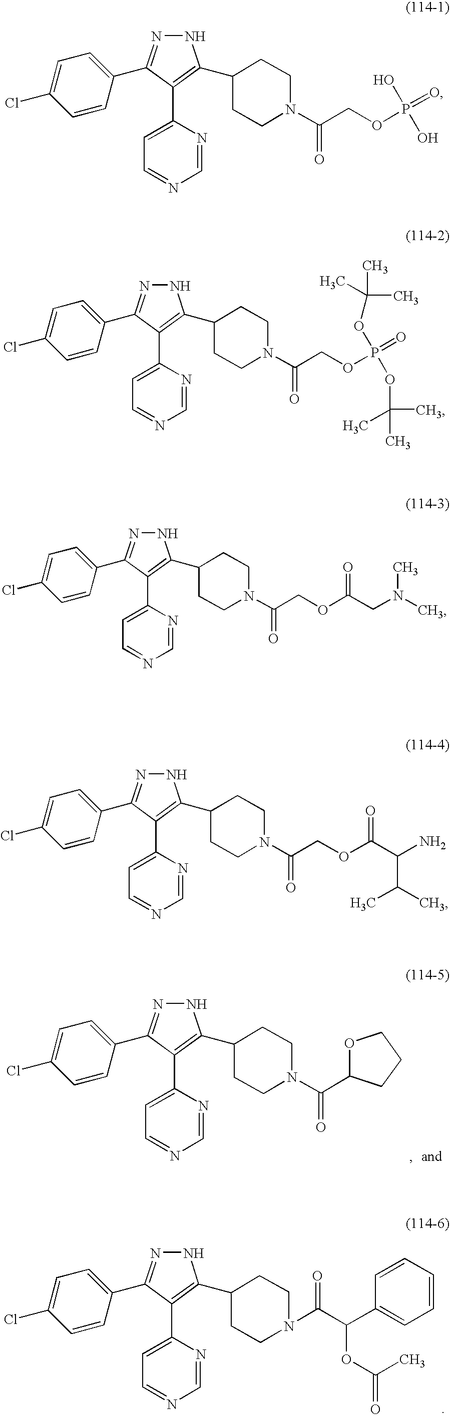

115. A compound, a tautomer of the compound, or a salt of the compound or tautomer, wherein:
the compound corresponds in structure to the following formula: 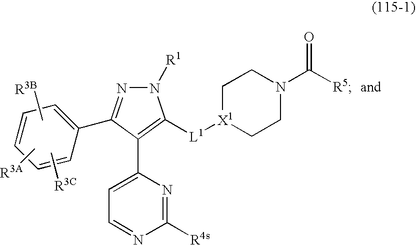
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—, —C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X⁴ is selected from the group consisting of nitrogen and carbon bonded to hydrogen; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{4s} is selected from the group consisting of hydrogen, alkyl, aminoalkyl, alkoxyalkyl, cycloalkylalkyl, arylalkyl, hetercycloalkylalkyl, heteroarylalkyl, amino, alkylamino, aminoalkylamino, alkoxyalkylamino, cycloalkylamino, arylamino, heterocycloalkylamino, heteroarylamino, hydroxy, alkoxy, aminoalkoxy, alkoxyalkoxy, cycloalkyloxy, aryloxy, heterocycloalkyloxy, heteroaryloxy, thiol, alkylthio, cycloalkylthio, arylthio, heterocycloalkylthio, heteroarylthio, aminosulfonyl, alkylsulfonyl, cycloalkylsulfonyl, arylsulfonyl, heterocycloalkylsulfonyl, and heteroarylsulfonyl, wherein:
any member of such group optionally is substituted with one or more substituents independently selected from the group consisting of hydroxy, cyano, and alkyl; and
R⁵ is alkylcarbonyloxyalkyl; and
each R^{a} is independently selected from the group consisting of hydrogen and alkyl.

116. A compound, tautomer of the compound, or salt of the compound or tautomer according to claim 115, wherein X¹ is carbon bonded to hydrogen.

117. A compound, tautomer of the compound, or salt of the compound or tautomer according to claim 116, wherein L¹ is a bond.

118. A compound, tautomer of the compound, or salt of the compound or tautomer according to claim 117, wherein R⁵ is methylcarbonyloxymethyl.

119. A compound, tautomer of the compound, or salt of the compound or tautomer according to claim 118, wherein the compound corresponds in structure to a formula selected from the group consisting of:

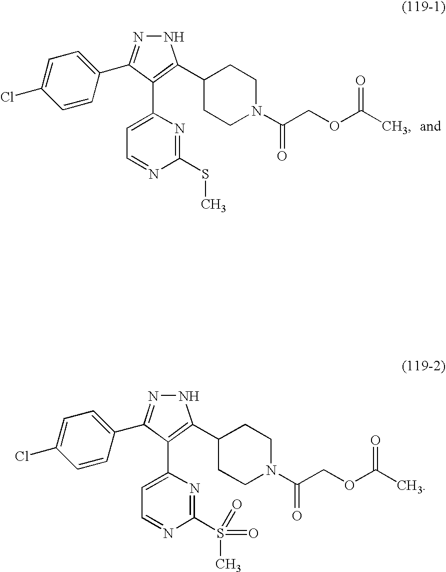

120. A compound, a tautomer of the compound, or a salt of the compound or tautomer, wherein:
the compound corresponds in structure to the following formula: 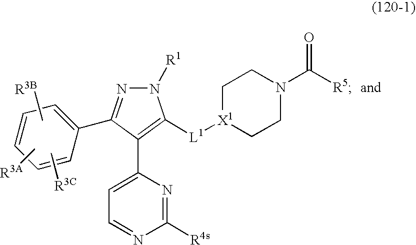
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—, —C(O)—, —O—C(O)—O—, —C(H)═C(H)—, —C≡C—, —N═N—, —N(R^{a})—N(R^{a})—, —N(R^{a})—C(O)—N(R^{a})—, C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{4s} is selected from the group consisting of hydrogen, C₁-C₆-alkyl, aminoalkyl, alkoxyalkyl, cycloalkylalkyl, hetercycloalkylalkyl, heteroarylalkyl, aminoalkylamino, alkoxyalkylamino, cycloalkylamino, heterocycloalkylamino, heteroarylamino, hydroxy, C₂-C₆-alkoxy, aminoalkoxy, alkoxyalkoxy, cycloalkyloxy, heterocycloalkyloxy, heteroaryloxy, thiol, C₂-C₆-alkylthio, cycloalkylthio, heterocycloalkylthio, heteroarylthio, aminosulfonyl, C₂-C₆-alkylsulfonyl, cycloalkylsulfonyl, heterocycloalkylsulfonyl, and heteroarylsulfonyl, wherein:
any member of such group optionally is substituted with one or more substituents independently selected from the group consisting of hydroxy, cyano, and alkyl; and
R⁵ is hydroxyalkyl; and
each R^{a} is independently selected from the group consisting of hydrogen and alkyl.

121. A compound, tautomer of the compound, or salt of the compound or tautomer according to claim 120, wherein X¹ is carbon bonded to hydrogen.

122. A compound, tautomer of the compound, or salt of the compound or tautomer according to claim 121, wherein L¹ is a bond.

123. A compound, tautomer of the compound, or salt of the compound or tautomer according to claim 122, wherein R⁵ is hydroxymethyl.

124. A compound, tautomer of the compound, or salt of the compound or tautomer according to claim 123, wherein the compound corresponds in structure to a formula selected from the group consisting of:

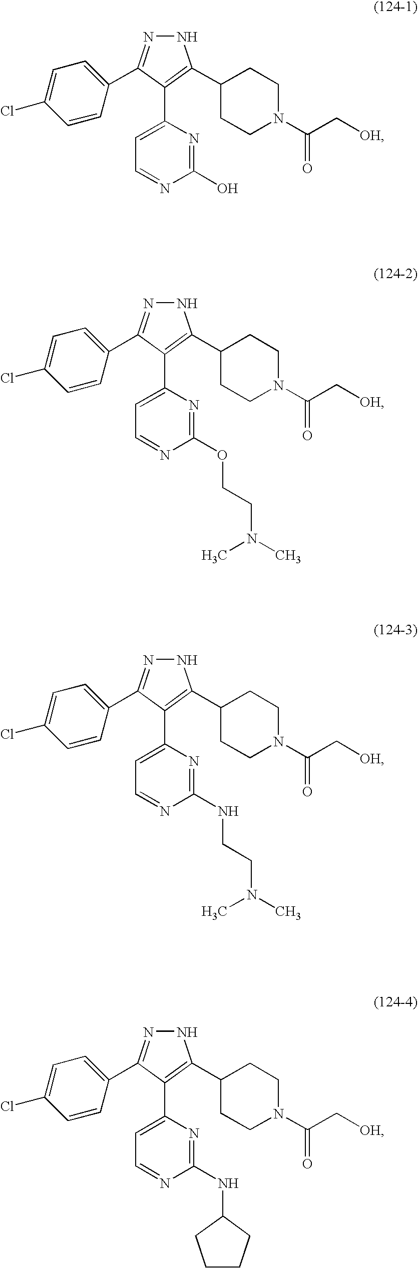

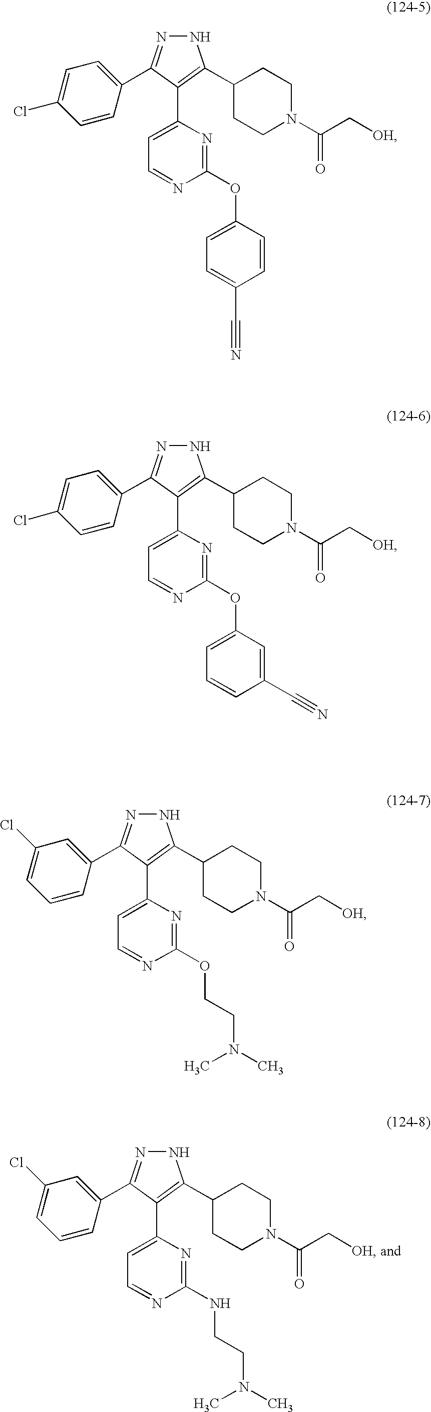

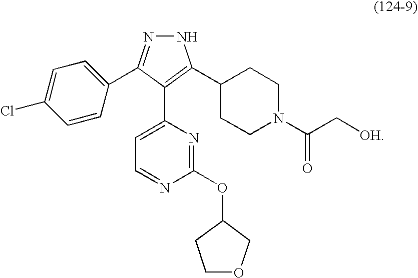

125. A compound, a tautomer of the compound, or a salt of the compound or tautomer, wherein:
the compound corresponds in structure to the following formula: 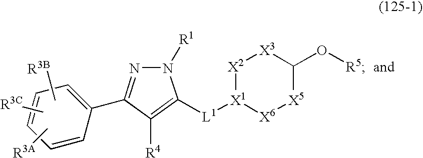
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C═C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is selected from the group consisting of pyridazinyl, pyrazinyl, pyrimidinyl, triazinyl, tetrazinyl, benzazinyl, benzodiazinyl, naphthyridinyl, pyridopyridinyl, pyrinyl, maleimidyl, pyridonyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

126. A compound, tautomer of the compound, or salt of the compound or tautomer according to claim 125, wherein the compound corresponds in structure to the following formula:

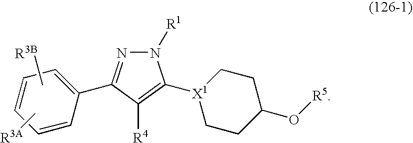

127. A compound, tautomer of the compound, or salt of the compound or tautomer according to claim 126, wherein the compound corresponds in structure to the following formula:

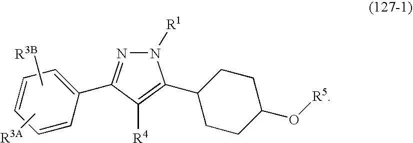

128. A compound, tautomer of the compound, or salt of the compound or tautomer according to claim 127, wherein:
the compound corresponds in structure to the following formula: 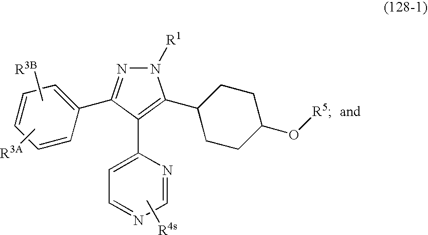
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

129. A compound, tautomer of the compound, or salt of the compound or tautomer according to claim 128, wherein the compound corresponds in structure to the following formula:

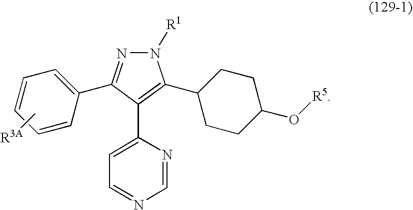

130. A compound, tautomer of the compound, or salt of the compound or tautomer according to claim 129, wherein R⁵ is selected from the group consisting of hydrogen, alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl.

131. A compound, tautomer of the compound, or salt of the compound or tautomer according to claim 129, wherein R⁵ is selected from the group consisting of hydrogen, alkenyl, and alkylcarbonylalkyl, wherein:
any member of such group optionally is substituted with one or more substituents independently selected from the group consisting of halogen, hydroxy, alkoxy, and haloalkoxy.

132. A compound, tautomer of the compound, or salt of the compound or tautomer according to claim 131, wherein the compound corresponds in structure to a formula selected from the group consisting of:

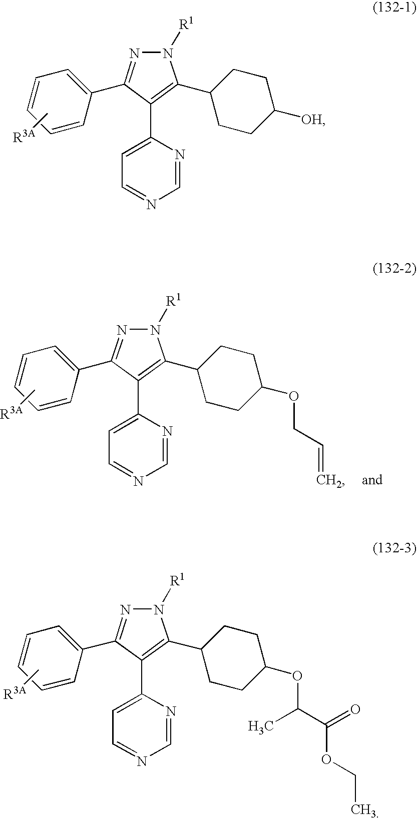

133. A compound, a tautomer of the compound, or a salt of the compound or tautomer, wherein:
the compound corresponds in structure to the following formula: 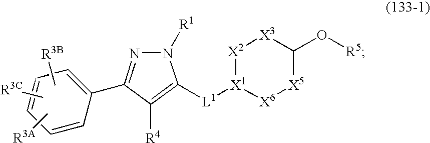
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁵ is selected from the group consisting of —CH₂— and —NH—, except that X⁵ is —CH₂— if X³ is —O— or X⁶ is —NH—; and
X⁶ is selected from the group consisting of-CH₂— and —NH—, except that X⁶ is —CH₂— if X² is —O— or X¹ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is pyridinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of alkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

134. A compound, tautomer of the compound, or salt of the compound or tautomer according to claim 133, wherein:
the compound corresponds in structure to the following formula: 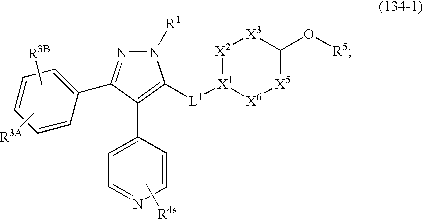
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

135. A compound, tautomer of the compound, or salt of the compound or tautomer according to claim 134, wherein the compound corresponds in structure to the following formula:

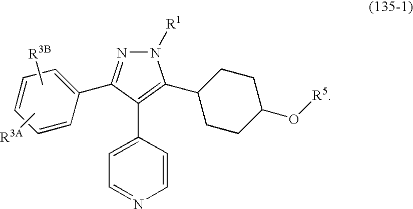

136. A compound, a tautomer of the compound, or a salt of the compound or tautomer, wherein:
the compound corresponds in structure to the following formula: 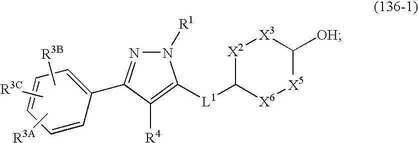
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X² is selected from the group consisting of —CH₂—, —NH—, and —O—, except that X² is —CH₂— if X³ is —O— or —NH—; and
X³ is selected from the group consisting of-CH₂—, —NH—, and —O—, except that X³ is —CH₂— if X² is —O— or —NH—; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is pyridinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
each R^{a} is independently selected from the group consisting of hydrogen and alkyl.

137. A compound, tautomer of the compound, or salt of the compound or tautomer according to claim 136, wherein:
the compound corresponds in structure to the following formula: 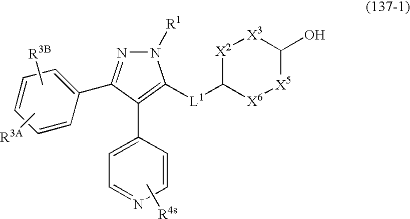
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

138. A compound, tautomer of the compound, or salt of the compound or tautomer according to claim 137, wherein the compound corresponds in structure to the following formula:

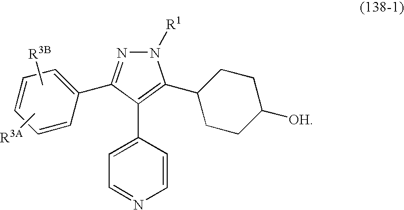

139. A compound, a tautomer of the compound, or a salt of the compound or tautomer, wherein:
the compound corresponds in structure to the following formula: 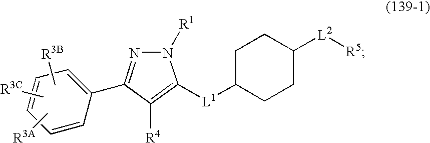
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is pyrimidinyl optionally substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of haloalkyl, hydroxyalkyl, alkenyl, alkynyl, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

140. A compound, tautomer of the compound, or salt of the compound or tautomer according to claim 139, wherein the compound corresponds in structure to the following formula:

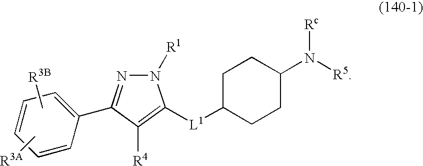

141. A compound, tautomer of the compound, or salt of the compound or tautomer according to claim 140, wherein:
the compound corresponds in structure to the following formula: 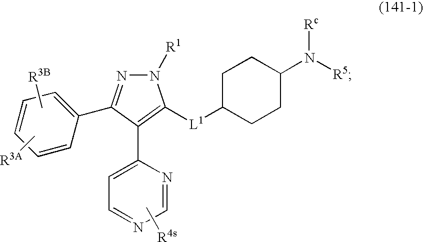
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

142. A compound, tautomer of the compound, or salt of the compound or tautomer according to claim 141, wherein the compound corresponds in structure to the following formula:

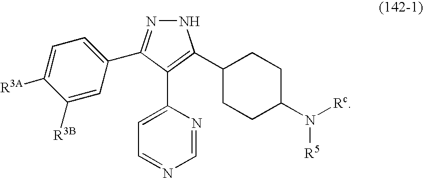

143. A compound, tautomer of the compound, or salt of the compound or tautomer according to claim 142, wherein R^{a} is alkyl.

144. A compound, tautomer of the compound, or salt of the compound or tautomer according to claim 143, wherein the compound corresponds in structure to the following formula:

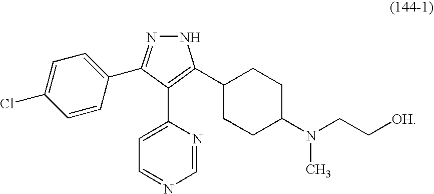

145. A compound, tautomer of the compound, or salt of the compound or tautomer according to claim 142, wherein R^{a} is hydrogen.

146. A compound, tautomer of the compound, or salt of the compound or tautomer according to claim 145, wherein the compound corresponds in structure to a formula selected from the group consisting of:

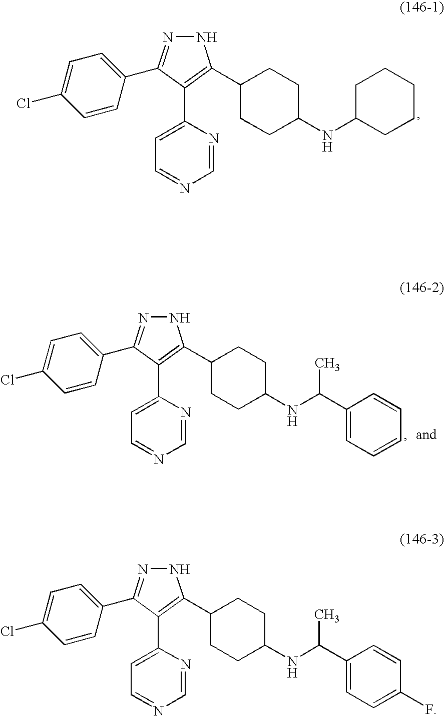

147. A compound, a tautomer of the compound, or a salt of the compound or tautomer, wherein:
the compound corresponds in structure to the following formula: 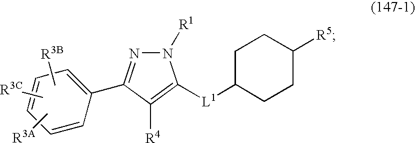
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is selected from the group consisting of pyrimidinyl and pyridinyl, wherein:
the pyrimidinyl or pyridinyl optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

148. A compound, tautomer of the compound, or salt of the compound or tautomer according to claim 147, wherein:
the compound corresponds in structure to the following formula: 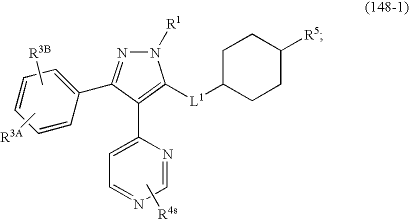
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

149. A compound, tautomer of the compound, or salt of the compound or tautomer according to claim 148, wherein R⁵ is hydroxyalkyl.

150. A compound, tautomer of the compound, or salt of the compound or tautomer according to claim 149, wherein the compound corresponds in structure to the following formula:

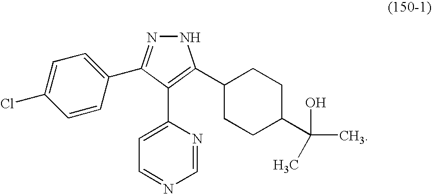

151. A compound, tautomer of the compound, or salt of the compound or tautomer according to claim 149, wherein R⁵ is heterocyclyl optionally substituted with one or more substituents independently selected from the group consisting of halogen, hydroxy, alkyl, haloalkyl, hydroxyalkyl, alkoxy, and haloalkoxy.

152. A compound, tautomer of the compound, or salt of the compound or tautomer according to claim 151, wherein:
the compound corresponds in structure to the following formula: 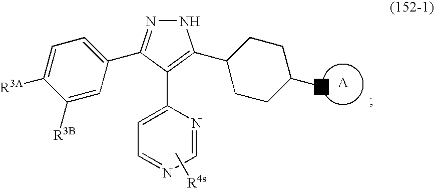
and
the ring structure A is a heterocyclyl ring that contains a nitrogen bonded to the cyclohexyl, and optionally is substituted with one or more substituents independently selected from the group consisting of halogen, hydroxy, alkyl, haloalkyl, hydroxyalkyl, alkoxy, and haloalkoxy.

153. A compound, tautomer of the compound, or salt of the compound or tautomer according to claim 151, wherein the compound corresponds in structure to the following formula:

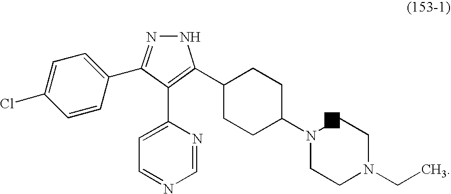

154. A compound, tautomer of the compound, or salt of the compound or tautomer according to claim 152, wherein:
R^{3A} is selected from the group consisting of halogen and haloalkyl; and
R^{3B} is selected from the group consisting of hydrogen, halogen, and haloalkyl.

155. A compound, tautomer of the compound, or salt of the compound or tautomer according to claim 154, wherein:
the compound corresponds in structure to the following formula: 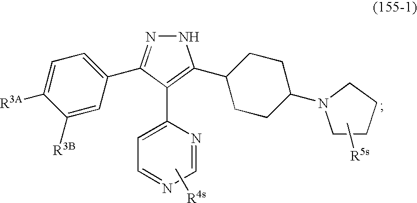
and
R^{5s} is selected from the group consisting of halogen, hydroxy, alkyl, haloalkyl, hydroxyalkyl, alkoxy, and haloalkoxy.

156. A compound, tautomer of the compound, or salt of the compound or tautomer according to claim 155, wherein the compound corresponds in structure to the following formula:

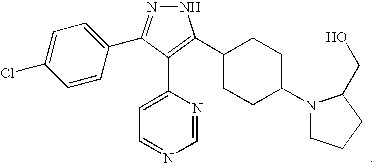

157. A compound, tautomer of the compound, or salt of the compound or tautomer according to claim 154, wherein:
the compound corresponds in structure to the following formula: 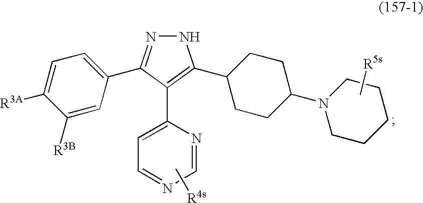
R^{5s} is selected from the group consisting of halogen, hydroxy, alkyl, haloalkyl, hydroxyalkyl, alkoxy, and haloalkoxy.

158. A compound, tautomer of the compound, or salt of the compound or tautomer according to claim 157, wherein the compound corresponds in structure to the following formula:

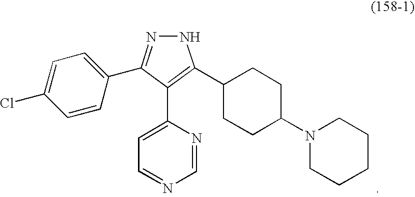

159. A compound, a tautomer of the compound, or a salt of the compound or tautomer, wherein:
the compound corresponds in structure to the following formula: 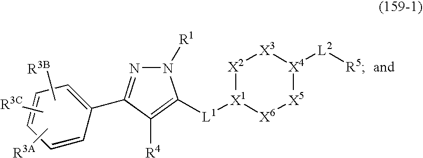
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —N(R^{a})—, —N(R^{a})—C(O)—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X¹ is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X¹ is —CH₂— if X³ is —O— or X⁶ is —NH—; and
X⁶ is selected from the group consisting of-CH₂— and —NH—, except that X⁶ is —CH₂— if X is —O— or X¹ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is selected from the group consisting of pyrimidinyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

160. A compound, tautomer of the compound, or salt of the compound or tautomer according to claim 159, wherein:
the compound corresponds in structure to the following formula: 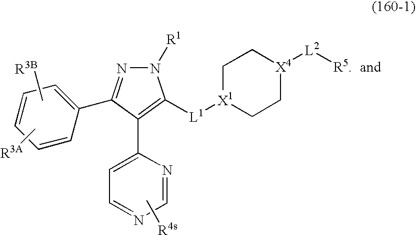
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

161. A compound, tautomer of the compound, or salt of the compound or tautomer according to claim 160, wherein:
the compound corresponds in structure to the following formula: 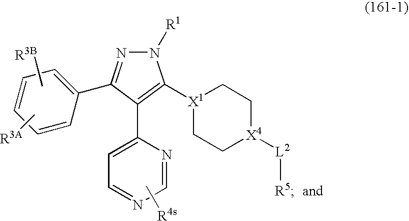
and
R¹ is selected from the group consisting of hydrogen and hydroxyalkyl.

162. A compound, tautomer of the compound, or salt of the compound or tautomer according to claim 161, wherein the compound corresponds in structure to the following formula:

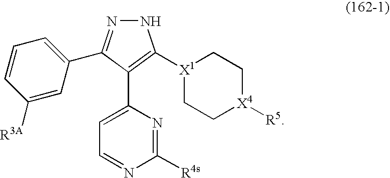

163. A compound, tautomer of the compound, or salt of the compound or tautomer according to claim 161, wherein the compound corresponds in structure to the following formula:

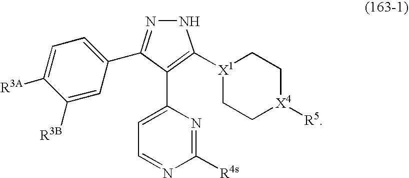

164. A compound, tautomer of the compound, or salt of the compound or tautomer according to claim 161, wherein the compound corresponds in structure to the following formula:

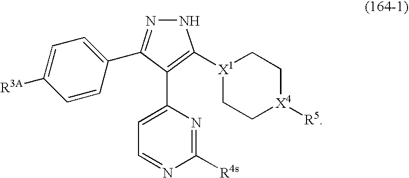

165. A compound, tautomer of the compound, or salt of the compound or tautomer according to claim 164, wherein the compound corresponds in structure to the following formula:

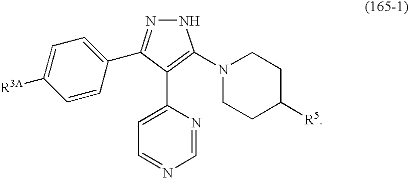

166. A compound, tautomer of the compound, or salt of the compound or tautomer according to claim 164, wherein the compound corresponds in structure to the following formula:

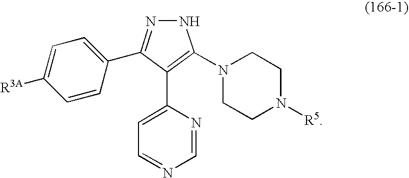

167. A compound, tautomer of the compound, or salt of the compound or tautomer according to claim 164, wherein the compound corresponds in structure to the following formula:

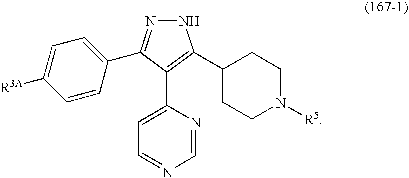

168. A compound, tautomer of the compound, or salt of the compound or tautomer according to claim 167, wherein the compound corresponds in structure to a formula selected from the group consisting of:

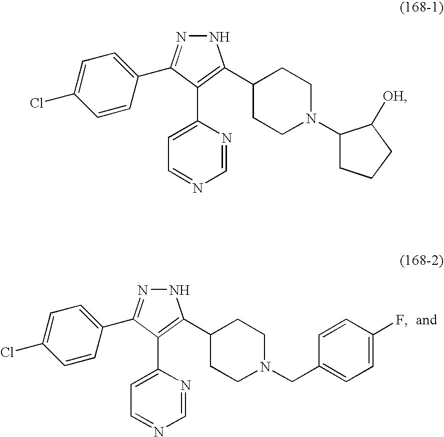

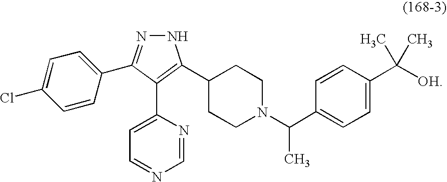

169. A compound, a tautomer of the compound, or a salt of the compound or tautomer, wherein:
the compound corresponds in structure to the following formula: 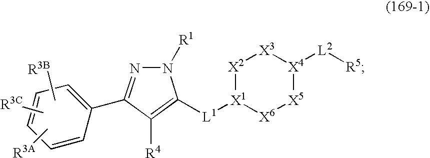
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, alkyl, alkoxy, and alkoxyalkyl, wherein:
any member of such group optionally is substituted with one or more substituents independently selected from the group consisting of halogen, hydroxy, and cyano; and
R^{3B} and R^{3C} are independently selected from the group consisting of hydrogen, halogen, hydroxy, cyano, alkyl, alkoxy, and alkoxyalkyl, wherein:
any member of such group optionally is substituted with one or more substituents independently selected from the group consisting of halogen, hydroxy, and cyano; and
R⁴ is selected from the group consisting of pyridinyl, pyrimidinyl, pyridazinyl, pyrazinyl, triazinyl, tetrazinyl, benzazinyl, benzodiazinyl, naphthyridinyl, pyridopyridinyl, pyrinyl, maleimidyl, pyridonyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is substituted methyl, wherein:
the methyl is substituted with:
two substituents independently selected from the group consisting of hydroxy, alkoxy, hydroxymethyl, hydroxyethyl, alkoxymethyl, alkoxyethyl, tetrahydrofuranyl, and tetrahydrofuranylmethyl, wherein any member of such group optionally is substituted with one or more substituents independently selected from the group consisting of halogen, hydroxy, alkyl, haloalkyl, hydroxyalkyl, alkoxy, and haloalkoxy, or
a substituent selected from the group consisting of alkoxyethoxy, hydroxyethoxy, alkoxypropoxy, and hydroxypropoxy, wherein the alkoxyethoxy, hydroxyethoxy, alkoxypropoxy, or hydroxypropoxy optionally is substituted with one or more substituents independently selected from the group consisting of halogen, hydroxy, alkyl, haloalkyl, hydroxyalkyl, alkoxy, and haloalkoxy, and
the methyl optionally is further substituted with hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

170. A compound, tautomer of the compound, or salt of the compound or tautomer according to claim 169, wherein the compound corresponds in structure to the following formula:

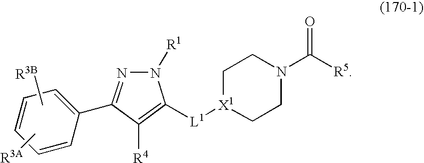

171. A compound, tautomer of the compound, or salt of the compound or tautomer according to claim 170, wherein:
the compound corresponds in structure to the following formula: 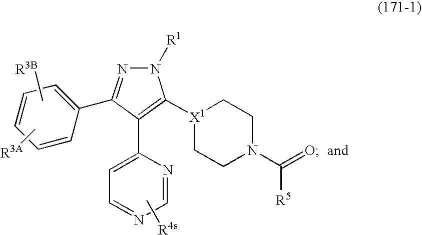
and
R^{4s} is selected from the group consisting of hydrogen, halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

172. A compound, tautomer of the compound, or salt of the compound or tautomer according to claim 171, wherein R⁵ is a radical selected from the group consisting of:

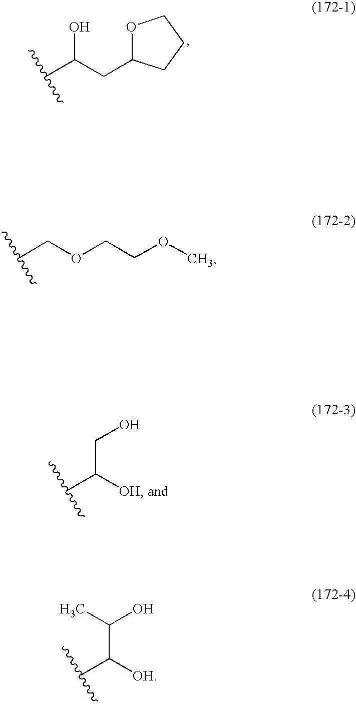

173. A compound, tautomer of the compound, or salt of the compound or tautomer according to claim 172, wherein the compound corresponds in structure to a formula selected from the group consisting of:

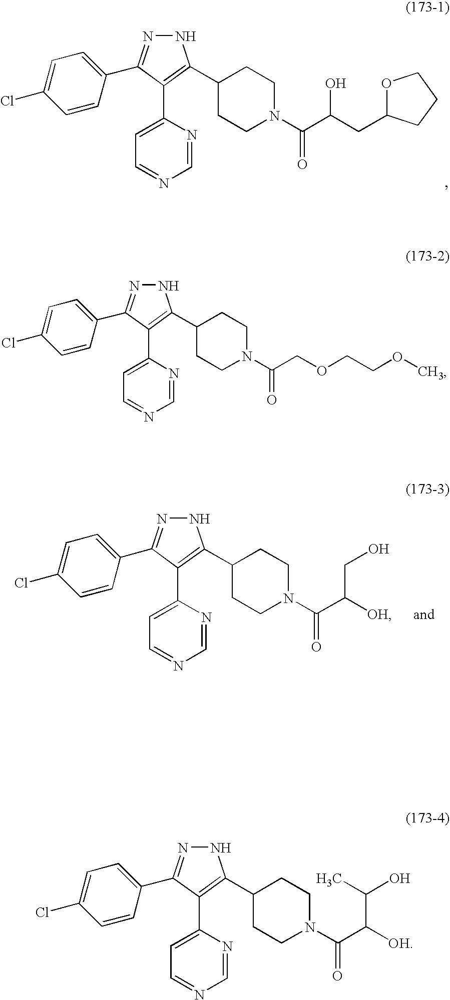

174. A compound, a tautomer of the compound, or a salt of the compound or tautomer, wherein:
the compound corresponds in structure to the following formula: 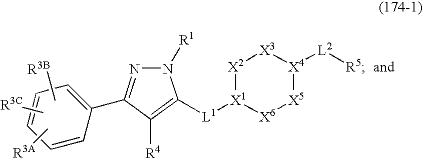
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N—N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of —CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of —CH₂— and —NH—, except that X⁶ is —CH₂— if X² is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R^{3B} and R¹ c are independently selected from the group consisting of hydrogen, halogen, hydroxy, cyano, amino, alkyl, aminoalkyl, monoalkylamino, dialkylamino, alkoxy, and alkoxyalkyl, wherein:
any carbon of any member of such group optionally is substituted with one or more substituents independently selected from the group consisting of halogen, hydroxy, and cyano; and
R⁴ is selected from the group consisting of pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group is substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy, and
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

175. A compound, tautomer of the compound, or salt of the compound or tautomer according to claim 174, wherein R⁴ is selected from the group consisting of pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group is substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy, and
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

176. A compound, tautomer of the compound, or salt of the compound or tautomer according to claim 175, wherein R⁴ is pyrimidinyl, wherein:
the pyrimidinyl is substituted with one or more substituents independently selected from the group consisting of heterocyclyloxy, heterocyclylalkoxy, cycloalkylamino, cyanoaryloxy, alkylaminoalkylamino, and carbocyclylalkylheterocyclylamino, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
the pyrimidinyl optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, amino, aminoalkyl, alkylamino, alkenylamino, alkynylamino, carbocyclylamino, aminocarbonyl, alkoxy, alkoxyalkyl, alkenyloxyalkyl, alkoxyalkylamino, alkylaminoalkoxy, alkoxycarbonyl, carbocyclyloxycarbonyl, heterocyclyloxycarbonyl, carbocyclylalkylamino, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylaminoalkylamino, alkylheterocyclylamino, heterocyclylalkylamino, alkylheterocyclylalkylamino, heterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

177. A compound, tautomer of the compound, or salt of the compound or tautomer according to claim 176, wherein R⁴ is pyrimidinyl substituted with a substituent independently selected from the group consisting of heterocyclyloxy, heterocyclylalkoxy, cycloalkylamino, cyanoaryloxy, alkylaminoalkylamino, and carbocyclylalkylheterocyclylamino, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy.

178. A compound, tautomer of the compound, or salt of the compound or tautomer according to claim 177, wherein R⁴ is pyrimidinyl substituted with a substituent independently selected from the group consisting of heterocyclyloxy, heterocyclylalkoxy, cycloalkylamino, cyanoaryloxy, dialkylaminoalkylamino, and carbocyclylalkylheterocyclylamino.

179. A compound, tautomer of the compound, or salt of the compound or tautomer according to claim 178, wherein:
the compound corresponds in structure to the following formula: 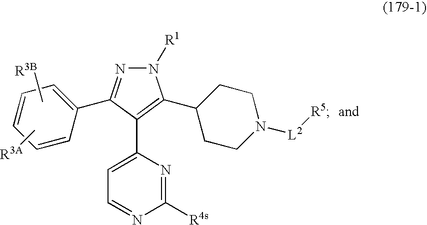
and
R^{4s} is selected from the group consisting of heterocyclyloxy, heterocyclylalkoxy, cycloalkylamino, cyanoaryloxy, dialkylaminoalkylamino, and carbocyclylalkylheterocyclylamino.

180. A compound, tautomer of the compound, or salt of the compound or tautomer according to claim 179, wherein the compound corresponds in structure to the following formula:

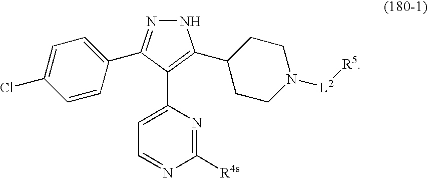

181. A compound, tautomer of the compound, or salt of the compound or tautomer according to claim 179, wherein R^{3A} is halogen.

182. A compound, tautomer of the compound, or salt of the compound or tautomer according to claim 179, wherein -L²-R⁵ is selected from the group consisting of hydrogen, methyl, and butyloxycarbonyl.

183. A compound, tautomer of the compound, or salt of the compound or tautomer according to claim 179, wherein -L²-R⁵ is hydroxymethylcarbonyl.

184. A compound, tautomer of the compound, or salt of the compound or tautomer according to claim 179, wherein R^{4s} is selected from the group consisting of tetrahydrofuranyloxy, cyanophenyloxy, morpholinylethyloxy, cyclopentylamino, dimethylaminoethylamino, and phenylmethylpiperidinylamino.

185. A compound, tautomer of the compound, or salt of the compound or tautomer according to claim 184, wherein the compound corresponds in structure to a formula selected from the group consisting of:

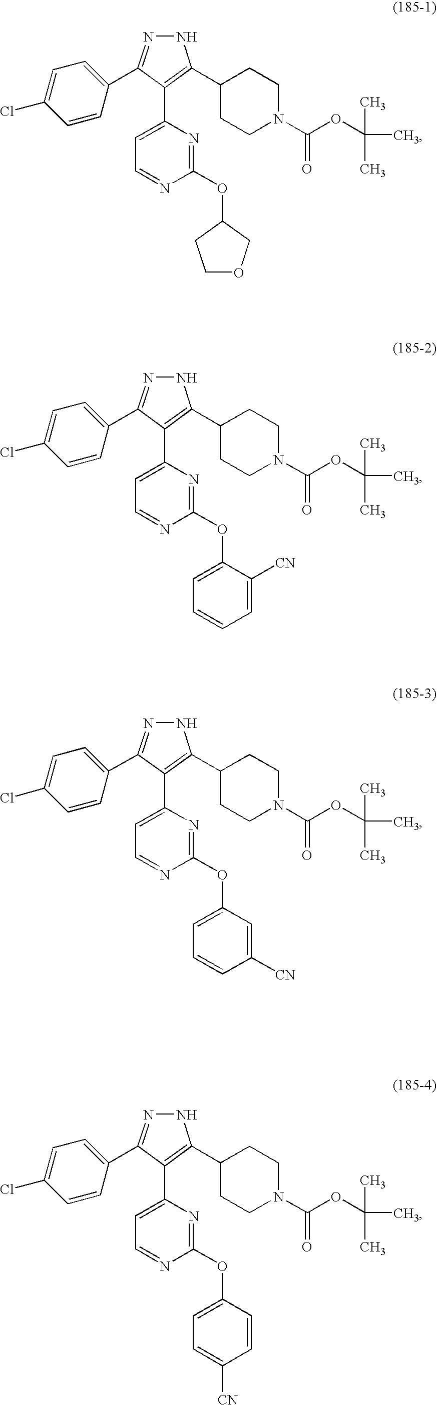

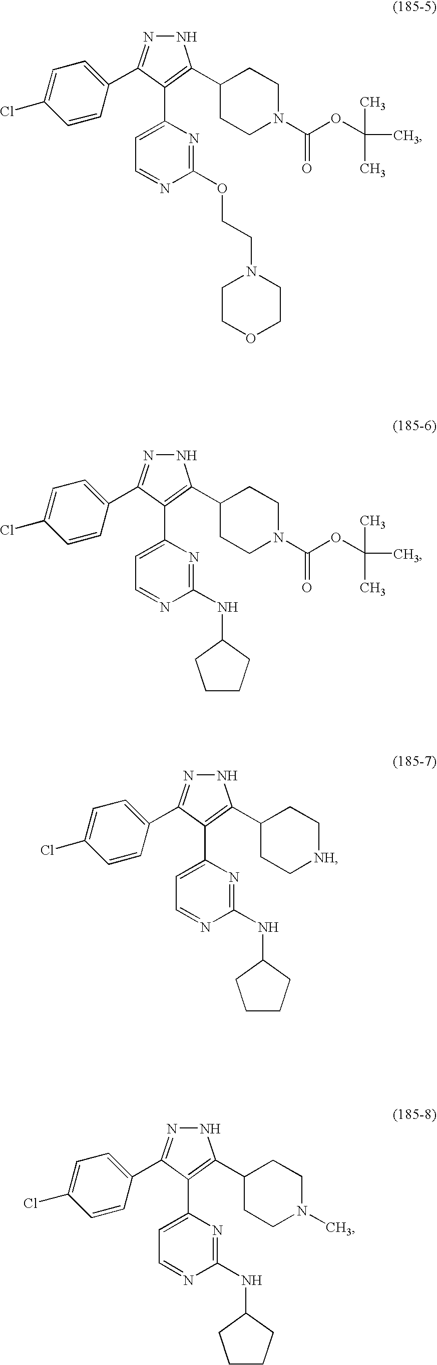

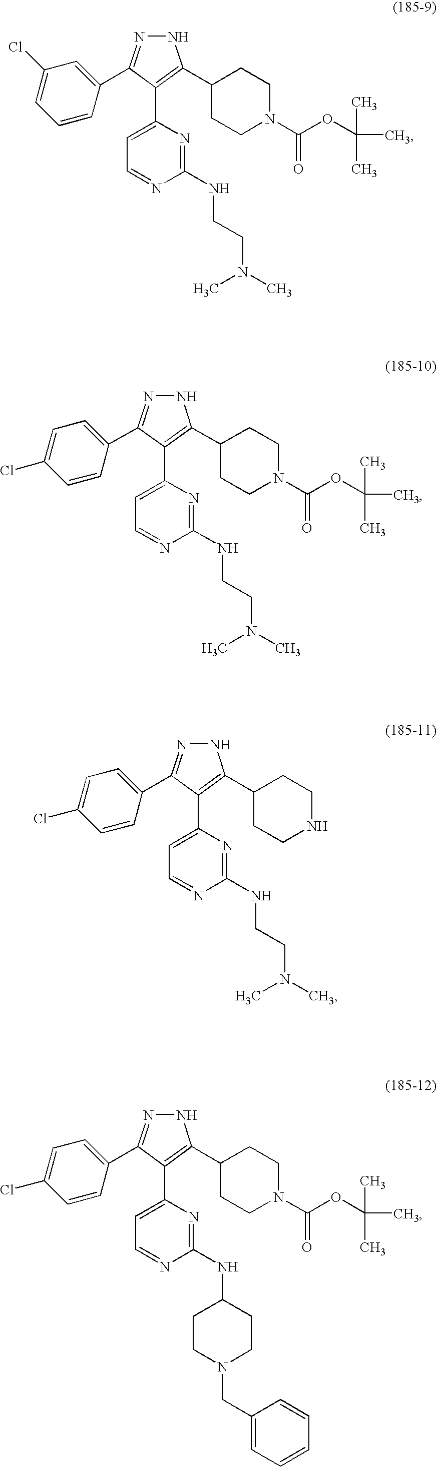

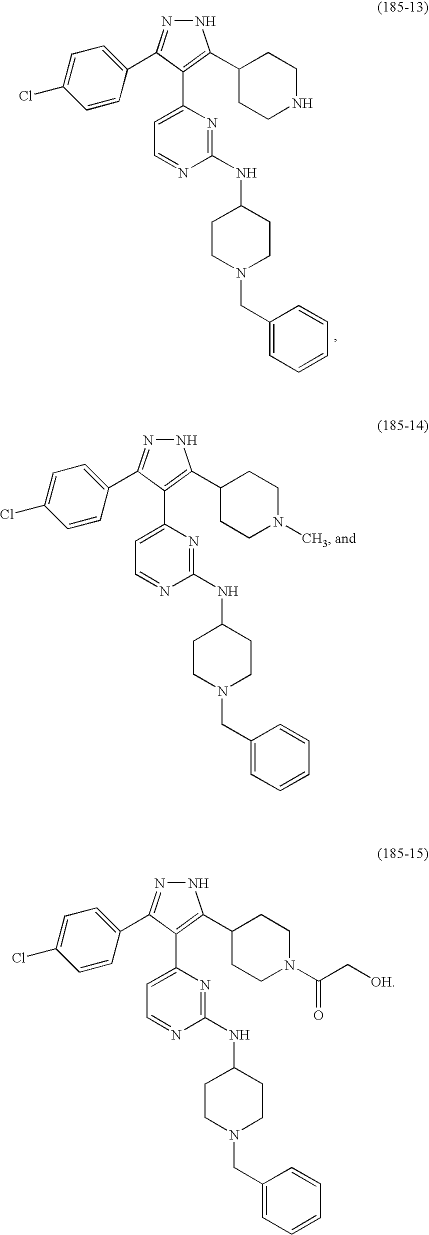

186. A compound, a tautomer of the compound, or a salt of the compound or tautomer, wherein:
the compound corresponds in structure to the following formula: 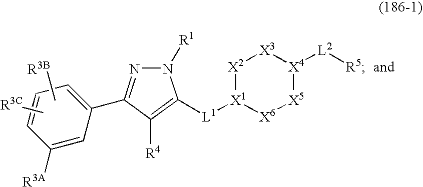
and
L¹ is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—, —C(O)—, —C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
L² is selected from the group consisting of a bond, —O—, —S—, —S(O)—, —S(O)₂—, —N(R^{a})—, —C(O)—, —C(O)—N(R^{a})—, —N(R^{a})—C(O)—, —C(O)—O—, —O—C(O)—, —O—C(O)—O—, —C(H)═C(H)—, —C≡C—, —N═N—, —N(R^{a})—N(R^{a})—, —N(R^{a})—C(O)—N(R^{a})—, —C(S)—N(R^{a})—, —N(R^{a})—C(S)—, —CH₂—, —O—CH₂—, —CH₂—O—, —S—CH₂—, and —CH₂—S—; and
X¹ is selected from the group consisting of nitrogen and carbon bonded to hydrogen, except that X¹ is carbon bonded to hydrogen if any of X², X³, X⁵, or X⁶ is —NH— or —O—; and
X² is selected from the group consisting of-CH₂—, —NH—, and —O—, except that X² is —CH₂— if X³ is —O— or —NH—; and
X³ is selected from the group consisting of —CH₂—, —NH—, and —O—, except that X³ is —CH₂— if X² is —O— or —NH—; and
X⁴ is selected from the group consisting of nitrogen and carbon bonded to hydrogen; and
X⁵ is selected from the group consisting of —CH₂— and —NH—, except that X⁵ is —CH₂— if X³ is —O— or X⁶ is —NH—; and
X⁶ is selected from the group consisting of-CH₂— and —NH—, except that is —CH₂— if X⁶ is —O— or X⁵ is —NH—; and
R¹ is selected from the group consisting of hydrogen, hydroxyalkyl, carboxyalkyl, aminoalkyl, aminocarbonylalkyl, and aminocarbonylaminoalkyl, wherein:
any amino nitrogen of any member of such group optionally is substituted with up to two independently selected alkyl; and
R^{3A} is selected from the group consisting of halogen, hydroxy, cyano, amino, alkyl, aminoalkyl, monoalkylamino, dialkylamino, alkoxy, and alkoxyalkyl, wherein:
of any member of such group of the alkyl, aminoalkyl, monoalkylamino, dialkylamino, alkoxy, or alkoxyalkyl optionally is substituted with one or more substituents independently selected from the group consisting of halogen, hydroxy, and cyano; and
R^{3B} and R^{3C} are independently selected from the group consisting of hydrogen, halogen, hydroxy, cyano, amino, alkyl, aminoalkyl, monoalkylamino, dialkylamino, alkoxy, and alkoxyalkyl, wherein:
of any member of such group of the alkyl, aminoalkyl, monoalkylamino, dialkylamino, alkoxy, or alkoxyalkyl optionally is substituted with one or more substituents independently selected from the group consisting of halogen, hydroxy, and cyano; and
R⁴ is selected from the group consisting of pyridinyl, pyrimidinyl, maleimidyl, pyridonyl, pyridazinyl, pyrazinyl, triazinyl, tetrazinyl, benzazinyl, benzodiazinyl, naphthyridinyl, pyridopyridinyl, pyrinyl, thiazolyl, isothiazolyl, thiazolylalkyl, isothiazolylalkyl, thiazolylamino, isothiazolylamino, thiomorpholinyl, the sulfoxide of thiomorpholinyl, and the sulfone of thiomorpholinyl, wherein:
any member of such group is substituted with one or more independently selected alkylaminoalkoxy optionally substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy, and
any member of such group optionally is substituted with one or more substituents independently selected from the group consisting of halogen, cyano, hydroxy, thiol, carboxy, nitro, alkyl, carboxyalkyl, alkylthio, alkylsulfinyl, alkylsulfonyl, alkylcarbonyl, carbocyclyl, carbocyclylalkyl, carbocyclylalkenyl, carbocyclyloxy, carbocyclylalkoxy, carbocyclyloxyalkyl, carbocyclylthio, carbocyclylsulfinyl, carbocyclylsulfonyl, heterocyclylthio, heterocyclylsulfinyl, heterocyclylsulfonyl, carbocyclylalkoxy, carbocyclylheterocyclyl, heterocyclylalkyl, heterocyclyloxy, heterocyclylalkoxy, amino, aminoalkyl, alkylamino, alkenylamino, alkynylamino, carbocyclylamino, heterocyclylamino, aminocarbonyl, alkoxy, alkoxyalkyl, alkenyloxyalkyl, alkoxyalkylamino, alkoxycarbonyl, carbocyclyloxycarbonyl, heterocyclyloxycarbonyl, alkoxycarbonylamino, alkoxycarbocyclylamino, alkoxycarbocyclylalkylamino, aminosulfinyl, aminosulfonyl, alkylsulfonylamino, alkoxyalkoxy, aminoalkoxy, aminoalkylamino, alkylaminoalkylamino, carbocyclylalkylamino, alkylaminoalkylaminoalkylamino, alkylheterocyclylamino, heterocyclylalkylamino, alkylheterocyclylalkylamino, carbocyclylalkylheterocyclylamino, heterocyclylheterocyclylalkylamino, alkoxycarbonylheterocyclylamino, alkylaminocarbonyl, alkylcarbonylamino, hydrazinyl, alkylhydrazinyl, and carbocyclylhydrazinyl, wherein:
any member of such group optionally is substituted with one or more substituents independently selected from the group consisting of alkyl, alkenyl, hydroxy, halogen, haloalkyl, alkoxy, haloalkoxy, keto, amino, nitro, cyano, alkylsulfonyl, alkylsulfinyl, alkylthio, alkoxyalkyl, carbocyclyloxy, heterocyclyl, and heterocyclylalkoxy; and
R⁵ is selected from the group consisting of hydrogen, hydroxy, alkyl, alkenyl, alkynyl, alkoxy, alkoxyalkyl, alkoxyalkoxyalkyl, alkylcarbonylalkyl, alkoxycarbonylalkyl, carbocyclyl, carbocyclylalkyl, heterocyclyl, and heterocyclylalkyl, wherein:
any member of such group optionally is substituted with one or more substituents independently selected from the group consisting of halogen, hydroxy, alkyl, haloalkyl, hydroxyalkyl, alkoxy, and haloalkoxy; and
each R^{a} is independently selected from the group consisting of hydrogen and alkyl.

187. A compound, tautomer of the compound, or salt of the compound or tautomer according to claim 186, wherein R^{3A} is halogen.

188. A compound, tautomer of the compound, or salt of the compound or tautomer according to claim 186, wherein -L²-R⁵ is selected from the group consisting of hydrogen, methyl, and butyloxycarbonyl.

189. A compound, tautomer of the compound, or salt of the compound or tautomer according to claim 186, wherein -L²-R⁵ is hydroxymethylcarbonyl.

190. A compound, tautomer of the compound, or salt of the compound or tautomer according to claim 186, wherein:
the compound corresponds in structure to the following formula: 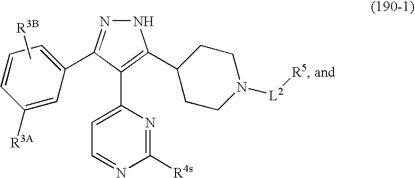
and
R^{4s} is dialkylaminoalkoxy.

191. A compound, tautomer of the compound, or salt of the compound or tautomer according to claim 190, wherein R^{4s} is dimethylaminoethyloxy.

192. A compound, tautomer of the compound, or salt of the compound or tautomer according to claim 191, wherein the compound corresponds in structure to a formula selected from the group consisting of:

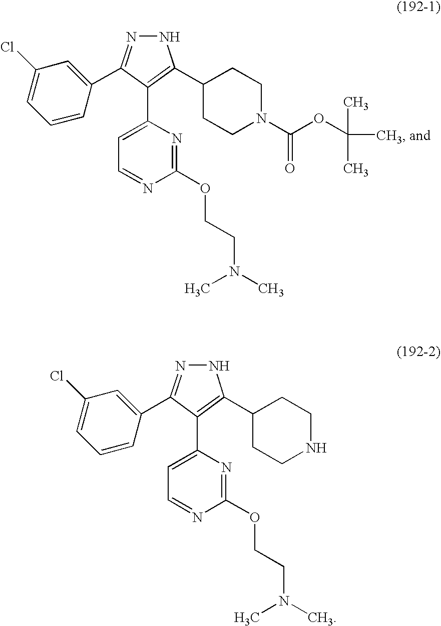

193. A pharmaceutical composition, wherein:
the pharmaceutical composition comprises a therapeutically-effective amount of a compound, a tautomer of the compound, or a pharmaceutically acceptable salt of the compound or tautomer; and
the compound is selected from the group of compounds recited in any one of claims 1, 51, 70, 76, 107, 110, 113, 115, 120, 125, 133, 136, 139, 147, 159, 169, 174, and 186.

194. A method for treating a p38 mediated, tumor necrosis factor mediated, or cyclooxygenase-2 mediated condition in a mammal, wherein:
the method comprises administering a therapeutically-effective amount of a compound, a tautomer of the compound, or a pharmaceutically acceptable salt of the compound or tautomer to the mammal; and
the compound is selected from the group of compounds recited in any one of claims 1, 51, 70, 76, 107, 110, 113, 115, 120, 125, 133, 136, 139, 147, 159, 169, 174, and 186.
